(19) United States
(12) Patent Application Publication
(10) Pub. No.: US 2024/0197906 A1
(43) Pub. Date: Jun. 20, 2024

(54) ANTI-cMET ANTIBODY DRUG CONJUGATES AND THERAPEUTIC USES THEREOF

(71) Applicant: Mythic Therapeutics, Inc., Waltham, MA (US)

(72) Inventors: Brian Fiske, Waltham, MA (US); Nimish Gera, Waltham, MA (US); William Comb, Melrose, MA (US); Amit Deshpande, Medford, MA (US)

(21) Appl. No.: 18/535,713

(22) Filed: Dec. 11, 2023

Related U.S. Application Data
(60) Provisional application No. 63/585,596, filed on Sep. 26, 2023.
(60) Provisional application No. 63/386,914, filed on Dec. 11, 2022.

(51) Int. Cl. A61K 47/68; A61K 45/06; A61P 35/00

(52) U.S. Cl. CPC A61K 47/6851; A61K 45/06; A61K 47/68031; A61K 47/6889; A61P 35/00

(57) ABSTRACT

This application relates to, inter alia, anti-cMET antibody drug conjugates (“ADCs”) having lower target-specific binding at low versus physiologic pH (“pH-ADCs”), pharmaceutical compositions including the pH-ADCs, methods of making the pH-ADCs, methods of selecting specific patient populations for cancer treatment with an anti-cMET PH-ADC, and methods of using the pH-ADCs to treat cancers, including cMET+/cMET-expressing or cMET-overexpressing cancers, particularly including cMET+/cMET-expressing or cMET-overexpressing non-small cell lung cancers (“NSCLCs”).

### CROSS-REFERENCE TO RELATED APPLICATIONS

This application claims the benefit of U.S. Provisional Application No. 63/386,914, filed Dec. 11, 2022, and U.S. Provisional Application No. 63/585,596, filed on Sep. 26, 2023, each of which is incorporated herein by reference in its entirety.

### SEQUENCE LISTING

This application contains a Sequence Listing that has been submitted electronically as an XML file named “45395-0062001_SL_ST26.” The XML file, created on Dec. 4, 2023, is 388,717 bytes in size. The material in the XML file is hereby incorporated by reference in its entirety.

### FIELD

This application relates to, inter alia, anti-cMET antibody drug conjugates (“ADCs”) having lower target-specific binding at low versus physiologic pH (“pH-dependent ADCs”), pharmaceutical compositions including the ADCs, methods of making the ADCs, methods of selecting specific patient populations for cancer treatment with an anti-cMET ADC, and methods of using the ADCs to treat cancers.

### SUMMARY

cMET (cellular mesenchymal-epithelial transition factor) is a receptor tyrosine kinase and proto-oncogene whose activity is upregulated across a wide range of cancers. cMET is also expressed in epithelial cells of multiple organs, including the liver, pancreas, prostate, kidney, muscle, and bone marrow, during both embryogenesis and adulthood. cMET regulates many physiological processes including cell proliferation and survival, migration and scattering, organ regeneration, tissue morphogenesis, and tissue remodeling.

In NSCLC, expression of cMET is upregulated by DNA amplification (2 to 5% of patient tumors) (Dong et al, MET-Targeted Therapies and Clinical Outcomes: A Systematic Literature Review. Molecular Diagnosis & Therapy (2022) 26:203-227), exon 14 skipping mutations (2 to 4%) (Fujino et al, Lung Cancer with MET exon 14 Skipping Mutation: Genetic Feature, Current Treatments, and Future Challenges. Lung Cancer (Auckl). 2021; 12: 35-50), and overexpression (30 to 60%) (Salgia, MET in Lung Cancer: Biomarker Selection Based on Scientific Rationale. Mol Cancer Ther. 2017; 16(4):555-565). cMET is also deregulated in many other cancers including gastric, head and neck, pancreatic, and colon cancer (Sierra 2011; Spigel D R, et al. Randomized Phase II trial of onartuzumab in combination with erlotinib in patients with advanced non-small-cell lung cancer. J Clin Oncol. 2013; 31(32):41054114; Resnick M B, et al. Epidermal growth factor receptor, cMET, B-catenin, and p53 expression as prognostic indicators in stage II colon cancer: a tissue microarray study. Clin Can Res. 2004; 10:3069-3075; Lee H E, et al. MET in gastric carcinomas: comparison between protein express and gene copy number and impact on outcome. Br J Can. 2012; 107(2):325-333). In NSCLC, cMET upregulation has been studied as a resistance mechanism for several approved EGFR-targeted kinase inhibitors, and this resistance may limit their effectiveness (Fernandes 2021). A non-exhaustive list of FDA-approved EGFR inhibitors include the following: afatinib (GILOTRIF), erlotinib (e.g., TARCEVA), osimertinib (e.g., TAGRISSO), neratinib (e.g., NERLYNX), cetuximab (e.g., ERBITUX), gefitinib (e.g., IRESSA), panitumumab (e.g., VECTIBIX), necitumumab (e.g., PORTRAZZA), vandetanib (e.g., CAPRELSA), mobocertinib (EXKIVITY), lapatinib (e.g., TYKERB), and dacomitinib (e.g., VIZIMPRO).

Several antibodies targeting cMET, with various mechanisms of action, have been evaluated in clinical trials but given limited activity, have failed to obtain FDA approval (Wolf J, et al. Capmatinib in MET Exon 14-Mutated or MET-Amplified Non-Small-Cell Lung Cancer. New England Journal of Medicine. Massachusetts Medical Society; 2020; 383:944-57; Scagliotti G, et al. A Randomized-Controlled Phase 2 Study of the MET Antibody Emibetuzumab in Combination with Erlotinib as First-Line Treatment for EGFR Mutation-Positive NSCLC Patients. Journal of Thoracic Oncology. Elsevier Inc; 2020; 15:80-90; Strickler J H, LoRusso P, Salgia R, Kang Y K, Yen C J, Lin C C, et al. Phase 1 dose-escalation and -expansion study of telisotuzumab (ABT-700), an Anti-c-met antibody, in patients with advanced solid tumors. Mol Cancer Ther. American Association for Cancer Research Inc.; 2020; 19:1210-7).

Recently, novel modalities such as bispecific antibodies and antibody drug conjugates (ADCs) have been generated against cMET. Amivantamab, an EGFR×cMET bispecific antibody, was approved in 2021 for patients with locally advanced or metastatic NSCLC with EGFR exon 20 insertion mutations (Chon K, et al. FDA Approval Summary: Amivantamab for the Treatment of Patients with Non-Small Cell Lung Cancer with EGFR Exon 20 Insertion Mutations. Clinical Cancer Research. AACR; 2023). Telisotuzumab-vedotin (Teliso-V), an MMAE conjugated ADC, is currently in late-stage development as monotherapy or as combination therapy, and recently received breakthrough designation for patients with locally advanced or metastatic non-squamous NSCLC with EGFR wild type (WT) status and high cMET expression (Coleman N, et al. Antibody-drug conjugates in lung cancer: dawn of a new era? NPJ Precis Oncol. Nature Research; 2023.). Although Teliso-V exhibited activity in cMET high expressing tumors, there remains a much broader population of NSCLC patients whose tumors express intermediate and low levels of expression, for which there are no promising therapies imminent. Accordingly, cMET remains a compelling target for second generation ADCs, of which many are in early clinical development (RC108, TR1801, BYON3521, ABBV-400, AZD9592 and REGN5093-M114).

And since tumors comprising even relatively low levels of cMET expression have been associated with poor patient outcome, there remains a need for cancer therapeutics that target solid tumors having levels of cMET expression and/or overexpression that are not addressable with current modalities. As used herein, a “cMET-positive” or “cMET-expressing” tumor is one that comprises at least some cells that can be detected using an IHC assay and would be assigned an IHC score of at least +1. And while there is not yet a universally accepted definition for the term “cMET overexpression”, as used herein, “cMET overexpression” is intended to mean a level of cMET expression in tumor tissue that is significantly higher than the level of cMET expression in non-cancerous tissue comprised of substantially the same type(s) and proportion(s) of cells that gave rise to the tumor tissue. cMET-positivity/expression and cMET overexpression can be measured by a variety of methods known in the art (e.g., IHC scoring of tumor samples, whole body imaging using suitably-labeled target-specific binding agents, etc.). In another embodiment, the anti-cMET ADCs are useful therapeutically for the treatment of cMET-expressing tumors in humans where the cMET is expressed at a level that is beneath the threshold of detection of current IHC assays, but still expressed at a level sufficient to permit the anti-cMET ADCs to exert a significant antitumor effect.

We hypothesized that engineering antibodies specifically for ADCs had the potential to augment therapeutic index by increasing tumor delivery and increasing ADC exposure, to benefit patients whose tumors express lower levels of antigen. Engineering pH-dependent binding in the antibody component of an anti-cMET ADC was posited to permit the release of the ADC from cMET in the acidic endolysosomal environment, enable receptor recycling and boost uptake and efficacy in cMET+/cMET-expressing or cMET-overexpressing cancer cells. As disclosed herein, we designed, produced, and demonstrated the superior performance of such pH-engineered anti-cMET antibodies and ADCs made therefrom.

Accordingly, the compositions and therapeutic methods disclosed herein target solid tumors expressing relatively high, moderate, and even low levels of cMET by using anti-cMET antibody drug conjugates (ADCs) that bind better (e.g., have a higher affinity as indicated by a lower Kd) to cMET at physiologic pH versus acidic pH (“pH-dependent, anti-cMET ADCs” or “pH-ADCs”). In some embodiments, the pH-ADCs bind well at physiologic pH and mildly acidic pH (e.g., about pH 6.3), but less well at endolysosomal/lysosomal pH (e.g., about pH 5.3). As a consequence of this pH-dependent, target-specific binding, the disclosed pH-ADCs exhibit superior internalization, and deliver greater amounts of toxic payload to cMET+/cMET-expressing and cMET-overexpressing tumor cells relative to corresponding non-pH-ADCs. Furthermore, because the pH-ADCs dissociate from cMET in the acidic environment of the endolysosomes and/or lysosomes, cMET is free to be recycled back to the surface of the tumor cell (i.e., rather than being degraded along with a specifically bound ADC), where it has the opportunity to engage once more with an extracellular pH-ADC. At the same time, in healthy cells expressing cMET, the FCRN pathway may permit the export of the internalized pH-ADC, contributing to a superior safety profile for the disclosed pH-ADCs. Taken together, the disclosed anti-cMET pH-ADCs may be less toxic and more effective (at lower doses and/or reduced dosing frequency) when compared with anti-cMET non-pH-ADCs.

Data presented herein demonstrate, for the first time, that pH-ADCs that specifically target cMET exhibit robust anti-tumor activity against tumors from patients diagnosed with NSCLC. For example, data demonstrating in vivo anti-tumor efficacy of anti-cMET pH-ADCs administered as monotherapy are provided in Examples 4, 5, and 6. Further, an ongoing human clinical trial is expected to confirm the improved safety, efficacy, and PK/PD parameters exhibited by the anti-cMET pH-ADCs in a host of preclinical studies. Early trial findings appear to be consistent with the preclinical safety and PK data disclosed herein.

As disclosed herein, cMET-positivity can be defined by a tumor immunohistochemistry (IHC) H-score of 1 to 149 and cMET-overexpression can be defined by a tumor IHC H-score of 150 to 300 when measured by a suitable assay (e.g., as described in Example 11). Briefly, an IHC staining procedure for cMET has been developed using the Ventana cMET CONFIRM (SP44) kit. Tissue samples are fixed (e.g., in formalin), embedded in was, sectioned, stained with the Ventana antibody, and then scored by determining the percentages of target tissue cells staining at various intensity levels of low to high (FIGS. 12-15). Additionally, tumor tissues including cMET+/cMET-expressing cancer cells (i.e., at least some cancer cells having an IHC score of +1 and/or a tumor tissue H-score of 50 to 149) or cMET-overexpressing cancer cells (i.e., at least some cancer cells having an IHC of +2/+3 and/or a tumor H-score of 150 to 300) may be assigned an IHC score as described in Example 11. As used herein, an H-score between 50 and 149 is equivalent to an IHC score of 1+, an H-score of 150 to 224 is equivalent to an IHC score of 2+, and an H-score of 225 to 300 is equivalent to an IHC score of 3+.

The anti-cMET pH-ADCs may be administered as single therapeutic agents or adjunctively with or to other treatments and/or therapeutics. Indeed, data presented herein demonstrate that xenograft tumors produced from cells from tumors that had exhibited resistance to other therapies were sensitive to anti-cMET pH-ADCs (see e.g., Example 5). Furthermore, the anti-cMET pH-ADCs of the present disclosure deliver more toxic payload to tumor cells while impacting healthy cells to a lesser extent relative to corresponding control non-pH ADCs. Accordingly, the anti-cMET pH-ADCs described herein provide significant benefits over current targeted and non-targeted approaches toward the treatment of cMET+ and/or cMET-overexpressing solid tumors.

Adjunctive therapies and/or therapeutics typically will be used at their regulatory agency-approved dose, route of administration, and frequency of administration, but may be used at lower dosages and/or less frequently. When administered as monotherapy, the anti-cMET pH-ADC will typically be administered on a schedule that provides therapeutic benefit. It is envisioned that anti-cMET pH-ADCs administered once every three, four, five, six, seven, or eight weeks will provide maximal therapeutic benefit, but more or less frequent administration may also be useful. When administered with other treatment modalities, the anti-cMET pH-ADC may be administered before, after, or simultaneously therewith.

Administration routes for the anti-cMET pH-ADCs may include, but not be limited to, intravenous infusion and/or injection, subcutaneous injection (e.g., as described in U.S. Pat. No. 10,799,597, to Immunomedics), and/or transdermal or other transcutaneous therapeutic delivery methods. The amount administered will depend upon the route of administration, the dosing schedule, the type of cancer being treated, the stage of the cancer being treated, and other parameters such as the age and weight of the patient, as is well known in the art. Specific exemplary dosing schedules expected to provide therapeutic benefit are provided in the Detailed Description. Generally, an amount of anti-cMET pH-ADC in the range of about 0.005 to about 20 mg/kg when administered intravenously from about once every two weeks to about once every eight weeks is expected to provide therapeutic benefit. In some embodiments, the amount is about 0.5 to about 15 mg/kg. In some embodiments, dosing every three weeks is sufficient to produce significant treatment benefits. In other embodiments, dosing every four weeks is sufficient to produce the treatment benefits.

In an aspect, the disclosure provides a method of treating a cMET+, cMET-overexpressing, and/or MET-amplified solid tumor cancer, comprising administering an effective amount of an anti-cMET antibody drug conjugate (ADC) to a human subject having said cancer, over a sufficient period of time to provide a therapeutic benefit, wherein the antibody component of the ADC exhibits cMET-specific pH-dependent binding. In one embodiment, the antibody comprises heavy and light chain CDRs present in the amino acid sequences as set forth in one of the following pairs: SEQ ID NOs: 15 & 16; SEQ ID NOs: 5 & 6; SEQ ID NOs: 7 & 8; SEQ ID NOs: 9 & 10; SEQ ID NOs: 11 & 12; SEQ ID NOs: 13 & 14; SEQ ID NOs: 15 & 16; SEQ ID NOs: 17 & 18; SEQ ID NOs: 19 & 20; SEQ ID NOs: 21 & 22; SEQ ID NOs: 23 & 24; SEQ ID NOs: 25 & 26; SEQ ID NOs: 27 & 28; SEQ ID NOs: 29 & 30; SEQ ID NOs: 31 & 32; and SEQ ID NOs: 33 & 34. In an embodiment, the CDRs are determined using the Kabat or IMGT system.

In an aspect, the disclosure provides a method of treating a cMET-positive solid tumor cancer having low or intermediate expression of cMET, comprising administering an effective amount of an anti-cMET antibody drug conjugate (ADC) to a human subject previously identified or selected as having said cancer, over a sufficient period of time to provide a therapeutic benefit, wherein the antibody component of the ADC exhibits cMET-specific pH-dependent binding. In one embodiment, the antibody comprises heavy and light chain CDRs present in the amino acid sequences as set forth in one of the following pairs: SEQ ID NOs: 15 & 16; SEQ ID NOs: 5 & 6; SEQ ID NOs: 7 & 8; SEQ ID NOs: 9 & 10; SEQ ID NOs: 11 & 12; SEQ ID NOs: 13 & 14; SEQ ID NOs: 17 & 18; SEQ ID NOs: 19 & 20; SEQ ID NOs: 21 & 22; SEQ ID NOs: 23 & 24; SEQ ID NOs: 25 & 26; SEQ ID NOs: 27 & 28; SEQ ID NOs: 29 & 30; SEQ ID NOs: 31 & 32; and SEQ ID NOs: 33 & 34. In an embodiment, the CDRs are determined using the Kabat or IMGT system.

In an aspect, the disclosure provides a method of treating a cMET-positive and/or cMET-overexpressing solid tumor cancer having a cMET immunohistochemistry (IHC) score of 1+, 2+, or 3+, comprising administering an effective amount of an anti-cMET antibody drug conjugate (ADC) to a human subject previously identified or selected as having said cancer, over a sufficient period of time to provide a therapeutic benefit, wherein the antibody component of the ADC exhibits cMET-specific pH-dependent binding. In one embodiment, the antibody comprises heavy and light chain CDRs present in the amino acid sequences as set forth in one of the following pairs: SEQ ID NOs: 15 & 16; SEQ ID NOs: 5 & 6; SEQ ID NOs: 7 & 8; SEQ ID NOS: 9 & 10; SEQ ID NOs: 11 & 12; SEQ ID NOs: 13 & 14; SEQ ID NOs: 17 & 18; SEQ ID NOs: 19 & 20; SEQ ID NOs: 21 & 22; SEQ ID NOs: 23 & 24; SEQ ID NOs: 25 & 26; SEQ ID NOs: 27 & 28; SEQ ID NOS: 29 & 30; SEQ ID NOs: 31 & 32; and SEQ ID NOs: 33 & 34. In an embodiment, the CDRs are determined using the Kabat or IMGT system.

In an embodiment of the foregoing methods, the cancer is a solid tumor cancer, including a non-small cell lung cancer (“NSCLC”), including a non-squamous NSCLC, a squamous NSCLC, and a “not otherwise specified” (NOS) NSCLC. In some embodiment, a biopsy from a tumor of said cancer and/or the entire tumor itself, comprises at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100% of cancer cells having a cMET expression level of at least a 1+, a 2+, or a 3+, as scored by an applicable and/or regulatory-agency approved immunohistochemistry (IHC) assay. In some embodiments, at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100% of the tumor cells have an IHC score of 1+ or 2+. In other embodiments, at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100% of the tumor cells have an IHC score of 2+. In still other embodiments, at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100% of the tumor cells have an IHC score of 1+.

In some embodiments, the disclosure provides a method of treating a cMET-positive and/or cMET-overexpressing solid tumor cancer having at least about a low or at least about an intermediate expression of cMET, comprising administering an effective amount of an anti-cMET antibody drug conjugate (ADC) to a human subject previously identified or selected as having said cancer, over a sufficient period of time to provide a therapeutic benefit, wherein the antibody component of the ADC exhibits cMET-specific pH-dependent binding (e.g., superior binding at about pH 7.4 versus about pH 5.4). In some embodiments, the antibody comprises heavy and light chain CDRs present in the amino acid sequences as set forth in one of the following pairs: SEQ ID NOs: 15 & 16; SEQ ID NOs: 5 & 6; SEQ ID NOs: 7 & 8; SEQ ID NOs: 9 & 10; SEQ ID NOs: 11 & 12; SEQ ID NOs: 13 & 14; SEQ ID NOs: 17 & 18; SEQ ID NOs: 19 & 20; SEQ ID NOs: 21 & 22; SEQ ID NOs: 23 & 24; SEQ ID NOs: 25 & 26; SEQ ID NOs: 27 & 28; SEQ ID NOS: 29 & 30; SEQ ID NOs: 31 & 32; and SEQ ID NOs: 33 & 34. In an embodiment, the CDRs are determined using the Kabat or IMGT system.

In some embodiments, the CDRs are as defined by the Kabat system. In other embodiments, the CDRs are as defined by the IMGT system. In still other embodiments, the CDRs are as defined by the Chothia system.

In some embodiments, the antibody comprises heavy chain and light chain variable regions comprising, consisting of, or consisting essentially of the amino acid sequences as set forth in one of the following pairs: SEQ ID NOs: 15 & 16; SEQ ID NOs: 5 & 6; SEQ ID NOs: 7 & 8; SEQ ID NOs: 9 & 10; SEQ ID NOs: 11 & 12; SEQ ID NOs: 13 & 14; SEQ ID NOs: 17 & 18; SEQ ID NOs: 19 & 20; SEQ ID NOs: 21 & 22; SEQ ID NOs: 23 & 24; SEQ ID NOs: 25 & 26; SEQ ID NOs: 27 & 28; SEQ ID NOs: 29 & 30; SEQ ID NOs: 31 & 32; and SEQ ID NOs: 33 & 34. In an embodiment, the CDRs are determined using the Kabat or IMGT system.

In an aspect, the disclosure provides a method of treating a cMET-positive and/or cMET-overexpressing solid tumor cancer having a cMET immunohistochemistry score of 1+, 2+, or 3+, comprising administering an effective amount of an anti-cMET antibody drug conjugate (ADC) to a human subject previously identified or selected as having said cancer, over a sufficient period of time to provide a therapeutic benefit, wherein the antibody component of the ADC exhibits cMET-specific pH-dependent binding, and optionally wherein the antibody comprises heavy and light chain CDRs present in the amino acid sequences as set forth in one of the following pairs: SEQ ID NOs: 15 & 16; SEQ ID NOs: 5 & 6; SEQ ID NOs: 7 & 8; SEQ ID NOs: 9 & 10; SEQ ID NOs: 11 & 12; SEQ ID NOs: 13 & 14; SEQ ID NOs: 17 & 18; SEQ ID NOs: 19 & 20; SEQ ID NOs: 21 & 22; SEQ ID NOs: 23 & 24; SEQ ID NOs: 25 & 26; SEQ ID NOs: 27 & 28; SEQ ID NOs: 29 & 30; SEQ ID NOs: 31 & 32; and SEQ ID NOs: 33 & 34. In an embodiment, the CDRs are determined using the Kabat or IMGT system.

Accordingly, in one aspect, the present disclosure provides pH-ADCs that specifically bind cMET with higher affinity at physiologic pH as compared to acidic pH (“anti-cMET pH-ADCs”). In some embodiments, the pH-ADC binds cMET well at physiologic pH and mildly acidic pH, but not well at the acidic pH found in endolysosomes and/or lysosomes. The anti-cMET pH-ADCs comprise cytotoxic and/or cytostatic agents linked by way of linkers to an antigen binding moiety or module that specifically binds cMET. In some embodiments, the antigen binding moiety is an antibody and/or an antigen binding fragment.

Antibodies and/or binding fragments composing the anti-cMET pH-ADCs generally comprise a heavy chain comprising a heavy chain variable region (VH) and a light chain comprising a light chain variable region (VL), each VH and VL having three complementarity determining regions (“CDRs”) referred to herein (in amino- to carboxy-terminal order) as VH CDRS (CDRH1, CDRH2, and CDRH3) and VL CDRs (CDRL1, CDRL2, and CDRL3). The amino acid sequences of exemplary CDRs, as well as the amino acid sequence of the VH and VL regions of the heavy and light chains of exemplary anti-cMET pH-antibodies and/or binding fragments that can compose the anti-cMET pH-ADCs are provided herein. Specific embodiments of anti-cMET pH-ADCs include, but are not limited to, an ADC comprising an anti-cMET pH-Ab module comprising one pair of heavy chain and light chain variable sequences having the sequences as set forth in SEQ ID NOs: 15 & 16; SEQ ID NOs: 5 & 6; SEQ ID NOs: 7 & 8; SEQ ID NOs: 9 & 10; SEQ ID NOs: 11 & 12; SEQ ID NOs: 13 & 14; SEQ ID NOs: 17 & 18; SEQ ID NOs: 19 & 20; SEQ ID NOS: 21 & 22; SEQ ID NOs: 23 & 24; SEQ ID NOs: 25 & 26; SEQ ID NOs: 27 & 28; SEQ ID NOs: 29 & 30; SEQ ID NOs: 31 & 32; and SEQ ID NOs: 33 & 34. In a particular embodiment, the ADC is MYTX-011, which is an ADC comprising an Ab module comprising a VH and a VL having the amino acid sequences as set forth in SEQ ID NOs: 15 & 16, respectively, which comprise VH CDRs and VL CDRs having the sequences as set forth in SEQ ID NOs: 236, 237, and 238, and SEQ ID NOs: 239, 240, and 241, respectively.

In particular embodiments, the anti-cMET pH-antibody module may include CDRs (e.g., as determined by the IMGT, Kabat, and/or Chothia system) and/or variable domains from antibodies exhibiting superior internalization (relative to non-pH-controls), notably including the following: MYT4309 (HCVD=SEQ ID NO: 242, LCVD=SEQ ID NO: 8); MYT4310 (HCVD=SEQ ID NO: 243, LCVD=SEQ ID NO: 8); MYT4311 (HCVD=SEQ ID NO: 244, LCVD=SEQ ID NO: 8); MYT4318 (HCVD=SEQ ID NO: 245, LCVD=SEQ ID NO: 8); MYT4319 (HCVD=SEQ ID NO: 229, LCVD=SEQ ID NO: 8); MYT4320 (HCVD=SEQ ID NO: 230, LCVD=SEQ ID NO: 8); MYT4322, HCVD=SEQ ID NO: 246, LCVD=SEQ ID NO: 8); MYT4323 (HCVD=SEQ ID NO: 247, LCVD=SEQ ID NO: 8); MYT4324 (HCVD=SEQ ID NO: 231, LCVD=SEQ ID NO: 8); MYT4325 (HCVD=SEQ ID NO: 9, LCVD=SEQ ID NO: 8); MYT4326 (HCVD=SEQ ID NO: 15, LCVD=SEQ ID NO: 16); MYT4327 (HCVD=SEQ ID NO: 15, LCVD=SEQ ID NO: 248); MYT4332 (HCVD=SEQ ID NO: 15, LCVD=SEQ ID NO: 249); MYT4334 (HCVD=SEQ ID NO: 15, LCVD=SEQ ID NO: 250); and MYT4336 (HCVD=SEQ ID NO: 15, LCVD=SEQ ID NO: 251), each disclosed in US 20220281984 A1, which is herein incorporated by reference in its entirety.

In some embodiments, the CDRs are as defined by the Kabat system. In other embodiments, the CDRs are as defined by the IMGT system. In other embodiments, the CDRs are as defined by the Chothia system. In still other embodiments, the CDRs are as defined by another suitable system. Now that the applicant has disclosed the plurality of heavy chain variable domains and light chain variable domains of the high-performing, pH-dependent anti-cMET antibodies, the skilled artisan using routine techniques can extract the sequences of the CDRs-irrespective of the precise system used to define said CDRs—and place them into different antibody contexts, frameworks, and/or formats.

In other particular embodiments, the anti-cMET pH-antibody module may include CDRs and/or variable domains from antibodies exhibiting superior internalization relative to non-pH-controls, notably including MYT4849 (SEQ ID NOs: 15 & 16); MYT5351 (SEQ ID NOs: 5 & 6); MYT4313 (SEQ ID NOs: 6 &7); MYT4325 (9 & 10); MYT4826 (SEQ ID NOs: 11 & 12); MYT4837 (SEQ ID NOs: 13 & 14); MYT4942 (SEQ ID NOs: 17 & 18); MYT5309 (SEQ ID NOs: 19 & 20); MYT5344 (SEQ ID NOs: 21 & 22); MYT5367 (SEQ ID NOs: 23 & 24); MYT4827 (SEQ ID NOs: 25 & 26); MYT4312 (SEQ ID NOs: 27 & 28); MYT4953 (SEQ ID NOs: 29 & 30); MYT4940 (SEQ ID NOS: 31 & 32); MYT4888 (SEQ ID NOs: 33 & 34); each disclosed in WO 2022/169975 ('975 PCT), the complete disclosures of which is incorporated herein.

Alternative versions of each of the foregoing antibodies may include one or more of the following modifications in their respective heavy chain sequences: 1) Triple Hinge (“TH”) only (e.g., as present in SEQ ID NO: 35); 2) “TH”+“LS” (e.g., as present in SEQ ID NO: 36); 3) “TH”+“YTE” (e.g., as present in SEQ ID NO: 37); 4) “TH”+“A118C” (e.g., as present in SEQ ID NO: 38); 5) “TH”+“LS”+“A118C” (e.g., as present in SEQ ID NO: 39); 6) “TH”+“YTE”+“A118C” (e.g., as present in SEQ ID NO: 40), each described in the '975 PCT.

As used herein, “Triple Hinge” (alternatively “TH”) refers to the substitution described in Wang J et al (2017) ABBV-399, a c-Met Antibody-Drug Conjugate that Targets Both MET-Amplified and c-Met-Overexpressing Tumors, Irrespective of MET Pathway Dependence, Clin Cancer Res, 23:992-1000. For example, a TH can be added to SEQ ID NO: 155 by making a lysine to cysteine substitution at amino acid position 105 and deleting the threonines at amino acid positions 106 and 108.

As used herein, “YTE” refers to the substitution described in Dall, W F et al., “Increasing the Affinity of a Human IgG1 for the Neonatal Fc Receptor: Biological Consequences” The Journal of Immunology (2002); 169:5171-5180). For example, a YTE can be added to SEQ ID NO: 155 by making a methionine to tyrosine substitution at amino acid position 135 (the “Y”), a serine to threonine substitution at amino acid position 137 (the “T”), and a threonine to glutamic acid substitution at amino acid position 139 (the “E”).

As used herein, “LS” refers to the substitution described in Zalevsky J et al., “Enhanced antibody half-life improves in vivo activity.” Nat Biotechnol. (2010) 28:157-9. For example, an LS can be added to SEQ ID NO: 155 by making a methionine to leucine substitution at amino acid position 311 (the “L”) and an asparagine to serine substitution at amino acid position 317 (the “S”).

As used herein, “A118C” refers to the A to C substitution as described in Junutula J. R., et al. Site-specific conjugation of a cytotoxic drug to an antibody improves the therapeutic index. Nat. Biotechnol. 2008; 26:925-932.). For example, an “A118C” can be added to SEQ ID NO: 155 by making an A to C substitution at amino acid position 1. And while the varying lengths of the different heavy chain variable domains may alter the precise location of the A to C substitution with respect to the complete heavy chain sequence, the amino acid position with respect to the heavy chain constant domain is 1. This concept is well-exemplified by the multiple sequence alignment of SEQ ID NO: 155 (a heavy chain constant domain with the “A”), SEQ ID NO: 156 (a heavy chain constant domain with a “C”), and SEQ ID NO: 38 (one of the complete heavy chain sequences containing the “A118C” substitution). Accordingly, as used herein, an “A118C mutation” or “A118C substitution” means the substitution of C for an A at the amino acid position of a given complete heavy chain sequence that corresponds to position 1 of its included heavy chain constant domain sequence.

| | | |
|---|---|---|
| SEQ 38 | QGTTVTVSSCSTKGPSVFPLAPSSKSTSGGTAALGCLVKDYFPEPVTVSWNSGAL | 165 |
| SEQ 155 | ---------ASTKGPSVFPLAPSSKSTSGGTAALGCLVKDYFPEPVTVSWNSGAL | 46 |
| SEQ 156 | ---------CSTKGPSVFPLAPSSKSTSGGTAALGCLVKDYFPEPVTVSWNSGAL | 46 |
| SEQ 38 | TSGVHTFPAVLQSSGLYSLSSVVTVPSSSLGTQTYICNVNHKPSNTKVDKRVEPK | 220 |
| SEQ 155 | TSGVHTFPAVLQSSGLYSLSSVVTVPSSSLGTQTYICNVNHKPSNTKVDKRVEPK | 101 |
| SEQ 156 | TSGVHTFPAVLQSSGLYSLSSVVTVPSSSLGTQTYICNVNHKPSNTKVDKRVEPK | 101 |
| SEQ 38 | SCD-CH-CPPCPAPELLGGPSVFLFPPKPKDTLMISRTPEVTCVVVDVSHEDPEV | 273 |
| SEQ 155 | SCDKTHTCPPCPAPELLGGPSVFLFPPKPKDTLMISRTPEVTCVVVDVSHEDPEV | 156 |
| SEQ 156 | SCD-CH-CPPCPAPELLGGPSVFLFPPKPKDTLYITREPEVTCVVVDVSHEDPEV | 154 |
| SEQ 38 | KFNWYVDGVEVHNAKTKPREEQYNSTYRVVSVLTVLHQDWLNGKEYKCKVSNKAL | 328 |
| SEQ 155 | KFNWYVDGVEVHNAKTKPREEQYNSTYRVVSVLTVLHQDWLNGKEYKCKVSNKAL | 211 |
| SEQ 156 | KFNWYVDGVEVHNAKTKPREEQYNSTYRVVSVLTVLHQDWLNGKEYKCKVSNKAL | 209 |
| SEQ 38 | PAPIEKTISKAKGQPREPQVYTLPPSREEMTKNQVSLTCLVKGFYPSDIAVEWES | 383 |
| SEQ 155 | PAPIEKTISKAKGQPREPQVYTLPPSREEMTKNQVSLTCLVKGFYPSDIAVEWES | 266 |
| SEQ 156 | PAPIEKTISKAKGQPREPQVYTLPPSREEMTKNQVSLTCLVKGFYPSDIAVEWES | 264 |
| SEQ 38 | NGQPENNYKTTPPVLDSDGSFFLYSKLTVDKSRWQQGNVFSCSVMHEALHNHYTQ | 438 |
| SEQ 155 | NGQPENNYKTTPPVLDSDGSFFLYSKLTVDKSRWQQGNVFSCSVMHEALHNHYTQ | 321 |
| SEQ 156 | NGQPENNYKTTPPVLDSDGSFFLYSKLTVDKSRWQQGNVFSCSVLHEALHSHYTQ | 319 |
| SEQ 38 | KSLSLSPG | 446 |
| SEQ 155 | KSLSLSPG | 329 |
| SEQ 156 | KSLSLSPG | 327 |

As used herein, “V205C” refers to a V to C substitution in the light chain constant domain as described in Shen et al., Nature Biotechnology Vol 30 No. 2 Feb. 2012 (a “V205C” substitution in the light chain constant region of the anti-Her2 mAb trastuzumab). And while the varying lengths of the different light chain variable domains may alter the precise location of the V to C substitution with respect to the complete light chain sequence, the amino acid position with respect to the light chain constant domain is 98. This concept is well-exemplified by the multiple sequence alignment of SEQ ID NO: 157 (LC constant domain with the “V”), SEQ ID NO: 158 (LC constant domain with the “C”), SEQ ID NO: 81 (LC with the “V”), SEQ ID NO: 82 (LC with the “C”), SEQ ID NO: 89 (LC with the “V”), and SEQ ID NO: 90 (LC with the “C”):

| SEQ 157 | ------------------------------------------------------- | |
|---|---|---|
| SEQ 158 | ------------------------------------------------------- | |
| SEQ 81 | DIQMTQSPSSLSASVGDRVTITCSVSSSVSSI---HLHWYQQKPGKAPKLLIYHT | 52 |
| SEQ 82 | DIQMTQSPSSLSASVGDRVTITCSVSSSVSSI---HLHWYQQKPGKAPKLLIYHT | 52 |
| SEQ 89 | DIVMTQSPDSLAVSLGERATINCKSSESVDSYANSHLHWYQQKPGQPPKLLIYRA | 55 |
| SEQ 90 | DIVMTQSPDSLAVSLGERATINCKSSESVDSYANSHLHWYQQKPGQPPKLLIYRA | 55 |
| SEQ 157 | ------------------------------------------------------- | |
| SEQ 158 | ------------------------------------------------------- | |
| SEQ 81 | SNLASGVPSRFSGSGSGTDFTLTISSLQPEDFATYYCQVYSGYPLTFGGGTKVEI | 107 |
| SEQ 82 | SNLASGVPSRFSGSGSGTDFTLTISSLQPEDFATYYCQVYSGYPLTFGGGTKVEI | 107 |
| SEQ 89 | STRESGVPDRFSGSGSGTDFTLTISSLQAEDVAVYYCQQSKEDPLTFGGGTKVEI | 110 |
| SEQ 90 | STRESGVPDRFSGSGSGTDFTLTISSLQAEDVAVYYCQQSKEDPLTFGGGTKVEI | 110 |
| SEQ 157 | -RTVAAPSVFIFPPSDEQLKSGTASVVCLLNNFYPREAKVQWKVDNALQSGNSQE | 54 |
| SEQ 158 | -RTVAAPSVFIFPPSDEQLKSGTASVVCLLNNFYPREAKVQWKVDNALQSGNSQE | 54 |
| SEQ 81 | KRTVAAPSVFIFPPSDEQLKSGTASVVCLLNNFYPREAKVQWKVDNALQSGNSQE | 162 |
| SEQ 82 | KRTVAAPSVFIFPPSDEQLKSGTASVVCLLNNFYPREAKVQWKVDNALQSGNSQE | 162 |
| SEQ 89 | KRTVAAPSVFIFPPSDEQLKSGTASVVCLLNNFYPREAKVQWKVDNALQSGNSQE | 165 |
| SEQ 90 | KRTVAAPSVFIFPPSDEQLKSGTASVVCLLNNFYPREAKVQWKVDNALQSGNSQE | 165 |
| SEQ 157 | SVTEQDSKDSTYSLSSTLTLSKADYEKHKVYACEVTHQGLSSPVTKSFNRGEC | 107 |
| SEQ 158 | SVTEQDSKDSTYSLSSTLTLSKADYEKHKVYACEVTHQGLSSPCTKSFNRGEC | 107 |
| SEQ 81 | SVTEQDSKDSTYSLSSTLTLSKADYEKHKVYACEVTHQGLSSPVTKSFNRGEC | 215 |
| SEQ 82 | SVTEQDSKDSTYSLSSTLTLSKADYEKHKVYACEVTHQGLSSPCTKSFNRGEC | 215 |
| SEQ 89 | SVTEQDSKDSTYSLSSTLTLSKADYEKHKVYACEVTHQGLSSPVTKSFNRGEC | 218 |
| SEQ 90 | SVTEQDSKDSTYSLSSTLTLSKADYEKHKVYACEVTHQGLSSPCTKSFNRGEC | 218 |

Accordingly, as used herein, a “V205C mutation” or “V205C substitution” means the substitution of a C for a V at the amino acid position of a given complete light chain sequence (LC) that corresponds to position 98 of its included light chain constant domain sequence.

In one specific embodiment, the pH-Ab of MYTX-011 is Q397, which comprises a heavy chain as set forth in SEQ ID NO: 75 and a light chain as set forth in SEQ ID NO: 82.

For therapeutic uses, it may be desirable to utilize anti-cMET pH-ADCs that bind cMET at physiologic pH with an affinity of at least 100 nM. Accordingly, in some embodiments, the anti-cMET pH-ADCs comprise an anti-cMET and/or anti-cMET binding fragment that binds cMET at physiologic pH with an affinity of at least about 100 nM, or even higher, for example, at least about 95 nM, 90 nM, 85 nM, 80 nM, 75 nM, 70 nM, 65 nM 60 nM, 55 nM, 50 nM, 45 nM, 40 nM, 35 nM, 30 nM, 25 nM, 20 nM, 15 nM, 10 nM, 7 nM, 6 nM, 5 nM, 4 nM, 3 nM, 2 nM, 1 nM, or greater. Affinity of anti-cMET pH-antibodies and/or binding fragments can be determined using techniques well known in the art or described herein, such as for example, Bio-Layer Interferometry (BLI) (e.g. Octet Red96), ELISA, isothermal titration calorimetry (ITC), surface plasmon resonance, flow cytometry, or fluorescent polarization assay. In one embodiment, the anti-cMET pH-antibody has at least about a 2-fold, 3-fold, 4-fold, 5-fold, 10-fold, 20-fold, 30-fold, 50-fold, or 100-fold, or greater affinity for cMET at physiologic pH versus pH 5.4. In some embodiments, the anti-cMET pH-antibody also has strong affinity for cMET at the slightly acidic pH conditions found in the tumor microenvironment (TME).

The anti-cMET pH-antibody may be, for example, a full-length, a bispecific, a dual variable domain, a multiple chain, or a single chain antibody, or another. Other suitable forms of antibody are disclosed in US 2022/0281984, incorporated by reference herein in its entirety.

The antibody may be of, or derived from, any isotype, including, for example, IgA, IgD, IgE, IgG (e.g., IgG1, IgG2, IgG3 or IgG4), IgM, or IgY. In some embodiments, the anti-cMET pH-antibody is an IgG (e.g., IgG1, IgG2, IgG3 or IgG4), in particular, IgG1. Antibodies may be of human or non-human origin (e.g., simians, equines, camelids, canines, felines, bovines, porcines, rodents, goats, rabbits, or avians). In specific embodiments, anti-cMET pH-ADCs comprise humanized antibodies and/or fully human antibodies, suitable for administration to humans.

Antigen binding fragments (ABFs) composing the anti-cMET pH-ADCs may include any fragment of an anti-cMET pH-antibody that retains the ability to specifically bind cMET in a pH-dependent manner. Specific examples of antibody binding fragments that may be included in the anti-cMET pH-ADCs include, but are not limited to, Fab, Fab′, F(ab′)2, Fv, scFv, scFv-Fc, VHH-scAb, VHH-Fab, Dual scFab, bispecific, multispecific, biparatopic, multiparatopic, and the like.

In some embodiments, the anti-cMET pH-antibody includes a single polypeptide. In some embodiments, the antigen binding fragment (ABF) is selected from a VH domain, a VHH domain, a VNAR domain, and a scFv. In some embodiments, the antibody is a BiTe, a (scFv)2, a nanobody, a nanobody-HSA, a DART, a TandAb, a scDiabody, a scDiabody-CH3, scFv-CH-CL-scFv, a HSAbody, scDiabody-HSA, or a tandem-scFv.

In some embodiments, the antibody includes two or more polypeptides. In some embodiments, the antibody is selected from the group of an antibody, a VHH-scAb, a VHH-Fab, a Dual scFab, a F(ab′)2, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)2-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a VHH-Fc KiH, a Fab-VHH-Fc, an Intrabody, a dock and lock, an ImmTAC, an IgG-IgG conjugate, a Cov-X-Body, a scFv1-PEG-scFv2, an Adnectin, a DARPin, a fibronectin, a DEP conjugate, and a PROTAB. In some embodiments, the ABPC or ABD includes a proteolysis-targeting antibody (PROTAB) (as described, e.g., in Marei et al, “Antibody targeting of E3 ubiquitin ligases for receptor degradation. Nature, 6 Oct. 2022). In some embodiments, the PROTAB may tether cell-surface E3 ubiquitin ligases to transmembrane proteins, resulting in target degradation both in vitro and in vivo.

Antibodies and/or ABFs may include modifications that alter the properties of the antibodies and/or ABFs, such as those that increase half-life, increase, or decrease agonistic capacity, and/or increase or decrease ADCC, as is well-known in the art.

The pH-ADC conjugate may include any cytotoxic and/or cytostatic agents known to inhibit the growth and/or replication of, and/or kill cells. Numerous such cytotoxic and/or cytostatic agents are known in the art and non-limiting examples include, but are not limited to, apoptosis regulators, cell cycle modulators, protein synthesis inhibitors, kinase inhibitors, DNA cross-linking agents, alkylating agents, intercalating agents, nuclear export inhibitors, topoisomerase I inhibitors, topoisomerase II inhibitors, mitochondria inhibitors, RNA/DNA antimetabolites and antimitotic agents.

Antimitotic/antiproliferative agents include, for example, natural products, such as vinca alkaloids (vincristine, vinblastine) and microtubule disruptors such as taxane (paclitaxel, docetaxel), epothilones, nocodazole, vinblastin, vinorelbine (NAVELBINE®), and epipodophyllotoxins (etoposide, teniposide); allocolchicine; auristatins, such as MMAE (monomethyl auristatin E) and MMAF (monomethyl auristatin F); halichondrin B; cemadotin; colchicine; any colchicine derivative; dolastatin-10; dolastatin-15; maytansine; maytansinoids, such as DM1 (N2′-deacetyl-N2′-(3-mercapto-1-oxopropyl)-maytansine); rhozoxin; paclitaxel or derivative thereof; docetaxel; thiocolchicine; and trityl cysteine.

Antimetabolites include, for example, purine analogs, folate antagonists (such as pralatrexate, methotrexate), pentostatin, cladribine, fludarabine and related inhibitors.

Hormones & hormone analogs (hormonal therapies) include, for example estrogen, tamoxifen, goserelin, bicalutamide, nilutamide, aromatase inhibitors (e.g., letrozole and anastrozole), anastrozole; exemestane; arzoxifene; bicalutamide; cetrorelix; degarelix; deslorelin; trilostane; dexamethasone; flutamide; raloxifene; fadrozole; toremifene; fulvestrant; letrozole; formestane; glucocorticoids; doxercalciferol; sevelamer carbonate; lasofoxifene; leuprolide acetate; megesterol; mifepristone; nilutamide; tamoxifen citrate; abarelix; prednisone; finasteride; rilostane; buserelin; luteinizing hormone releasing hormone (LHRH); Histrelin; trilostane or modrastane; fosrelin; and goserelin.

Apoptosis regulators include, for example, caspase-targeting drugs, caspase-regulators, BCL-2 family members, TNF family members, Toll family members, and/or NF-kappa-B proteins.

Cell cycle modulators include, for example, Paclitaxel; Nab-Paclitaxel; Docetaxel; Vincristine; Vinblastine; ABT-348; AZD-1152; MLN-8054; VX-680; Aurora A-specific kinase inhibitors; Aurora B-specific kinase inhibitors and pan-Aurora kinase inhibitors; AZD-5438; BMI-1040; BMS-032; BMS-387; CVT-2584; flavopyridol; GPC-286199; MCS-5A; PD0332991; PHA-690509; seliciclib (CYC-202, R-roscovitine); ZK-304709; AZD4877, ARRY-520; GSK923295A.

Protein synthesis inhibitors include, for example, Amikacin; Arbekacin; Bekanamycin; Dibekacin; Dihydrostreptomycin; Streptomycin; Neomycin; Framycetin; Paromomycin; Ribostamycin; Kanamycin; Tobramycin; Spectinomycin; Hygromycin B; Paromomycin; Gentamicin; Netilmicin; Sisomicin; Isepamicin; Verdamicin; Astromicin; Tetracycline; Doxycycline; Chlortetracycline; Clomocycline; Demeclocycline; Lymecycline; Meclocycline; Metacycline; Minocycline; Oxytetracycline; Penimepicycline; Rolitetracycline; Tetracycline; Glycylcyclines; Tigecycline; Oxazolidinone; Eperezolid; Linezolid; Posizolid; Radezolid; Ranbezolid; Sutezolid; Tedizolid.

Kinase inhibitors include, for example, Afatinib (e.g., GILOTRIF®); Axitinib; Bosutinib; Cetuximab (e.g., ERBITUX®), Panitumumab (e.g., VECTIBIX®), Necitumumab (e.g., PORTRAZZA®), Vandetanib (e.g., CAPRELSA®), Mobocertinib (EXKIVITY®), Dacomitinib (e.g., VIZIMPRO®); Crizotinib; Dasatinib; Erlotinib (e.g., TARCEVA®); Fostamatinib; Gefitinib (e.g., IRESSA®); Ibrutinib; Imatinib; Lapatinib (e.g., TYKERB®); Lenvatinib; Mubritinib; Neratinib (e.g., NERLYNX®); Nilotinib; Osimertinib (e.g., TAGRISSO®), Pazopanib; Pegaptanib; Sorafenib; Sunitinib; SU6656; Vandetanib; Vemurafenib; CEP-701 (lesaurtinib); XL019; INCB018424 (ruxolitinib); ARRY-142886 (selemetinib); ARRY-438162 (binimetinib); PD-325901; PD-98059; AP-23573; CCI-779; everolimus; RAD-001; rapamycin; temsirolimus; ATP-competitive TORC1/2 inhibitors including PI-103, PP242, PP30, Torin 1; LY294002; XL-147; CAL-120; ONC-21; AEZS-127; ETP-45658; PX-866; and the like.

DNA cross-linking/damaging agents include, for example, such as actinomycin, amsacrine, busulfan, carboplatin, chlorambucil, cisplatin, cyclophosphamide (CYTOXAN®), dactinomycin, daunorubicin, doxorubicin, DEBDOX, epirubicin, iphosphamide, melphalan, merchlorethamine, mitomycin C, mitoxantrone, nitrosourea, procarbazine, taxol, Taxotere, teniposide, etoposide, and triethylenethiophosphoramide.

DNA-hypomethylating agents, such as guadecitabine (SGI-110), oral decitabine and cedazuridine (ASTX727).

Alkylating agents include, for example, nitrogen mustards (cyclophosphamide, chlormethine, uramustine, Melphalan, Chlorambucil, Ifosfamide, and Bendamustine), Nitrosoureas (e.g., Carmustine, Lomustine, Streptozocin), and Alkyl sulfonates (e.g., Busulfan). Alkylating-like agents include, for example, Cisplatin, Carboplatin, Dicycloplatin, Eptaplatin, Lobaplatin, Miriplatin, Nedaplatin, Oxaliplatin, Picoplatin, Satraplatin, and Triplatin tetranitrate.

Intercalating agents include, for example, anthracyclines, including doxorubicin, daunorubicin, epirubicin, idarubicin, pirarubicin, aclarubicin, and mitoxantrone.

Enzymes such as L-asparaginase which systemically metabolizes L-asparagine and deprives cells which do not have the capacity to synthesize their own asparagine (i.e., a cancer or tumor starvation approach). Further example include crisantaspase (ERWINASE®) and GRASPA® (ERY-001, ERY-ASP), calaspargase pegol, and pegaspargase.

Nuclear export inhibitors include, for example, callystatin A; delactonmycin; KPT-185; kazusamycin A; leptolstatin; leptofuranin A; leptomycin B; ratjadone; and Verdinexor.

Topoisomerase I inhibitors include, for example, camptothecins, including synthetic derivatives thereof, including irinotecan and topotecan, and other analogs (e.g., NSC 100880, NSC 603071, and the like); morpholinisoxorubicin; and SN-38.

Topoisomerase II inhibitors include, for example, etoposide, doxorubicin, mitoxantrone, teniposide, novobiocin, merbarone, and aclarubicin.

Mitochondria inhibitors include, for example, pancratistatin; phenpanstatin; rhodamine-123; edelfosine; d-alpha-tocopherol succinate; compound 11 β; aspirin; ellipticine; berberine; cerulenin; GX015-070 (Obatoclax®; IH-Indole, 2-(2-((3,5-dimethyl-IH-pyrrol-2-yl)methylene)-3-methoxy-2H-pyrrol-5-yl)-); celastrol (tripterine); metformin; Brilliant green; ME-344.

RNA/DNA antimetabolites include, for example, L-alanosine; 5-azacytidine; 5-fluorouracil; acivicin; aminopterin derivative; L-aspartic acid (NSC 132483); aminopterin derivative; antifolate PT523; Baker's soluble antifol (NSC 139105); dichlorallyl lawsone ((2-(3,3-dichloroallyl)-3-hydroxy-1,4-naphthoquinone); brequinar; ftorafur ((pro-drug; 5-fluoro-I-(tetrahydro-2-furyl)-uracil); 5,6-dihydro-5-azacytidine; methotrexate; methotrexate derivative; PALA ((N-(phosphonoacetyl)-L-aspartate); pyrazofurin; trimetrexate.

In some embodiments, the following types of cancer are treated using the indicated combinations of chemotherapeutic agents: breast cancer (cyclophosphamide, methotrexate, 5-fluorouracil, and vinorelbine=“CMF”; or doxorubicin and cyclophosphamide=“AC”), Hodgkin's lymphoma (docetaxel, doxorubicin, and cyclophosphamide=“TAC”; doxorubicin, bleomycin, vinblastine, and dacarbazine=“ABVD”; mustine, vincristine, procarbazine, and prednisolone=“MOPP”), non-Hodgkin's lymphoma (cyclophosphamide, doxorubicin, vincristine, and prednisolone=“CHOP” or “R-CVP”), germ cell tumor (bleomycin, etoposide, and cisplatin=“BEP”), stomach cancer (epirubicin, cisplatin, and 5-fluorouracil=“ECF”; or epirubicin, cisplatin, and capecitabine=“ECX”), bladder cancer (methotrexate, vincristine, doxorubicin, and cisplatin=“MVAC”), lung cancer (cyclophosphamide, doxorubicin, vincristine, and vinorelbine=“CAV”), colorectal cancer (5-fluorouracil, folinic acid, and oxaliplatin=“FOLFOX”), pancreatic cancer (gemcitabine and 5-fluorouracil), and bone cancer (doxorubicin, cisplatin, methotrexate, ifosfamide, and etoposide=“MAP”/“MAPIE”). In a particular embodiment, the cytotoxic and/or cytostatic agent of the anti-cMET pH-ADC is a membrane-permeable antimitotic agent, including, for example, an auristatin, including, but not limited to, monomethyl auristatin E (“MMAE”).

The linkers linking the cytotoxic and/or cytostatic agents to the anti-cMET portion of the ADC may be short, long, rigid, flexible, hydrophobic, or hydrophilic in character, or may comprise elements having a variety of characteristics, such as elements of rigidity, elements of hydrophilicity, etc. The linker may be chemically stable in serum and/or the bloodstream, or it may include elements that provide for the release of the cytotoxic and/or cytostatic agents prior to entering cells. In some embodiments, the linkages provide for release of the agents upon internalization of the anti-cMET pH-ADC within the cell. In some particular embodiments, the linkers may be cleaved and/or immolated or otherwise broken down inside cells. A wide variety of useful ADC linkers is known in the art, and any such linkers, as well as others yet to be developed, may be used to link the cytotoxic and/or cytostatic agents to the anti-cMET portion of the pH-ADCs described herein.

In some embodiments, the anti-cMET pH-ADC is MYTX-011, which is a cMET-targeted val-cit-monomethyl auristatin E (vcMMAE) ADC that was specifically designed to address the shortcomings of existing therapies, including other cMET-targeted ADCs. As described herein, “vcMMAE” is a protease cleavable linker (maleimidocaproyl-valinecitrulline-p-aminobenzyloxycarbonyl (mc-vc-PAB)) attached to the small molecule anti-mitotic agent monomethyl auristatin E (MMAE). In some embodiments, the anti-cMET pH-ADCs disclosed herein drive responses for the majority of cMET+/cMET-expressing and/or cMET-overexpressing NSCLC patients, even those whose tumors express lower levels of cMET than those treatable with current cMET-targeted therapies.

The anti-cMET pH-ADCs of the present disclosure encompass, for example, any antibody that comprises a light chain comprising the VL-CDRs SVSSSVSSIHLH (SEQ ID NO: 239), HTSNLAS (SEQ ID NO: 240), and QVYSGYPLT (SEQ ID NO: 241); and a heavy chain comprising the VH-CDRs GYTFTDYYMH (SEQ ID NO: 236), RVNPNRRGTTYNOKFEG (SEQ ID NO: 237), and ARANWLDY (SEQ ID NO: 238).

MYTX-011 is an anti-cMET pH-ADC comprising a triple hinge (TH) IgG1 format and a vcMMAE linker-toxin, complementarity determining regions (CDRs) comprising the VH CDR 1, 2, and 3 sequences at set forth in SEQ ID NO: 236 (GYTFTDYYMH), SEQ ID NO: 237 (RVNPNRRGTTYNQKFEG), and SEQ ID NO: 238 (ARANWLDY), respectively, and the VL CDR 1, 2, and 3 sequences as set forth in SEQ ID NO: 239 (SVSSSVSSIHLH), SEQ ID NO: 240 (HTSNLAS), and SEQ ID NO: 241 (QVYSGYPLT), respectively, which mediate the pH-dependent binding. MYTX-011 comprises site-specific conjugation of the linker-toxin at an engineered cysteine residue (i.e., a “V205C substitution”), which results in a drug-to-antibody ratio (DAR) of 2 (Shen et al., Nature Biotechnology Vol 30 No. 2 Feb. 2012; and US 20220281984 A1) (FIG. 1A presents a schematic; vcMMAE=mc-vc-PAB-MMAE, CAS No. 646502-53-6). The complete heavy chain variable domain (VH) and light chain variable domain (VL) sequences of the antibody component of MYTX-011 (i.e., Q397) are as set forth in SEQ ID NO: 15 and SEQ ID NO: 16, respectively, and the entire Q397 heavy chain (HC) and light chain (LC) sequences comprise the amino acid sequences as set forth in SEQ ID NO: 75 and SEQ ID NO: 82, respectively. The entire “Q397noV205C” heavy chain (HC) and light chain (LC) sequences comprise the amino acid sequences as set forth in SEQ ID NO: 75 and SEQ ID NO: 81, respectively. Preclinical data indicate that MYTX-011 may be applied to a variety of cMET-positive/cMET-expressing and/or cMET-overexpressing cancers, including NSCLC. As a non-limiting example attesting to the safety of MYTX-011, two doses administered once every three weeks (Q3W) in cynomolgus monkeys resulted in no MYTX-011-related cardiovascular, respiratory, or central nervous system effects.

Other features and advantages of the invention will be apparent from the following detailed description and figures, and from the claims.

### BRIEF DESCRIPTION OF THE FIGURES

The patent or application file contains at least one drawing executed in color. Copies of this patent or patent application publication with color drawing(s) will be provided by the Office upon request and payment of the necessary fee. Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1A presents a schematic diagram of MYTX-011 and FIGS. 1B to 1E present binding and internalization data for anti-cMET pH-Abs according to this disclosure.

FIG. 1B is a graph showing percent dissociation of pH-dependent anti-cMET mAbs from human cMET at pH 5.4 measured using BLI. Anti-cMET mAbs were immobilized on AHC sensors, associated with recombinant human cMET at pH 7.4, and allowed to dissociate at pH 5.4. The inverse of the dissociation values was calculated by normalizing maximum response to one across all samples, taking the inverse of this value, and reporting it as a percentage. Greyscale traces represent different anti-cMET mAb variants.

FIG. 1C is a graph showing fold increase in internalization of mAb variants and MYTX-011 mAb (each “pH-Abs”) over non-pH engineered parent mAb in cMET+Detroit-562 cancer cells.

FIG. 1D is an Octet plot showing pH-dependent binding of MYTX-011 as measured by BLI. MYTX-011 was immobilized on an AHC sensor and tested for binding to human cMET under two conditions: association and dissociation at pH 7.4 (darker grey) or association at pH 7.4 and dissociation at pH 5.4 (light grey).

FIG. 1E is a graph illustrating the internalization of MYTX-011 in NCI-H1975 cells. MYTX-011 was incubated with pHrodo reagent for 30 min and the complex added to NCI-H1975 cells and incubated at 37ºC for 24 hours. Fold change was measured by subtracting the IgG control MFI from the MYTX-011 and parent ADC MFI and calculating the ratio of their differences.

FIG. 2 presents graphs showing the distribution of EBC-1 cells in Sub-G1, G1, S, and G2-M Phases after treatment with MYTX-011. MMAE=Monomethyl auristatin E; RC5-ADC=non-binding control.

FIG. 3 is a graph showing cell proliferation (% viability) of NCI-H441 cells after treatment with Q397-noV205C. Individual data points represent duplicate wells and bars represent the mean of the two data points. HGF=positive control, hepatocyte growth factor; RC1=comparative Ab having variable regions matching those of telisotuzumab; RC2=comparative Ab having variable regions matching those of emibetuzumab; RC3=comparative Ab having variable regions matching the 5D5 anti-human cMET antibody; Q397-noV205C=Q397 with V at position 98 of the polypeptide sequence encoding its light chain constant domain (i.e., SEQ ID NO: 157); Q397=the anti-cMET IgG antibody component of MYTX-011. Accordingly, the term “noV205C” means that the antibody has a valine and not a cysteine at amino acid position 98 of its light chain constant domain sequence.

FIG. 4 presents a graph showing the fold change in phosphorylation of ERK1/2 and Akt1 in NCI-H441 cells after treatment with Q397-noV205C.

FIG. 5A presents representative micrographs showing cMET-specific antibody staining of sections of cMET+ xenograft tumors. Panels A-D: anti-cMET antibody staining (clone SP44) of tumors from H1650, H1975, H1373, and EBC-1 xenograft mouse models, respectively. Panels E-H show hematoxylin and eosin (H & E) staining of the same xenograft tissues.

FIG. 5B presents additional micrographs. Panels (A-E): SP44 IVD staining of 3 biological replicate tumors from NCI-H1650, NCI-H2122, NCI-H1373, NCI-H1975, and EBC-1 xenograft mouse models, respectively. Lower panels (F-J) show the representative H & E staining of tumors from NCI-H1650, NCI-H2122, NCI-H1373, NCI-H1975, and EBC-1 xenograft mouse models, respectively. Scale bar=50 μm.

FIGS. 6A-6D present graphs showing tumor volume (TV) in EBC-1 tumor-bearing mice treated with MYTX-011. Panels A & B: Median TV curves for pilot efficacy and dose titration studies, respectively. Panels C & D: TV data corresponding to individual mice.

FIGS. 7A & 7B present graphs showing survival in EBC 1 tumor-bearing mice treated with MYTX-011 in the pilot efficacy study (A) and the dose titration study (B).

FIGS. 8A-8D present graphs showing TV in H1975 tumor-bearing mice treated with MYTX-011. Panels A & B: Median tumor growth curves for pilot efficacy and dose titration studies, respectively. C & D: Tumor volume data corresponding to individual mice, n=8 mice per group for all groups except n=7 for the MYTX-011 4 mg/kg group.

FIGS. 9A & 9B present graphs showing the effect of MYTX-011 on H1975 mouse survival.

FIGS. 10A-10D present graphs showing TV in H1373 treated with MYTX-011.

FIGS. 11A & 11B present graphs showing the impact of MYTX-011 on H1373 mouse survival.

FIG. 12 presents a graph showing median TV (mm³) for the indicated days after administration of test articles (including MYTX-011) to CTG-1353 mice and a representative micrograph showing cMET staining. For each of FIGS. 12-15, the following abbreviations apply: RC5-ADC=RC5-ADC-(DAR2); vehicle control=PBS; and TV=tumor volume.

FIG. 13A presents a graph showing median TV (mm³) for the indicated days after administration of test articles to CTG-3414 mice.

FIG. 13B presents micrographs showing the levels of cMET overexpression in CTG-3413 tumors. The middle image is of a tumor tissue section exhibiting both regions of high levels of cMET overexpression and low levels of cMET positivity, and the top and bottom images are magnified views of the left and right rectangular insets, respectively. Images and insets are representative of cMET IHC demonstrating heterogeneous expression of cMET in CTG-3414 PDX. Scale Bar=50 μm.

FIG. 14 presents a graph showing median TV (mm³) for the indicated days after administration of test articles to CTG-2669 mice and a micrograph showing cMET staining.

FIG. 15A presents a graph showing median TV (mm³) for the indicated days after administration of test articles to CTG-2533 mice and a micrograph showing cMET staining.

FIG. 15B presents a graph showing mean TV (mm³) for the indicated days after administration of test articles to CTG-2082, a PDX model negative for cMET expression.

FIG. 16 presents micrographs of cMET-stained sections from cMET low H2122 tumors.

FIG. 17 presents a graphical overlay of mean (±SD) total mAb and free MMAE after a single IV dose of MYTX-011 at 2.7 mg/kg to female and males cynomolgus monkeys.

FIG. 18 presents a graphical overlay of mean MYTX-011, total mAb, and free MMAE multiple IV doses of MYTX-011 at 6, 12, and 18 mg/kg to cynomolgus monkeys.

FIG. 19 presents graphs showing average MYTX-011 and total mAb exposure after multiple IV doses of MYTX-011 in male and female cynomolgus monkeys (6, 12, and 18 mg/kg).

FIG. 20 presents graphs showing mean AUC after multiple IV doses of MYTX-011 in male and female cynomolgus monkeys (6, 12, and 18 mg/kg).

FIG. 21 presents graphs showing tumor volume in NCI-H2122 tumor-bearing mice treated with MYTX-011 and the indicated test articles. A & B: Median tumor growth curves for single-dose and repeat-dose efficacy studies, respectively. C&D: Tumor volume data corresponding to individual mice. Abbreviations: RC6-ADC=RC6-ADC-(DAR3.7); RC1-ADC=RC1-ADC-(DAR3.01); RC5-ADC=RC5-ADC-(DAR1.96), Vehicle control=PBS. For single-dose efficacy study n=7 mice/group, for repeat dose efficacy study n=8 mice/group.

FIG. 22 presents graphs showing survival in NCI-H2122 tumor-bearing mice treated with MYTX-011. Panels A & B: Survival curves for single-dose and repeat dose efficacy studies, respectively.

FIG. 23A shows representative images from NCI-H1975 cells treated with for 24 hours with mAbs conjugated with a red pH-sensitive dye. Images are from two independent experiments (N=4 fields of views (FOV)/experiment).

FIG. 23B reports the quantification of the images in A (i.e., the time course of mAb uptake by live cell imaging in NCI-H1975 cells). MYTX-011 mAb (filled circles), parental mAb (open triangles), non-binding IgG (open squares), and untreated (X).

FIG. 23C presents representative fluorescence images of NCI-H1975 cells expressing the lysosomal marker LAMP1-RFP treated with AF488-conjugated MYTX-011 or parent mAb. Insets show the magnification of the regions where co-localization occurs. These fluorescent images demonstrate the delivery of the mAbs to LAMP1+ organelles (e.g., lysosomes).

FIG. 23D presents a graph showing the co-localization % of LAMP1-RFP and mAbs-AF488 measured at different time point.

FIG. 23E presents a graph showing single lysosome intensity of fluorescent-conjugated mAbs (i.e., the image quantitation of LAMP-1 associated mAbs). Statistical comparisons of data were performed using the Kruskal-Wallis test (ns: not significant, *<0.05, **<0.01, ****<0.0001).

FIG. 24A presents a graph showing the log IC50 values (mg/ml) of MYTX-011, a non-pH-dependent parent ADC (non-pH-ADC control), and a non-binding ADC for 62 different cancer cell lines. Statistical comparisons were performed using one-way ANOVA+Tukey's post-hoc test. ****<0.0001.

FIG. 24B presents a graph showing the max killing (%).

FIG. 24C presents a graph showing a correlation between the in vitro potency of MYTX-011 and both cMET mRNA and protein expression in cancer cell lines (Spearman's r [IC50 vs Protein]: −0.7; r [IC50 vs RNA]: −0.52).

FIG. 24D presents a waterfall plot representing the relationship between cMET expression and sensitivity to the treatment of the 62 cancer cell lines.

FIG. 24E presents a dot plot showing the influence of cell-line inherent MMAE sensitivity on ADC cytotoxicity. Data were compared using Kruskal-Wallis test; ns: not significant, ***<0.001.

FIG. 24F is a plot showing the distribution of IC50 values of MYTX-011 for the indicated cell lines.

FIG. 25A presents a graph showing tumor volume in head & neck cancer cell line Detroit-562 tumor-bearing mice treated with MYTX-011 (6 mg/kg body weight; solid squares), non-targeting control ADC (6 mg/kg body weight; solid circles), or vehicle control (open circles).

FIG. 25B presents a graph showing tumor volume in head & neck cancer cell line KYSE-150 tumor-bearing mice treated with MYTX-011 (6 mg/kg body weight; solid squares), non-targeting control ADC (6 mg/kg body weight; solid circles), or vehicle control (open circles).

FIG. 25C presents a graph showing tumor volume in esophageal cancer cell line FaDu tumor-bearing mice treated with MYTX-011 (6 mg/kg body weight; solid squares), non-targeting control ADC (6 mg/kg body weight; solid circles), or vehicle control (open circles).

FIG. 26A presents a graph showing tumor volume in gastric cancer cell line NUGC-4 tumor-bearing mice treated with MYTX-011 (6 mg/kg body weight; solid squares), non-targeting control ADC (6 mg/kg body weight; solid circles), or vehicle control (open circles).

FIG. 26B presents a graph showing tumor volume in gastric cancer cell line SNU-16 tumor-bearing mice treated with MYTX-011 (6 mg/kg body weight; solid squares), non-targeting control ADC (6 mg/kg body weight; solid circles), or vehicle control (open circles).

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

Many modifications and other aspects disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosure. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used herein, the term “and/or” includes any and all combinations of one or more of the associated listed items. Expressions such as “at least one of,” when preceding a list of elements, modify the entire list of elements and do not modify the individual elements of the list.

Reference to “a/an” chemical compound, protein, and antibody each refers to one or more molecules of the chemical compound, protein, and antibody rather than being limited to a single molecule of the chemical compound, protein, and antibody. Furthermore, the one or more molecules may or may not be identical, so long as they fall under the category of the chemical compound, protein, and antibody. Thus, for example, “an” antibody is interpreted to include one or more antibody molecules of the antibody, where the antibody molecules may or may not be identical (e.g., different isotypes and/or different antigen binding sites as may be found in a polyclonal antibody).

As used herein, the term “antigen-binding protein construct” (“ABPC”) is (i) a single polypeptide that includes at least one ABD or (ii) a complex of two or more polypeptides (e.g., the same or different polypeptides) that together form at least one ABD. Non-limiting examples and aspects of antigen-binding protein constructs are described herein. Additional examples and aspects of antigen-binding protein constructs are known in the art.

A “multi-specific antigen-binding protein construct” is an antigen-binding protein construct that includes two or more different ABDs that collectively specifically bind two or more different epitopes. The two or more different epitopes may be epitopes on the same antigen or on different antigens. When the different epitopes are present on the same antigen, the multi-specific antigen-binding protein construct is called a “multi-paratopic antigen-binding protein construct”. In some aspects, the antigen is present on the surface of the cell. In some aspects, a multi-specific antigen-binding protein construct binds two different epitopes (i.e., a “bispecific ABPC”). In some aspects, a multi-specific antigen-binding protein construct binds three different epitopes (i.e., a “trispecific ABPC”).

An “Antigen-Binding Protein” or “ABD” is one or more protein domain(s) (e.g., formed from amino acids from a single polypeptide or formed from amino acids from two or more polypeptides (e.g., the same or different polypeptides) that is capable of specifically binding to one or more different antigen(s). In some examples, an ABD can bind to an antigen or epitope with specificity and affinity similar to that of naturally-occurring antibodies. In some embodiments, the ABD can be an antibody or a fragment thereof. In some embodiments, an ABD can include an alternative scaffold. Non-limiting examples of ABDs are described herein. Additional examples of ABDs are known in the art. In some examples, an ABD can bind to a single antigen.

The term “antibody” is used herein in its broadest sense and includes certain types of immunoglobulin molecules that include one or more ABDs that specifically bind to an antigen or epitope. An antibody specifically includes, e.g., intact antibodies (e.g., intact immunoglobulins, e.g., human IgG (e.g., IgG1, IgG2, IgG3, IgG4)), antibody fragments, and multi-specific antibodies. One example of an ABD is an ABD formed by a VH-VL dimer. An antibody also includes a single polypeptide. In some embodiments, the antibody is selected from a VH domain, a VHH domain, a VNAR domain, and a scFv. In some embodiments, the antibody is a BiTe, a (scFv)2, a nanobody, a nanobody-HSA, a DART, a TandAb, a scDiabody, a scDiabody-CH3, scFv-CH-CL-scFv, a HSAbody, scDiabody-HSA, or a tandem-scFv. In some embodiments, the antibody includes two or more polypeptides. In some embodiments, the antibody includes a VHH-scAb, a VHH-Fab, a Dual scFab, a F(ab′)2, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)2-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a VHH-Fc KiH, a Fab-VHH-Fc, an Intrabody, a dock and lock, an ImmTAC, an IgG-IgG conjugate, a Cov-X-Body, a scFv1-PEG-scFv2, an Adnectin, a DARPin, a fibronectin, a DEP conjugate, and a PROTAB. Additional examples of an antibody are described herein or known in the art.

The phrase “endosomal/lysosomal pathway” refers to a network of endosomes (early endosomes, multi-vesicular bodies, late endosomes, and lysosomes) in the cytoplasm of a mammalian cell, wherein molecules internalized through cell-mediated internalization processes, e.g., pinocytosis, micropinocytosis, receptor-mediated endocytosis, and/or phagocytosis, are sorted.

Once the endosomes in the endosomal/lysosomal pathway are purified or isolated, assays for a target protein (e.g., an antigen-binding protein construct described herein) can be performed using methods known in the art (ELISA, Western blot, immunofluorescence, and immunoprecipitation followed by an assay for protein concentration), and can be used to determine the concentration or relative level of the target protein in the endosomes. Alternatively, endosomes in the endosomal/lysosomal pathway can be imaged using immunofluorescence microscopy using an detectably-labelled antibody (e.g., a fluorophore-labelled, a dye-labelled, or a GFP-labelled antibody, e.g., CellLight™ Early Endosome-GFP) that specifically binds to a characteristic protein present in the endosomes (e.g., EEA1 for early endosomes) and a fluorophore-labelled antibody that specifically binds to the protein of interest (e.g., an antigen-binding protein construct), and the level of the target protein in the endosomes can be determined by quantitation of the overlap in the fluorescence emissions of the two different antibodies.

The phrase “endolysosomal delivery” refers to rate of accumulation over time or the total accumulation at a specific timepoint of an antigen-binding protein construct (e.g., any of the antigen-binding protein constructs described herein) in the endosomal/lysosomal pathway in a mammalian cell (e.g., any of the exemplary target mammalian cells described herein).

An exemplary method to calculate the increase in endolysosomal delivery of a pH-engineered ABPC variant as compared to its corresponding starting ABPC from cellular fluorescence data is to measure the ratio of the variant's mean fluorescence intensity minus the mean fluorescence intensity of a non-binding IgG control, then all divided by the variant's corresponding starting ABPC's mean fluorescence intensity minus the mean fluorescence intensity of the IgG control.

An exemplary assay for measuring endolysosomal delivery of any of the ABPCs described herein include those which involve labeling of an ABPC with a fluorescent dye, followed by incubation of the labeled ABPC with cells and measurement of cellular fluorescence as an indicator of endolysosomal delivery of the ABPC (e.g., as described generally in Wustner, Traffic 7(6): 699-715, 2006). Alternatively, pH-sensitive dyes which preferentially fluoresce at acidic pH but not neutral pH can be used to label any of the ABPCs described herein, which can then be incubated with cells and the cellular fluorescence measured as an indicator of delivery of the ABPC into acidic endolysosomal compartments.

The term “population” when used before a noun means two or more of the specific noun. For example, the phrase “a population of cancer cells” means “two or more cancer cells.” Non-limiting examples of cancer cells are described herein.

The phrase “cytostatic to a cell” refers to a direct or indirect decrease in the proliferation (cell division) of the cell (e.g., a cancer cell) in vivo or in vitro. When an agent is cytostatic to a cell, the agent can, e.g., directly or indirectly result in cell cycle arrest of the cell (e.g., a cancer cell). In some examples, an agent that is cytostatic to a cell can reduce the number of cells in a population of the cells that are in S phase (as compared to the number of cells in a population of the cells that are in S phase prior to contact with the agent). In some examples, an agent that is cytostatic to a cell can reduce the percentage of the cells in S phase by at least 20%, at least 40%, at least 60%, or at least 80%.

The phrase “cytotoxic to a cell” refers to the inducement, directly or indirectly, in the death (e.g., necrosis or apoptosis) of the cell (e.g., a mammalian cell, e.g., a cancer cell).

“Affinity” refers to the strength of the sum total of non-covalent interactions between an antigen-binding site and its binding partner (e.g., an antigen or epitope). Unless indicated otherwise, as used herein, “affinity” refers to intrinsic binding affinity, which reflects a 1:1 interaction between members of an ABD and an antigen or epitope. The affinity of a molecule X for its partner Y can be represented by the dissociation equilibrium constant (KD). Affinity can be measured by common methods known in the art, including those described herein. Affinity can be determined, for example, using surface plasmon resonance (SPR) technology (e.g., BIACORE®) or biolayer interferometry (e.g., FORTEBIO®).

The term “epitope” means a portion of an antigen that is specifically bound by an ABD through a set of physical interactions between: (i) all monomers (e.g. individual amino acid residues, sugar side chains, and post-translationally modified amino acid residues) on the portion of the ABD that specifically binds the antigen and (ii) all monomers (e.g. individual amino acid residues, sugar side chains, post-translationally modified amino acid residues) on the portion of the antigen that is specifically bound by the ABD. Epitopes can, e.g., consist of surface-accessible amino acid residues, sugar side chains, phosphorylated amino acid residues, methylated amino acid residues, and/or acetylated amino acid residues and may have specific three-dimensional structural characteristics, as well as specific charge characteristics. Conformational and non-conformational epitopes are distinguished in that binding to the former, but not the latter, may be lost in the presence of denaturing solvents. In some embodiments, an epitope is defined by a linear amino acid sequence of at least about 3 to 6 amino acids, or about 10 to 15 amino acids. In some embodiments, an epitope refers to a portion of a full-length protein or a portion thereof that is defined by a three-dimensional structure (e.g., protein folding). In some embodiments, an epitope is defined by a discontinuous amino acid sequence that is brought together via protein folding. In some embodiments, an epitope is defined by a discontinuous amino acid sequence that is brought together by quaternary structure (e.g., a cleft formed by the interaction of two different polypeptide chains). The amino acid sequences between the residues that define the epitope may not be critical to three-dimensional structure of the epitope. A conformational epitope may be determined and screened using assays that compare binding of antigen-binding protein construct to a denatured version of the antigen, such that a linear epitope is generated. An epitope may include amino acid residues that are directly involved in the binding, and other amino acid residues, which are not directly involved in the binding.

Methods for identifying an epitope to which an ABD specifically binds are known in the art, e.g., structure-based analysis (e.g. X-ray crystallography, NMR, and/or electron microscopy) (e.g. on the antigen and/or the antigen-ABD complex) and/or mutagenesis-based analysis (e.g. alanine scanning mutagenesis, glycine scanning mutagenesis, and homology scanning mutagenesis) wherein mutants are measured in a binding assay with a binding partner, many of which are known in the art.

The term “paratope” means a portion of an ABD that specifically binds to an antigen through a set of physical interactions between: (i) all monomers (e.g. individual amino acid residues, sugar side chains, posttranslationally modified amino acid residues) on the portion of the ABD that specifically binds the antigen and (ii) all monomers (e.g. individual amino acid residues, sugar side chains, posttranslationally modified amino acid residues) on the portion of the antigen that is specifically bound by the ABD. Paratopes can, e.g., consist of surface-accessible amino acid residues and may have specific three-dimensional structural characteristics, as well as specific charge characteristics. In some embodiments, a paratope refers to a portion of a full-length ABD or a portion thereof that is defined by a three-dimensional structure (e.g., protein folding). In some embodiments, a paratope is defined by a discontinuous amino acid sequence that is brought together via protein folding. In some embodiments, an epitope is defined by a discontinuous amino acid sequence that is brought together by quaternary structure (e.g., a cleft formed by the interaction of two different polypeptide chains). The amino acid sequences between the residues that define the paratope may not be critical to three-dimensional structure of the paratope. A paratope may comprise amino acid residues that are directly involved in the binding, and other amino acid residues, which are not directly involved in the binding.

Methods for identifying a paratope to which an ABD specifically binds are known in the art, e.g., structure-based analysis (e.g., X-ray crystallography, NMR, and/or electron microscopy) (e.g. on the ABD, and/or the ABD-antigen complex), and/or mutagenesis-based analysis (e.g., alanine scanning mutagenesis, glycine scanning mutagenesis, and homology scanning mutagenesis) wherein mutants are measured in a binding assay with a binding partner, many of which are known in the art.

The phrase “present on the surface of a mammalian cell” means (1) an antigen that physically attached to or at least partially embedded in the plasma membrane of a mammalian cell (e.g., a transmembrane protein, a peripheral membrane protein, a lipid-anchored protein (e.g., a GPI-anchor), an N-myristoylated protein, or a S-palmitoylated protein) or (2) an antigen that is stably bound to its cognate receptor, where the cognate receptor is physically attached to the plasma membrane of a mammalian cell (e.g., a ligand bound to its cognate receptor, where the cognate receptor is physically attached to the plasma membrane). Non-limiting methods for determining the presence of antigen on the surface of a mammalian cell include fluorescence-activated cell sorting (FACS), immunohistochemistry, cell-fractionation assays and Western blotting.

The phrase “control ABPC” or “control antigen-binding protein construct” means (i) an ABPC that is capable of specifically binding to cMET or an epitope of cMET presented on the surface of a mammalian cell (e.g., a target mammalian cell), where one or both of the following is true: (a) the dissociation rate of the first ABD at a pH of ^{˜}4.0 to ^{˜}6.5 is no more than 3-fold faster than the dissociation rate at a pH of ^{˜}7.0-^{˜} 8.0; or (b) the dissociation constant (KD) of the first ABD at a pH of ^{˜}4.0-^{˜} 6.5 is no more than 3-fold greater than the KD at a pH of ^{˜}7.0-^{˜}8.0.

The term “extracellular space” means the liquid exterior to the plasma membrane of a mammalian cell.

The term “endolysosomal space” means the fluid encapsulated by the vesicles and organelles that make-up the endosomal/lysosomal pathway in a mammalian cell.

The phrase “a reduced level” or “a decreased level” can be a reduction or decrease of at least a 1% (e.g., ≥2%, ≥4%, ≥6%, ≥8%, ≥10%, ≥12%, ≥14%, ≥16%, ≥18%, ≥20%, ≥22%, ≥24%, ≥26%, ≥30%, ≥35%, ≥40%, ≥45%, ≥50%, ≥55%, ≥60%, ≥65%, ≥70%, ≥75%, ≥80%, ≥85%, ≥90%, ≥95%, or ≥99%) reduction as compared to a reference level or value.

The term “cell killing potency” refers to the ability of an agent to induce, directly or indirectly, the apoptosis and/or necrosis of a mammalian cell, measured as a rate over time or at a relevant timepoint. Methods for determining the cell killing potency of a cell are known in the art (e.g., trypan blue staining, microscopy, fluorescence-assisted cell sorting, and assays to detect markers of apoptosis (e.g., Annexin V)). In non-limiting examples, cell killing potency can be measured, e.g., by cell killing at a single concentration of an agent, by the IC50 of the agent, or by the ratio of an agent's dissociation constant KD on mammalian cells divided by its IC50. In some non-limiting examples, the IC50s and/or the KD ratios described herein are compared to those of a control ABPC, and, optionally, demonstrate that the ABPCs described herein have a higher cell killing potency as compared to the control ABPC.

The term “toxin liberation” refers to the ability of a mammalian cell (e.g., a non-cancerous mammalian cell or a cancer cell) to internalize (e.g., via pinocytosis and/or receptor-mediated endocytosis) any of the ABPCs described herein (e.g., any of ABPCs or control ABPCs described herein) that are conjugated to a toxin, and subsequently release the toxin conjugated to the ABPC, measured as a rate over time or at a specific timepoint. Toxin liberation can be assessed using a variety of different exemplary assays, e.g., ELISA, immunofluorescence, cell killing assays, cell cycle arrest assays, DNA damage assays, mass spectrometry, HPLC, and/or an isotope-labeled toxin.

The phrase “target cell” or “target mammalian cell” or “mammalian target cell” means a mammalian cell that has at least one cMET present on its surface. In some examples, a mammalian target cell can be a cancer cell. In some embodiments of a target mammalian cell can have a total of about the following (each ±about 10%): 1-10E6, 1-9E6, 1-8E6, 1-7E6, 1-6E6, 1-5E6, 1-4E6, 1-3E6, 1-2E6, 1-1E6, 1-800,000, 1-600,000, 1-400,000, 1-200,000, 1-100,000, 1-80,000, 1-80,000, 1-75,000, 1-70,000, 1-65,000, 1-60,000, 1-55,000, 1-50,000, 1-45,000, 1-40,000, 1-35,000, 1-30,000, 1-25,000, 1-20,000, 1-15,000, 1-10,000, 1-7,500, 1-5,000, 1-4,000, 1-3,000, 1-2,000, 1-1,000, 1-500, 1-100, 1-50, or 1-10, or any of the ranges of numbers recited in US 2022/0281984, which is incorporated by reference herein in its entirety) of the cMET present on the plasma membrane of the target mammalian cell. Current IHC tests have an approximate threshold of detection of between about 1,000 and about 5,000 cMET molecules per cell.

The phrase “antigen density” means the number of cMET present on the surface of a target mammalian cell or the average number of cMET on the surface of a population of particular type of target mammalian cells. It can be measured, e.g., using the Quantibright bead kit or radiolabel (e.g., BD Biosciences PE Phycoerythrin Fluorescence Quantitation Kit, catalog #340495).

The phrase “amino acid substituted with a histidine” means the substitution of an amino acid residue that is not histidine in a reference polypeptide sequence with a histidine. Non-limiting methods for substituting an amino acid residue in a reference polypeptide with a histidine are described herein. Additional methods for substituting an amino acid residue in a reference polypeptide with a histidine are known in the art.

The phrase “amino acid substituted with an alanine” means the substitution of an amino acid residue that is a histidine in a reference polypeptide sequence with an alanine. Non-limiting methods for substituting a histidine in a reference polypeptide with an alanine are described herein. Additional methods for substituting a histidine in a reference polypeptide with an alanine are known in the art.

As used herein, the terms “treat”, “treatment”, and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage”, “managing”, and “management” refer to the beneficial effects that a subject derives from a therapy which does not necessarily result in a cure of the disease.

Treating includes effective cancer treatment with an effective amount of a therapeutic agent (e.g., an anti-cMET pH-ADC, e.g., MYTX-011) or combination of therapeutic agents. Treating herein includes, inter alia, adjuvant therapy, neoadjuvant therapy, metastatic cancer therapy, and non-metastatic cancer therapy (e.g., locally advanced cancer therapy). The treatment may be first-line treatment (e.g., the patient may be naïve to prior treatment or may have received prior systemic therapy), or second line, third line, or a later treatment.

The terms “prevent”, “preventing”, and “prevention” refer to reducing the likelihood of the onset (or recurrence) of a disease, disorder, condition, or associated symptom(s) (e.g., a cancer).

As used herein, the term “treatment-related AE” refers to an AE that is judged by an investigator to have occurred as a result of a treatment.

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human. In one embodiment, the subject is a mammal, e.g., a human, diagnosed with a condition or disorder. In another embodiment, the subject is a mammal, e.g., a human, at risk of developing a condition or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

pH-Antibody Drug Conjugates (pH-ADCs) that Bind to cMET and cMET Overexpressing Cells

The present disclosure relates to pH-ADCs that bind specifically to human cMET, compositions comprising such pH-ADCs, anti-cMET pH-antibodies and/or binding fragments (BFs) that can comprise the ADCs, polynucleotides encoding anti-cMET pH-antibodies and/or BFs that comprise such ADCs, host cells and/or expression systems capable of producing the pH-antibodies and/or BFs, methods and compositions useful for making the pH-antibodies, BFs, and pH-ADCs, and a variety of methods of us of such pH-ADCs in the treatment of cancer.

Data provided herein demonstrate that anti-cMET pH-antibody drug conjugates (“pH-ADCs”) exhibit potent antitumor effects, against cMET+ solid tumors, especially those with an IHC-score of 2+ and 3+ when measured by immunohistochemistry with the SP44 antibody. In particular, the disclosed anti-cMET pH-ADCs demonstrated efficacy against tumors characterized by mutations not well-addressed by prior ADCs targeting cMET (see e.g., Example 5). In some embodiments, the anti-cMET pH-ADCs are effective against even 1+ cMET+, including cMET-overexpressing tumors (see e.g., Example 11). In other embodiments, the anti-cMET pH-ADCs are effective against tumors having heterogeneous levels of cMET overexpression (see e.g., Example 6).

The term “cMET MYTX-ADC staining procedure” is used throughout to describe the evaluation of tissue/tumor cMET levels, and is described in Example 11. An “H-score”, “IHC score” or other suitable scoring system may be used to express the staining results. In particular embodiments, the CONFIRM™ anti-Total c-MET (SP44) Rabbit Monoclonal Primary Antibody system (Ventana Medical) is used in the practice of this staining procedure. In other embodiments, other approved tests, existing now or later developed, may be used to evaluate the level of cMET expression.

Utilization of the H-score approach facilitates the determination of cMET+ intensity variation and percent positivity within a given tumor type and across tumor types. It also permits the determination of thresholds for positive staining. And as the skilled person will appreciate, H-scores may be determined manually (e.g. by a pathologist), or by software-enabled, automated image processing and analysis. Briefly, multiple sections of tissue (e.g. tumor) are assessed to provide the overall percentage of cells (0-100) in a tumor having staining intensities ranging from 0-3+. It is important to note that some tumors may be homogeneous (e.g. +2 throughout), whereas others may be heterogeneous (e.g. patches of +1 and +3). The staining procedure shows cMET protein expression both in the surface of the cell as well as in the cytoplasm. Fixed fields of ˜100 cells are visually inspected and a staining score is assigned to each cell in view of surface staining as follows: 0=none; 1+=weak; 2+=moderate; 3+=strong.

H-score calculation. H-score=1*(% 1+ cells)+2*(% 2+ cells)+3*(% 3+ cells).

Tumors may also be assigned an IHC score of 0, 1+, 2+, or 3+, which is distinct from H-score values. While the H-score refers to a weighted sum of the staining intensity for all individual cells that were inspected and scored, a tumor IHC score refers to the overall staining of a specific area within a tumor sample. For example, a tumor having an H-Score of 150 could be comprised of pluralities of 1+ and 2+ cells (e.g., 20% 1+ cells and 65% 2+ cells), and given portions of the tumor might have a tumor IHC score of either 1+ or 2+.

As used herein, IHC 0 is assigned in cases where no cell in a fixed field is stained; IHC 1+ where overall staining is low; IHC 2+ where most cells are moderately stained; and IHC 3+ where most cells are strongly stained. In other embodiments, IHC 2+ is assigned if at least 15% of the cells in a fixed field are moderately stained and IHC 3+ is assigned if at least 15% of the cells in a fixed field are strongly stained.

In another embodiment, “low” tumor cMET expression refers to tumors with cMET expression of 1+ scored by a relevant cMET IHC assay in at least 15%, 25%, 50%, or 75% of tumor cells; “intermediate” tumor cMET overexpression refers to tumors with cMET overexpression of 2+ scored by a relevant cMET IHC assay in at least 15%, 25%, 50%, or 75% of tumor cells; and “high” tumor cMET overexpression refers to tumors with cMET overexpression of 3+ scored by a relevant cMET IHC assay in at least 15%, 25%, 50%, or 75% of tumor cells.

As used herein, an IHC score of 1+ is equivalent to an H-score between 50 and 149, an IHC score of 2+ is equivalent to an H-score of between 150 and 224, and an IHC score of 3+ is equivalent to an H-score of ≥225. In another embodiment, an IHC score of 1+ is equivalent to an H-score>about 50 to 149.

Accordingly, in a first aspect, this disclosure provides ADCs that bind cMET specifically and in a pH-dependent manner (“anti-cMET pH-ADCs”). The anti-cMET pH-ADCs comprise cytotoxic and/or cytostatic agents conjugated by way of linkers to an antigen binding moiety (ABM) that binds cMET specifically, in a pH-dependent manner. In the case of MYTX-011, the ABM (Q397) specifically binds the Sema domain of human cMET well at physiologic pH, but substantially less well at acidic pH (e.g. in the lysosomal environment). In other anti-cMET pH-ADCs, the antigen binding moiety may be any other moiety capable of specifically binding cMET at physiologic but not acidic pH. In some embodiments, the ABM is an antibody or an ABF.

In a particular embodiment, the cytotoxic and/or cytostatic component of the anti-cMET pH-ADC is a cell-permeating antimitotic agent, such as, for example, an auristatin. Specific examples of cell-permeating auristatins include, but are not limited to, dolastatin-10 and monomethyl auristatin E (“MMAE”).

As known by those of skill in the art, antibodies and ABMs consist of modules, for example, VH CDRs, VH domains, VL CDRs and VL domains. Throughout this disclosure, it is intended that each and every particular embodiment(s) may be combined with each and every other particular embodiment(s) as though each combination were expressly described one at a time. As a non-limiting example, each variant of a “VH CDR1” may be combined with each variant of a corresponding “VH CDR2” or “VH CDR3”, and/or each variant of a “VL CDR1” may be combined with each variant of a corresponding “VL CDR2” or “VL CDR3”, and so on.

Analogously, the pH-ADCs disclosed herein also consist of modules, for example, antibodies (also shortened to “A”), linkers (“L”), and cytotoxic and/or cytostatic agents (“D”), from which the disclosed pH-ADCs are built. As with the Ab and ABM modules, all particular embodiments may be combined with each other as though each particular combination were expressly described one at a time.

Moreover, the pH-ADCs described herein may be provided in the form of salts, for example but not solely, salts that are pharmaceutically acceptable. As is appreciated by the skilled artisan, suitably acidic and/or basic functional groups may be reacted with inorganic bases and/or acids to form an acceptable salt. Alternatively or additionally, inherently charged compounds may be reacted with suitable counter ions (e.g. halide) to produce a salt.

Common acids for forming addition salts include inorganic acids (e.g. HCl, HBr, H₂SO₄, HI, etc.) and organic acids (e.g. para-toluenesulfonic acid, methanesulfonic acid, oxalic acid, para-bromophenyl-sulfonic acid, carbonic acid, citric acid, succinic acid, and the like). Common base addition salts include ammonium and alkali or alkaline earth metal hydroxides, carbonates, bicarbonates, and other such bases.

pH-Antibodies to cMET

In particular embodiments, the ABP is an antibody or an ABF. As used herein, the term “anti-cMET pH-antibody” or “anti-cMET pH-Ab” refers to an immunoglobulin molecule that binds specifically to cMET in a pH-dependent manner. The anti-cMET pH-Ab has a relatively high affinity for cMET at physiologic pH, yet a substantially lower affinity for cMET at acidic pH, in particular, pH of about 5.4 or less. A pH-dependent antibody particularly well-suited to deliver payloads (i.e., a pH-dependent ADC) is one that binds well at both physiologic pH and the slightly acidic pH conditions present in a typical tumor microenvironment (TME), but not well at pH 5.4.

As the skilled artisan appreciates, antibodies of a certain format comprise heavy chain variable domain (HCVD) CDRs and light chain variable domain (LCVD) CDRs (also called “hypervariable” regions). The less variable portions of the HCVD and LCVD are commonly referred to as the framework (FR). Now that applicant has disclosed CDRs that confer such cMET-specific pH-dependent binding, the skilled artisan will appreciate a variety of reasonable and routine FR variations that can be made whilst holding the CDRs constant.

Anti-cMET pH-ADCs comprise pH-antibodies generally comprising a heavy chain (HC) comprising a variable region (VH) having three CDRs (VH CDR1, VH CDR2, and VH CDR3), and a light chain (LC) comprising variable region (VL) having three CDRs (VL CDR1, VL CDR2, and VL CDR3). The amino acid sequences of particular CDRs, VH and VL regions that may be included in ABPs composing the anti-cMET pH-ADCs are provided herein.

Anti-cMET pH-ADCs may comprise antibody modules consisting of monoclonal, polyclonal, human-engineered, or naturally-modified antibodies, for example but not solely, humanized, chimeric, primatized, SC, and bispecific antibodies, and the like.

Anti-cMET pH-ADCs may comprise full-length antibodies and cMET-binding ABFs that are capable of specifically binding cMET at physiologic but not acidic pH. In some embodiments, the ABF may be a Fab fragment, an Fv fragment, or an scFv fragment.

In some embodiments, the anti-cMET pH-ADCs comprise bispecific, multispecific, biparatopic, or multiparatopic antibodies.

In some embodiments, the anti-cMET pH-ADCs comprise derivatized antibodies.

In some embodiments, the antic-cMET pH-antibodies or ABFs have been modified to modify at least one non-variable region-mediated effector function (e.g. Fc receptor binding, which can impact phagocytosis, opsonization, and/or ADCC).

In some embodiments, the disclosed anti-cMET pH-antibodies can serve more than one purpose. For example, the pH-antibodies may be used for IHC assays of tumor biopsies obtained from patients to whom the disclosed pH-ADCs have been (or will be) administered. The Ventana SP44 clone, and antibodies having comparable properties may be prepared or acquired and the staining procedure modified such that the IHC method has comparable or better diagnostic power as compared with the Ventana assay. Moreover, anti-cMET antibodies other than SP44 may be used for the IHC assay.

Examples of anti-cMET pH-antibodies that can be used for diagnostic, theranostic, therapeutic, and/or other purposes include, but are not limited to, antibodies disclosed in US 2022/0281984 and WO 2022/169975. The complete disclosures of these applications are incorporated herein by reference, including the amino acid sequences for the CDRs, HCs (entire and variable regions), and LCs (entire and variable regions).

In other embodiments, the anti-cMET pH-antibodies are administered for treatment purposes, either as components of ADCs, or before/after/concurrently with the ADCs.

### MYTX-011 and Related Antibodies for Treatment Purposes

The anti-cMET pH-ADC is called “MYTX-011”, which is an ADC comprised of the cMET-targeting, pH-antibody Q397 conjugated to the small molecule anti-mitotic agent monomethyl auristatin E (MMAE) via a valine citrulline linker (“maleimidocaproyl-valinecitrulline-p-aminobenzyloxycarbonyl” or “mc-vc-PAB”). MYTX-011 binds specifically to tumor cell-surface cMET (at both physiologic and slightly acidic pH), is internalized, and then rapidly releases cMET at acidic pH (e.g., the pH conditions typical of lysosomes). The MMAE payload is released after the ADC is internalized, either before, during, or after the ADC dissociates from cMET, resulting in inhibition of microtubule function and disruption of essential processes and cell death. And since MMAE is cell-permeable, the internalized MMAE can then diffuse outside of the cell and into other, “bystander” cells. MYTX-011 is cytotoxic to cancer cells expressing high, moderate, and low levels of cMET-expression/cMET-overexpression and demonstrates antitumor activity in cell derived xenograft (CDX) and patient derived xenograft (PDX) animal models of cancer.

### The Antibody Portion of MYTX-011

“Q397” is a pH-dependent, humanized anti-cMET antibody, whose general structure was disclosed in WO 2022/169975 A1. Prior to this, the heavy chain variable (VH) and light chain variable (VL) regions of Q397 were disclosed in US 2022/0281984 (as SEQ ID NO: 146 and SEQ ID NO: 284 of that application, respectively). The disclosures of the foregoing applications are incorporated by reference herein in their entireties. Specifically, Q397 comprises a heavy chain comprising the amino acid sequence set forth in SEQ ID NO: 75 and a light chain comprising the amino acid sequence set forth in SEQ ID NO: 82. The amino acid sequences for the heavy and light chain variable regions of Q397 are as set forth in SEQ ID NO: 15 and SEQ ID NO: 16, respectively.

Heavy chain of Q397 (and “Q397noV205C”) with
triple hinge (underlined), constant domain
(ITALICIZED), and variable heavy chain region
(BOLD) (SEQ ID NO: 75):
QVQLVQSGAEVKKPGASVKVSCKASGYTFTDYYMHWVRQAPGQGLEWM
GRVNPNRRGTTYNQKFEGRVTMTTDTSTSTAYMELRSLRSDDTAVYYC
ARANWLDYWGQGTTVTVSSASTKGPSVFPLAPSSKSTSGGTAALGCLV
KDYFPEPVTVSWNSGALTSGVHTFPAVLQSSGLYSLSSVVTVPSSSLG
TQTYICNVNHKPSNTKVDKRVEPKSCDCHCPPCPAPELLGGPSVFLFP
PKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHY
TQKSLSLSPG
Light chain of Q397 with constant domain
(ITALICIZED), a Cysteine (C) (underlined) at
amino acid position 98 of the constant domain,
and variable light chain region (BOLD) (SEQ ID
NO: 82):
DIQMTQSPSSLSASVGDRVTITCSVSSSVSSIHLHWYQQKPGKAPKLL
IYHTSNLASGVPSRFSGSGSGTDFTLTISSLQPEDFATYYCQVYSGYP
LTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKV
YACEVTHQGLSSPCTKSFNRGEC
Light chain of “Q397-noV205C” with constant
domain (ITALICIZED), Valine (V) (underlined) at
amino acid position 98 of the constant domain,
and variable light chain region (BOLD) (SEQ ID
NO: 81):
DIQMTQSPSSLSASVGDRVTITCSVSSSVSSIHLHWYQQKPGKAPKLL
IYHTSNLASGVPSRFSGSGSGTDFTLTISSLQPEDFATYYCQVYSGYP
LTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKV
YACEVTHQGLSSPVTKSFNRGEC

The anti-cMET pH-ADCs of the present disclosure encompass any antibody that comprises a light chain comprising CDR-L1, CDR-L2 and CDR-L3 contained within the amino acid sequence set forth in SEQ ID NO: 16. Namely, the VL CDR sequences are SVSSSVSSIHLH (CDR-L1, SEQ ID NO: 239), HTSNLAS (CDR-L2, SEQ ID NO: 240), and QVYSGYPLT (CDR-L3, SEQ ID NO: 241); and a heavy chain comprising CDR-H1, CDR-H2 and CDR-H3 contained within the amino acid sequence set forth in SEQ ID NO: 15. Namely, the VH CDR sequences are GYTFTDYYMH (CDR-H1, SEQ ID NO: 236), RVNPNRRGTTYNOKFEG (CDR-H2, SEQ ID NO: 237), and ARANWLDY (CDR-H3, SEQ ID NO: 238).

Detecting cMET expression typically involves contacting a biological sample with one or more anti-cMET antibody, and detecting whether the sample is positive for cMET expression, or whether the sample has altered expression as compared to a control sample.

Other Exemplary Anti-cMET pH-Antibodies

In one embodiment, the anti-cMET antibody comprises the CDRs any one of an anti-cMET pH-ADC comprising one pair of heavy and light chain variable sequences as set forth in SEQ ID NOs: 15 & 16; 5 & 6; 7 & 8; 9 & 10; 11 & 12; 13 & 14; 17 & 18; 19 & 20; 21 & 22; 23 & 24; 25 & 26; 27 & 28; 29 & 30; 31 & 32; and 33 & 34. In particular embodiments, the anti-cMET pH-antibody may include CDRs and/or variable domains from antibodies exhibiting superior internalization (relative to non-pH-controls), such pH-antibodies notably including: MYT4326 (HCVD=SEQ ID NO: 15, LCVD=SEQ ID NO: 16); MYT4309 (HCVD=SEQ ID NO: 242, LCVD=SEQ ID NO: 8); MYT4310 (HCVD=SEQ ID NO: 243, LCVD=SEQ ID NO: 8); MYT4311 (HCVD=SEQ ID NO: 244, LCVD=SEQ ID NO: 8); MYT4318 (HCVD=SEQ ID NO: 245, LCVD=SEQ ID NO: 8); MYT4319 (HCVD=SEQ ID NO: 229, LCVD=SEQ ID NO: 8); MYT4320 (HCVD=SEQ ID NO: 230, LCVD=SEQ ID NO: 8); MYT4322 (HCVD=SEQ ID NO: 246, LCVD=SEQ ID NO: 8); MYT4323 (HCVD=SEQ ID NO: 247, LCVD=SEQ ID NO: 8); MYT4324 (HCVD=SEQ ID NO: 231, LCVD=SEQ ID NO: 8); MYT4325 (HCVD=SEQ ID NO: 9, LCVD=SEQ ID NO: 8); MYT4327 (HCVD=SEQ ID NO: 15, LCVD=SEQ ID NO: 248); MYT4332 (HCVD=SEQ ID NO: 15, LCVD=SEQ ID NO: 249); MYT4334 (HCVD=SEQ ID NO: 15, LCVD=SEQ ID NO: 250); and MYT4336 (HCVD=SEQ ID NO: 15, LCVD=SEQ ID NO: 251) (each disclosed in US 20220281984 A1). In a specific embodiment, the anti-cMET pH-antibody comprises HC CDRs 1-3 having the sequences as set forth in SEQ ID NOs: 236, 237, and 238, respectively; and the LC CDRs 1-3 having the sequences as set forth in SEQ ID NOs: 239, 240, and 241, respectively.

### Expression Systems and Methods of Making the Antibodies

Anti-cMET pH-antibodies can be prepared by recombinant expression, for example, as disclosed in US 20220281984 A1. To express the anti-cMET pH-antibodies, DNAs encoding heavy and light chains are inserted into expression vectors such that the corresponding genes are operatively linked to transcriptional and translational control sequences. As used herein, “operatively linked” means that an gene encoding an antibody is ligated into a vector such that vector control sequences exert their intended transcriptional and translational regulatory functions over the genes. The antibody light and heavy chain genes may be expressed in the same or different expression vectors. In cases where two expression vectors are used, the first vector may encode a heavy chain polypeptide and the second may encode a light chain polypeptide. The two vectors may comprise identical or separate selectable markers. Further, recombinant expression vectors carrying the anti-cMET pH-antibody chain gene sequence(s) can also carry regulatory sequences such as those that regulate vector replication in host cells and selectable marker genes. The vector(s) may then be transferred into suitable host cells by standard methods (e.g. transient transfections or stable cell generation) to produce the disclosed pH-antibodies.

Recombinant techniques may also be employed to remove portions of the DNA encoding the heavy and/or light chains that are not required for pH-dependent, cMET-specific binding. Molecules expressed from such truncated cMET-binding molecules are also encompassed by the pH-antibodies of the present disclosure.

After an anti-cMET pH-antibody or pH-dependent binding fragment thereof has been produced by recombinant expression, it can be purified by any method known in the art (e.g., chromatography, including ion exchange, affinity, and size exclusion chromatography, and the like), centrifugation, differential solubility, or the like. Once isolated, the anti-cMET pH-antibody or fragment may be further purified.

Specific Anti-cMET pH-Antibody Drug Conjugates

In specific embodiments, the anti-cMET pH-ADCs are compounds according to structural formula (I): [D-L-XY]n-Ab or salts thereof, where each “D” represents a cytotoxic and/or cytostatic agent (“drug”); each “L” represents a linker; “Ab” represents a pH-dependent anti-cMET antigen binding moiety; each “XY” represents a linkage formed between a functional group R^{x} on the linker and a “complementary” functional group R^{y} on the antigen binding moiety; and n represents the number of drugs linked to Ab of the ADC. In particular embodiments, n=2 and the linkers are conjugated to an anti-cMET pH-Ab via cysteine residues.

In some particular embodiments, each D is the same and/or each L is the same. Specific embodiments of constituent D and L elements are described in greater detail below.

In some embodiments, at least one polypeptide of any of the antibodies described herein is conjugated to the toxin, the radioisotope, or the drug via a non-cleavable linker. In some embodiments, the conjugated toxin, radioisotope, or drug is released during lysosomal and/or late endosomal degradation of the antibody.

Non-limiting examples of cleavable linkers include: hydrazone linkers, peptide linkers, disulfide linkers, and thioether linkers. See, e.g., Carter et al., Cancer J. 14(3):154-169, 2008; Sanderson et al., Clin. Cancer Res. 11(2 Pt1):843-852, 2005; Chari et al., Acc. Chem. Res. 41(1):98-107, 2008; Oflazoglu et al., Clin. Cancer Res. 14(19): 6171-6180, 2008; and Lu et al., Int. J. Mol. Sci. 17(4): 561, 2016.

Non-limiting examples of non-cleavable linkers include: maleimide alkane-linkers and maleimide cyclohexane linker (MMC) (see, e.g., those described in McCombs et al., AAPS J. 17(2):339-351, 2015).

In some embodiments, any of the antibodies described herein is cytotoxic or cytostatic to the target mammalian cell.

In some embodiments, the antibodies provided herein can comprise one or more amino acid substitutions to provide a conjugation site (e.g., conjugated to a drug, a toxin, a radioisotope). In some embodiments, the antibodies provided herein can have one conjugation site. In some embodiments, the antibodies described herein can have two conjugation sites. In some embodiments, the antibodies provided herein can have three or more conjugation sites. A non-limiting example of an amino acid substitution to produce a conjugation site (e.g., “a triple hinge” conjugation site) is described in U.S. Patent Application No. 2017/0348429, which is incorporated herein by reference in its entirety. For example, a lysine to cysteine substitution at amino acid position 105 and deletion of a threonine at amino acid positions 106 and 108 of SEQ ID NO: 155 or SEQ ID NO: 189 can provide a “triple hinge” conjugation site in any of the antibodies described herein. In some embodiments, an alanine to a cysteine substitution at amino acid position 1 of SEQ ID NO: 155 or SEQ ID NO: 189 can provide a conjugation site for any of the antibodies described herein. In some embodiments, a valine to cysteine substitution at amino acid position 98 of SEQ ID NO: 157 can provide a conjugation site for any of the antibodies described herein.

Naturally-occurring cysteine amino acids can also provide a conjugation (e.g., conjugated to a drug, a toxin, a radioisotope.). In some embodiments, the antibodies provided herein can have a drug, a toxin, or a radioisotope conjugated at one or more (e.g., one, two, three, or four) naturally-occurring conjugation sites. In some embodiments, the cysteine at amino acid position 103 of SEQ ID NO: 155 or 189 is a naturally occurring conjugation site. In some embodiments, the cysteine at amino acid position 109 of SEQ ID NO: 155 or 189 is a naturally occurring conjugation site. In some embodiments, the cysteine at amino acid position 112 of SEQ ID NO: 155 or SEQ 189 is a naturally-occurring conjugation site. In some embodiments, the cysteine at amino acid position 107 of SEQ ID NO: 157 is a naturally-occurring conjugation site.

In some embodiments, the antibodies provided herein can have a drug, a toxin, or a radioisotope conjugated at one or more (e.g., two, three, or four) naturally occurring conjugation sites, e.g., the cysteine at amino acid position 103, the cysteine at cysteine at amino acid position 109, and/or the cysteine at amino acid position 112 of SEQ ID NO: 155 or SEQ 189, and/or the cysteine at amino acid position 107 of SEQ ID NO: 157. In some embodiments, the antibodies provided herein can have a drug, a toxin, or a radioisotope conjugated at one or more (e.g., two, three, or four) naturally occurring conjugation sites and one or more (e.g., two, or three) engineered conjugation sites (e.g., engineered by amino acid substitutions, deletions, additions, etc.).

Conjugation through engineered cysteines is achieved by methods known in the art. Briefly, engineered cysteine-containing antibody is prepared for conjugation by treatment with a reducing agent, for example, tris (2-carboxyethyl) phosphine (TCEP), Dithiothreitol (DTT), or 2-Mercaptoethanol (BME). In the reduction reaction the reducing reagent with disulfide bonds in the antibody, breaking interchain disulfides and removing disulfide caps from the engineered cysteines. An optional reoxidation step, achieved by exposure of the solution to air, or an oxidizing agent such as dehydroascorbic acid, allows reformation of the interchain disulfide bonds, leaving the engineered cysteines with a thiolate reactive group. Conjugation with a maleimide functionality on the linker-payload, maleimide-vc-PAB-MMAE, is achieved by reaction with the payload in buffered solution, containing cosolvent such as ethanol, dimethylacetamide (DMA), or dimethyl sulfoxide (DMSO). The crude conjugated antibody solution is purified by size exclusion chromatography, or selective filtration methods, such as tangential flow filtration. In this step, residual unreacted payload, reducing agent and oxidizing agents are removed from the reaction mixture, and the conjugated ADC product may be transferred into a desirable formulation buffer.

Conjugation through hinge cysteines is achieved by similar methods, using antibodies with, or without, additional engineered cysteine conjugation sites. Briefly, the antibody is prepared for conjugation by treatment with a reducing agent, for example, tris (2-carboxyethyl) phosphine (TCEP) or Dithiothreitol (DTT). The reducing strength and concentration of the reducing agent are selected such that some or all of the interchain disulfide bonds are reduced leaving free cysteines for conjugation. The solution may be directly conjugated in the presence of excess reducing agent. Conjugation with a maleimide functionality on the linker-payload, maleimide-vc-PAB-MMAE, is achieved by reaction with the payload in buffered solution, containing cosolvent such as ethanol, dimethylacetamide (DMA), or dimethyl sulfoxide (DMSO). Unreacted linker-payload may be rendered non-reactive by addition of a sacrificial thiolate molecule such as acetyl-cysteine. The crude conjugated antibody solution may be further purified by methods known in the art, including hydrophobic interaction chromatography, ion-exchange chromatography, or mixed-mode chromatography such as ceramic hydroxyapatite chromatography. Isolation of chromatography fractions allows selection of the desired antibody to payload ratio and removal of unreacted antibody, protein aggregates and fragments, and payload-related reaction side products. The purified antibody drug conjugate may be further purified and by size exclusion chromatography, or selective filtration methods, such as tangential flow filtration. In this step the conjugated ADC product may also be transferred into a desirable formulation buffer.

In some examples, an antibody conjugate can be made comprising an antibody linked to monomethyl auristatin E (MMAE) via a valine-citrulline (vc) linker (hereafter, Met-IgG-DC). Conjugation of the antigen-binding protein construct with vcMMAE begins with a partial reduction of the Met-IgG followed by reaction with maleimidocaproyl-Val-Cit-PAB-MMAE (vcMMAE). The Met-IgG (10 mg/mL) is partially reduced by addition of TCEP (molar equivalents of TCEP:mAb is 2:1) followed by incubation at 4° C. overnight. The reduction reaction is then warmed to 25° C. To conjugate all of the thiols, vcMMAE is added to a final vcMMAE:reduced Cys molar ratio of 1:10. The conjugation reaction is carried out in the presence of 10% v/v of Dimethylacetamide (DMA) and allowed to proceed at 25° C. for 60 minutes.

In some examples, an antibody conjugate (ADC) is made comprising the Met-binding IgG (hereafter, Met-IgG) described herein linked to monomethyl auristatin E (MMAE) via a valine-citrulline (vc) linker (hereafter, Met-IgG-DC). Conjugation of the antigen-binding protein construct with vcMMAE begins with a partial reduction of the Met-IgG followed by reaction with maleimidocaproyl-Val-Cit-PAB-MMAE (vcMMAE). The Met-IgG (10 mg/ml) is reduced by addition of DTT (molar equivalents of DTT:mAb is 100:1) followed by incubation at 25° C. overnight. The reduced Met-IgG (10 mg/mL) is then re-oxidized by exposure to DHAA (molar equivalents of DHAA:mAb is 10:1) followed by incubation at 25º C for 2 hours. To conjugate all of the thiols, vcMMAE is added to a final vcMMAE:mAb molar ratio of 4:1. The conjugation reaction is carried out in 10% v/v DMA and allowed to proceed at 25° C. for 3 hours.

In a particular exemplary embodiment of the anti-cMET pH-ADC, each “D” is the same and is a cell-permeating auristatin (e.g. MMAE); each “L” is the same and is a linker cleavable by a endolysosomal and/or lysosomal enzyme; each “XY” is a linkage formed between a maleimide and a sulfhydryl group; “Ab” is an antibody comprising six CDRs corresponding to the six CDRs of antibody MYTX-011, or an antibody that competes for binding cMET with such an antibody; and n is 2.

In a particular exemplary embodiment, the ADC according to formula (I) has the structure of formula (II):

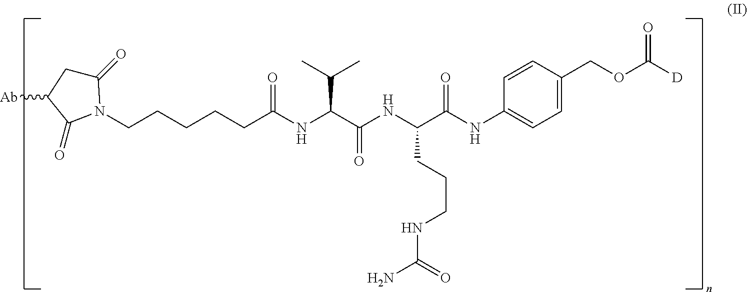

In one embodiment, the Ab in the compound of formula (II) is Q397.

In a particular embodiment, the compound of formula (I) has the following structure (III):

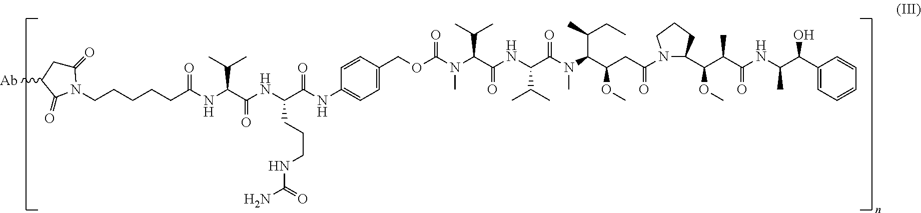

or a pharmaceutically acceptable salt thereof, wherein n has an average value of 2, or is equal to 2, and the Ab is a full length anti-cMET pH-antibody. In some embodiments, the anti-cMET pH-antibody is Q397.
Cytotoxic and/or Cytostatic Agents

The cytotoxic and/or cytostatic agents may be any agents known to inhibit the growth and/or replication of and/or kill cells, and in particular cancer and/or tumor cells. Non-limiting examples of classes of such agents include, by way of example and not limitation, radionuclides, alkylating agents, DNA cross-linking agents, DNA intercalating agents (e.g., groove binding agents such as minor groove binders), cell cycle modulators, apoptosis regulators, kinase inhibitors, protein synthesis inhibitors, mitochondria inhibitors, nuclear export inhibitors, topoisomerase I and II inhibitors, RNA/DNA antimetabolites, and antimitotic agents.

As mentioned, anti-cMET pH-ADCs generally comprise an anti-cMET antigen binding moiety, such as an anti-cMET antibody and/or binding fragment, having one or more cytotoxic and/or cytostatic agents, which may be identical or different, linked thereto by identical or different linker(s). Each antibody may also be conjugated or linked to multiple different cytotoxic or cytostatic agents, each agent having the same or different biological effects.

In some embodiments, the antibodies provided herein can be conjugated to a drug (e.g., a chemotherapeutic drug, a small molecule), a toxin, or a radioisotope. Non-limiting examples of drugs, toxins, and radioisotopes (e.g., known to be useful for the treatment of cancer) are known in the art. Particular non-limiting examples of drugs/agents are provided below.

Angiogenesis Inhibitors: ABT-869; AEE-788; axitinib (AG-13736); AZD-2171; CP-547,632; IM-862; pegaptamib; sorafenib; BAY43-9006; pazopanib (GW-786034); vatalanib (PTK-787, ZK-222584); sunitinib; SU-11248; VEGF trap; vandetanib; ABT-165; ZD-6474; DLL4 inhibitors.

Apoptosis Regulators: AT-101 ((−)gossypol); G3139 or oblimersen (Bcl-2-targeting antisense oligonucleotide); IPI-194; IPI-565; GX-070 (Obatoclax®); HGS1029; GDC-0145; GDC-0152; LCL-161; LBW-242; venetoclax; TRAIL or death receptor targeting agents (e.g., DR4 and DR5) such as ETR2-ST01, GDC0145, HGS-1029, LBY-135, PRO-1762; caspase-targeting drugs, caspase-regulators, BCL-2 family members, TNF family members, Toll family members, and/or NF-kappa-B proteins.

Antimitotic Agents: allocolchicine; auristatins, such as MMAE (monomethyl auristatin E) and MMAF (monomethyl auristatin F); halichondrin B; cemadotin; colchicine; any colchicine derivative; dolastatin-10; dolastatin-15; maytansine; maytansinoids, such as DM1 (N2′-deacetyl-N2′-(3-mercapto-1-oxopropyl)-maytansine); rhozoxin; paclitaxel or derivative thereof; docetaxel; thiocolchicine; trityl cysteine; vinblastine sulfate; vincristine sulfate.

Alkylating Agents: asaley; AZQ; BCNU (N,N′-Bis(2-chloroethyl)-N-nitrosourea); busulfan (1,4-butanediol dimethanesulfonate); (carboxyphthalato)platinum; CBDCA; CCNU; CHIP (iproplatin; NSC 256927); chlorambucil; chlorozotocin; cisplatin; clomesone; cyanomorpholinodoxorubicin; cyclodisone; dianhydrogalactitol (5,6-diepoxydulcitol); fluorodopan ((5-[(2-chloroethyl)-(2-fluoroethyl)amino]-6-methyl-uracil); hepsulfam; hycanthone; indolinobenzodiazepine dimer DGN462; melphalan; methyl CCNU ((I-(2-chloroethyl)-3-(trans-4-methylcyclohexane)-I-nitrosourea); mitomycin C; mitozolamide; nitrogen mustard ((bis(2-chloroethyl) methylamine hydrochloride); PCNU ((I-(2-chloroethyl)-3-(2,6-dioxo-3-piperidyl)-I-nitrosourea)); piperazine alkylator ((1-(2-chloroethyl)-4-(3-chloropropyl)-piperazine dihydrochloride)); piperazinedione; pipobroman (N,N′-bis(3-bromopropionyl) piperazine); porfiromycin (N-methylmitomycin C); spirohydantoin mustard; triglycidylisocyanurate; tetraplatin; thio-tepa (N,N′,N″-tri-1,2-ethanediylthio phosphoramide); triethylenemelamine; uracil nitrogen mustard; bis(3-mesyloxy propylamine hydrochloride.

Alkylating Antineoplastic Agents: Carboquone; Carmustine; Chlornaphazine; Chlorozotocin; Duocarmycin; Evofosfamide; Fotemustine; Glvrfosfamide; Lomustine; Mannosulfan; Nimustine; Phenanthriplatin; Pipobroman; Ranimustine; Semustine; Streptozotocin; ThioTEPA; Treosulfan; Triaziquone; and Triethylenemelamine.

DNA Alkylating-like Agents: Cisplatin; Carboplatin; Nedaplatin; Oxaliplatin; Satraplatin; Triplatin tetranitrate; Procarbazine; altretamine; dacarbazine; mitozolomide; temozolomide.

DNA replication and repair inhibitors: Altretamine; Bleomycin; Dacarbazine; Dactinomycin; Mitobronitol; Mitomycin; Pingyangmycin; Plicamycin; Procarbazine; Temozolomide; ABT-888 (veliparib); olaparib; KU-59436; AZD-2281; AG-014699; and BSI-201.

Cell Cycle Modulators: Paclitaxel; Nab-Paclitaxel; Docetaxel; Vincristine; Vinblastine; ABT-348; AZD-1152; MLN-8054; VX-680; Aurora A-specific kinase inhibitors; Aurora B-specific kinase inhibitors and pan-Aurora kinase inhibitors; AZD-5438; BMI-1040; BMS-032; BMS-387; CVT-2584; flavopyridol; GPC-286199; MCS-5A; PD0332991; PHA-690509; seliciclib (CYC-202, R-roscovitine); ZK-304709; AZD4877, ARRY-520; GSK923295A.

Kinase Inhibitors: Afatinib; Axitinib; Bosutinib; Crizotinib; Dasatinib; Erlotinib; Fostamatinib; Gefitinib; Ibrutinib; Imatinib; Lapatinib; Lenvatinib; Mubritinib; Nilotinib; Pazopanib; Pegaptanib; Sorafenib; Sunitinib; SU6656; Vandetanib; Vemurafenib; CEP-701 (lesaurtinib); XL019; INCB018424 (ruxolitinib); ARRY-142886 (selemetinib); ARRY-438162 (binimetinib); PD-325901; PD-98059; AP-23573; CCI-779; everolimus; RAD-001; rapamycin; temsirolimus; ATP-competitive TORC1/2 inhibitors including PI-103, PP242, PP30, Torin 1; LY294002; XL-147; CAL-120; ONC-21; AEZS-127; ETP-45658; PX-866; and the like.

Proteasome Inhibitors: Bortezomib; Carfilzomib; Epoxomicin; and Ixazomib.

Protein Synthesis Inhibitors: Amikacin; Arbekacin; Bekanamycin; Dibekacin; Dihydrostreptomycin; Streptomycin; Neomycin; Framycetin; Paromomycin; Ribostamycin; Kanamycin; Tobramycin; Spectinomycin; Hygromycin B; Paromomycin; Gentamicin; Netilmicin; Sisomicin; Isepamicin; Verdamicin; Astromicin; Tetracycline; Doxycycline; Chlortetracycline; Clomocycline; Demeclocycline; Lymecycline; Meclocycline; Metacycline; Minocycline; Oxytetracycline; Penimepicycline; Rolitetracycline; Tetracycline; Glycylcyclines; Tigecycline; Oxazolidinone; Eperezolid; Linezolid; Posizolid; Radezolid; Ranbezolid; Sutezolid; Tedizolid.

Peptidyl transferase inhibitors: Azidamfenicol; Chloramphenicol; Thiamphenicol; Florfenicol; Pleuromutilins; Retapamulin; Tiamulin; Valnemulin; Azithromycin; Clarithromycin; Dirithromycin; Erythromycin; Flurithromycin; Josamycin; Midecamycin; Miocamycin; Oleandomycin; Rokitamycin; Roxithromycin; Spiramycin; Troleandomycin; Tylosin; Ketolides; Telithromycin; Cethromycin; Solithromycin; Clindamycin; Lincomycin; Pirlimycin; Streptogramins; Pristinamycin; Quinupristin/dalfopristin; Virginiamycin.

Histone deacetylase (HDAC) inhibitors: Vorinostat; Romidepsin; Chidamide; Panobinostat; Valproic acid; Belinostat; Mocetinostat; Abexinostat; Entinostat; SB939 (pracinostat); Resminostat; Givinostat; Quisinostat; thioureidobutyronitrile (Kevetrin™); CUDC-10; CHR-2845 (tefinostat); CHR-3996; 4SC-202; CG200745; ACY-1215 (rocilinostat); etc.

Topoisomerase I Inhibitors: camptothecin and an array derivatives and analogs (e.g., NSC 100880, NSC 603071, and the like); morpholinisoxorubicin; SN-38.

Topoisomerase II Inhibitors: doxorubicin; amonafide (benzisoquinolinedione); m-AMSA (4′-(9-acridinylamino)-3′-methoxymethanesulfonanilide); anthrapyrazole derivative ((NSC 355644); etoposide (VP-16); pyrazoloacridine, 9-methoxy-N,N-dimethyl-5-nitro-, monomethanesulfonate); bisantrene hydrochloride; daunorubicin; deoxydoxorubicin; mitoxantrone; menogaril; N,N-dibenzyl daunomycin; oxanthrazole; rubidazone; teniposide.

DNA Intercalating Agents: anthramycin; chicamycin A; tomaymycin; DC-81; sibiromycin; pyrrolobenzodiazepine derivative; SGD-1882.

RNA/DNA Antimetabolites: L-alanosine; 5-azacytidine; 5-fluorouracil; acivicin; aminopterin derivative; L-aspartic acid (NSC 132483); aminopterin derivative; antifolate PT523; Baker's soluble antifol (NSC 139105); dichlorallyl lawsone ((2-(3,3-dichloroallyl)-3-hydroxy-1,4-naphthoquinone); brequinar; ftorafur ((pro-drug; 5-fluoro-I-(tetrahydro-2-furyl)-uracil); 5,6-dihydro-5-azacytidine; methotrexate; methotrexate derivative; PALA ((N-(phosphonoacetyl)-L-aspartate); pyrazofurin; trimetrexate.

DNA Antimetabolites: 3-HP; 21-deoxy-5-fluorouridine; 5-HP; a-TGDR (a-T-deoxy-6-thioguanosine); aphidicolin glycinate; ara C (cytosine arabinoside); 5-aza-2′-deoxycytidine; β-TGDR (P-2′-deoxy-6-thioguanosine); cyclocytidine; guanazole; hydroxyurea; inosine, glycodialdehyde; macbecin II; pyrazoloimidazole; thioguanine; thiopurine.

Mitochondria Inhibitors: pancratistatin; phenpanstatin; rhodamine-123; edelfosine; d-alpha-tocopherol succinate; compound 11 β; aspirin; ellipticine; berberine; cerulenin; GX015-070 (Obatoclax®; IH-Indole, 2-(2-((3,5-dimethyl-IH-pyrrol-2-yl)methylene)-3-methoxy-2H-pyrrol-5-yl)-); celastrol (tripterine); metformin; Brilliant green; ME-344.

Nuclear Export Inhibitors: callystatin A; delactonmycin; KPT-185; kazusamycin A; leptolstatin; leptofuranin A; leptomycin B; ratjadone; and Verdinexor.

Hormonal Therapies: anastrozole; exemestane; arzoxifene; bicalutamide; cetrorelix; degarelix; deslorelin; trilostane; dexamethasone; flutamide; raloxifene; fadrozole; toremifene; fulvestrant; letrozole; formestane; glucocorticoids; doxercalciferol; sevelamer carbonate; lasofoxifene; leuprolide acetate; megesterol; mifepristone; nilutamide; tamoxifen citrate; abarelix; prednisone; finasteride; rilostane; buserelin; luteinizing hormone releasing hormone (LHRH); Histrelin; trilostane or modrastane; fosrelin; goserelin.

Any of the foregoing agents may be included in the disclosed anti-cMET pH-ADCs.

In some embodiments, the anti-cMET pH-ADC may include a membrane-permeating cytotoxic and/or cytostatic agent that is cytotoxic and/or cytostatic to both cMET+ tumors and cMET-negative tumor cells. Such pH-ADCs may exhibit a potent and effective “bystander effect”, whereby cells surrounding cMET+ cells, irrespective of their cMET expression levels, are impacted due to their proximity to the cMET+ cells.

In a particular embodiment, the agent is a cell-permeable antimitotic agent.

In another particular embodiment, the agent is a cell-permeable auristatin, such as, for example, MMAE.

In some embodiments, the linkers linking the agent to the pH-antibody may be cleavable or noncleavable.

Cleavable linkers may include enzymatically or chemically unstable or degradable linkages, which may be stable in the bloodstream, but become unstable when the ADCs are internalized into cells. Notable cleavable linkers include acid-labile groups that remain intact during systemic circulation in the blood's physiologic pH (about pH 7.3-7.5) and release the agent once the ADC is taken into pH 5.0-6.5 endosomal and pH 4.5-5.0 lysosomal compartments of the cell. Cleavable linkers may also include a disulfide group, such that they are stable at physiologic pH, but then release the agent upon internalization of the ADC.

Linkers may also include those that are cleavable by specific enzymes and include, for example, regions that are acted upon by enzymes present inside cells. Such linkers are typically more stable in plasma when compared to chemically labile linkers. Release of a drug/payload from an antibody occurs due to specific action of lysosomal proteases, which may be overexpressed in particular tumor cells.

In particular embodiments, the cleavable element is a dipeptide selected from Val-Cit, Val-Ala, and any other suitable dipeptide.

Multiple dipeptide-based cleavable linkers have been used to link antibodies to drugs including auristatin/auristatin family members, campotothecin, doxorubicin, mitomycin, and tallysomycin (see, e.g., Dubowchik et al, 1998, J. Org. Chem. 67: 1866-1872; Dubowchik et al, 1998, Bioorg. Med. Chem. Lett. 8(21):3341-3346; Walker et al, 2002, Bioorg. Med. Chem. Lett. 12:217-219; Walker et al, 2004, Bioorg. Med. Chem. Lett. 14:4323-4327; and Francisco et al, 2003, Blood 102: 1458-1465, Dornina et al, 2008, Bioconjugate Chemistry 19: 1960-1963, each of which is incorporated herein by reference in its entirety). Any such dipeptide linkers, or modified versions thereof, may be included in the ADCs described herein. Notable clinically-important examples include, but are not limited to, Brentuximab Vedotin SGN-35 (ADCETRIS™), Celldex Therapeutics glembatumumab (CDX-011) (anti-NMB, Val-Cit-MMAE), and Cytogen PSMA-ADC (PSMA-ADC-1301) (anti-PSMA, Val-Cit-MMAE), and SGN-75 (anti-CD-70, VC-MMAF).

Non-cleavable linkers may also be included in the pH-ADCs disclosed herein. Unlike cleavable linkers, the release of drug/payload from non-cleavable linkers does not depend on differential environmental properties. Instead, payload “release” is proposed to accompany antibody degradation in the lysosomes. As such, inclusion of non-cleavable linkers gives rise to amino acid drug metabolites, which tend to be more hydrophilic and less membrane permeable compared to drugs liberated from cleavable linkers. Accordingly, ADCs including non-cleavable linkers are associated with reduced bystander effects and non-target-specific toxicities relative to ADCs including cleavable linkers. On the other hand, ADCs that include noncleavable linkers tend to be more stable in the bloodstream versus ADCs having cleavable linkers.

Linker-drug modules may be conjugated to antibodies via a variety of attachment groups to generate ADCs. Generally, such groups may be electrophiles, e.g. maleimide groups, activated disulfides, active esters including NHS esters, acid halides, alkyl and benzyl halides such as haloacetamides, and haloformates.

As regards selecting a linker for a particular ADC, factors to consider include, for example, the site of attachment to the antibody and drug properties, including structural constraints and lipophilicity. For a review, see Nolting, Chapter 5 “Linker Technology in Antibody-Drug Conjugates,” In: Antibody-Drug Conjugates: Methods in Molecular Biology, vol. 1045, pp. 71-100, Laurent Ducry (Ed.), Springer Science & Business Medica, LLC, 2013.

In specific embodiments, the linker is selected to impact, enhance, and/or augment the cytotoxic bystander effect of the anti-cMET pH-ADCs.

Anti-cMET pH-ADC

As described throughout the specification, MYTX-011 is an ADC comprised of the cMET-targeting antibody Q397 conjugated to the potent cytotoxin MMAE through a vc linker. MYTX-011 is being tested in a Phase I clinical trial (see Example 10) with a DAR of ^{˜}2.0.

Methods of Producing Anti-cMET pH-Antibody Drug Conjugates

The pH-ADCs described herein may be produced using well-known synthetic techniques. Generally, pH-ADCs according to formula (I) may be prepared as follows: D-L-R^{x}+Ab-R^{y} à (I) [D-L-XY]ₙ-Ab; where D, L, Ab, XY and n are as defined supra, and R^{x} and R^{y} are complementary groups that can form covalent linkages with one another. In particular embodiments, the pH-antibody is engineered to include amino acid residues for conjugation. For example, a C may be included at the amino acid position corresponding to amino acid position 98 of the light chain constant domain sequence set forth in SEQ ID NO: 158, resulting in a DAR 2.0 pH-ADC.

### Compositions

The pH-ADCs described herein may be formulated as compositions comprising the pH-ADC plus one or more pharmaceutically acceptable carrier, excipient and/or diluent. The form and precise components of the composition will depend upon the intended uses and route of administration.

The compositions may be disposed in a sterile vial or a pre-loaded syringe.

In some embodiments, the compositions are formulated for different routes of administration (e.g., intravenous, subcutaneous, intramuscular, or intratumoral). In some embodiments, the compositions can include a pharmaceutically acceptable carrier (e.g., phosphate buffered saline). Single or multiple administrations of any of the compositions described herein can be given to a subject depending on, for example: the dosage and frequency as required and tolerated by the patient. A dosage of the pharmaceutical composition should provide a sufficient quantity of the antibody to effectively treat or ameliorate conditions, diseases, or symptoms.

Also provided herein are methods of treating a subject having a cancer (e.g., any of the cancers described herein) that include administering a therapeutically effective amount of at least one of any of the compositions or pharmaceutical compositions provided herein.

Also provided herein are kits that include any of the pH-ADCs described herein, any of the compositions described herein, or any of the pharmaceutical compositions described herein. In some embodiments, the kits can include instructions for performing any of the methods described herein. In some embodiments, the kits can include at least one dose of any of the compositions described herein. In some embodiments, the kits can provide a syringe for administering any of the pharmaceutical compositions described herein.

The compositions may be supplied in bulk form for multiple administrations. Pharmaceutical compositions may take the form of lyophilized formulations or aqueous solutions, comprising selected excipients, including but not limited to buffering agents, preservatives, stabilizing agents, non-ionic detergents, and antioxidants. See, Remington's Pharmaceutical Sciences, 16th edition (Osol, ed. 1980) and Remington: The Science and Practice of Pharmacy, 22^{nd} Edition (Edited by Allen, Loyd V. Jr., 2012).

Suitable buffering agents include acetate, citrate, fumarate, gluconate, lactate, oxalate, succinate, and tartrate buffers.). Buffering agents may also include, histidine buffers, phosphate buffers, and Tris.

Preservatives are typically included in amounts from about 0.2%-1% (w/v). Suitable preservatives may include benzyl alcohol, phenol, meta-cresol, methyl or propyl paraben, octadecyldimethylbenzyl ammonium chloride, benzalconium halides, catechol, hexamethonium chloride, and alkyl parabens, resorcinol, cyclohexanol, and 3-pentanol. Isotonicifiers (“stabilizers”) may also be added to ensure isotonicity and include sugar alcohols, including arabitol, erythritol, glycerin, mannitol, sorbitol, and xylitol. Stabilizers may include the foregoing isotonicifiers and/or amino acids (e.g. alanine, asparagine, arginine, glycine, glutamine, histidine, lysine, ornithine, L-leucine, glutamic acid, 2-phenylalanine, threonine), or any other suitable stabilizer.

Non-ionic surfactants or detergents (or “wetting agents”) may be added to reduce surface adsorption and to help solubilize the ADC and to protect the ADC against aggregation.

A particular embodiment of a composition suitable for administration via intravenous infusion comprises 10 mg/ml anti-cMET pH-ADC, 20 mM histidine, pH 5.5, 8.8% (w/v) trehelose dihydrate, 0.02% (w/v) polysorbate 80. The composition may be in the form of a lyophilized powder that, upon reconstitution with a suitable amount of sterile water or other acceptable solution for infusion or injection provides the above composition.

### Methods of Use

Data provided herein demonstrate that anti-cMET pH-ADCs exert potent antitumor activity against cMET+/cMET-expressing and cMET-overexpressing tumors in vivo. Accordingly, the pH-ADCs and/or pharmaceutical compositions comprising the pH-ADCs may be used therapeutically to treat cMET+ and/or cMET-overexpressing tumors.

Generally, the methods involve administering a therapeutically effective amount of the pH-ADCs to a patient having a cMET+ or cMET-overexpressing tumor. Any method known to skilled artisans for assessing cMET receptor protein expression levels in a cell may be used. A particular method for determining cMET expression levels is detailed in Example 15.

The IHC score (0, 1+, 2+, and 3+) and/or H-scores (0-300) may be assessed by suitable methods known by skilled artisans. In some embodiments, patients having tumors exhibiting any detectable amount of cMET expression are selected for treatment. In one embodiment, patients with H-scores>100 or 150 and/or IHC scores 1+, 2+ and 3+ are selected for treatment.

Patients selected for the pH-ADC treatments may have any cMET+ and/or cMET-overexpressing solid tumor (including tumors exhibiting HGF overexpression and/or having abnormal HGF/cMET signaling or expression). More particular examples include: breast cancers; cervical cancers; colorectal cancers; gastric carcinomas; head and neck cancers; lung cancers; pancreatic cancers; ovarian cancers; stomach cancers; kidney cancers; adrenal cancers; gastro/esophageal cancers; gliomas; liver cancers; medulloblastomas; melanomas; myxoid liposarcomas; prostate cancer; salivary gland tumors; sarcomas; parathyroid gland adenocarcinomas; endometrial cancers; epithelioid mesotheliomas; appendix carcinomas; goblet cell carcinomas; anaplastic large cell lymphoma (ALCL); and/or any malignancy including (e.g. relapsed, advanced, and/or refractory subtypes of the recited cancers).

In one classification system, lung cancer encompasses adenocarcinoma (acinar, micropapillary, mixed, papillary, solid, and lepidic mucinous or nonmucinous), squamous cell carcinoma, large cell carcinoma (e.g., NSCLC (e.g., advanced or non-advanced, LCNEC, LCNEM, NSCLC—not otherwise specified/adenosquamous carcinoma, sarcomatoid carcinoma, adenosquamous carcinoma, and large-cell neuroendocrine carcinoma); and small cell lung cancer or SCLC. In another classification system, lung cancer may be categorized as a preinvasive lesion, a minimally invasive adenocarcinoma, or an invasive adenocarcinoma.

More typically, lung cancer is categorized as either non-small cell lung cancer (“NSCLC”) or small cell lung cancer (“SCLC”) or. NSCLCs may be subclassified as squamous or non-squamous (nsNSCLC), and an example of a nsNSCLC is adenocarcinoma.

The cancer may be naive to treatment, or may be relapsed and/or refractory, or a metastasis of a cMET+ or cMET-overexpressing tumor. As disclosed herein (e.g., Examples 5 & 6), cMET+ cancer cells resistant to prior treatments may be sensitive to the anti-cMET pH-ADCs.

In one embodiment, the anti-cMET pH-ADCs provide clinical benefit on Objective Response Rate (ORR), Progression-Free Survival (PFS), Duration of Response (DOR), and/or Overall Survival (OS). In some embodiments, the benefit is a Complete Response (CR), a Partial Response (PR), or stable disease (SD) (each benefit as defined by RECIST version 1.1 criteria).

Methods for assessing therapeutic benefit are detailed in the Examples, and include, for example, (1) the Response Evaluation Criteria In Solid Tumors (RECIST) version 1.1; (2) the Eastern Cooperative Oncology Group (ECOG) Performance Status; (3) immune-related response criteria (irRC); (4) disease evaluable by assessment of tumor antigens; (5) Kaplan-Meier estimates for OS and PFS; and/or patient reported outcome scales.

Important clinical trial endpoints include tumor shrinkage (an OR) and time to disease progression. RECIST criteria are often used in trials where OR is the primary endpoint, and/or when stable disease, tumor progression, or time to progression analyses are undertaken.

Secondary outcome measures for determining the therapeutic benefit of the anti-cMET pH-ADCs may include ORR, PFS, DOR, and Depth of Response (DpR).

The therapeutic benefit of the anti-cMET pH-ADCs may also be determined using any one or combinations of the following tumor antigens and/or biomarkers: 5-HIAA, acid phosphatase, ApoE, ACTA2, ACTH, ADGRE1, EMR1, AIF1, AKR1C1, AKR1C2, alkaline phosphatase, alpha-fetoprotein (AFP), CA-125, alpha-HCG, and alpha-TSH, ANGPTL4, ANGPTL4, beta-HCG, BNIP3, C1QA, C1QB, C-212, CA15-3, CA-195, CA19-9, CA-549, CADM1, calcitonin, catecholamines, cathepsin-D, CCL5, CCL5, CCR5, CD1 Ib, CD1 Ic, CD16, CD19, CD3, CD3, CD4, CD40, CD45 (PTPRC), CD49D (ITGA4), CD5, CD68, CD7, CD74, CD8, CD80, CD86, CDCP1, CDH11, CEA, chromagranin-A, c-Myc, COL6A3, COL7A1, CSF1R, CTGF, CTLA-4, CTSD, CTSS, CXCL10, CXCL10, CXCL11, DDIT4, EGFR, EGLN, EGLN3, ERA (estrogen receptor assay), ERBB2 (HER2/neu), F4/80), ferritin, gastrin, GZMB, hCG, HLA-DR, HMOX1, HVA, IFI6, IFNG, IGFBP3, IL10RA1, IL-6, KISS1R, KRT33A, LDHI-5, LOX, LRRC15, LUM, Ly86, MCPT8, MHC-Class II, MMP10, MMP14, MS4A7, NOG, NSE (neuron specific enolase), pancreatic polypeptide, PD-1, PDGFRA, PDK1, PDL-1, PFKFB3, PGF, PGK1, PIK3AP1, PIK3CD, PLAP, PLOD2, PLP, PRA (progesterone receptor A), proinsulin C-peptide, PSA, SCC, SERPINE1, SMA, plasma soluble cMET (sMET) levels, STAT1, STC2, TCF4, TCRa, TCRy5, TDT, TGF, TGFB1, TGFB2, TGFBR1, thyroglobulin, TP A, VEGFA, and/or WNT5A. Other exemplary markers include FGFR-1, FGFR-2, FGFR-3, FGFR-4, CLDN18.2 (Claudin 18.2), TP53, EPHA1 (Ephrin A2), Integrin αvβ5, Integrin αvβ6, Mucin-16 (MUC-16, aka CA-125), MSLN (Mesothelin), TGFBR2, ALK, ROS1, NTRK-1, NTRK-2, NTRK-3, CUL5 (Cullin5). Such antigens and/or biomarkers may be evaluated at the DNA, RNA or protein level using DNA/RNA sequencing, gene microarrays, PCR, flow cytometry or IHC methods known to skilled artisans.

One particular therapeutic benefit comprises a Complete Response (CR). Another benefit comprises a Partial Response (PR).

### Adjunctive Therapies

In some embodiments, the anti-cMET pH-ADCs may be used with or adjunctive to other anti-cancer agents and/or treatment regimens. In some embodiments, the anti-cMET and other agent(s) may be co-formulated or separately formulated, and administered via single or different dosing regimens.

Other agents include, for example, alkylating agents, angiogenesis inhibitors, antibodies, antimetabolites, antimitotics, antiproliferatives, antivirals, aurora kinase inhibitors, ALK kinase inhibitors (e.g., crizotinib (XALKORI®), ceritinib (ZYKADIA®), and alectinib (ALECENSA®), apoptosis promoters (for example, Bcl-2 family inhibitors), activators of death receptor pathway, Bcr-Abl kinase inhibitors, BiTE antibodies, ADCs, CDK inhibitors, cell cycle inhibitors, Cox-2 inhibitors, DVDs, ErbB2 receptor inhibitors, GF inhibitors, HSP-90 inhibitors, HDAC inhibitors, hormones, immunologicals, inhibitors of inhibitors of apoptosis proteins (IAPs), intercalating antibiotics, kinesin inhibitors, Jak2 inhibitors, mTOR inhibitors, microRNAs, MEK inhibitors, NSAIDs, PARP inhibitors, platinum chemotherapeutics, polo-like kinase (Plk) inhibitors, PI3K inhibitors, proteasome inhibitors, purine analogs, pyrimidine analogs, RTK inhibitors, retinoids/deltoids plant alkaloids, siRNAs, topoisomerase inhibitors, ubiquitin ligase inhibitors, and the like, and/or combinations thereof.

Anti-cMET pH-ADCs may also be used to enhance the efficacy of radiation therapy, e.g., external beam, internal (i.e., brachy therapy) and systemic radiation therapy.

Anti-cMET pH-ADCs may be administered with or adjunctive to the following: ABRAXANE™ (ABI-007), ABT-100 (farnesyl transferase inhibitor), ADVEXIN®, ALTOCOR® or MEVACOR® (lovastatin), AMPLIGEN®, APTOSYN® (exisulind), AREDIA® (pamidronic acid), arglabin, L-asparaginase, atamestane (1-methyl-3,17-dione-androsta-1,4-diene), AVAGE® (tazarotene), AVE-8062 (combreastatin derivative) BEC2 (mitumomab), cachectin or cachexin (TNF), canvaxin, CEAVAC® (cancer vaccine), CELEUK® (celmoleukin), CEPLENE® (histamine dihydrochloride), CERVARIX® (HPV vaccine), CHOP® [C: CYTOXAN® (cyclophosphamide); H: ADRIAMYCIN® (hydroxydoxorubicin); O: Vincristine (ONCOVIN®); P: prednisone], CYPAT™ (cyproterone acetate), combrestatin A4P, DAB(389)EGF or TransMID-107R™ (diphtheria toxins), dacarbazine, dactinomycin, 5,6-dimethylxanthenone-4-acetic acid (DMXAA), eniluracil, EVIZON™ (squalamine lactate), DIMERICINE® (T4N5 liposome lotion), discodermolide, DX-895 If (exatecan mesylate), enzastaurin, EPO906 (epithilone B), GARDASIL®, GASTRIMMUNE®, GENASENSE®, GMK (ganglioside conjugate vaccine), GVAX® (prostate cancer vaccine), halofuginone, histrelin, hydroxycarbamide, ibandronic acid, IGN-101, IL-13-PE38, IL-13-PE38QQR (cintredekin besudotox), IL-13-pseudomonas exotoxin, interferon-a, interferon-γ, JUNO VAN™ or MEPACT™ (mifamurtide), lonafarnib, 5,10-methylenetetrahydrofolate, miltefosine, NEOVASTAT® (AE-941), NEUTREXIN® (trimetrexate glucuronate), NIPENT® (pentostatin), ONCONASE® (a ribonuclease enzyme), ONCOPHAGE®, ONCOVAX® (IL-2 Vaccine), ORATHECIN™ (rubitecan), OSIDEM®, OVAREX® MAb, paclitaxel, PANDIMEX™, panitumumab, PANVAC®-VF (vaccine), pegaspargase, PEG Interferon A, phenoxodiol, procarbazine, rebimastat, REMOVAB® (catumaxomab), REVLIMID® (lenalidomide), RSR13 (efaproxiral), SOMATULINE® LA (lanreotide), SORIATANE® (acitretin), staurosporine, talabostat (PT100), TARGRETIN® (bexarotene), TAXOPREXIN® (DHA-paclitaxel), TELCYTA® (canfosfamide, TLK286), temilifene, TEMODAR® (temozolomide), tesmilifene, thalidomide, THERATOPE® (STn-KLH), thymitaq, TNFERADE™, TRACLEER® or ZAVESCA® (bosentan), tretinoin (Retin-A), tetrandrine, TRISENOX®, VIRULIZEM®, ukrain, vitaxin, XCYTRTN® (motexafin gadolinium), XINLAY™ (atrasentan), XYOTAX™ (paclitaxel poliglumex), YONDELIS® (trabectedin), ZD-6126, ZINECARD® (dexrazoxane), ZOMETA′ (zolendronic acid), zorubicin, and combinations thereof.

Adjunctive therapies are typically administered according to their FDA label and/or manufacturer's recommendations, and it is envisioned that the anti-cMET pH-ADCs may produce optimal therapeutic benefit when administered once every two weeks, once every three weeks, or once every four weeks. In some embodiments, more or less frequent administration may also provide benefits.

### Administration Regimens and Dosages

The amount of anti-cMET pH-ADC administered will depend upon multiple factors, including, e.g., the particular tumor type and stage, the mode/route and frequency of administration, the nature of the ADC payload, and patient parameters including age, weight, and the like. Skilled artisans will appreciate how to optimize dosages using routine practices. In some embodiments, doses are estimated from in vivo animal models or clinical trials.

In some embodiments, administration of the anti-cMET pH-ADCs is via a route appropriate for the condition being treated. In some embodiments, the anti-cMET pH-ADC is administered via a parenteral route (e.g. infusion, intramuscular, subcutaneous, intravenous (IV), intradermal, intrathecal, bolus, intratumoral injection or epidural. In one embodiment, the anti-cMET pH-ADC is provided as a frozen liquid or a lyophilized powder in a vial. Each vial may contain, e.g., 0.5 mg, 1 mg, 5 mg, 10 mg, 50 mg, or 100 mg of the anti-cMET pH-ADC. In one embodiment, liquid is brought to a suitable administration volume with sterile water for injection (WFI) to provide a solution containing 10 mg/ml anti-cMET pH-ADC. In some embodiments, infusion is optimally performed once every 14 days, once every 21 days, or once every 28 days. In some optimal embodiments, the infusion is performed once every 21 days.

In one exemplary embodiment, an anti-cMET ADC is administered once every 21 days at about 0.3, 0.6, 0.9, 1.2, 1.5, 1.6, 1.8, 1.9, 2.1, 2.2, 2.4, 2.7, 3.0, 3.3, 3.6, 3.9, 4.2, 4.5, 4.8, 5.0, 5.2, 5.5, 5.8, 6.0, 6.2, 6.5, 6.8, 7.0, 7.2, 7.5, 7.8, 8.0, 8.2, 8.5, 8.8, 9.0, 9.2, 9.5, 9.8, or about 10.0 mg/kg of the subject's body weight. In one embodiment, the administration is at about 1.0 to about 8.0 mg/kg, at about 2.0 to about 7.0 mg/kg, at about 3.0 to about 6.0 mg/kg, or is at about 4.0 to about 5.5 mg/kg. In a particular embodiment, administration proceeds until disease remission, unacceptable toxicity, or disease progression.

In one embodiment, the cancer is a cMET+ or cMET-overexpressing NSCLC adenocarcinoma and the anti-cMET ADC is MYTX-011, administered at between about 2.0 and about 10.0 mg/kg or between about 4.0 mg/kg and about 8 mg/kg every 21 days. In other embodiments, the patient has an H-score between 150 to 224 or >225 and/or a tumor IHC score of 2+ or 3+. In another embodiment, the cancer is a squamous cell NSCLC carcinoma (scNSCLC), the anti-cMET pH-ADC is MYTX-011 administered at between about 4.0 and 6.0 mg/kg every 21 days, and the patient has an H-score between 100 to 224 or an IHC score of between about 1+ and about 2+. In another embodiment, an anti-cMET pH-ADC is administered once every 28 days.

In embodiments where additional agents are administered with the anti-cMET PH-ADC, the agent may be selected from cabazitaxel, colcemid, colchicine, cryptophycin, democolcine, docetaxel, nocodazole, paclitaxel, taccalonolide, taxane, and vinblastine.

In particular embodiments, an anti-cMET pH-ADC is used adjunctive to TARCEVA″ (erlotinib) or GILOTRIF® (afatinib) to treat NSCLC. Erlotinib may be administered orally at 150 mg/day, or afatinib may be administered orally at 40 mg/day, each until disease progression or no longer tolerated by the patient. In one embodiment, the afatinib is administered to patients with tumors having EGFR exon 19 deletions or exon 21 (L858R) substitution mutations. In another embodiment, erlotinib is administered to patients with tumors having EGFR-mutated adenocarcinoma. In a particular embodiment, erlotinib is administered to patients with EGFR wildtype adenocarcinoma tumors.

In another embodiment, an anti-cMET pH-ADC is used adjunctive to TAGRISSO® (osimertinib). The typical adult dose of osimertinib for NSCLC is 80 mg orally once a day. Osimertinib may be used as an adjuvant therapy after tumor resection whose tumors have epidermal growth factor receptor (EGFR) exon 19 deletions or exon 21 L858R mutations. Osimertinib may also be used for treatment of patients with metastatic NSCLC whose tumors have EGFR exon 19 deletions or exon 21 L858R mutations, and for treatment of patients with metastatic EGFR T790M mutation-positive NSCLC, whose disease has progressed on or after EGFR tyrosine kinase inhibitor (TKI) therapy.

In other embodiments, an anti-cMET pH-ADC is used adjunctive to one of the following agents to treat NSCLC (each pairing represents a specific embodiment): osimertinib (TAGRISSO®); IRESSA″ (gefitinib); OPDIVO® (nivolumab); OPDIVO® (nivolumab) and YERVOY® (ipilimumab); pembrolizumab (KEYTRUDA″); cisplatin to treat NSCLC; carboplatin; veliparib; veliparib and pemetrexed; cetuximab; ipilimumab (YERVOY″); radiation; AVASTIN® (bevacizumab); gemcitabine (GEMZAR®); afatinib (GIOTRIF®); axitinib (INLYTA®); bosutinib (BOSULIF®); crizotinib (XALKORI®); dasatinib (SPRYCE®); erlotinib (TARCEVA®); gefitinib (IRESSA®); imatinib (GLEEVEC®); lapatinib (TYVERB®); nilotinib (TASIGNA®); pazopanib (VOTRIENT®); ponatinib (ICLUSIG®); radotinib (SUPECT®); regorafenib (STIVARGA®); sorafenib (NEXAVAR®); sunitinib (SUTENT®); toceranib (PALLADIA®); and vatalanib.

In yet other embodiments, an anti-cMET pH-ADC is used adjunctive to one of the following agents to treat the indicated cancer (each pairing with each indication represents a specific embodiment): GEMZAR® to treat pancreatic, ovarian, breast, or NSCLC cancer; paclitaxel albumin-stabilized nanoparticle formulation (ABRAXANE″) to treat breast or lung cancer; ABRAXANE® plus GEMZAR® to treat pancreatic cancer; AVASTIN® (bevacizumab) to treat colorectal, lung, or ovarian cancer; or FOLFIRINOX (or FOLFIRI or FOLFOX or irinotecan (optionally ONIVYDE®) or 5-FU or capecitabine) to treat colorectal cancer.

For each of the foregoing, the anti-cMET pH-ADC may be administered at about 1.0 to about 10.0 mg/kg, about 2.0 to about 9.0 mg/kg, about 3.0 to about 8.0 mg/kg, about 4.0 to about 7.0 mg/kg, about 4.5 to 6.5 mg/kg, about 4.5 to 6.0 mg/kg, or about 5.0 mg/kg of body weight once every 14 or 21 days. The anti-cMET pH-ADC/adjunctive therapy is continued until disease remission, disease progression, or until it is no longer tolerated by the patient.

As will be appreciated by skilled artisans, the various agents described above may be administered at their recommended or customary doses, or the doses may be adjusted to maximize therapeutic benefit and/or to optimize patient response.

In alternative embodiments, all numbers expressing % purity, quantities of ingredients, and the like, used throughout this disclosure, are modified by the term “about”.

### Patient Selection

ADCs described herein may be administered to patients with cMET+ and cMET-overexpressing tumors, including but not solely, any solid tumor. Patients may be selected based upon the level of cMET present in their tumors, as classified by IHC assay (see Example 11). Briefly here, cMET levels are measured using the Ventana cMET (SP44) kit. Tissue samples are stained and then scored by determining the percentages of cells staining at various intensity levels. FIGS. 4A & 4B present representative sections having IHC scores of 1+, 2+, and 3+.

The anti-cMET pH-ADCs described herein may be used for a variety of purposes, and in one particular embodiment, the pH-ADCs may be used to treat cMET+, cMET-overexpressing, and/or MET-amplified tumors in humans. The pH-ADCs may also be used to treat tumors carrying Exon 14 mutations of the MET gene.

The anti-cMET pH-ADCs may also be used to treat patients having tumors carrying EGFR Exon 19 deletions and/or EGFR Exon 21 mutations (L858R) (Castañeda-González J P et al. Multiple mutations in the EGFR gene in lung cancer: a systematic review. Transl Lung Cancer Res. 2022 October; 11(10):2148-2163, which is herein incorporated by reference in its entirety).

The anti-cMET pH-ADCs may be used to treat a subject, including a human patient, previously identified or selected as having low-expression, intermediate expression, cMET-amplification, cMET exon mutation, or having cMET-positive and EGFR kinase inhibitor resistant cancer. In some embodiments, the method comprises the step of determining that the subject or human subject has low cMET expression, intermediate cMET overexpression, high cMET overexpression, cMET-amplification, a cMET exon mutation, or a cMET-positive and EGFR kinase inhibitor resistant cancer, and selecting the subject or human patient for treatment.

The anti-cMET pH-ADCs may also be used to treat a subject, including a human patient, wherein the subject or patient has unresectable, metastatic, or advanced solid tumors. In some embodiments, the subject or human patient was previously non-responsive to prior treatment and/or their cancer progressed on the prior treatment. In still other embodiments, the subject or human patient was non-responsive and/or had cancer that progressed upon treatment with an EGFR kinase inhibitor.

In some embodiments, a human patient (female or male) is selected for treatment with the anti-cMET pH-ADCs of the disclosure because the patient has histologically or cytologically confirmed locally advanced, recurrent, or metastatic NSCLC and has received available standard of care therapy, with any number of prior therapies. The patient also has at least one measurable cancer lesion per RECIST 1.1 and ideally an ECOG performance status of 0 or 1. Acute effects of prior therapy or surgical procedures should be resolved to ≤ Grade 1 or baseline (except alopecia, stable immune related toxicity such as hypothyroidism on hormone replacement, adrenal insufficiency on ≤ 10 mg daily prednisone (or equivalent) or anemia). The patient's cardiac LVEF is ≥50% by either echocardiogram or MUGA scan and there is adequate organ function as defined by: a) absolute neutrophil count (ANC) ≥ 1500 cells/mm³ (without growth factors within 1 week of first dose); b) Platelet count≥ 75,000/mm³ (platelet transfusion not allowed within 1 week prior to first dose of study drug administration); c) Hb≥ 9 g/dl (RBC transfusion not allowed within 1 week prior to first dose); d) International normalized ratio (INR) activated partial thromboplastin time (aPTT) and prothrombin time (PT) ≤ 1.5× treatment facility's ULN (but patient's on stable anticoagulant doses may be treated); e) calculated creatinine clearance>30 mL/min as calculated by the Modified Cockcroft-Gault formula; f) Total bilirubin≤ 1.5×ULN, ≤2.0×ULN for patients with Gilbert syndrome; g) AST and ALT≤ 2.5×ULN for patients without liver metastases; h) alkaline phosphatase≤ 1.5×ULN unless clearly attributable to a non-hepatic source; i) serum albumin≥ 3.0 g/dL j) HbA1C<7.5%. Diabetic patients should be on a stable dose of antidiabetic medications.

In some embodiments, patients are selected because they do not have an actionable EGFR mutation, optionally wherein their tumors have other driver mutations. Patients must have progressed on at least 1 line of prior therapy in the locally advanced/metastatic setting (multiple lines of tyrosine kinase inhibitor (TKI) for the same actionable mutation count as 1 line of therapy). Maintenance therapy is not considered a separate line of therapy. Adjuvant and neoadjuvant therapies count as 1 line of therapy if given within 6 months of treatment with the disclosed pH-ADCs. Patients without any actionable gene alteration must have progressed on (or be considered ineligible for), or be intolerant to, platinum-based chemotherapy and immune checkpoint inhibitor (as monotherapy or in combination with chemotherapy).

Patients with actionable gene alterations (other than EGFR) for which immune checkpoint inhibitor therapy is not SOC (e.g., anaplastic lymphoma kinase [ALK] translocation) must have progressed on (or be considered ineligible for), or be intolerant to, anticancer therapy targeting driver gene alterations and platinum-based chemotherapy.

Patients with actionable gene alterations (other than EGFR) for which immune checkpoint inhibitor is SOC must have progressed on (or be considered ineligible for), or be intolerant to, anticancer therapy targeting driver gene alternation and platinum-based chemotherapy, and also progressed on (or be considered ineligible for), or be intolerant to, immune checkpoint inhibitor (as monotherapy or in combination with platinum-based chemotherapy).

In some embodiments, the selected patients have histologically or cytologically confirmed locally advanced, recurrent (and not a candidate for curative therapy), or metastatic non-squamous NSCLC; and tumor sample with high cMET overexpression by IHC (3+ with tumor cell positivity of ≥50%) confirmed by central laboratory testing.

In some embodiments, the selected patients have histologically or cytologically confirmed locally advanced, recurrent (and not a candidate for curative therapy), or metastatic non-squamous NSCLC; and tumor sample with intermediate cMET expression by IHC (3+ with tumor cell positivity of ≥25% to <50%) confirmed by central laboratory testing.

In some embodiments, the selected patients have histologically or cytologically confirmed locally advanced, recurrent (and not a candidate for curative therapy), or metastatic squamous NSCLC; and tumor sample with cMET overexpression by IHC (2+ with tumor cell positivity of ≥25%) confirmed by central laboratory testing.

In some embodiments, the selected patients have histologically or cytologically confirmed locally advanced, recurrent (and not a candidate for curative therapy), or metastatic NSCLC; tumor sample that does not meet cMET IHC entry criteria for Cohorts A and B (for subjects with non-squamous NSCLC) and C (for subjects with squamous NSCLC) based on central laboratory testing; known MET amplification or exon 14 skipping mutations, respectively, performed in a CLIA-certified laboratory in the United States (US) or equivalently accredited diagnostic laboratory outside the US; and subjects with MET exon 14 skipping mutations must have received MET TKI therapy, if available and considered SOC.

In some embodiments, the selected patients have histologically or cytologically confirmed locally advanced, recurrent (and not a candidate for curative therapy), or metastatic NSCLC; evidence of cMET expression by IHC as documented in medical records; evidence of negative cMET expression by IHC would be excluded; and have previously received and had disease progression following either a cMET-targeted ADC or antibody therapy.

Response to MYTX-011 and other anti-cMET pH-ADCs disclosed herein may correlate with cMET expression at both the genomic and protein level.

### EMBODIMENTS

The following listing of exemplary aspects supports and is supported by the disclosure provided herein. Each “method of treating” embodiment may be reformulated to produce “use” embodiments. For example, a “method of treating condition Y comprising the step of administering an effective amount of composition X” is intended to include the phrases “use of a composition X for the treatment of condition Y”, “use of a composition X in the manufacture of a medicament for treating condition Y”, and the like.

Embodiment 1. A method of treating a cMET+, cMET-overexpressing, and/or MET-amplified solid tumor cancer, comprising administering an effective amount of an anti-cMET antibody drug conjugate (ADC) to a human subject having said cancer, over a sufficient period of time to provide a therapeutic benefit, wherein the antibody component of the ADC exhibits cMET-specific pH-dependent binding, and optionally wherein the antibody comprises heavy and light chain CDRs present in the amino acid sequences as set forth in one of the following pairs: SEQ ID NOs: 15 & 16; SEQ ID NOs: 5 & 6; SEQ ID NOs: 7 & 8; SEQ ID NOs: 9 & 10; SEQ ID NOs: 11 & 12; SEQ ID NOs: 13 & 14; SEQ ID NOs: 17 & 18; SEQ ID NOs: 19 & 20; SEQ ID NOs: 21 & 22; SEQ ID NOs: 23 & 24; SEQ ID NOs: 25 & 26; SEQ ID NOs: 27 & 28; SEQ ID NOs: 29 & 30; SEQ ID NOs: 31 & 32; and SEQ ID NOs: 33 & 34; optionally wherein the CDRs are determined using the Kabat or the IMGT system.

Embodiment 2. A method of treating a cMET-positive or cMET-overexpressing solid tumor cancer having low or intermediate expression of cMET, comprising administering an effective amount of an anti-cMET antibody drug conjugate (ADC) to a human subject previously identified or selected as having said cancer, over a sufficient period of time to provide a therapeutic benefit, wherein the antibody component of the ADC exhibits cMET-specific pH-dependent binding, and optionally wherein the antibody comprises heavy and light chain CDRs present in the amino acid sequences as set forth in one of the following pairs: SEQ ID NOs: 15 & 16; SEQ ID NOs: 5 & 6; SEQ ID NOs: 7 & 8; SEQ ID NOs: 9 & 10; SEQ ID NOs: 11 & 12; SEQ ID NOs: 13 & 14; SEQ ID NOs: 17 & 18; SEQ ID NOs: 19 & 20; SEQ ID NOs: 21 & 22; SEQ ID NOs: 23 & 24; SEQ ID NOs: 25 & 26; SEQ ID NOs: 27 & 28; SEQ ID NOs: 29 & 30; SEQ ID NOs: 31 & 32; and SEQ ID NOs: 33 & 34; optionally wherein the CDRs are determined using the Kabat or the IMGT system.

Embodiment 3. A method of treating a cMET-positive or cMET-overexpressing solid tumor cancer having a cMET immunohistochemistry score of 1+, 2+, or 3+, and/or an H-score of at least about 50 to 300, comprising administering an effective amount of an anti-cMET antibody drug conjugate (ADC) to a human subject previously identified or selected as having said cancer, over a sufficient period of time to provide a therapeutic benefit, wherein the antibody component of the ADC exhibits cMET-specific pH-dependent binding, and optionally wherein the antibody comprises heavy and light chain CDRs present in the amino acid sequences as set forth in one of the following pairs: SEQ ID NOs: 15 & 16; SEQ ID NOs: 5 & 6; SEQ ID NOs: 7 & 8; SEQ ID NOs: 9 & 10; SEQ ID NOs: 11 & 12; SEQ ID NOs: 13 & 14; SEQ ID NOS: 17 & 18; SEQ ID NOs: 19 & 20; SEQ ID NOs: 21 & 22; SEQ ID NOs: 23 & 24; SEQ ID NOs: 25 & 26; SEQ ID NOs: 27 & 28; SEQ ID NOs: 29 & 30; SEQ ID NOs: 31 & 32; and SEQ ID NOs: 33 & 34; optionally wherein the CDRs are determined using the Kabat or the IMGT system.

Embodiment 4. The method of any one of embodiments 1-3, wherein the cancer is non-small cell lung cancer (“NSCLC”), optionally selected from non-squamous NSCLC, squamous NSCLC, and not otherwise specified NSCLC. Optionally, the heavy chain and the light chain of the antibody component of the ADC comprises the heavy chain CDRs and the light chain CDRs present in SEQ ID NO: 15 and SEQ ID NO: 16, respectively, and/or the heavy chain comprises the sequence set forth in SEQ ID NO: 15 and the light chain comprises the sequence set forth in SEQ ID NO: 16.

Embodiment 5. The method of any one of embodiments 1-4, wherein a biopsy from a tumor of said cancer and/or the entire tumor itself, comprises at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100% of cancer cells having a cMET expression level of at least a 1+, a 2+, or a 3+, as scored by an applicable and/or regulatory-agency approved immunohistochemistry (IHC) assay.

Embodiment 6. The method of embodiment 5, wherein at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100% of the tumor cells have an IHC score of 1+ or 2+.

Embodiment 7. The method of embodiment 6, wherein at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100% of the tumor cells have an IHC score of 2+.

Embodiment 8. The method of embodiment 6, wherein at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100% of the tumor cells have an IHC score of 1+.

Embodiment 9. The method of embodiment 5, 6, or 8, wherein at least about 1%, 2%, 3%, 4%, 5%, 6%, 7%, 8%, 9%, 10%, 15%, or about 20% of the tumor cells have an IHC score of 1+ and wherein no more than about 1%, 2%, 3%, 4% or 5% of the tumor cells have an IHC score of 2+ or 3+.

Embodiment 10. The method of embodiment 9, wherein no more than about 1%, 2%, 3%, 4%, 5%, 6%, 7%, 8%, 9%, 10%, 15%, or about 20% of the tumor cells have an IHC score of 2+ and wherein no more than about 1% have an IHC score of 3+.

Embodiment 11. The method of embodiment 10, wherein no tumor cells have an IHC score of greater than 2+.

Embodiment 12. The method of embodiment 11, wherein no tumors cells have an IHC score of greater than 1+.

Embodiment 13. The method of any one of embodiments 1 to 5, further comprising the steps of determining an H-score for the tumor, wherein the H-score is between about 10 and about 250.

Embodiment 14. The method of embodiment 13, wherein the H-score is between about 20 and about 225.

Embodiment 15. The method of embodiment 14, wherein the H-score is between about 20 and about 200.

Embodiment 16. The method of embodiment 15, wherein the H-score is between about 20 and about 175.

Embodiment 17. The method of embodiment 16, wherein the H-score is between about 20 and about 150.

Embodiment 18. The method of embodiment 17, wherein the H-score is no more than

about 100.

Embodiment 19. The method of embodiment 18, wherein the H-score is no more than about 90.

Embodiment 20. The method of embodiment 18, wherein the H-score is no more than about 80.

Embodiment 21. The method of embodiment 18, wherein the H-score is no more than about 70.

Embodiment 22. The method of embodiment 18, wherein the H-score is no more than about 50.

Embodiment 23. The method of any one of embodiments 1-22, wherein the tumor is homogeneous for cMET expression and/or MET amplification.

Embodiment 24. The method of any one of embodiments 1-22, wherein the tumor is heterogeneous for cMET expression and/or MET amplification.

Embodiment 25. The method of embodiment 24, wherein at least about 10%, 20%, or 30% of the tumor cells have a first frequently occurring IHC score selected from one of 0, 1+, 2+, and 3+ and at least about 10%, 20%, or 30% of the tumor cells have a second frequently occurring IHC score selected from one of 0, 1+, 2+, and 3+, wherein the first and second frequently occurring IHC scores are different.

Embodiment 26. The method of embodiment 25, wherein the first and second IHC scores are selected from the following pairs of scores: (0, +1), (0, +2), (0, +3), (+1, +2), (+1, +3), and (+2, +3).

Embodiment 27. The method of embodiment 25 or 26, wherein at least about 10%, 20%, or 30% of the tumor cells have a third frequently occurring IHC score, wherein the third score is distinct from the first and second frequently occurring IHC scores.

Embodiment 28. The method of any one of the preceding embodiments, wherein the cancer has developed resistance to targeted therapies against one or more actionable mutation present in one or more gene selected from the group consisting of EGFR, ALK, KRAS, ROS, BRAF, NTRK1/2/3, MET, RET, ERBB2, and any other gene known to have an actionable mutation associated with the cancer.

Embodiment 29. The method of embodiment 28, wherein the actionable mutation is selected from one or more of the following: a) an EGFR gene mutation selected from an exon 20 T790M substitution, an exon 20 C797X substitution, an exon 21 L858R substitution, and an exon 19 deletion, optionally wherein the exon 19 deletion is the E746_A750 deletion as determined by a regulatory agency approved test; b) an ALK gene rearrangement; c) a KRAS gene mutation, optionally wherein the mutation is an exon 1 G12C substitution; d) a BRAF gene mutation, optionally wherein the mutation is in Exon 15 V600E substitution; e) a MET gene mutation, optionally wherein the mutation is an ex14 skipping mutation; f) a ROS1 gene rearrangement, optionally where the ROS1 gene is fused with a gene or portion of a gene selected from one of the following: CD74, EZR, SDC4, SLC34A2, CCCKC6, TFG, SLMAP, MYO5C, FIG, LIMA1, CLTC, GOPC, ZZCCHC8, CEP72, MLL3, KDELR2, LRIG3, MSN, MPRIP, WNK1, SLC6A17, TMEM106B, FAM135B, TPM3, and TDP52L1; g) a fusion of the NTRK1, 2, or 3 gene; and h) a RET1 gene rearrangement, optionally where the rearrangement is a fusion with KIF5B or CCDC6.

Embodiment 30. The method of any one of embodiments 1 to 27, wherein the cancer has an amplification, mutation, or overexpression of the ERBB2 gene, optionally wherein the ERBB2 gene has insertions in exon 20 and/or nucleotide substitutions encoding amino acid substitutions selected from one or more of the following: L755S, G776C, G660D, R678Q, E693K, and Q709L.

Embodiment 31. The method of any one of embodiments 1 to 27, wherein the anti-cMET ADC is administered as a monotherapy or adjunctive to an additional anticancer agent, wherein the additional agent is administered according to its regulatory agency-approved dosing regimen.

Embodiment 32. The method of embodiment 31, wherein the additional anticancer agent is an inhibitor and/or targeting agent of EGFR, ALK, KRAS, ROS, BRAF, NTRK1/2/3, MET, RET, or ERBB2.

Embodiment 33. The method of embodiment 31, wherein the additional anticancer agent is selected from osimertinib (TAGRISSO®), afatinib (GIOTRIF®), axitinib (INLYTA®), bosutinib (BOSULIF®), crizotinib (XALKORI®), dasatinib (SPRYCE®), erlotinib (TARCEVA®), gefitinib (IRESSA®), imatinib (GLEEVEC®), lapatinib (TYVERB®), nilotinib (TASIGNA®), pazopanib (VOTRIENT®), ponatinib (ICLUSIG®), radotinib (SUPECT®), regorafenib (STIVARGA®), sorafenib (NEXAVAR®), sunitinib (SUTENT®), toceranib (PALLADIA®), and vatalanib.

Embodiment 34. The method of embodiment 31, wherein the additional anticancer agent is capable of inhibiting EGFR comprising an exon 21 L858R substitution or an exon 19 E746_A750 deletion.

Embodiment 35. The method of embodiment 32, wherein the ALK inhibitor is selected from alectinib, brigatinib, lorlatinib, ceritinib, and crizotinib; the KRAS inhibitor is selected from sotorasib and adagrasib; the BRAF inhibitor is selected from dabrafinib, vemurafenib, and trametinib; the MET inhibitor is selected from tepotinib, crizotinib, and capmatinib; the ROS1 inhibitor is selected from entrectinib, crizotinib, ceritinib, and lorlatinib; the NTRK1/2/3 inhibitor is selected from larotrectinib and entrecteinib; the RET inhibitor is selected from selpercatinib, pralsetinib, and cabozantinib; the ERBB2 targeting agent is selected from transtuzumab-deruxtecan and trastuzumab-emtansine.

Embodiment 36. The method of embodiment 32, wherein the additional anticancer agent is an inhibitor of PD1, optionally an anti-PD1 antibody, optionally wherein the anti-PD1 antibody is pembrolizumab (Keytruda), nivolumab (Opdivo), or cemiplimab (Libtayo).

Embodiment 37. The method of embodiment 32, wherein the additional anticancer agent is an inhibitor of PD-L1, optionally an anti-PD-L1 antibody such as durvalumab, or azetolizumab.

Embodiment 38. The method of any one of the preceding embodiments, wherein the cancer is resistant to prior treatment with an anti-cMET antibody, an anti-cMET ADC, a chemotherapy, a small molecule directed against cMET, and/or a radiation therapy.

Embodiment 39. The method of any one of the preceding embodiments, wherein the anti-cMET ADC is administered in an amount ranging from about 0.5 mg/kg to about 6.0 mg/kg, about 2.0 mg/kg to about 5.0 mg/kg, or about 4.5 mg/kg once every three weeks (Q3W).

Embodiment 40. The method of any one of the preceding embodiments, wherein the anti-cMET ADC comprises an anti-cMET antibody linked the drug component of the ADC by way of a linker.

Embodiment 41. The method of embodiment 40, wherein the anti-cMET antibody is a full-length antibody.

Embodiment 42. The method of embodiment 40, wherein the anti-cMET antibody binds to cMET in vitro or in vivo with at least a 5-fold, 6-fold, 7-fold, 8-fold, 9-fold, 10-fold, 30-fold, 100-fold, 300-fold, or 1000-fold greater affinity at physiologic pH than it does at pH 5.4.

Embodiment 43. The method of any one of embodiments 40-42, wherein the anti-cMET antibody comprises a VH chain comprising the amino acid sequences as set forth in SEQ ID NO: 236, SEQ ID NO: 237, and SEQ ID NO: 238 and a VL chain comprising the amino acid sequences as set forth in SEQ ID NO: 239, SEQ ID NO: 240, and SEQ ID NO: 241.

Embodiment 44. The method of any one of embodiments 40-43, wherein the anti-cMET antibody is an IgG1 antibody.

Embodiment 45. The method of embodiment 42, wherein the anti-cMET antibody comprises a VH chain comprising the amino acid sequence of SEQ ID NO: 15 and a VL chain comprising the amino acid sequence of SEQ ID NO: 16.

Embodiment 46. The method of embodiment 45, wherein the anti-cMET antibody is an IgG1 antibody.

Embodiment 47. The method of embodiment 43, wherein the anti-cMET antibody comprises a heavy chain comprising the amino acid sequence of SEQ ID NO: 75 and a light chain comprising the amino acid sequence of SEQ ID NO: 82.

Embodiment 48. The method of embodiment 40, wherein the linker is cleavable by a lysosomal enzyme, optionally wherein the enzyme is Cathepsin B.

Embodiment 49. The method of embodiment 40, wherein the linker comprises a peptide selected from the group consisting of Cit-Cit; Cit-Val; Val-Cit; Cit-Ala; Ala-Cit; Cit-Asn; Asn-Cit; Cit-Ser; Ser-Cit; Cit-Lys; Lys-Cit; Cit-Asp; Asp-Cit; Ala-Ala; Glu-Val; Val-Glu; Ala-Val; and Val-Ala; and salts thereof.

Embodiment 50. The method of any one of the preceding embodiments, wherein the drug component of the ADC is a microtubule inhibitor, optionally wherein the inhibitor is an auristatin.

Embodiment 51. The method of embodiment 50, wherein the microtubule inhibitor is an auristatin.

Embodiment 52. The method of embodiment 51, wherein the ADC comprises a compound having a structure of formula (I): [D-L-XY]n-Ab or salts thereof, where each “D” represents a cytotoxic and/or cytostatic agent (“drug”); each “L” represents a linker; “Ab” represents a pH-dependent anti-cMET antigen binding moiety; each “XY” represents a linkage formed between a functional group Rx on the linker and a “complementary” functional group Ry on the antigen binding moiety; and n represents the number of drugs linked to Ab of the ADC; optionally wherein the compound has the following structure (II):

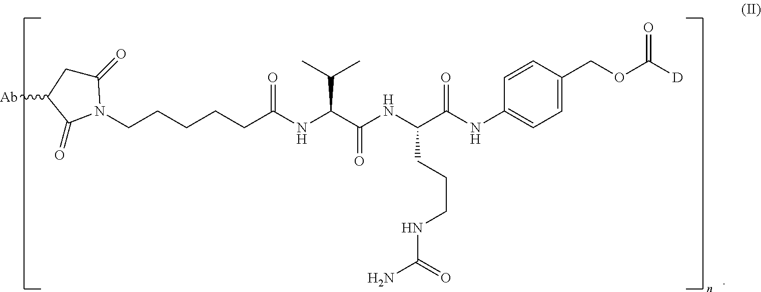

Embodiment 53. The method of embodiment 52, wherein the anti-cMET antibody is Q397.

Embodiment 54. The method of embodiment 53, wherein the compound has the following structure (III):

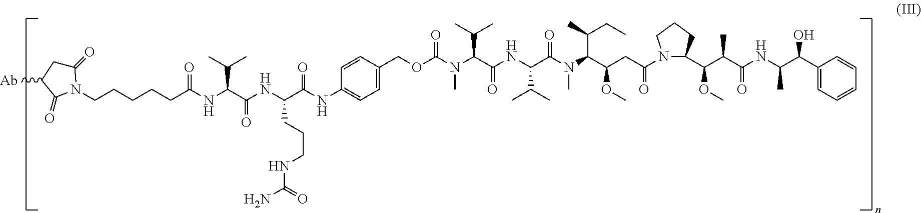

or a pharmaceutically acceptable salt thereof, wherein n has an average value of 2, or is equal to 2, and the Ab is a full length anti-cMET pH-antibody.

Embodiment 55. The method of embodiment 54, wherein the anti-cMET antibody is Q397.

Embodiment 56. The method of any one of the preceding embodiments, wherein the human subject is a patient diagnosed with cMET-overexpressing, MET amplified and/or MET ex14 skipping mutation non-small cell lung cancer (“NSCLC”) comprising the step of administering to the patient an effective amount of an anti-cMET antibody drug conjugate (“ADC”) for a period of time sufficient to provide one or more therapeutic benefit(s), wherein the antibody component of the ADC exhibits cMET-specific pH-dependent binding, and optionally wherein the antibody comprises heavy and light chain CDRs present in the amino acid sequences as set forth in one of the following pairs: SEQ ID NOs: 15 & 16; SEQ ID NOs: 5 & 6; SEQ ID NOs: 7 & 8; SEQ ID NOs: 9 & 10; SEQ ID NOs: 11 & 12; SEQ ID NOs: 13 & 14; SEQ ID NOs: 17 & 18; SEQ ID NOs: 19 & 20; SEQ ID NOs: 21 & 22; SEQ ID NOs: 23 & 24; SEQ ID NOs: 25 & 26; SEQ ID NOs: 27 & 28; SEQ ID NOs: 29 & 30; SEQ ID NOs: 31 & 32; and SEQ ID NOs: 33 & 34.

Embodiment 57. The method of embodiment 56, wherein the cancer has resisted prior treatment with a microtubule inhibitor.

Embodiment 58. The method of embodiment 57, wherein the microtubule inhibitor is an auristatin.

Embodiment 59. The method of embodiment 56, wherein the cancer has resisted prior treatment with an anti-cMET ADC, an anti-cMET antibody, or a small molecule targeting cMET.

Embodiment 60. The method of any one of embodiments 56-59, wherein the anti-cMET ADC is administered as monotherapy.

Embodiment 61. The method of any one of embodiments 56-60, wherein the anti-cMET ADC is administered adjunctive to an additional anticancer agent, wherein the additional agent is administered according to its regulatory agency-approved dosing regimen.

Embodiment 62. The method of any one of the preceding embodiments, wherein the anti-cMET ADC is effective against non-squamous cell and/or squamous cell NSCLC.

Embodiment 63. The method of embodiment 62, wherein the NSCLC is wildtype for human EGFR.

Embodiment 64. The method of embodiment 63, wherein the NSCLC is mutated for human EGFR.

Embodiment 65. The method of embodiment 63 or 64, wherein the NSCLC has resisted at least 1, 2, 3, 4, 5, 6, 7, 8, or more prior therapeutic regimen(s).

Embodiment 66. The method of embodiment 65, wherein the NSCLC has resisted at least 2 regimens.

Embodiment 67. The method of embodiment 66, wherein the NSCLC has resisted at least 3 regimens.

Embodiment 68. The method of embodiment 67, wherein the NSCLC has resisted at least 4 regimens.

Embodiment 69. The method of embodiment 68, wherein the NSCLC has resisted at least 5 regimens.

Embodiment 70. The method of embodiment 69, wherein the NSCLC has resisted at least 6 regimens.

Embodiment 71. The method of embodiment 70, wherein the NSCLC has resisted at least 7 regimens.

Embodiment 72. A pharmaceutical composition comprising MYTX-011 and a pharmaceutically acceptable carrier.

Embodiment 73. A method of treating a non-squamous non-small cell lung cancer (“NSCLC”) tumor that expresses c-Met, comprising administering to a human subject having said NSCLC tumor an effective amount of the pharmaceutical composition of embodiment 72, wherein ≥25% of neoplastic cells from tumor tissue of the c-Met expressing non-squamous NSCLC from the subject have 2+ membrane or membrane+cytoplasmic staining when assessed by c-Met immunohistochemistry (IHC).

Embodiment 74. Use of the composition of embodiment 72 for treating NSCLC in a human subject.

Embodiment 75. The method or use of any one of the preceding embodiments, wherein the tumor comprises cells having an FGFR3 amplification, a MET Exon 14 mutation, or an EML4-ALK fusion.

Embodiment 76. The method or use of embodiment 75, wherein the subject has received at least 2 prior treatment regimens for the NSCLC.

Embodiment 77. The method or use of embodiment 76, wherein the subject's tumor is positive for a MET Exon 14 mutation.

Embodiment 78. The method or use of embodiment 75, wherein the tumor has progressed after prior treatment with tyrosine kinase inhibitor, optionally osimertinib.

Embodiment 79. A kit comprising: (i) the pharmaceutical composition of embodiment 72; and (ii) instructions for performing any of the methods of embodiments 1-71, 73, or 75-78.

Embodiment 80. A pharmaceutical composition comprising a therapeutically effective amount of a pH-dependent, anti-cMET antibody drug conjugate, comprising two (2) heavy chains, each having the sequence set forth in SEQ ID NO: 75 and two (2) light chains, each having the sequence set forth in SEQ ID NO: 82, wherein each light chain constant domain comprises the sequence set forth in SEQ ID NO: 158, wherein each light chain constant domain comprises a linker-drug conjugated thereto at the cysteine (C) located at amino acid position 98 of SEQ ID NO: 158; optionally wherein each linker-drug comprises, consists essentially of, or consists of vcMMAE.

Embodiment 81. The composition of embodiment 81, which remains effective for treating cancer when frozen and rethawed up to at least 2 times, further comprising L-Histidine; L-Histidine HCl, monohydrate; D (+)-Trehalose dihydrate; and Polysorbate 80.

From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

MYTX-011 is a cMET-targeted val-cit-monomethyl auristatin E (vcMMAE) antibody-drug conjugate (ADC) with a fully humanized IgG1. The drug to antibody ratio (DAR) for MYTX-011 is 2:1. MYTX-011 binds to cMET with high affinity and specificity and has been engineered to enhance the internalization and delivery of the cytotoxic payload to cancer cells. In the initial clinical trial, single and multiple IV doses of MYTX-011 will be given to late-stage cancer patients. MYTX-011 is being developed for the treatment of cMET+/cMET-expressing and cMET-overexpressing non-small cell lung cancer (NSCLC). MYTX-011 has been investigated in nonclinical pharmacology, pharmacokinetic, and toxicology studies.

The nonclinical pharmacology of MYTX-011 was evaluated to determine its binding affinity and pharmacologic effects. MYTX-011 was assessed for species selectivity, as well as binding affinity to cMET using ELISA and BLI assays. The pH-dependent binding and internalization of MYTX-011 was also assessed. Cell cycle arrest and cytotoxic activity of MYTX-011 was assessed in cMET+ tumor cell lines. The potential for the anti-cMET antibody portion of MYTX-011 to enhance cellular proliferation and cell signaling in cMET+ tumor cell lines was assessed. Fc effector function activities of the anti-cMET antibody portion of MYTX-011 including antibody-dependent cellular cytotoxicity (ADCC), complement-dependent cytotoxicity (CDC), and antibody-dependent cellular phagocytosis (ADCP) were also evaluated. The efficacy of MYTX-011 was assessed in murine xenograft models of non-small cell lung cancer.

The nonclinical safety profile of MYTX-011 was evaluated in vivo in cynomolgus monkeys. The monkey was selected as the pharmacologically relevant nonclinical species because of similar cMET protein sequence homology and similar binding affinity of MYTX-011 to monkey cMET, as compared to human cMET. MYTX-011 does not bind to mouse or rat cMET.

The IV route of exposure was selected for the in vivo monkey studies since it is the intended route of clinical exposure. The once every three-week (Q3W) dosing regimen used in the repeat-dose monkey toxicity studies was selected to align with the clinical dosing regimen.

The nonclinical PK and TK of MYTX-011 was evaluated in monkeys. Analytical methods were developed and validated for the quantitation of intact MYTX-011, total mAb, and MMAE, and for the detection of anti-drug antibodies (ADA) to support TK evaluations. Plasma stability of MYTX-011 was assessed in monkey and human serum.

MYTX-011 was specifically designed to address the shortcomings of existing therapies, including other anti-cMET ADCs, by driving responses for the majority of cMET+/cMET-overexpressing NSCLC patients whose tumors express lower levels of cMET than those treatable with current cMET-targeted therapies. Design of MYTX-011 includes clinically precedented triple hinge (TH) IgG1 format and linker/toxin (i.e., vcMMAE), complementarity determining regions (CDRs) that mediate pH-dependent binding, and site-specific conjugation at an engineered cysteine residue (“V205C”, corresponding to a cysteine (C) at position 98 of the light chain constant domain set forth in SEQ ID NO: 157), which results in a drug-to-antibody ratio (DAR) of 2 (Shen et al., Nature Biotechnology Vol 30 No. 2 Feb. 2012). MYTX-011 is being developed for the treatment of cMET+, cMET-overexpressing, and MET-amplified cancers, including non-small cell lung cancer (NSCLC).

MYTX-011 has nanomolar binding affinity for both human and cynomolgus monkey cMET. MYTX-011 did not bind to rat or mouse cMET. MYTX-011 demonstrated pH-dependent binding, showing enhanced dissociation from recombinant human cMET at pH 5.4 as compared to pH 7.4. In addition, MYTX-011 can be effectively internalized into cancer cells expressing cMET, and “Q397-noV205C” (the antibody portion of MYTX-011 except that it lacks the “V205C” substitution in both of its light chains) had minimal to no ADCC, ADCP or CDC. MYTX-011 also has potent activity in vitro in cMET+ and cMET-overexpressing human lung cancer cell lines and in vivo in cMET+ and cMET-expressing murine xenograft models of non-small cell lung cancer.

Two doses of MYTX-011 administered once every three weeks in cynomolgus monkeys resulted in no MYTX-011-related cardiovascular, respiratory, or central nervous system effects.

Cross Species cMET Protein Sequence Homology

The predicted human cMET protein sequence was compared to mouse, rat, and cynomolgus monkey sequences. Mouse, rat, and monkey protein sequences were 87.02%, 85.71%, and 98.46% homologous to human cMET, respectively.

The following Examples, which highlight certain features and properties of exemplary embodiments of anti-cMET ADCs and methods of using these ADCs to treat patients are provided for purposes of illustration, and not limitation.

### Example 1. Preparation of MYTX-011

MYTX-011 is an antibody drug conjugate (ADC) that exhibits pH-dependent binding comprised of the antibody Q397 conjugated to the cytotoxic microtubule inhibitor monomethylauristatin E (MMAE) via a cleavable valine-citrulline (vc) linker. Q397 is a humanized IgG1 monoclonal antibody that specifically binds to cMET in a pH-dependent manner. Q397 is composed of two heavy chains of 442 amino acids (as set forth in SEQ ID NO: 75), disulfide bonded to two Kappa light chains of 215 amino acids (as set forth in SEQ ID NO: 82), with a total molecular weight of 143,988 Da (theoretical mass excluding glycosylation). Engineered cysteines have been introduced to the light chains at position 98 of the constant domain (i.e., the light chain constant domain having the polypeptide sequence of SEQ ID NO: 158), which may be capped by disulfide bond formation with thiol-containing molecules, such as cysteine or glutathione. In an exemplary MYTX-011 aqueous solution (e.g. 20 mM histidine buffer, 8% (w/v) sucrose, pH 6.0) absorbance at 280 nm yields an extinction coefficient of 1.48 mL/(mg*cm). Non-denaturing size exclusion chromatography demonstrates monomeric purity of intact Q397.

Q397 for the preclinical studies described below was prepared using techniques essentially as described in WO 2022/169975 A1 (to Mythic Therapeutics). Briefly, expression plasmids containing SEQ ID NO: 75 and SEQ ID NO: 82 were transiently transfected into Expi293 cells. After allowing for about four to five days of protein expression, cell culture supernatants were collected, and Q397 was purified using a routine chromatography technique.

Q397 for the clinical studies described herein was prepared substantially as follows. A CHO K1 host cell was thawed and passaged in CD CHO medium for stable transfections. The heavy chain plasmid and the light chain plasmid were added at a ratio of 1:1 and 1:2 to the CHO-K1 host cell line for transfection. After electroporation, cells of each transfection were added to 10 mL pre-warmed CD CHO medium and incubated in spin tubes in a Kuhner shaker (36.5° C., 75% humidity, 6% CO2, 225 rpm). About 24 hours after transfection, 10 mL of “Selection 1” medium was added to each spin tube. The pools were passaged in “Selection 2” medium every 2-3 days. After cells recovered from selection pressure, both pools were used for cloning.

The major components of the light chain plasmid includes the following genes or regulatory elements in the following order: Regulatory Element (improve target gene expression), HuCMV promoter, light chain coding sequence, TK polyadenylation signal, SV40 early promotor, blasticidin resistance gene (selectable marker), SV40 polyadenylation signal, pUC origin, amp resistance gene. The major components of the heavy chain plasmid includes the following: Regulatory Element, HuCMV promoter, heavy chain coding sequence, EMCV IRES, Zeocin resistance gene, TK polyadenylation signal, pUC origin, amp resistance gene.

Stable pools were produced and plated into 96-well plates at one cell per well, and the monoclonality was documented by consecutive single cell imaging of all wells on Day 0, Day 1, and Day 5 after plating. About 50 clones were isolated and screened in fed-batch cultures in spin tubes. A single clone was selected to produce the master cell bank (MCB).

A single frozen vial of cells is expanded by either shaker culture or cell bags. A larger volume of culture medium is inoculated with the expanded cultures and the cultures expanded further in a bioreactor under standard conditions (e.g. 5% CO2, 36° C. incubator). The cultures are harvested and clarified of cells and debris. The Q397 is purified through a Protein A affinity column, followed by anion exchange chromatography, cation exchange chromatography, viral filtration, ultrafiltration/diafiltration, and final filtration.

Payload conjugation to Q397. vcMMAE is a linker-payload intermediate used in the manufacture of MYTX-011. “vcMMAE” is a protease cleavable linker (maleimidocaproyl-valinecitrulline-p-aminobenzyloxycarbonyl, mc-vc-PAB) attached to the small molecule anti-mitotic agent monomethyl auristatin E (MMAE). In the ADC manufacturing process, vcMMAE is covalently linked to Q397 via the cysteine at a position corresponding to amino acid position 98 of its light chain constant domain (SEQ ID NO: 158).

MYTX-011 of DAR 2.0 was prepared by multi-step process comprising: 1) reduction of Q397, 2) a first ultrafiltration/diafiltration (UF/DF), 3) re-oxidation, 4) a second UF/DF, 5) conjugation to vcMMAE, 6) quenching, 7) a third UF/DF, and 8) formulation.

Briefly, Q397 in Tris-EDTA (about pH 7.6-8.0) is mixed with reduction buffer (50 mM PB buffer, pH 7.8). The reducing agent (RA) is added to yield an RA/Q397 molar ratio of about 130-200. The solution containing reduced Q397 is then subjected to UF/DF membrane (e.g., with a MW cutoff of 30 kD). The product is buffer exchanged into re-oxidation buffer 50 mM PB, 2 mM EDTA, pH 7.2 and filtered through a 0.2 μm filter. The UF/DF 1 pool is then transferred to a reactor for re-oxidation in buffer (50 mM PB, 2 mM EDTA, pH7.2), and (L)-Dehydroascorbic acid (DHAA) solution in DMA is next added to the reactor. Re-oxidation proceeds for several hours. Next, a second UF/DF is performed and product is then buffer exchanged into conjugation buffer (50 mM PB, 2 mM EDTA, pH 7.4) and filtered through a 0.2 μm filter. The filtered, re-oxidized Q397 is then conjugated to the linker-payload vcMMAE. VcMMAE powder is weighed, dissolved in DMA, and added into the reactor. The vcMMAE/Q397 molar ratio is about 4.4-4.8 in a final DMA concentration of about 10% (v/v). After several hours, the conjugation reaction is quenched in N-Acetyl-L-cysteine (NAC) dissolved in WFI. Product is filtered again and buffer exchanged into diafiltration buffer and filtered through a 0.2 μm filter.

The filtered MYTX-011 is then formulated to 10.0 mg/mL MYTX-011, 20 mM histidine buffer, 8.8% (w/v) trehalose, 0.02% (w/v) polysorbate 80, pH 5.5.

### Example 2. Anti-cMET pH-ADC Binds to Recombinant and Cellular cMET and is Efficiently Internalized by Cells Having Elevated cMET Expression

Bio-Layer Interferometry (BLI). The binding and binding affinity of MYTX-011 to mouse, rat, cynomolgus monkey, and human cMET was assessed by ELISA or BLI assays. MYTX-011 was captured as ligand onto the surface of an AHC sensor tip through binding to immobilized anti-human Fc-specific antibodies followed by association with either recombinant human or cynomolgus monkey cMET as analyte in running buffer. Association was measured by an increase in the thickness of the layer on the tip of the AHC sensor in the presence of analyte and dissociation was measured by the loss of thickness in the absence of analyte. Briefly, MYTX-011, in buffer containing 2.7 mM potassium chloride, 4.3 mM disodium phosphate, 1.4 mM monopotassium phosphate, 135 mM sodium chloride, and 0.05% tween-20 (PBST) at pH 7.4, was immobilized onto the surface of an AHC sensor tip followed by a buffer-only step to establish a stable baseline. Association with analyte was achieved in a separate well in 1×PBST (pH 7.4) plus 0.1% Bovine Serum Albumin (BSA). The following analyte concentrations and incubations times were tested: Recombinant human cMET at 80, 60, and 20 nM for 120 seconds, or recombinant cynomolgus monkey cMET at 100, 62.5, and 31.2 nM for 60 seconds. Following association, sensor tips were moved to wells containing buffer 1×PBST pH 7.4 plus 0.1% BSA only and dissociation was measured for 240 seconds for recombinant human cMET or 180 seconds for recombinant cynomolgus monkey cMET. pH-dependent binding of MYTX-011 to human cMET was assessed using conditions listed above with the following changes: analyte concentration was 50 nM recombinant human cMET; association was performed in 1×PBST pH 7.4 for 120 seconds; and the dissociation was performed in either 1×PBST pH 7.4 or 1×PBST pH 5.4 for 300 seconds.

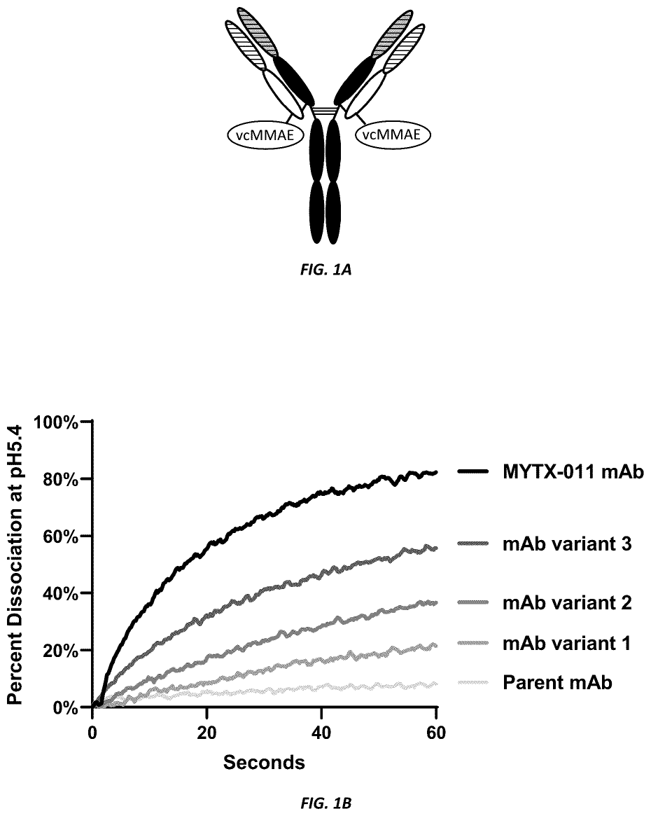

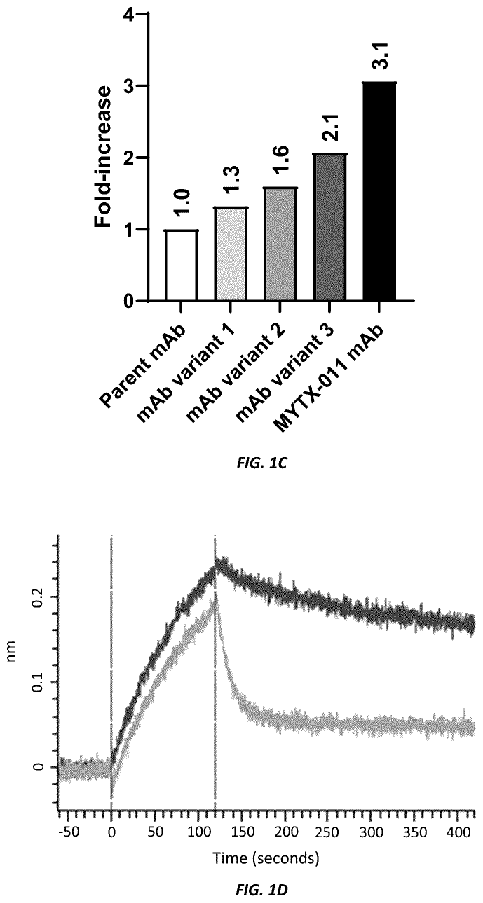

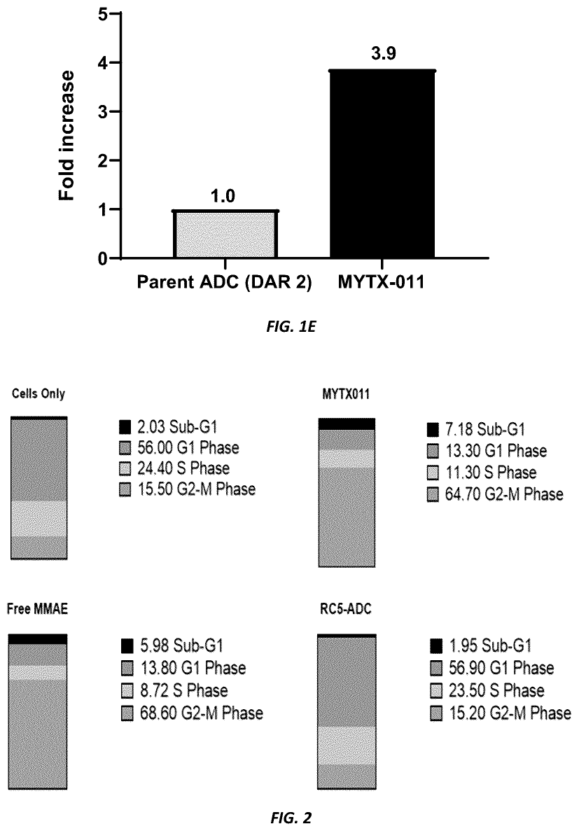

As measured by the above BLI assay, binding affinities (KD) of MYTX-011 mAb to recombinant human and cynomolgus monkey cMET were 4.0 nM and 9.44 nM, respectively. In contrast, MYTX-011 mAb did not bind to mouse or rat cMET. Further, MYTX-011 mAb bound to recombinant human and cynomolgus monkey cMET with similar affinity at pH 7.4 and MYTX-011 mAb showed greater dissociation from human cMET at pH 5.4 compared to pH 7.4. The percent dissociation (from human cMET) of multiple pH-dependent anti-cMET mAbs at pH 5.4 was then evaluated (as described above), yielding the dissociation curves presented in FIG. 1B. As regards its binding on cMET+ NCI-H1975 cancer cells, MYTX-011 bound with sub-nanomolar affinity (IC50=0.4 nM) at pH 7.4 and binding was maintained at pH 6.4 (IC50=0.5 nM) (DNS), which is below estimates of an acidic tumor microenvironment (TME).

Internalization Assays. Detroit-562 or NCI-H1975 cells were seeded at 5000 cells/well a day prior to treatment. pHrodo reagent (Thermo Fisher Scientific), a secondary anti-huIgG Fab conjugated to a pH-sensitive dye that fluoresces only after internalization, was diluted to 60 nM in media. Twenty (20) nM of the hIgG1 component of MYTX-011, MYTX-011 mAb, parent and isotype control antibodies or 21 nM of MYTX-011, parent ADC and non-binding ADC were diluted 1:1 with pHrodo reagent. After incubation for 30 minutes, media was removed from pre-seeded Detroit-562 or NCI-H1975 cells, pHrodo-antibody/ADC complex was added to the plate and incubated at 37° C. for 24 hours. Cells were trypsinized using TrypLE (Thermo Fisher Scientific) and washed twice using ice cold FACS buffer pH 7.4. Fluorescence was analyzed using Attune NxT flow cytometer (Thermo Fisher Scientific) and data was analyzed using FlowJo. Fold change was calculated by subtracting median fluorescent intensity (MFI) of the isotype control/non-binding ADC from MFI of MYTX-011 antibody or MYTX-011 and parent antibody or ADC and taking the ratio.

Internalization of MYTX-011 mAb in Detroit-562 cancer cells. The internalization assay was conducted as described above and the results are shown in FIG. 1C. Surprisingly, the enhanced dissociation observed for pH-dependent antibody variants at pH 5.4 (FIG. 1B) correlated with an increase in internalization over parent antibody, confirming that greater pH dependence leads to enhanced accumulation of antibody inside cMET positive cancer cells (FIG. 1C)

Internalization of MYTX-011 in NCI-1975 cancer cells. The internalization assay was conducted as described above. Briefly, cells were plated a day prior to the experiment date. Test articles were prepared as follows: Human IgG1, MYTX-011, and RC2-ADC-(DAR2) were diluted to 20 nM using cell media. “RC2-ADC-(DAR2)” is a comparative ADC comprising a mAb component having variable regions matching those of emibetuzumab and conjugated to vcMMAE via one cysteine on each light chain. pHrodo reagent was diluted 3× (60 nM) concentration to that of test articles and were mixed at 1:1 ratio. The cells were treated and processed as described above, and as shown in FIG. 1E, fluorescently labeled MYTX-011 yielded an impressive 3.9 fold increase in signal in the NCI-1975 cells relative to non-pH engineered comparator RC2-ADC-(DAR2) (FIG. 1E).

Overall Conclusions. MYTX-011 bound to recombinant human and cynomolgus monkey cMET with similar affinity at pH 7.4, exhibited greater dissociation from human cMET at pH 5.4 compared to pH 7.4, and showed about 3.9× higher internalization relative to non-pH engineered RC2-ADC-(DAR2). This enhanced internalization helps to explain why the anti-cMET, pH-dependent ADC, MYTX-011, delivers superior amounts of toxic payload relative to an appropriate, non-pH-dependent, control anti-cMET ADC. Applicant envisions that any and all such anti-cMET pH-antibodies exhibiting comparable pH-dependent binding characteristics coupled with superior internalization may be particularly useful in the practice of the methods as disclosed herein. In some embodiments, the binding characteristics include greater dissociation from human cMET at about pH 5.4 versus about pH 7.4, and maintenance of good binding (e.g., comparable to binding at pH 7.4) at pH conditions found within a tumor microenvironment (e.g., between about pH 6.4 and about pH 7.4). In some embodiments, the anti-cMET pH-antibody binds to cMET with substantially the same affinity and/or kinetics at pH 6.4 as it does at pH 7.4.

Non-limiting examples of such anti-cMET ADCs (e.g., anti-cMET pH-ADCs) include those comprising heavy chain variable and light chain variable sequences as set forth in one of the following pairs of heavy and light chain sequences: SEQ ID NOs: 15 & 16; SEQ ID NOs: 5 & 6; SEQ ID NOs: 7 & 8; SEQ ID NOs: 9 & 10; SEQ ID NOs: 11 & 12; SEQ ID NOs: 13 & 14; SEQ ID NOS: 17 & 18; SEQ ID NOs: 19 & 20; SEQ ID NOs: 21 & 22; SEQ ID NOs: 23 & 24; SEQ ID NOs: 25 & 26; SEQ ID NOs: 27 & 28; SEQ ID NOs: 29 & 30; SEQ ID NOs: 31 & 32; and SEQ ID NOs: 33 & 34.

In some embodiments, the anti-cMET pH-ADC comprises a heavy chain constant (CH1-CH2-CH3) sequence of SEQ ID NO: 155 or SEQ ID NO: 189 comprising one or more of the following: (i) a lysine to cysteine substitution at amino acid position 105 and deletion of a threonine at amino acid positions 106 and 108; (ii) a methionine to tyrosine substitution at amino acid position 135, a serine to threonine substitution at amino acid position 137, and a threonine to glutamic acid substitution at amino acid position 139; (iii) a methionine to leucine substitution at amino acid position 311 and an asparagine to serine substitution at amino acid position 317; and (iv) an alanine to a cysteine substitution at amino acid position 1; and/or an LC constant domain sequence of SEQ ID NO: 157 comprising a val to cys substitution at amino acid position 98.

### Example 3. In Vitro Potency of Anti-cMET pH-ADC in Tumor Cell Lines

Cell Cycle Arrest Assay. Briefly, EBC-1 cells were cultured in standard culture media (Eagle's minimum essential medium+10% fetal bovine serum) and conditions recommended by the vendor. Cells were plated at a density of 2×10⁴ cells per well and incubated at 37° C. for 24 hours. Media was removed and test articles (10 nM MYTX-011, MMAE toxin, or a non-binding control ADC) were added to the cells at a final concentration of 10 nM. Plates were incubated for 24 hours at 37° C. At the end of the culture period, test articles were removed from the plate. Cells were trypsinized, washed, and fixed in 70% ethanol for 30 minutes. Cells were washed, stained with propidium iodide staining using a cell cycle analysis kit (Abcam), and assessed by flow cytometry.

An increase in the G2-M fraction was observed in EBC-1 cells 24 hrs after treatment with MYTX-011 and MMAE as compared to untreated cells (FIG. 2). The percentage of untreated cells in G2-M phase was 15.5%. For cells treated with MYTX-011, the percentage of cells in G2-M phase was 64.7%, which was comparable to MMAE treatment (68.6%). Accordingly, MYTX-011 treatment of cMET+ EBC-1 cells resulted in G2-M phase cell cycle arrest, similar to that of MMAE treatment, and no impact on the cell cycle phase distribution was observed with the non-binding control RC5-ADC-(DAR2).

Cellular cytotoxicity assay. NCI-H1975 and NCI-H1373 cells were cultured in standard culture media (RPMI-1640+10% fetal bovine serum) using conditions recommended by the vendor. The following test articles were evaluated in this assay: RC1=comparative Ab having variable regions matching those of telisotuzumab; RC2=comparative Ab having variable regions matching those of emibetuzumab; RC3=comparative Ab having variable regions matching the 5D5 anti-human cMET antibody; and Q397-noV205C=Q397 with V at position 98 of its light chain constant domain; Q397=the anti-cMET IgG antibody component of MYTX-011.

Cells were plated and incubated overnight at 37° C. The starting concentration of MYTX-011, RC1 ADC-(DAR3.1), RC1-ADC-(DAR2), RC2 ADC-(DAR2), and RC5 ADC-(DAR2) was 100 nM; a 9-point, 1:5 dilution series was generated. Serially diluted test articles were added to the cells in duplicate the next day. Plates were incubated for 5 days at 37° C. with a range of concentrations (0.000256-100 nM) of MYTX-011. At the end of the culture period, viability was assessed using CCK-8. Absorbance was measured using a plate reader. MYTX-011 depleted viable NCI-H1975 and NCI-H1373 cells with a maximum depletion of 90.36% and 65.92%, respectively, relative to the untreated control at 100 nM. MYTX-011 thus demonstrated cytotoxic activity in cMET+ NCI-H1975 and NCI-H1373 cells. No cytotoxicity was observed after treatment with the non-binding control, RC5-ADC-(DAR2). MYTX-011 demonstrated cytotoxic activity similar to that of RC1-ADC-(DAR3.1) in NCI-H1975 and NCI-H1373 cells.

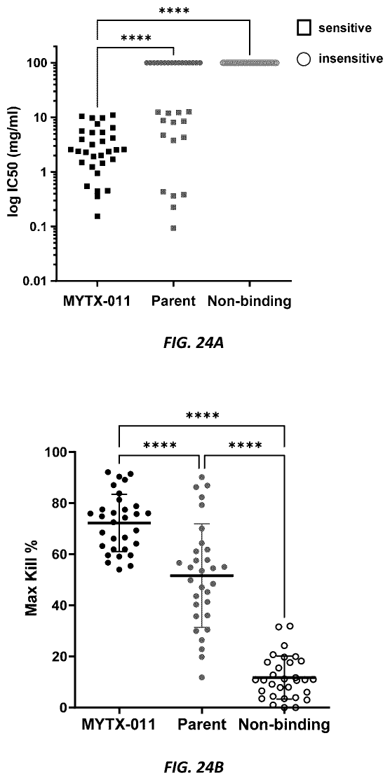

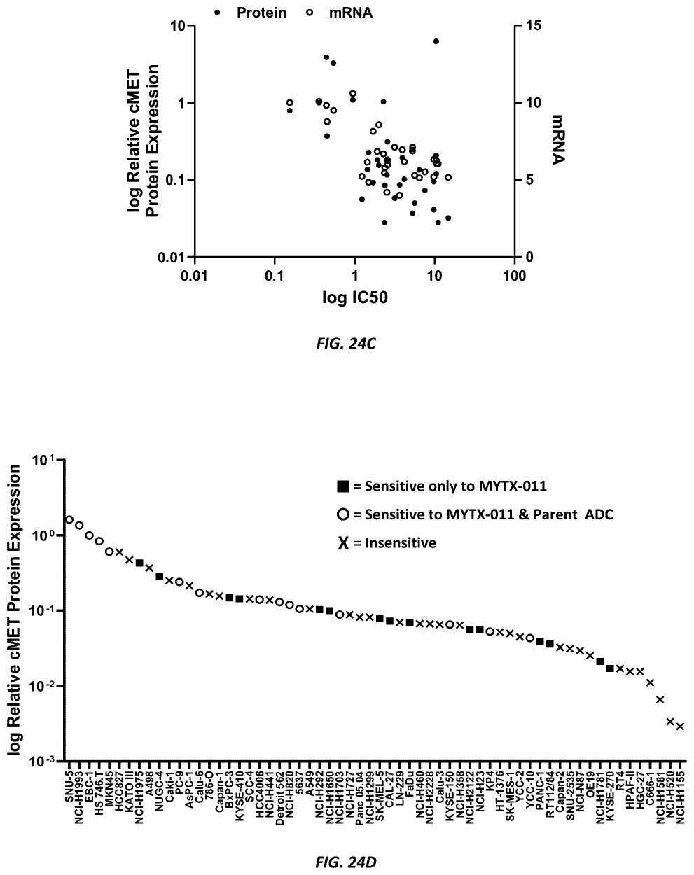

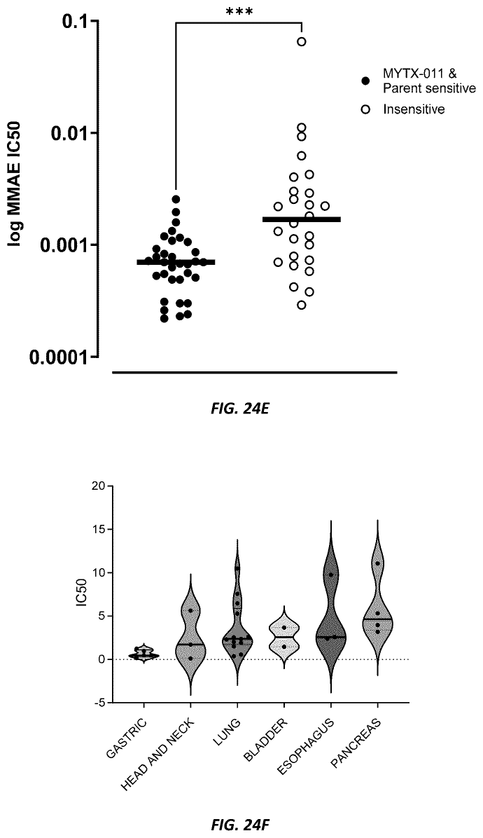

MYTX-011 has also demonstrated cytotoxicity against cells from a wide variety of other cancer types, including the following: SNU-5 (amplification), H1993 (amplification), EBC-1 (amplification), HS746T (amplification & Exon14 skipping), MKN45 (amplification), H1975, NUGC-4, PC-9, Calu-6, BxPC-3, KYSE-410, HCC4006, Detroit 562, H1650, H820, 5637, H292, H1703, Panc 05.04, SK-MEL-5, CAL-27, FaDu, KYSE-150, H2122, H23, KP4, YCC-2, YCC-10, PANC-1, RT112/84, H1781, and KYSE-270. See FIGS. 24A-24E and the disclosure related thereto.

As cMET agonism can be an oncogenic driver, Q397-noV205C (comprising the sequences set forth in SEQ ID NO: 75 and SEQ ID NO: 81), essentially the mAb component of MYTX-011, was assessed for its potential to enhance cellular proliferation and cell signaling. Q397 itself (i.e., the actual mAb component of MYTX-011) was not used because the C at position 98 of its light chain constant domain (SEQ ID NO: 158) may cause undesirable aggregation. The effects of Q397-noV205C treatment on proliferation of the cMET expressing human lung adenocarcinoma cell line, NCI-H441, and on cMET receptor signaling in NCI H441 cells using phosphorylated-Akt1 (pAkt1) and phosphorylated-ERK1/2 (pERK1/2) as measures of pathway activation were assessed.

Cell Proliferation Assay. Briefly: NCI-H441 cells were cultured in standard culture media (RPMI-1640 Medium+10% fetal bovine serum) and conditions recommended by the vendor. NCI-H441 cells were serum starved for approximately 24 hours at 37° C. Cells were treated with 10 nM test articles, and plates were incubated for 4 days at 37° C. Each sample was plated in duplicate technical replicates. At the end of the culture period, proliferation was measured using CellTiter-Glo. Luminescence was measured using a plate reader at 578 nm.

Cell Signaling Assay. Briefly: NCI-H441 cells were cultured in standard culture media (RPMI-1640 Medium+10% fetal bovine serum) using conditions recommended by the vendor. For the cell signaling assay, NCI-H441 cells were seeded in 6-well plates at 300,000 cells per well in culture media. The next day, the cells were washed with serum-free medium twice and then replenished with 3 mL RPMI+0.2% fetal bovine serum.

The following day, cells were treated with 50 ng/ml HGF or 10 μg/mL of test articles and incubated for 30 minutes at 37° C. Following the 30-minute stimulation, the treatment medium was aspirated, the cells were washed 2 times in an excess volume of ice-cold phosphate buffered saline and collected by cell scraping on ice in 250 μL ice-cold lysis buffer per well and transferred to pre-chilled tubes. Cell lysates were incubated on ice for 10 minutes with occasional vortexing, and then centrifuged at maximum speed for 5 minutes to clear the lysate. Lysates were frozen at −20° C. until analysis. Lysates were thawed on ice for 1 hour with occasional vortexing, and protein concentrations were measured using the BCA protein assay according to the manufacturer's instructions. Lysates were diluted in lysis buffer to final concentration 0.5 mg/mL, and 100 μL of each sample was assessed using commercially available ELISA kits to detect total ERK1/2 and Akt1 and their phosphorylated forms. ELISAs were performed and analyzed according to the manufacturer's instructions.

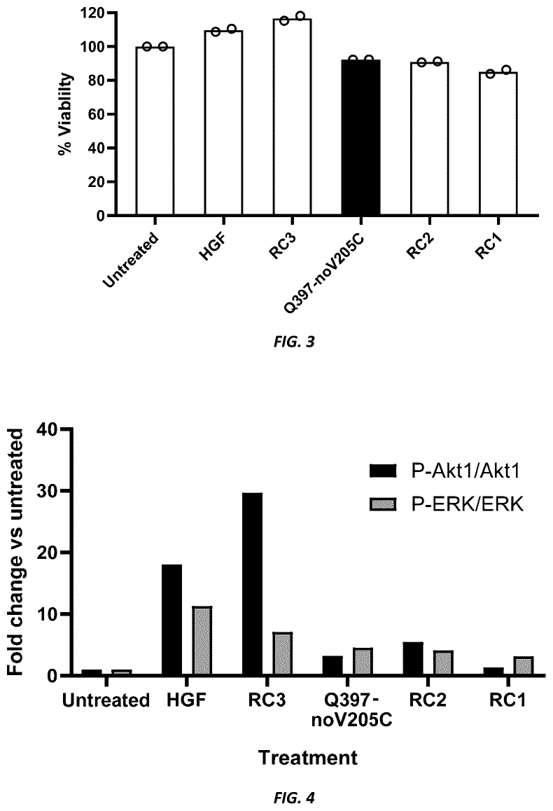

Results. For FIGS. 3 & 4, abbreviations including RC1, RC2, and RC3 are as defined above, and treatment induced phosphorylation was represented as a ratio of phospho-protein to total protein and normalized to untreated controls (n=1 pooled from 2 technical replicates). HGF=positive control, hepatocyte growth factor; P-Akt1/Akt1=ratio of pAkt1 to total Akt1 levels; P-ERK/ERK=ratio of phosphorylated ERK1/2 to total ERK1/2 levels.

Conclusions. Q397-noV205C treatment did not increase the proliferation of cMET+ NCI-H441 cells, in contrast to cMET agonist controls HGF and RC3 (FIG. 3). Q397-noV205C treatment of NCI-H441 cells caused minimal phosphorylation of ERK1/2 or Akt1, downstream markers of cMET signaling, compared with HGF and RC3 (FIG. 4).

MYTX-011 Fc Effector Functions. MYTX-011 was assessed for ADCC, ADCP, and CDC effector functions in vitro. Q397=Anti-cMET antibody of MYTX-011; Q397-noV205C (also referred to as MYT6878)=Anti-cMET antibody of MYTX-011 without the “V205C” substitution in the light chain; “V205C”=a valine to cysteine change at amino acid position 98 of the light chain constant domain sequence set forth in SEQ ID NO: 157; vcMMAE=val-cit-monomethyl auristatin E.

Complement Dependent Cytotoxic Assay. EBC-1 cells were incubated with serially diluted test articles: Q397-noV205C or non-binding antibody. For the technical control, Raji cells, which expresses CD20 on the cell surface, were incubated with serially diluted test articles: anti-CD20 (research grade recombinant anti-CD20 rituximab, catalog number A2009) or non-binding antibody (human IgG1 isotype control antibody, catalog number BE0297). Briefly, EBC-1 and Raji cells were added to a 96-well U-bottom plate at a density of 2.5×10⁴ cells/well. Serially diluted test articles were added to the plate. The starting concentration of all test articles was 100 nM. A 3-fold dilution was performed for 9 concentration points and added to the test plate in duplicates. The plate was then incubated for 30 minutes at 37° C. At the end of the culture period, either heat inactivated or pooled human complement sera at a final concentration of 30% diluted in culture media (RPMI) was added to the plate. The plate was then incubated at 37ºC for two hours and cell lysis was assessed using CellTiter-Glo.

Conclusions. Minimal ADCC activity and no ADCP activity was observed after treatment of target expressing EBC-1 cells with Q397-noV205C in the presence of PBMCs from all donors as compared to the IgG1 isotype control (DNS). Q397-noV205C did not induce EBC-1 cell lysis in the presence of 30% human complement, indicating Q397-noV205C lacks CDC activity (DNS).

### Example 4. In Vivo Functional Activity of MYTX-011 in Tumors Expressing cMET

MYTX-011 was assessed for in vivo activity in NCI-H2122 (“H2122”), NCI-H1975 (“H1975”), NCI-H1373 (“H1373”), and NCI-H1650 (“H1650”) human lung adenocarcinoma tumors and in EBC-1 human lung squamous cell carcinoma tumors in mouse xenograft models. These models were specifically chosen to test the ability of MYTX-011 to address a wide range of clinically important cMET+/cMET-overexpressing tumor types.

Tumor cell culture. EBC-1 human lung squamous carcinoma cells were cultured in EMEM containing 100 units/mL penicillin G sodium, 100 mg/ml streptomycin sulfate, and 25 mg/ml gentamicin. The medium was supplemented with 10% fetal bovine serum and 2 mM glutamine. The cells were cultured in tissue culture flasks in a humidified incubator at 37° C., in an atmosphere of 5% CO₂ and 95% air. The human lung adenocarcinoma NCI-H1373 cell line was maintained as suspension cultures in 90% RPMI 1640 medium and 10% heat inactivated fetal bovine serum according to ATCC recommendations. The cells were cultured in tissue culture flasks in a humidified incubator at 37° C., in an atmosphere of 5% CO2 and 95% air. The human lung adenocarcinoma NCI-H1975 cell line was maintained as suspension cultures in 90% RPMI 1640 medium and 10% heat inactivated fetal bovine serum according to ATCC. The cells were cultured in tissue culture flasks in a humidified incubator at 37° C., in an atmosphere of 5% CO2 and 95% air. Human lung adenocarcinoma NCI-H1650 cells were cultured in RPMI-1640 medium containing 100 units/mL penicillin G sodium, 100 mg/mL streptomycin sulfate, and 25 mg/ml gentamicin. The medium was supplemented with 10% fetal bovine serum and 2 mM glutamine. The cells were cultured in tissue culture flasks in a humidified incubator at 37° C. in an atmosphere of 5% CO2 and 95% air.

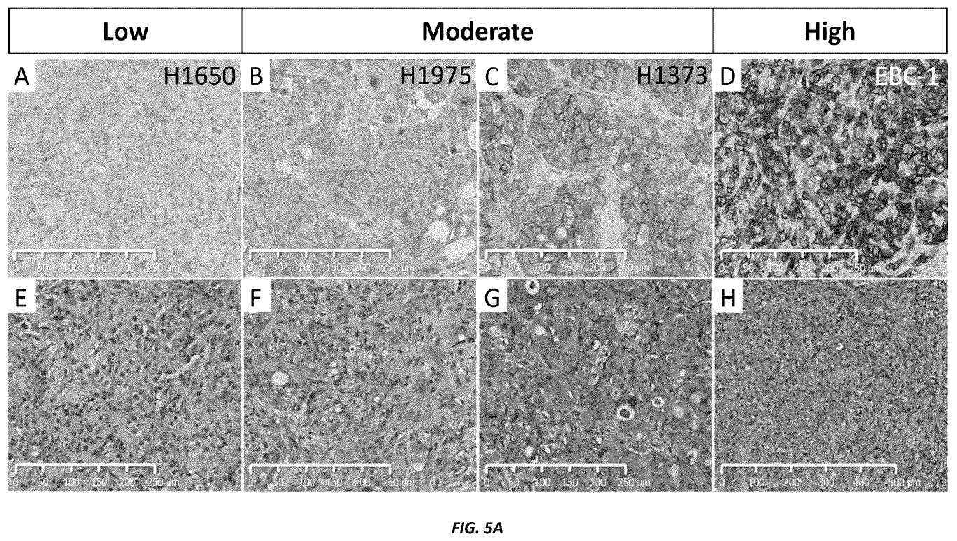

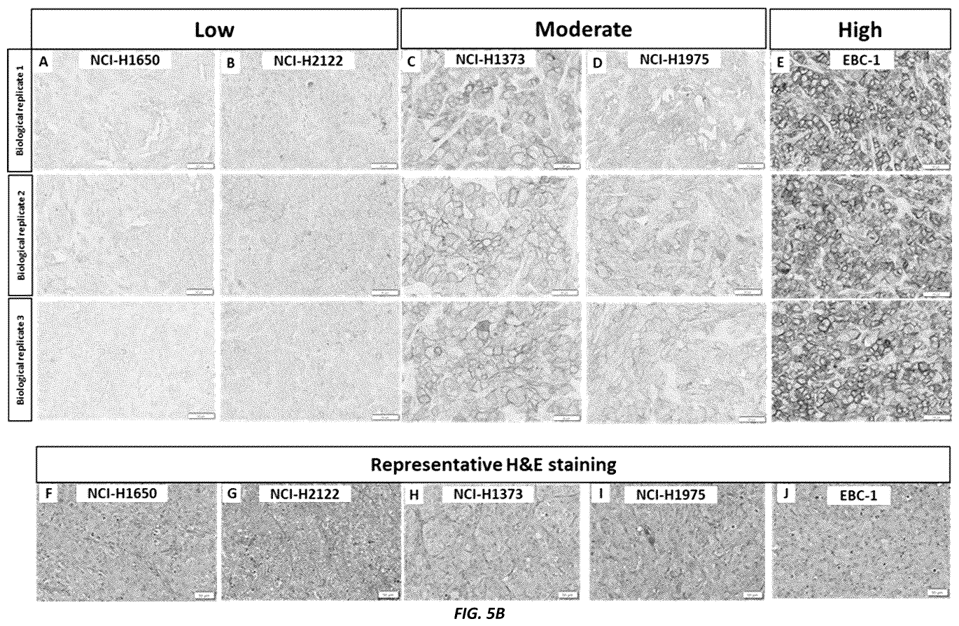

Tumor Cell Implantation. For all studies, cells used for implantation were harvested during log phase growth and resuspended in cold PBS containing 50% Matrigel™ (BD Biosciences). Each mouse was injected subcutaneously in the right flank with 5×10⁶ cells (in a 0.1 mL cell suspension). Tumors were allowed to grow until all were between 100-150 mm³. FFPE tumor blocks were then prepared from untreated mice bearing H2122, H1975, H1373, EBC-1, and H1650 tumors of 108-288 mm³ in size. Sections (4 μm) from the FFPE blocks (xenograft tissue and control cell pellets) were collected on positively charged slides and were stained for the optimized cMET (SP-44) conditions (1:50, CC1 Standard), isotype, and H&E stain. Anti-cMET antibody clone SP44 was used. All IHC staining was carried out on a DISCOVERY ULTRA® system v21.00.0019. The intensity of staining in the tumors was compared, and models were assessed as either low, moderate, or high relative expression of cMET. Staining was also carried out in H1650 and H1975 FFPE cell pellets. Ramos cells (cMET negative) and SNU-5 cells (cMET high expression) were included as negative and positive control FFPE cell pellets, respectively. The H2122, H1373, H1650, H1975, and EBC-1 xenograft tumors all demonstrated positive cMET staining (FIGS. 5A & 5B) with the anti-cMET antibody (SP44 clone) and negative isotype control antibody staining (data not shown).

The large relative difference observed in staining intensity between the negative and positive control samples indicated that the IHC staining is specific for cMET expression. The human lung cancer xenograft models characterized in this study showed different levels of staining intensity and were qualitatively categorized as having low (H2122 and H1650), moderate (H1975 and H1373), or high (EBC-1) cMET overexpression.

Activity of MYTX-011 in the cMET+ Mouse Xenograft Model EBC-1. The efficacy of MYTX-011 was assessed in the cMET-expressing EBC-1 human lung squamous cell carcinoma xenograft model using female SCID mice. The EBC-1 xenograft model in mice was selected because of the squamous cell carcinoma NSCLC origin and presence of MET gene amplification which results in cMET overexpression. EBC-1 has been previously characterized to express high levels of cMET and be dependent on cMET receptor for growth and survival.

Briefly, at age 8 weeks, SCID mice were injected subcutaneously (SC) with the EBC-1 human tumor cell line. When tumors reached an average size of 100 to 150 mm³, intravenous (IV) treatment with MYTX-011 and control articles was initiated. ADC test article dosage was normalized to administer equivalent amounts of MMAE payload. In the pilot efficacy study, single doses of MYTX-011 at 2 mg/kg and 4 mg/kg were compared to a vehicle control (PBS). In the dose titration study, a single dose of MYTX-011 at 0.5 mg/kg, 1 mg/kg, and 2 mg/kg was compared with single doses of a RC1-ADC-(DAR3.01) at 0.325 mg/kg, 0.65 mg/kg and 1.3 mg/kg and a vehicle control (PBS) group. RC1 is an antibody with variable regions from telisotuzumab (CAS 1781223-80-0) and RC1-ADC is the corresponding ADC comprising RC1 conjugated to MMAE via hinge-conjugation (DAR ^{˜}3.1).

Tumors were measured with calipers biweekly. Body weight was measured every day for 5 days, then biweekly for the pilot efficacy study. Animals were weighed biweekly post-randomization for the dose titration study. Animals were euthanized when tumors reached 2000 mm³ or at the end of the study (28 days for the pilot; 75 days for the dose titration).

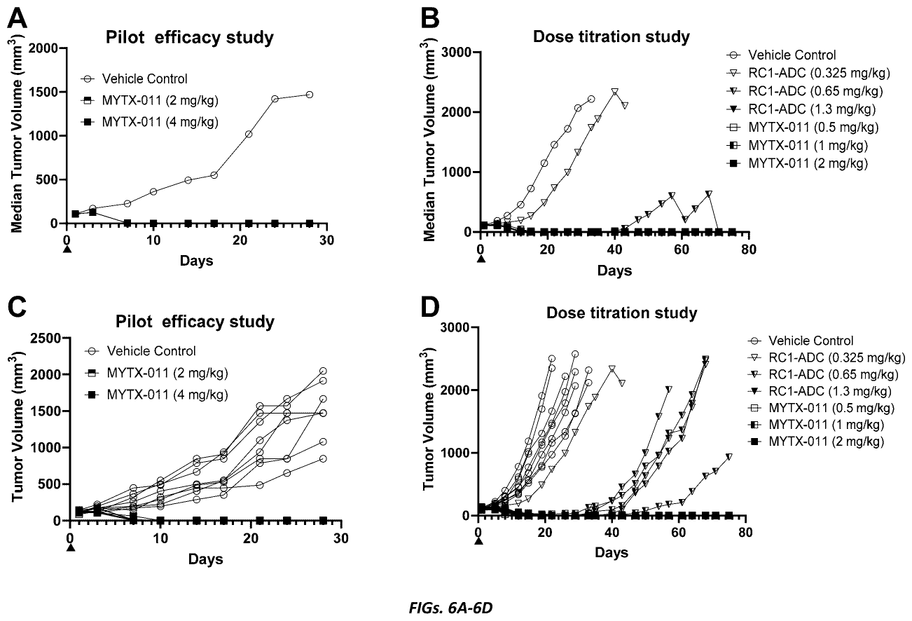

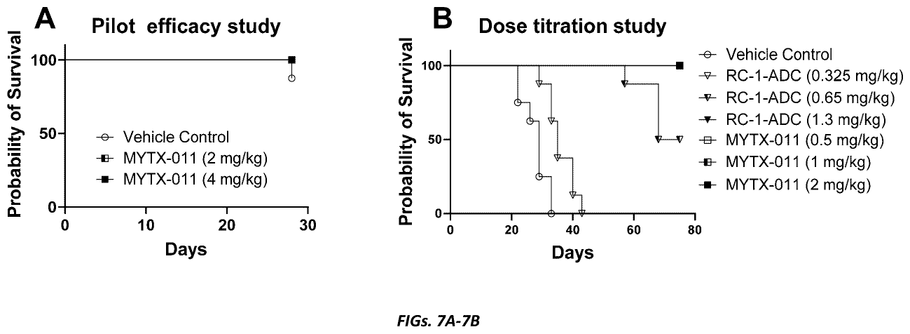

MYTX-011 treatment significantly decreased the growth of EBC-1 xenograft tumors across all doses tested (FIGS. 6A & 6C and FIGS. 6B & 6D). MYTX-011 was effective at reducing EBC-1 tumor growth when administered at lower doses on an MMAE equivalents basis as compared to RC1-ADC-(DAR3.01). Further, MYTX-011 administered at 0.5 mg·kg, 1 mg/kg, and 2 mg/kg substantially increased survival compared to RC1-ADC-(DAR3.1) administered at 0.325 mg/kg and 0.65 mg/kg (FIG. 7B). There was no mortality, body weight changes or clinical observations associated with any of the treatments.

Activity of MYTX-011 in the cMET+ Mouse Xenograft Model H1975. The efficacy of MYTX-011 was assessed in the cMET-expressing H1975 human lung adenocarcinoma xenograft model using female CB.17 SCID mice. The H1975 xenograft model in mice was selected because of its adenocarcinoma non-small cell lung cancer origin and the presence of oncogenic EGFR L858R and T790M mutations. H1975 has been characterized to express moderate levels of cMET receptor (Gymnopoulos 2020 and disclosed supra). Reference compound 5 (RC5) is an antibody with variable regions from an anti-HIV gp120 antibody disclosed in U.S. Pat. No. 5,652,138.

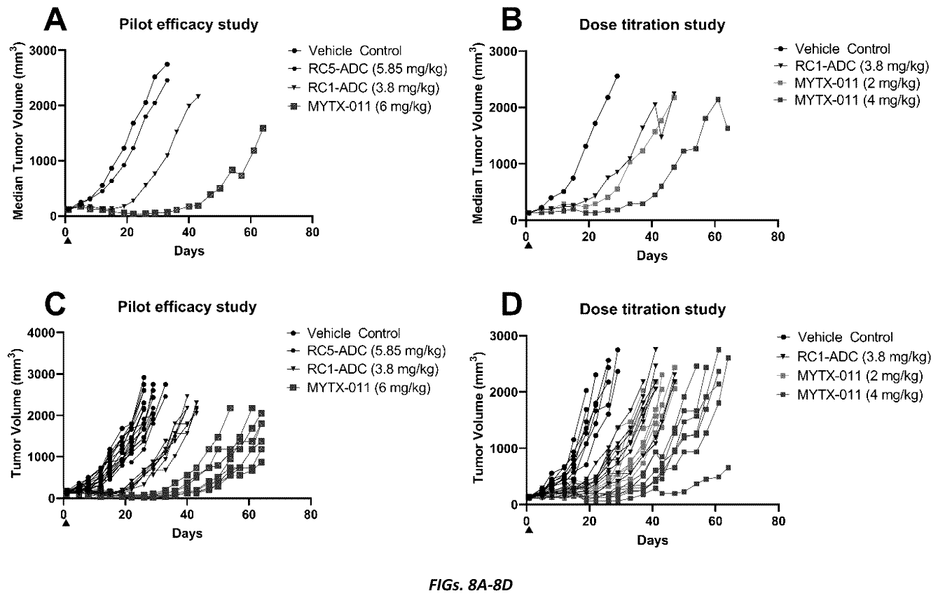

In the pilot efficacy study, a single dose of MYTX-011 was administered at 6 mg/kg and compared with the nonbinding control RC5-ADC-(DAR2) administered at 5.85 mg/kg, and to the RC1-ADC-(DAR3.1) at 3.8 mg/kg (FIGS. 8A & 8C). In the dose titration study, a single dose of MYTX-011 was administered at 2 and 4 mg/kg and RC1-ADC-(DAR3.1) was administered at 3.8 mg/kg (FIGS. 8B & 8D).

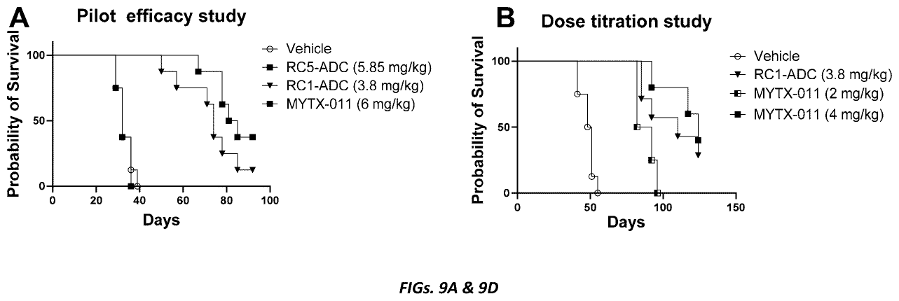

In the pilot efficacy study, tumor volumes were compared on Day 26. MYTX-011 administered at 6 mg/kg resulted in significantly decreased tumor volume (FIGS. 8A & 8C) and increased survival (FIG. 9A) compared with the non-binding, MMAE-containing control RC5-ADC-(DAR2) administered at 5.85 mg/kg (P<0.0001, both comparisons). In addition, MYTX-011 administered at 6 mg/kg showed significantly reduced tumor volume and increased survival compared with RC1-ADC-(DAR3.1) at 3.8 mg/kg (P<0.0001 and P=0.0001, respectively).

In the dose titration study, tumor volumes were measured and compared on Day 19. MYTX-011 administered at 2 or 4 mg/kg showed significantly reduced tumor volume (P=0.0005 and 0.0002, respectively) and increased survival (P<0.0001 and P=0.0017, respectively) compared with the vehicle control group (FIGS. 8B & 8D). Furthermore, there was a significant decrease in tumor volumes and a significant increase in survival with MYTX-011 treatment at 4 mg/kg compared with 2 mg/kg (P=0.0057 and 0.0021, respectively). MYTX-011 administered at 4 mg/kg, but not 2 mg/kg showed significantly reduced tumor volume and increased survival compared with RC1 ADC-(DAR3.1) at 3.8 mg/kg (P<0.0001 and P=0.0017, respectively). There was no mortality, body weight changes or clinical observations associated with any of the treatments.

H1975 Conclusions. Tumor growth in the moderate-cMET-expressing H1975 xenograft model at 26 days was significantly decreased by MYTX-011 compared with the non-binding ADC control, indicating that the observed effects are on-target. MYTX-011 significantly inhibited H1975 tumor growth and increased survival in a dose dependent manner in mice at 2 and 4 mg/kg. Efficacy of MYTX-011 against H1975 xenograft tumors was significantly greater than that observed with RC1-ADC-(DAR3.1) at the equivalent toxin dose.

Activity of MYTX-011 in the cMET+ Mouse Xenograft Model H1373. The efficacy of MYTX-011 was assessed in the cMET-expressing H1373 human lung adenocarcinoma xenograft model using female CB.17 SCID mice. The H1373 xenograft model in mice was selected because of its adenocarcinoma non-small cell lung cancer origin and the presence of the oncogenic KRAS G12C mutation. H1373 has been shown to express moderate cMET levels (Gymnopoulos 2020).

In the pilot efficacy study, a single dose of MYTX-011 administered at 6 mg/kg was compared with the non-binding control RC5-ADC-(DAR2) administered at 5.85 mg/kg, and RC1-ADC-(DAR3.1) administered at 3.8 mg/kg. In the dose titration study, a single dose of MYTX-011 was administered at 2 and 4 mg/kg, and RC1-ADC-(DAR3.1) was administered at 3.8 mg/kg.

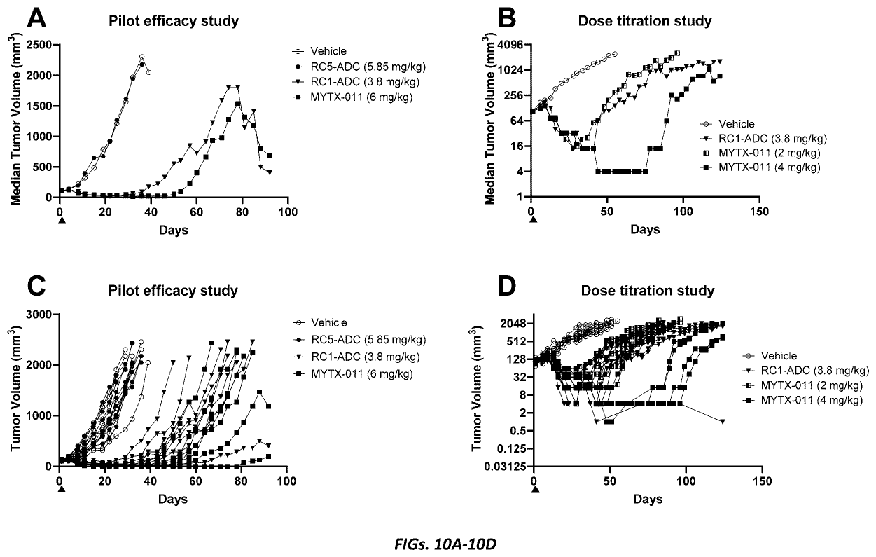

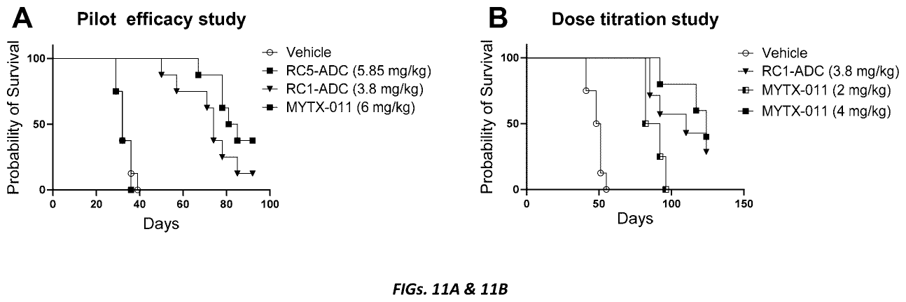

In the pilot efficacy study, tumor volumes were compared on Day 29. MYTX-011 administered at 6 mg/kg resulted in significantly decreased tumor volume (FIGS. 10A & 10C and FIGS. 10B & 10D) and increased survival (FIG. 11A) compared with the nonbinding, MMAE-containing control RC5-ADC-(DAR2) administered at 5.85 mg/kg (P<0.0001, both comparisons). MYTX-011 administered at 6 mg/kg did not show a statistically significant reduction in tumor volume or increase in survival compared with RC1-ADC-(DAR3.1) at 3.8 mg/kg.

In the dose titration study, tumor volumes were measured and compared on Day 41. MYTX-011 administered at 2 or 4 mg/kg showed significantly reduced tumor volume (P=0.0005 and 0.0004, respectively) and increased survival (P<0.0001 and P=0.0009, respectively) compared with the vehicle control group. Furthermore, there was a significant increase in median survival with MYTX-011 treatment at 4 mg/kg compared with 2 mg/kg (P=0.0055), however, there was no statistically significant reduction in tumor volume at the higher dose. MYTX-011 administered at 4 mg/kg did not show significantly increased survival compared with RC1-ADC-(DAR3.1) administered at 3.8 mg/kg.

H1373 Conclusions. Tumor growth in the moderate-cMET-expressing H1373 xenograft model at 29 days was significantly decreased by MYTX-011 compared with the non-binding ADC control, RC5-ADC-(DAR2), indicating that the observed effects are on-target. MYTX-011 substantially inhibited H1373 xenograft tumor growth and increased survival in mice at 2 mg/kg and 4 mg/kg. In this model and at these dose levels, a dose-dependent effect on tumor volume was not observed at Day 41, however, 4 mg/kg resulted in a statistically significant longer median survival than 2 mg/kg, suggesting the effects of MYTX-011 are dose dependent.

Overall conclusions for this example. All models were insensitive to the non-targeting control ADC and the following models were susceptible to MYTX-011 (expression by FACS/IHC score): EBC-1 (134608/3+), H1373 (46450/2+), H1975 (42360/2+), and H2122 (1237/1+). The H1650 cells (1891/1+) did not appear to respond to MYTX-011 in vivo, potentially indicating that H1650 cells are more resistant to the MMAE cargo.

### Example 5: Antitumor Activity of Anti-cMET pH-ADCs in Models of Human NSCLC in Immunocompromised Mice

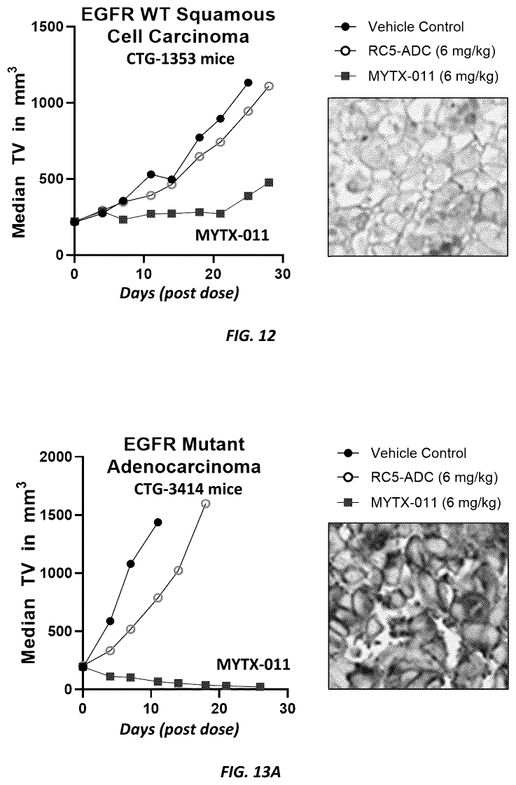

The efficacy of anti-cMET pH-ADCs (in this case, MYTX-011) was evaluated in five different non-small cell lung cancer (NSCLC) patient-derived xenograft (PDX) models (CTG-2669, CTG-3414, CTG-1353, CTG-2533, and CTG-2082), selected based on criteria such as (1) histopathology, (2) the presence of T790M EGFR mutation which confers TKI resistance, (3) presence of D1028N exon 14 skipping MET mutation, and (4) having a score of 2+ based on the anti-cMET (clone SP44) IHC assay. PDX model characteristics are as follows: CTG-1353 (squamous cell carcinoma, EGFR wildtype, about intermediate cMET expression overall); CTG-2533 & CTG-2669 (both adenocarcinoma, EGFR wildtype, and about intermediate cMET overexpression overall); and CTG-3414 (adenocarcinoma, EGFR E746_A750del & T790M, and about high cMET overexpression overall) (Champions Oncology). As regards the mutations, the presence of the EGFR T790M mutation is known to confer thymidine kinase inhibitor (TKI) resistance. And finally, an SP44 IHC negative PDX model of squamous cell carcinoma, CTG-2082 (EGFR WT and MET WT), was also included. As detailed further below, 4 PDX models showed significant sensitivity to a single 6 mg/kg dose of the anti-cMET pH-ADC. Notably, the responsive models included 2 EGFR wildtype adenocarcinomas, 1 EGFR squamous cell carcinoma, and 1 EGFR mutant adenocarcinoma, derived from a patient that had progressed on osimertinib (a third-generation EGFR TK inhibitor). See FIGS. 12-15.

Pre-study Animals. When sufficient stock animals reached 1000-1500 mm³, tumors were harvested for re-implantation into pre-study animals. Pre-study animals were then implanted unilaterally on the left flank with tumor fragments harvested from stock animals. Each animal was implanted from a specific passage lot and documented.

Dosing. Mice were subcutaneously implanted with patient-derived xenograft (PDX) tumor fragments. Pre-study tumor volumes were recorded for each experiment beginning seven to ten days after implantation. When tumors reached an average size of 150-300 mm³, animals were intravenously administered a single dose of MYTX-011 at 6 mg/kg or RC5-ADC-(DAR2) at 6 mg/kg. A vehicle control (PBS) group was included in each study. Tumors were measured with calipers twice weekly. Animal body weight was measured biweekly post-randomization. Animals were euthanized when tumors reached 2000 mm³ or at the end of the study (Day 28 for CTG-1353, Day 52 for CTG-2082, D26 for CTG-2533, D42 for CTG-2669 and D26 for CTG-3414).

Satellite Study Animals. After randomization of the animals in the study groups, tumor growth in 5 non-randomized pre-study animals was monitored until satellite tumors were collected for comparison to animals treated with the test article. Tumor volumes were measured twice weekly until 3 tumors were within the 150-300 mm³ range (i.e. as close as possible to the MTV of the randomized groups). Whole tumors were placed in 10% neutral buffered formalin (NBF) for 18-24 hours, transferred to 70% ethanol and stored at room temperature. Formalin fixed samples were then paraffin embedded for subsequent analysis.

Data Analysis. Beginning at Day 0, animals were observed daily and weighed twice weekly using a digital scale; data including individual and mean gram weights (Mean Weight±SEM), mean percent weight change versus Day 0 (% vDO) was recorded for each group and % vDO plotted at study completion. Single agent or combination groups reporting a mean % vDO>20% and/or >10% mortality were considered above the maximum tolerated dose (MTD) for that treatment on the evaluated regimen. Maximum mean % vDO (weight nadir) for each treatment group was reported at study completion.

Tumor Measurements. Tumor dimensions were also measured twice weekly by digital caliper and data including individual and mean estimated tumor volumes (Mean TV±SEM) recorded for each group; tumor volume was calculated using the formula (1): TV=width×length×0.52. At study completion, percent tumor growth inhibition (% TGI) values were calculated and reported for each treatment group (T) versus control (C) using initial (i) and final (f) tumor measurements by the formula (2): % TGI=1−(Tf−Ti)/(Cf−Ci)*100. Individual mice reporting a tumor volume less than or equal to 30% of the Day 0 measurement for two consecutive measurements were considered partial responders (PR). Individual mice lacking palpable tumors (0.00 mm³ for two consecutive measurements) were classified as complete responders (CR); a CR that persisted until study completion were considered a tumor-free survivor (TFS). Tumor doubling time (DT) was determined for the vehicle treated groups using the formula DT=(Df−Di)*log 2/(log TVf−log TVi) where D=Day and TV=Tumor Volume.

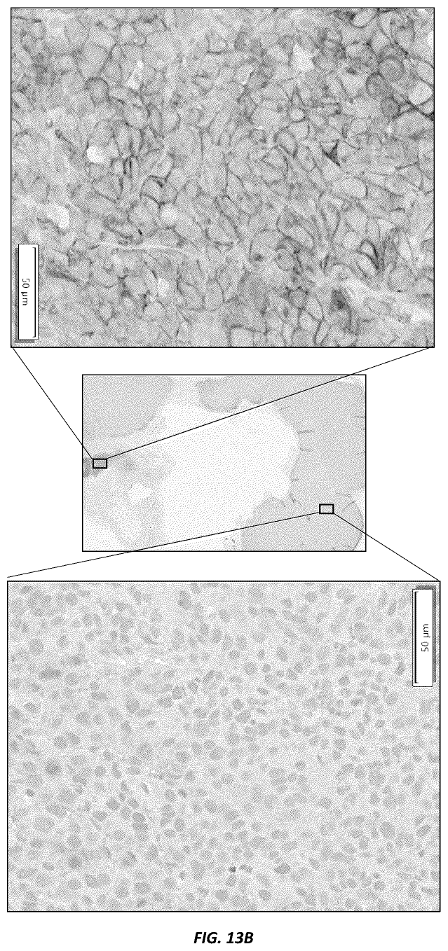

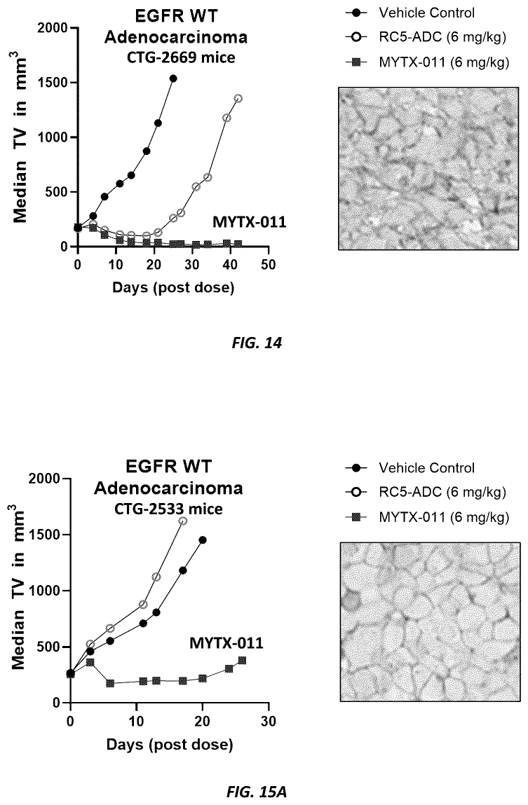

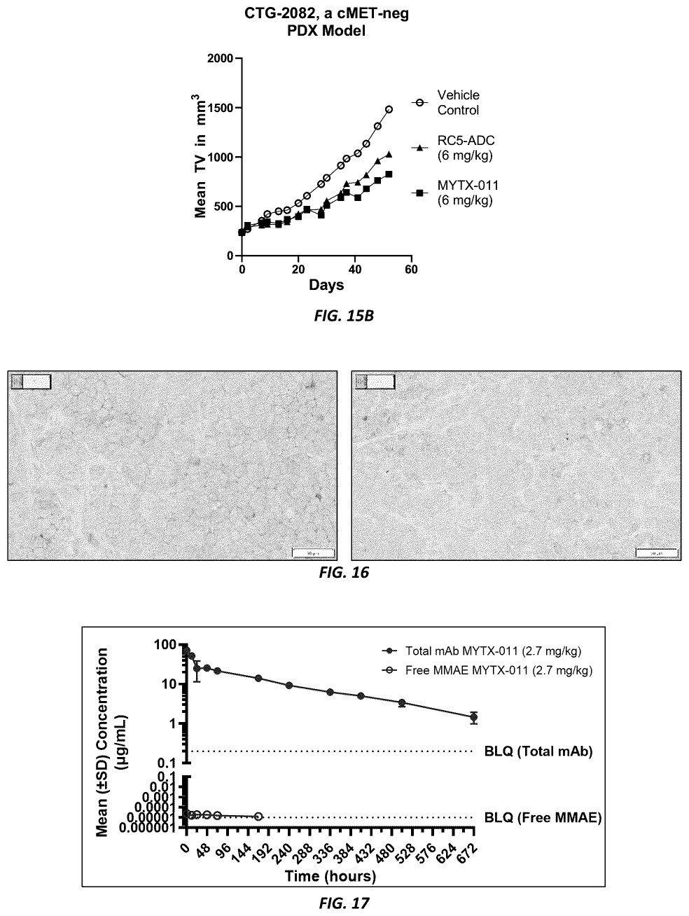

Results. Expression of cMET was assessed by IHC on tumors isolated from 5 PDX models using SP44 cMET IVD assay. As illustrated in FIG. 12 (see micrograph, right), the CTG-1353 model retained its overall 2+ grading of IHC, and exhibited heterogenous cMET expression. One exemplary section exhibited ^{˜}20% area of 1+ grading, another was homogeneously a “weaker” 2+, and yet another section was heterogeneous (data not shown, “DNS”). And as illustrated by FIG. 13A, some CTG-3414 tumor exhibited high levels of cMET overexpression. Interestingly, one PDX tumor exhibited high heterogeneity with <5% area with 3+ cMET expression (see, e.g., FIG. 13B), whereas another exhibited ^{˜}50% cMET positive with 3+ grading and ˜50% completely negative (DNS). As shown in FIG. 14, CTG-2669 retained its 2+ grading; however, an evaluation of other samples indicated substantial heterogeneity of cMET overexpression, with one tumor entirely negative for cMET (DNS). Furthermore, CTG-2533 (FIG. 15A) faithfully modeled consistent 2+ scoring among all tumors evaluated. As regards efficacy, MYTX-011, administered at 6 mg/kg, exhibited significant activity compared to vehicle control and RC5-ADC-(DAR2) in these models. And finally, administration at 6 mg/kg of MYTX-011 did not significantly reduce the size of cMET negative CTG-2082 tumors relative to control (FIG. 15B).

In conclusion, MYTX-011 treatment significantly decreased the growth of different NSCLC PDX tumors compared to non-binding and vehicle controls. Notably, MYTX-011 did not significantly impact tumor volume in the cMET-negative, CTG-2082 PDX model. And finally, no mortality, body weight changes, or clinical observations were associated with any of the treatments. Taken together, these data show that MYTX-011 is effective against a variety of human NSCLC tumors, including those characterized by heterogeneous cMET overexpression.

### Example 6. Efficacy of MYTX-011 in a Low cMET NSCLC Mouse Model

Among the many exceptional features of MYTX-011 (and other anti-cMET pH-ADCs described herein) is that it exhibits efficacy against cMET+ tumors having expression levels as low as 1+ measured by the IHC scoring methods described herein. In this example, the efficacy of MYTX-011 was tested in a cMET low, EGFR wildtype animal model.

Briefly, 43 Nude (Envigo #069) female mice, 6 weeks old were procured and housed in 10 cages of 4 and 1 cage of 3. Animals were randomized into 4 groups of 8 (based on tumor volume after each reached about 125-150 mm³) as follows: 1) vehicle (PBS); 2) Test114-MMAE, DAR 4 (6 mg/kg); TestF-MMAE, DAR 3.5 (6.85 mg/kg); Test114 (naked mAb, 6 mg/kg); MYTX-011, DAR 2.0 (6 mg/kg); each inoculation performed subQ, IV, and qdX1 (i.e. a single dose study). NCI-H2122 NSCLC cells were grown in RPMI-1640 (+10% FBS) for inoculation of 7e6 cells per animal in 100 μl serum free medium/Geltrex (1:1), subcutaneously in R-Flank. Tumor measurements/body weights were captured via electronic caliper and scales twice weekly beginning 3 days post inoculation. Each animal was administered a single IV injection of test article (fixed volume of 200 μl per mouse) immediately after randomization. The study continued until the tumors reached a max tumor volume of about 1500 mm³.

Results/Conclusions. As shown in FIG. 16, sections from H2122 tumors show substantially lower levels of cMET expression as compared to the 2+ and 3+ sections presented in FIGS. 12, 14, and 15A. In general, H2122 tumors stained heterogeneously. One tumor was entirely negative for overexpression, and others were primarily 1+ with patches that were ^{˜}2+. And remarkably, MYTX-011 appeared to exert tumor shrinkage for several days after the single administration, so it is envisioned that multiple administrations would be even more effective against such cMET low tumors. Further, MYTX-011 was significantly more effective than a non-pH-engineered comparator ADC (i.e., TestF-MMAE, DAR 3.5) carrying about 1.75 fold more drug payload (i.e., the MMAE). To Applicant's knowledge, this is the first disclosure of a cMET-targeting ADC that demonstrates this degree of efficacy against tumors having such low levels of cMET expression.

Moreover, MYTX-011 exerted bystander activity in both the cMET low H2122 CDX model of this example and the cMET high CTG-3414 PDX model of the prior example. And considering that each of these models exhibited a substantial degree of heterogeneity of cMET overexpression throughout the tumor (as assessed by IHC; see e.g., FIG. 13B), it is envisioned that MYTX-011 will be effective against a variety of cMET+/cMET-overexpressing tumors, including those expressing relatively low levels of cMET and/or those comprising heterogenous levels of cMET overexpression.

### Example 7. Pharmacokinetics (PK) and Toxicokinetics (TK) of MYTX-011

Safety Pharmacology. Cardiovascular, CNS, and respiratory safety pharmacology endpoints were incorporated into a 25-day repeat-dose IV toxicity study in monkeys.

There were no functional cardiovascular (ECGs, blood pressure, heart rate), CNS (daily clinical and weekly detailed observations) or respiratory system (daily clinical and weekly detailed observations) findings observed in monkeys during 22 days of once every three weeks IV administration of MYTX-011 at 18 mg/kg/week.

PK and TK. The pharmacokinetics (PK) and toxicokinetics (TK) of MYTX-011, and its components, were studied in cynomolgus monkeys after single and multiple doses.

TABLE 1
List of Completed Pharmacokinetic/Toxicokinetic
Studies for MYTX-011

| Species | Dose Schedule | Route | Doses | GLP Status |
|---|---|---|---|---|
| Cyno Monkey | Single Dose | IV | 2.7 mg/kg | Non-GLP |
| Cyno Monkey | Every 3 Weeks | IV | 6, 12, and | Non-GLP |
| | | | 18 mg/kg | |
| Cyno Monkey | Every 3 Weeks | IV | 6, 12, and | GLP |
| | | | 18 mg/kg | |

PK of MYTX-011 after Administration of a Single Intravenous Dose in Female Cynomolgus Monkeys. Pharmacokinetic profile of MYTX-011 was determined in female cynomolgus monkeys after administration of a single intravenous (IV) dose. In total, 3 animals were utilized in this study and were all dosed at 2.7 mg/kg. Blood samples were collected for evaluation of the pharmacokinetic parameters at the following timepoints: pre-dose (0), 0.25, and 12 hours post-dose on Day 1 and on Days 2, 3, 4, 7, 10, 14, 17, 21, and 28 post-dose. FIG. 17 presents the data for the total mAb and free MMAE.

As outlined in Table 2, following a single IV injection of MYTX-011 to female cynomolgus monkeys, the total mAb concentrations increased rapidly to a maximum concentration of 70.9 μg/mL at a dose of 2.7 mg/kg. Average area under the curve from 0 to the last measurable timepoint (AUClast) for the total mAb was calculated to be 6810 hr·μg/mL at a dose of 2.7 mg/kg. Average half-life (T1/2) of the total mAb was estimated to be 158-hours at a dose of 2.7 mg/kg. Concentrations of free MMAE were collected over a 28-day period but were not measurable beyond 168-hours post-dose in all study animals. The average Cmax concentration of free MMAE following administration of a single IV injection of MYTX-011 to female cynomolgus monkeys at a dose of 2.7 mg/kg was 0.0254 μg/mL.

TABLE 2
Summary of PK Data for Total mAb After
a Single IV Administration of MYTX-011

| | Molecule (Analyte) | | |
|---|---|---|---|
| | MYTX-011 (Total mAb) | | |
| | Dose (mg/kg) | | |
| | 2.7 | | |
| Animal ID | N | Mean | SD |
| Cₘₐₓ (μg/mL) | 3 | 70.9 | 9.93 |
| Tₘₐₓ (hr) | 3 | 0.25 | 0 |
| Cₗₐₛₜ (μg/mL) | 3 | 1.45 | 0.468 |
| Tₗₐₛₜ (hr) | 3 | 672 | 0 |
| T_{1/2} (hr) | 3 | 158 | 31 |
| AUC_{0-240 h} (hr · μg/mL) | 3 | 4860 | 407 |
| AUCₗₐₛₜ (hr · μg/mL) | 3 | 6810 | 673 |
| AUC_{inf} (hr · μg/mL) | 3 | 7160 | 789 |
| AUC Extrap % | 3 | 4.72 | 2.13 |
| CL (mL/hr) | 3 | 0.761 | 0.0895 |
| Vz (mL) | 3 | 172 | 24.6 |

CL = clearance; Clast = last observed quantifiable concentration; Cmax = maximum concentration; Tmax = time of maximum concentration; *Data should be interpreted with caution due to high extrapolation percent.

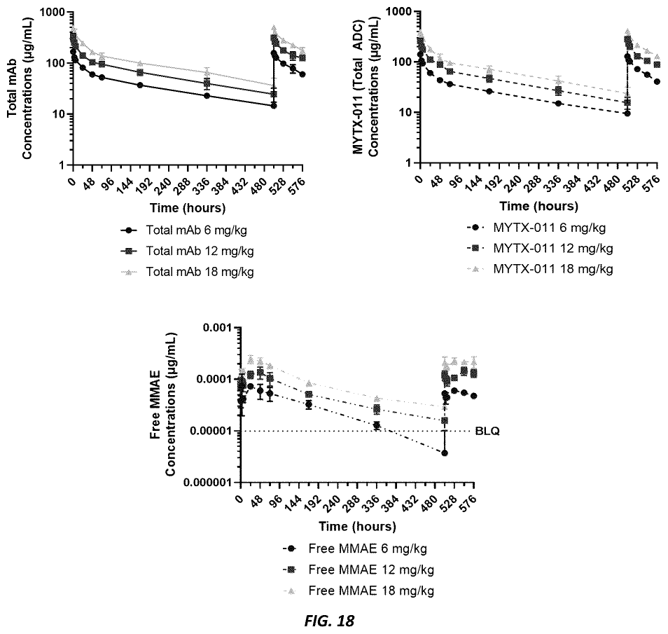

Toxicokinetics of MYTX-011 After Administration of Multiple IV Doses Across a Dose Range in Male Cynomolgus Monkeys. Following once every 3-week IV bolus injection of MYTX-011 to male cynomolgus monkeys, Cₘₐₓ and AUC_{0-72 h} increased in a dose proportional manner for MYTX-011 across the tested dose range on Day 1 and Day 22. For the total mAb, Cₘₐₓ and AUC_{0-72 h} increased in a dose proportional manner across the tested dose range on Day 1 and Day 22. Little to no accumulation in systemic exposure of MYTX-011 or the total mAb, as measured by AUC_{0-72 hr}, was observed following repeated administration at 6, 12, and 18 mg/kg. As observed in published literature (Okamoto 2020; Kinoshita 2021; Takahashi 2020), the exposure of the MYTX-011 (total ADC), as measured by Cmax and AUC0-72 hrs was generally lower than that observed with the total mAb. Summary of MYTX-011 toxicokinetics is summarized in Table 3 and TK profiles of all analytes are summarized in FIG. 18.

Following once every three-week IV bolus injection of MYTX-011 to male cynomolgus monkeys, on Day 1, free MMAE Cₘₐₓ increased in a slightly less than dose proportional manner between 6 and 12 mg/kg and was dose proportional between 6 and 18 mg/kg. On Day 22, Cₘₐₓ values increased in a more than dose proportional manner. This observation is potentially due to the decreased exposure in free MMAE observed in the 6 mg/kg group on Day 22. For AUC_{0-72 hr} on Day 1, there was a dose proportional increase in exposure between 6 and 12 mg/kg and a more than dose proportional increase in exposure between 6 and 18 mg/kg. On Day 22, there was a slightly more than dose proportional increase in exposure between doses of 6 and 12 mg/kg and a more than dose proportional increase between 6 and 18 mg/kg for free MMAE.

TABLE 3
Summary of MYTX-011 (Total ADC) Toxicokinetic Parameters in Male Cynomolgus
Monkeys Following Multiple Intravenous Doses at 6, 12, and 18 mg/kg

| | | | | | | | | | AUC_{0-72 hr}/ | | | | |
|---|---|---|---|---|---|---|---|---|---|---|---|---|---|
| | | | | Cₘₐₓ/ | | | | | Dose | | | | |
| | | | | Dose | | | AUCₗₐₛₜ | AUC_{0-72 hr} | (hr*kg* | AUC_{inf} | CL | | |
| Dose | | | Cₘₐₓ | (kg*μg/ | Tₘₐₓ^{a} | Tₗₐₛₜ^{a} | (hr*μg/ | (hr*μg/ | μg/mL/ | (hr*μg/ | (mL/hr/ | V_{z} | |
| (mg/kg) | Day | Statistic | (μg/mL) | mL/mg) | (hr) | (hr) | mL) | mL | mg) | mL) | kg) | (mL/kg) | R^{b} |
| 6 | 1 | N | 3 | 3 | 3 | 3 | 3 | 3 | 3 | 3 | 3 | 3 | NA |
| | | Mean | 141 | 23.5 | 0.25 | 504 | 12,700 | 4240 | 707 | 15,700 | 0.383 | 123 | NA |
| | | SD | 7.55 | 1.26 | (0.25-0.25) | (504-504) | 682 | 240 | 40.1 | 650 | 0.0156 | 9.54 | NA |
| | | CV % | 5.35 | 5.35 | NA | NA | 5.36 | 5.67 | 5.67 | 4.15 | 4.06 | 7.75 | NA |
| | 22 | N | 3 | 3 | 3 | 3 | 3 | 3 | 3 | NA | NA | NA | 3 |
| | | Mean | 129 | 21.4 | 0.25 | 72 | 4920 | 4920 | 819 | NA | NA | NA | 1.16 |
| | | SD | 4.93 | 0.822 | (0.25-0.25) | (72-72) | 197 | 197 | 32.9 | NA | NA | NA | 0.0418 |
| | | CV % | 3.83 | 3.83 | NA | NA | 4.01 | 4.01 | 4.01 | NA | NA | NA | 3.61 |
| 12 | 1 | N | 3 | 3 | 3 | 3 | 3 | 3 | 3 | 3 | 3 | 3 | NA |
| | | Mean | 260 | 21.7 | 0.25 | 504 | 23,100 | 8040 | 670 | 27,900 | 0.436 | 131 | NA |
| | | SD | 25.2 | 2.10 | (0.25-0.25) | (504-504) | 2420 | 166 | 13.8 | 4180 | 0.0627 | 8.04 | NA |
| | | CV % | 9.68 | 9.68 | NA | NA | 10.4 | 2.07 | 2.07 | 15 | 14.4 | 6.14 | NA |
| | 22 | N | 3 | 3 | 3 | 3 | 3 | 3 | 3 | NA | NA | NA | 3 |
| | | Mean | 280 | 23.4 | 0.25 | 72 | 9500 | 9500 | 792 | NA | NA | NA | 1.18 |
| | | SD | 4.04 | 0.337 | (0.25-0.25) | (72-72) | 549 | 549 | 45.7 | NA | NA | NA | 0.0580 |
| | | CV % | 1.44 | 1.44 | NA | NA | 5.77 | 5.77 | 5.77 | NA | NA | NA | 4.91 |
| 18 | 1 | N | 3 | 3 | 3 | 3 | 3 | 3 | 3 | 3 | 3 | 3 | NA |
| | | Mean | 391 | 21.7 | 0.25 | 504 | 35,600 | 12,400 | 690 | 43,200 | 0.429 | 128 | NA |
| | | SD | 55.5 | 3.09 | (0.25-0.25) | (504-504) | 5250 | 1260 | 69.7 | 8570 | 0.0959 | 9.38 | NA |
| | | CV % | 14.2 | 14.2 | NA | NA | 14.8 | 10.1 | 10.1 | 19.8 | 22.4 | 7.32 | NA |
| | 22 | N | 3 | 3 | 3 | 3 | 3 | 3 | 3 | NA | NA | NA | 3 |
| | | Mean | 416 | 23.1 | 0.25 | 72 | 15,100 | 15,100 | 837 | NA | NA | NA | 1.22 |
| | | SD | 22.1 | 1.23 | (0.25-0.25) | (72-72) | 745 | 745 | 41.4 | NA | NA | NA | 0.0845 |
| | | CV % | 5.33 | 5.33 | NA | NA | 4.95 | 4.95 | 4.95 | NA | NA | NA | 6.94 |

NA—Not applicable
AUC_{Tlast} after the first dose is equivalent to AUC_{0-504 hr}
^{a}Median (minimum - maximum), median value only reported if actual collection interval.
^{b}R = AUC_{0-72 hr} Day 22/AUC_{0-72 hr} Day 1Systemic exposure (AUC_{0-72 hr} and Cₘₐₓ) to free MMAE was less than systemic exposure to MYTX-011. Mean metabolite:parent (M:P) ratios based on AUC_{0-72 hr} were 0.00000102, 0.00000102, and 0.00000122 at 6, 12, and 18 mg/kg, respectively, on Day 1 and were 0.000000778, 0.000000916, and 0.00000102 at 6, 12, and 18 mg/kg, respectively, on Day 22. Mean M:P ratios based on Cₘₐₓ were 0.000000670, 0.000000535, and 0.000000702 at 6, 12, and 18 mg/kg, respectively, on Day 1, and were 0.000000477, 0.000000555, and 0.000000596 at 6, 12, and 18 mg/kg, respectively, on Day 22.

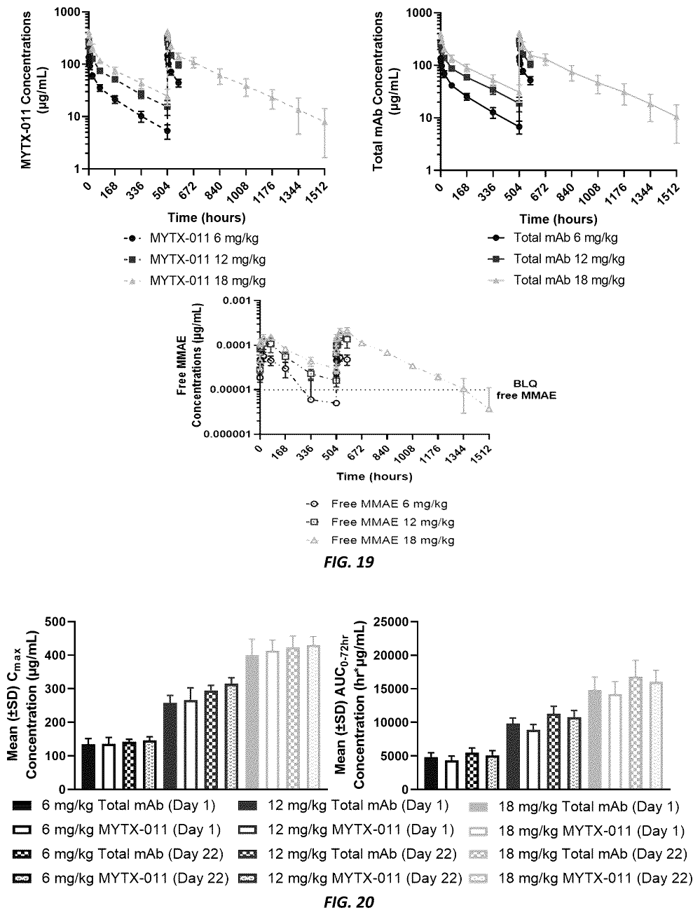

Toxicokinetics of MYTX-011 After Administration of Multiple Doses in the GLP Toxicity Study. In the GLP toxicology study, no differences were observed between gender upon repeat dosing of MYTX-011. Following once every 3 weeks IV bolus injection of MYTX-011, Cₘₐₓ and AUC_{0-72 hr} for MYTX-011 increased in an approximately dose proportional manner on both Day 1 and 22. On Day 1, AUC_{0-504 hr} and AUC_{inf} for MYTX-011 increased in a slightly more than dose proportional manner on Day 1. For the total mAb, Cₘₐₓ and AUC_{0-72 hr} increased in a dose proportional manner on both Day 1 and 22. On Day 1, AUC_{0-504 hr} and AUC_{inf} for MYTX-011 increased in a slightly more than dose proportional manner on Day 1. For free MMAE, Cₘₐₓ and AUC_{0-72 hr} increased in a dose proportional manner on both Day 1 and a more than dose proportional manner on Day 22. AUC_{0-504 hr} and AUC_{inf} for MYTX-011 increased in a slightly more than dose proportional manner on Day 1. Summary of MYTX-011 toxicokinetics is summarized in Table 4 and TK profiles of all analytes are summarized in FIG. 19.

Following repeat dosing of MYTX-011 at doses of 6, 12, and 18 mg/kg, systemic exposure (AUC_{0-72 hr} and AUC_{0-504 hr}) of MYTX-011, total mAb, and free MMAE did not appear to change after successive doses. Overall exposure of MYTX-011 (total ADC) and total mAb (the Ab component of MYTX-011) was similar suggesting a stable molecule, as depicted in FIG. 20.

As shown in Table 4, on Day 1, mean T_{1/2} values for MYTX-011 were 148, 162, and 155 (N=1) hours at 6, 12, and 18 mg/kg, respectively. On Day 22, MYTX-011 mean T_{1/2} at 18 mg/kg was 199 hours. For the total mAb on Day 1, T_{1/2} values were 148 and 158 at dose of 6 and 12 mg/kg, respectively. On Day 22, total mAb T_{1/2} was 210 hours at a dose of 18 mg/kg. For free MMAE, mean T_{1/2} values were 147, and 166 (N=1) hours at 12 and 18 mg/kg, respectively. On Day 22, mean T_{1/2} for free MMAE at 18 mg/kg was 213 hours.

Mean CL values for MYTX-011 on Day 1 were 0.499, 0.417, and 0.410 mL/hr/kg at 6, 12, and 18 mg/kg, respectively. On Day 22, mean CL values were 0.727, 0.655, and 0.570 mL/hr/kg at 6, 12, and 18 mg/kg, respectively. Mean Vz values for MYTX-011 on Day 1 were 117, 111, and 115 ml/kg at 6, 12, and 18 mg/kg, respectively. On Day 22, mean Vz values were 54.8, 52.3, and 70.1 ml/kg at 6, 12, and 18 mg/kg, respectively.

Systemic exposure (AUC_{0-72 hr}, AUC_{0-504 hr}, and Cₘₐₓ) to free MMAE was <systemic exposure to MYTX-011. Mean M:P ratios for systemic exposure were all <0.00000104.

TABLE 4
a. Summary of MYTX-011 (total ADC) toxicokinetic parameters

| | | | | Cₘₐₓ/ | | | | | |
|---|---|---|---|---|---|---|---|---|---|
| | | | | Dose | | | | AUCₗₐₛₜ | AUC_{0-72 hr} |
| Dose | | | Cₘₐₓ | (kg · μg/ | Tₘₐₓ^{a} | Tₗₐₛₜ^{a} | T_{1/2}^{b} | (hr · μg/ | (hr · μg/ |
| (mg/kg) | Day | Statistic | (μg/mL) | mL/mg) | (hr) | (hr) | (hr) | mL) | mL) |
| 6 | 1 | N | 6 | 6 | 6 | 6 | 3 | 6 | 6 |
| | | Mean | 136 | 22.7 | 0.083 | 504 | 148 | 11,100 | 4390 |
| | | SD | 19.0 | 3.17 | (0.083-0.083) | (504-504) | 16.1 | 1760 | 602 |
| | | CV % | 13.9 | 13.9 | NA | NA | 10.9 | 15.9 | 13.7 |
| | 22 | N | 6 | 6 | 6 | 6 | NA | 6 | 6 |
| | | Mean | 147 | 24.5 | 0.083 | 72 | NA | 5120 | 5120 |
| | | SD | 9.95 | 1.66 | (0.083-0.083) | (72-72) | NA | 690 | 690 |
| | | CV % | 6.76 | 6.76 | NA | NA | NA | 13.5 | 13.5 |
| 12 | 1 | N | 6 | 6 | 6 | 6 | 3 | 6 | 6 |
| | | Mean | 267 | 22.3 | 0.083 | 504 | 162 | 25,000 | 8920 |
| | | SD | 35.5 | 2.96 | (0.083-0.083) | (504-504) | 6.78 | 1960 | 773 |
| | | CV % | 13.3 | 13.3 | NA | NA | 4.19 | 7.85 | 8.66 |
| | 22 | N | 6 | 6 | 6 | 6 | NA | 6 | 6 |
| | | Mean | 315 | 26.3 | NA | 72 | NA | 10,800 | 10,800 |
| | | SD | 17.8 | 1.48 | (0.083-0.18) | (72-72) | NA | 985 | 985 |
| | | CV % | 5.65 | 5.65 | NA | NA | NA | 9.14 | 9.14 |
| 18 | 1 | N | 10 | 10 | 10 | 10 | 1 | 10 | 10 |
| | | Mean | 415 | 23.0 | 0.083 | 504 | 155 | 39,400 | 14,300 |
| | | SD | 30.8 | 1.71 | (0.083-0.083) | (504-504) | NA | 5620 | 1800 |
| | | CV % | 7.43 | 7.43 | NA | NA | NA | 14.3 | 12.6 |
| | 22 | N | 10 | 10 | 10 | 10 | 4 | 10 | 10 |
| | | Mean | 432 | 24.0 | 0.083 | 72 | 199 | 34,400 | 16,100 |
| | | SD | 23.9 | 1.33 | (0.083-0.083) | (72-1008) | 62.1 | 26,000 | 1710 |
| | | CV % | 5.52 | 5.52 | NA | NA | 31.2 | 75.7 | 10.6 |

b. Additional MYTX-011 (total ADC) toxicokinetic parameters

| | | | AUC_{0-72 hr}/ | | | | |
|---|---|---|---|---|---|---|---|
| | | | Dose | AUC_{0-504 hr} | AUC_{inf} | CL | |
| Dose | | | (hr · kg · | (hr · μg/ | (hr · μg/ | (mL/hr/ | V_{z} |
| (mg/kg) | Day | Statistic | μg/mL/mg) | mL) | mL) | kg) | (mL/kg) |
| 6 | 1 | N | 6 | 6 | 6 | 6 | 6 |
| | | Mean | 732 | 11,100 | 12,400 | 0.499 | 117 |
| | | SD | 100 | 1760 | 2210 | 0.0952 | 12.7 |
| | | CV % | 13.7 | 15.9 | 17.9 | 19.1 | 10.8 |
| | 22 | N | 6 | NA | NA | 6 | 6 |
| | | Mean | 853 | NA | NA | 0.727 | 54.8 |
| | | SD | 115 | NA | NA | 0.126 | 7.17 |
| | | CV % | 13.5 | NA | NA | 17.3 | 13.1 |
| 12 | 1 | N | 6 | 6 | 6 | 6 | 6 |
| | | Mean | 744 | 25,000 | 29,200 | 0.417 | 111 |
| | | SD | 64.4 | 1960 | 3970 | 0.0542 | 5.58 |
| | | CV % | 8.66 | 7.85 | 13.6 | 13.0 | 5.01 |
| | 22 | N | 6 | NA | NA | 6 | 5 |
| | | Mean | 898 | NA | NA | 0.655 | 52.3 |
| | | SD | 82.1 | NA | NA | 0.132 | 4.10 |
| | | CV % | 9.14 | NA | NA | 20.2 | 7.84 |
| 18 | 1 | N | 10 | 10 | 9 | 9 | 9 |
| | | Mean | 795 | 39,400 | 44,300 | 0.410 | 115 |
| | | SD | 99.8 | 5620 | 4300 | 0.0396 | 9.78 |
| | | CV % | 12.6 | 14.3 | 9.71 | 9.66 | 8.49 |
| | 22 | N | 10 | 4 | NA | 10 | 10 |
| | | Mean | 896 | 52,400 | NA | 0.570 | 70.1 |
| | | SD | 94.8 | 12,000 | NA | 0.190 | 25.1 |
| | | CV % | 10.6 | 23.0 | NA | 33.3 | 35.8 |

NA—Not applicable
^{a}Median (minimum - maximum), median value only reported if actual collection interval.
^{b}T½ was not calculated for animals with less than three half-lives worth of data.

All anti-MYTX-011 antibody samples collected from vehicle- and MYTX-011-treated animals on Days 1, 15, 22, 43, and 64 were negative for anti-drug antibody (“ADA”) response. MYTX-011 is unexpectedly stable in plasma from monkey and human, which is consistent with the low circulating levels of the active small molecule release products. After incubation with cyno plasma for 168-hours, formation of free MMAE was <0.138%, and in human plasma was <0.246%, respectively. This exceptional stability may translate to lower levels of side effects for human patients treated with MYTX-011 versus non-pH-engineered anti-cMET ADC carrying the same toxic payload. In some cases, MYTX-011 will deliver greater efficacy and lower toxicity relative to a cMET-targeting non-pH-ADC at comparable levels of exposure in a patient.

MYTX-011 was assessed in repeat-dose toxicity studies shown in Table 5. The IV route and Q3W dosing regimen were selected to align with the clinical trial.

TABLE 5
Overview of the MYTX-011 cyno monkey toxicology program

| | Concentrations | GLP |
|---|---|---|
| Study | or Doses | Compliance |
| 25-Day IV Tox | 6, 12, 18 mg/kg/dose | Non-GLP |
| 25-Day IV Tox with a | 6, 12, 18 mg/kg/dose | GLP |
| 6-Week Recovery | | |

MYTX-011 was administered to monkeys once every three weeks via IV injection for up to 25 days (two total doses). MYTX-011 was well-tolerated at 18 mg/kg/dose. Primary MYTX-011-related findings in the pivotal repeat dose toxicity study were limited to minimal to moderate decreases in RBC mass parameters, reticulocytes, neutrophils, and/or white blood cells at ≥12 mg/kg/dose. The primary microscopic changes included atypical mitotic figures in multiple organs at ≥6 mg/kg and mild to moderate decreased hematopoiesis in the bone marrow at 18 mg/kg/dose. All MYTX-011 changes were fully reversible by the end of the six-week recovery period. The clinical pathology and microscopic findings were most likely MMAE-payload related toxicities (i.e., neutropenia, decreases in bone morrow hematopoiesis, and atypical mitotic figures in tissues and organs). The highest non-severely toxic dose (HNSTD) in this study was considered to be 18 mg/kg/dose (the highest dose tested).

Species Selection. The monkey was selected as the pharmacologically relevant nonclinical species because of similar cMET protein sequence homology and similar binding affinity of MYTX-011 to monkey cMET, as compared to human cMET. MYTX-011 does not bind to mouse or rat cMET.

Evaluation of vcMMAE Linker/Payload and cMET Monoclonal Antibody. The linker/payload of MYTX-011 (vcMMAE) has been well-characterized non-clinically and/or clinically for several ADCs (e.g., brentuximab vedotin, polatuzumab vedotin, enfortumab vedotin). The nonclinical and clinical toxicities of MMAE-containing ADCs also have been well-described in the literature (Fisher 2021; Saber and Leighton 2015). The reported primary nonclinical target organs for of MMAE-containing ADCs include the hematopoietic system, liver, and male reproductive system (Fisher, 2021).

Tolerability of MYTX-011 was assessed in a single dose monkey PK study. Three (3) female monkeys were administered MYTX-011 IV at 2.7 mg/kg/dose and subsequently followed for 28 days to assess systemic exposure of MYTX-011. There were no MYTX-011-related clinical signs, or changes in body weight, clinical chemistry, or hematology parameters.

Repeat Dose Toxicity. MYTX-011 was evaluated for potential toxicity in a non-GLP 25-day repeat-dose cynomolgus monkey toxicity study and in a GLP 25-day repeat-dose cynomolgus monkey toxicity study with a 6-week recovery period. In both studies, MYTX-011 was administered once every three weeks (total of 2 doses) via slow bolus injection at 6, 12, or 18 mg/kg/dose. There was no toxicity observed in the non-GLP repeat-dose toxicity study that was not observed in the GLP repeat-dose toxicity study.

In the pivotal GLP repeat-dose toxicity study, male and female monkeys (3/sex/group) were administered 0 (vehicle control), 6, 12, or 18 mg/kg/dose MYTX-011. Additional animals (2/sex/group) at 0 and 18 mg/kg/dose were assessed after a 6-week recovery period. All animals survived to the scheduled necropsy (Day 25). There were no MYTX-011-related clinical observations or changes in qualitative food consumption, body weights, ophthalmic findings, electrocardiology changes, blood pressure, heart rate, coagulation, clinical chemistry, urinalysis, macroscopic findings, or organ weights.

MYTX-011-related changes in hematology parameters included generally dose-dependent decreases in neutrophils, reticulocytes, and red blood cell (RBC) mass, and increased platelets at ≥12 mg/kg/dose and increased red blood cell distribution width (RDW) at 18 mg/kg/dose. The transiently decreased neutrophils correlated with histologic findings of decreased cellularity of the bone marrow and all changes in hematology parameters at 18 mg/kg were reversed by the end of the six-week recovery period.

MYTX-011-related microscopic findings included generally dose-dependent, minimal to mild increased atypical mitotic figures in the several tissues/organs at ≥6 mg/kg/dose. MYTX-011-related microscopic findings also included minimal to mild increased cellularity of tingible body macrophages and mild to moderate decreased hematopoiesis of the bone marrow at 18 mg/kg; minimal apoptosis in the thymus and uterus, minimal multinucleated hepatocytes in the liver, and minimal mixed cell infiltration of the trachea at ≥12 mg/kg/dose. Additionally, local effects at the IV administration site and draining lymph node included non-dose-dependent minimal to moderate increased atypical mitoses and minimal to mild apoptosis/necrosis of the epithelium at ≥6 mg/kg/dose. No MYTX-011-related microscopic findings were noted at the end of the six-week recovery period indicating full reversibility.

In conclusion, administration of MYTX-011 when given by intravenous bolus injection once every three weeks (Q3W) for two doses to male and female monkeys was well-tolerated at dose levels up to 18 mg/kg/dose. Hematology and microscopic changes were fully reversible by the end of the six-week recovery period. In this study the highest non-severely toxic dose (HNSTD) was 18 mg/kg/dose. This dose level corresponded to intact ADC mean Cₘₐₓ and AUC values (sexes combined) of 415 μg/mL and 39,400 hr·μg/mL, respectively.

Local findings at the IV administration site and draining lymph node included non-dose-dependent minimal to moderate increased atypical mitoses and minimal to mild apoptosis/necrosis of the epithelium at ≥6 mg/kg/dose. Apoptosis/necrosis and increased mitotic figures of greater severity than mild were considered related to local/perivascular exposure to MYTX-011 during dosing, as opposed to being systemic exposure related. No MYTX-011-related microscopic findings at the injections site were noted at the end of the six-week recovery period indicating full reversibility.

Relationship of Doses to Pharmacokinetics. MYTX-011 exposure in monkeys in the pivotal 25-day repeat-dose toxicity study, as defined by Cₘₐₓ and AUC, increased with increasing dose over the dose range tested, and exposure increased in an approximately dose-proportional manner. There were no apparent sex-related differences in exposure. Anti-MYTX-011 antibody response was assessed in samples collected from vehicle- and MYTX-011-treated animals on Days 1, 15, 22, 43, and 64. No anti-drug antibodies (ADAs) were detected in any animals at any timepoint during the study.

The exposure margin (based on AUC_{0-504 hr} on Day 22; 39400 μg·hr/mL of intact ADC) at 18 mg/kg/dose (highest dose tested; HNSTD) in the 25-day repeat-dose monkey study is 12.44-fold, relative to the projected exposure (3168 μg·hr/mL) at the 1 mg/kg clinical starting dose.

### REFERENCES

Chang H P, et al. Whole-Body Pharmacokinetics and Physiologically Based Pharmacokinetic Model for Monomethyl Auristatin E (MMAE). JCM 2021 Mar. 23; 10(6):1332.
DeGeorge et al., 1998; ICH S9, Nonclinical Evaluation for Anticancer Pharmaceuticals, March 2010; US FDA 2005.
DepMap, Broad (2022): DepMap 22Q2. doi.org/10.6084/m9.figshare.19700056.v2
Fernandes M, et al. When the MET receptor kicks in to resist targeted therapies. Oncogene. 2021; 40(24):4061-4078.
Fisher, J. E., Jr. Considerations for the Nonclinical Safety Evaluation of Antibody-Drug Conjugates. Antibodies 2021, 10, 15.
Han, T., et al., CYP3A-mediated drug-drug interaction potential and excretion of brentuximab vedotin, an antibody-drug conjugate, in patients with CD30-positive hematologic malignancies. J Clin Pharmacol, 2013. 53(8): p. 866-877.
Kinoshita, T., et al., Safety and pharmacokinetics of polatuzumab vedotin in Japanese patients with relapsed/refractory B-cell non-Hodgkin lymphoma: a phase 1 dose-escalation study. Japanese Journal of Clinical Oncology, 2021. 51(1):p. 70-77.
Okamoto, H., et al., Pharmacokinetics of trastuzumab deruxtecan (T-DXd), a novel anti-HER2 ADC, in HER2-positive tumor-bearing mice. Xenobiotica, 2020. 50 (10): p. 1242-1250.
Pastuskovas, C., et al., Tissue distribution, metabolism, and excretion of the antibody-drug conjugate Herceptin-monomethyl auristatin E in rats. PAACR 2005.
Saber, H., Leighton J., An FDA oncology analysis of antibody drug conjugates. Regulatory Toxicology and Pharmacology, 2015. 71:p. 444-452.
Salgia R. MET in Lung Cancer: Biomarker Selection Based on Scientific Rationale. Mol Cancer Ther. 2017; 16(4):555-565.
Sierra J R, Tsao M S. c-MET as a potential therapeutic target and biomarker in cancer. Ther Adv Med Oncol. 2011; 3(1 Suppl):S21-35.
Shen B Q, Xu K, Liu L, et al. Conjugation site modulates the in vivo stability and therapeutic activity of antibody-drug conjugates. Nat Biotechnol. 2012; 30(2): 184-189.
Takahashi, S., et al., A phase I study of enfortumab vedotin in Japanese patients with locally advanced or metastatic urothelial carcinoma. Inves. New Drugs, 2020. 38:p. 1056-1066.

### Example 8. Determination of Soluble cMET Levels in Human Plasma

Various researchers have demonstrated that administration of an anti-cMET antibody to patients is associated with increases in the plasma levels of soluble cMET (sMet). Since such increased levels are associated with poor treatment outcomes, it would be desirable to provide an antibody that results in lower increases—or even decreases—in the plasma levels of sMet. Applicant envisions that the anti-cMET pH-antibodies as disclosed herein will result in relatively lower increases, and even decreases, in sMet levels relative to their non-pH-engineered controls. In some embodiments, the anti-cMET pH-antibodies result in plasma cMET levels that are lower (e.g. at least about 1.5-, 2.0-, 3.0-, 4.0-, 5.0-, 10-, 30-, 100-fold) as compared to the levels that would result from administration of non-pH-engineered control Ab and/or ADC.

To test whether (and to what extent) the anti-cMET pH-ADCs differ in their ability to increase plasma levels of soluble cMET, relative to non-pH-engineered anti-cMET antibodies (e.g. onartuzumab, emibetuzumab, and/or ABT-700), an sMet assay is performed (see, e.g. Shewang Wen. BioMed Research International, 2015).

In the first study, an sMet assay was performed on plasma samples from 10 NSCLC patients and 4 healthy donors. Briefly, the concentration of sMet in plasma was quantitatively measured using human c-Met (soluble) ELISA kit (ThermoFisher, Cat #KH02031) according to the manufacturer's instruction. Briefly, 100 μL of plasma samples (after 1:100 dilution) and standards were added into the wells precoated with capture antibody. After incubating for 2 hours at room temperature (RT), the plates were washed 4 times with wash buffer. A total of 100 μL of biotinylated anti-Hu cMET solution was then added to the wells. The plates were slowly shaken at RT for 1 hour. After washing the plates 4 times, 100 μL of Streptavidin-Horseradish Peroxidase Working Solution was added to the wells. The plates were incubated for 30 minutes at RT and then washed 4 times. Next, 100 μL of stabilized chromogen was added to each well and incubated for 30 minutes at RT and in the dark. After adding 100 μL of Stop Solution to each well and incubating for 30 minutes, plate was read on a microplate reader at 450 nM. The concentration of sMet was calculated using a standard curve and the data shown in Table 6 represent the final plasma concentration multiplied by the dilution factor of 100.

TABLE 6
Soluble cMET (sMet) Assay Results

| Plasma Sample | sMet (ng/mL) | Ave. sMet (ng/mL) |
|---|---|---|
| NSCLC 1 | 455.0 | 417.38 |
| NSCLC 2 | 410.4 | |
| NSCLC 3 | 447.2 | |
| NSCLC 4 | 311.8 | |
| NSCLC 5 | 340.2 | |
| NSCLC 6 | 375.7 | |
| NSCLC 7 | 499.6 | |
| NSCLC 8 | 518.6 | |
| NSCLC 9 | 383.0 | |
| NSCLC 10 | 432.3 | |
| Healthy 1 | 371.1 | 384.75 |
| Healthy 2 | 365.5 | |
| Healthy 3 | 364.7 | |
| Healthy 4 | 437.7 | |

In another study, the assay will be used to compare sMet plasma levels of subjects and/or patients treated with the disclosed anti-cMET pH-ADC versus those treated with a suitable non-pH-engineered control Ab and/or ADC. In this study, treatment with pH-engineered MYTX-011 is associated with lower plasma sMet levels than treatment with a non-pH-engineered anti-cMET antibody (e.g. emibetuzumab).

### Example 9. Formulation of Anti-cMET pH-ADC for Clinical Use

The anti-cMET pH-ADC drug product (DP) is provided as a sterile liquid for injection filled at 10 mg/ml into 50 mL borosilicate glass vials with a flurotec-coated chlorobutyl rubber stopper and an aluminum-plastic seal. The target fill volume for the DP is 10.95 ml (to deliver 10.0 mL). Each vial contains ^{˜}110 mg of anti-cMET pH-ADC. The DP manufacturing process consists of drug substance (DS) thawing, pooling, and mixing, sterile filtration, aseptic filling, stoppering, capping, external vial washing, visual inspection, packaging, and frozen storage. Anti-cMET pH-ADC DS is 0.22 μm sterile filtered at 10 mg/mL and filled into 50 mL glass vials. The target fill volume is 10.95 mL to allow an extractable volume of 10 mL/vial. In addition to anti-cMET pH-ADC, the formulation contains L-Histidine; L-Histidine HCl, monohydrate; D (+)-Trehalose dihydrate; and Polysorbate 80.

### Example 10. A Phase 1 Multicenter Dose Escalation and Dose Expansion Study of MYTX-011 in Subjects with NSCLC

Study Description. Title: A Phase 1 Multicenter Dose Escalation and Dose Expansion Study of Antibody-Drug Conjugate MYTX-011 in Subjects with Non-Small Cell Lung Cancer—KisMET-01™. This is an ongoing, open label multi-center study to evaluate the safety, tolerability, pharmacokinetics and preliminary effective of the investigational drug MYTX-011 in patients with locally advanced, recurrent, or metastatic NSCLC. The study will be conducted in 2 parts. Part 1 will assess the safety and tolerability of MYTX-011 and identify the dose to be studied in Part 2. Part 2 will include subjects with NSCLC with cMET overexpression and/or MET amplification/exon 14 skipping mutations, populations with a current unmet need.

### Patient Selection: Diagnosis and Main Criteria for Inclusion/Exclusion

Archival tumor biopsy samples, or tissue from a new biopsy during screening, must be provided prior to enrollment for subjects in Part 2. Baseline tumor biopsies are recommended, but optional for subjects in Part 1. Confirmation of cMET status by IHC at a central laboratory is required for subjects enrolled in A-D of Part 2 before the subject can be enrolled in the study.

### Main Inclusion Criteria for Part 1

Have histologically or cytologically confirmed locally advanced, recurrent, or metastatic NSCLC and have received available standard of care therapy. There is no limit on the number of prior therapies that can have been received.

Patient has at least one measurable lesion per RECIST 1.1.

ECOG performance status 0 or 1.

Females and males, aged ≥18 years at time of informed consent.

Resolution of acute effects of prior therapy or surgical procedures to ≤ Grade 1 or baseline (except alopecia, stable immune related toxicity such as hypothyroidism on hormone replacement, adrenal insufficiency on ≤ 10 mg daily prednisone (or equivalent) or anemia.

Cardiac LVEF≥ 50% by either echocardiogram or MUGA scan.

Adequate organ function as defined as: a) absolute neutrophil count (ANC)≥1500 cells/mm³ (without growth factors within 1 week of first dose); b) Platelet count≥75,000/mm³ (platelet transfusion not allowed within 1 week prior to first dose of study drug administration); c) Hb≥9 g/dL (RBC transfusion not allowed within 1 week prior to first dose); d) International normalized ratio (INR) activated partial thromboplastin time (aPTT) and prothrombin time (PT)≤1.5× institution's ULN. Patients on stable anticoagulant dose are permitted to enroll; e) calculated creatinine clearance>30 mL/min as calculated by the Modified Cockcroft-Gault formula; f) Total bilirubin≤ 1.5×ULN, ≤ 2.0×ULN for patients with Gilbert syndrome; g) AST and ALT≤ 2.5×ULN for patients without liver metastases; h) alkaline phosphatase≤ 1.5×ULN unless clearly attributable to a non-hepatic source; i) serum albumin≥ 3.0 g/dl j) HbA1C<7.5%.

Diabetic patients must be on a stable dose of antidiabetic medications.

For women of childbearing potential and men with partners of childbearing potential, agreement to use a highly effective method of birth control for the duration of the study treatment and for at least 6 months after the last dose of study drug.

Able to provide informed consent, and willing and able to comply with study protocol.

### Main Inclusion Criteria for Part 2 (Cohorts A-E):

Known to not have an actionable EGFR mutation. Subjects with or without other driver mutations are permitted to enroll.

Must have progressed on at least 1 line of prior therapy in the locally advanced/metastatic setting. Note: multiple lines of tyrosine kinase inhibitor (TKI) for the same actionable mutation count as 1 line of therapy. Maintenance therapy is not considered a separate line of therapy. Adjuvant and neoadjuvant therapies count as 1 line of therapy if given within 6 months of study entry.

Subjects without any actionable gene alteration: must have progressed on (or be considered ineligible for), or be intolerant to, platinum-based chemotherapy and immune checkpoint inhibitor (as monotherapy or in combination with chemotherapy).

Subjects with actionable gene alterations (other than EGFR) for which immune checkpoint inhibitor therapy is not standard of care (e.g., anaplastic lymphoma kinase [ALK] translocation): must have progressed on (or be considered ineligible for), or be intolerant to, anticancer therapy targeting driver gene alterations and platinum-based chemotherapy.

Subjects with actionable gene alterations (other than EGFR) for which immune checkpoint inhibitor is standard of care: must have progressed on (or be considered ineligible for), or be intolerant to, anticancer therapy targeting driver gene alternation and platinum-based chemotherapy, and also progressed on (or be considered ineligible for), or be intolerant to, immune checkpoint inhibitor (as monotherapy or in combination with platinum-based chemotherapy).

Part 2. Cohort A: Have histologically or cytologically confirmed locally advanced, recurrent (and not a candidate for curative therapy), or metastatic non-squamous NSCLC; and tumor sample with high cMET expression by IHC (3+ with tumor cell positivity of ≥50%) confirmed by central laboratory testing. Cohort B: Have histologically or cytologically confirmed locally advanced, recurrent (and not a candidate for curative therapy), or metastatic non-squamous NSCLC; and tumor sample with intermediate cMET expression by IHC (3+ with tumor

cell positivity of ≥25%-<50%) confirmed by central laboratory testing. Cohort C: Have histologically or cytologically confirmed locally advanced, recurrent (and not a candidate for curative therapy), or metastatic squamous NSCLC; and tumor sample with cMET overexpression by IHC (2+ with tumor cell positivity of ≥25%) confirmed by central laboratory testing. Cohort D: Have histologically or cytologically confirmed locally advanced, recurrent (and not a candidate for curative therapy), or metastatic NSCLC; tumor sample that does not meet cMET IHC entry criteria for Cohorts A and B (for subjects with non-squamous NSCLC) and C (for subjects with squamous NSCLC) based on central laboratory testing; known MET amplification or exon 14 skipping mutations, respectively, performed in a CLIA-certified laboratory in the United States (US) or equivalently accredited diagnostic laboratory outside the US; and subjects with MET exon 14 skipping mutations must have received MET TKI therapy, if available and considered standard of care. Cohort E: Have histologically or cytologically confirmed locally advanced, recurrent (and not a candidate for curative therapy), or metastatic NSCLC; evidence of cMET expression by IHC as documented in medical records; evidence of negative cMET expression by IHC would be excluded; and have previously received and had disease progression following either a cMET-targeted ADC or antibody therapy. Must have been on the cMET-targeted ADC or antibody therapy for at least 12 weeks before progression and did not discontinue treatment due to intolerable toxicity.

For all subjects enrolled in Part 1 and Part 2: Subject has at least one (1) measurable lesion per RECIST 1.1. ECOG performance status 0 or 1. Adults, aged ≥18 years at time of informed consent. Resolution of acute effects of prior therapy or surgical procedures to ≤ Grade 1 or baseline and adequate organ function. For women of childbearing potential and men with partners of childbearing potential, agreement to use a highly effective method of birth control for the duration of the study treatment and for at least 6 months after the last dose of study drug. Able to provide informed consent, and willing and able to comply with study protocol requirements.

### Main Exclusion Criteria

NSCLC with adeno-squamous histology (for Cohorts A, B, and C only; subjects with NSCLC with adeno-squamous histology are allowed in Cohorts D and E).

Radiation to the lung within 6 weeks prior to screening or major surgery within 28 days prior to the first dose of study drug administration. For all other sites (except lung and brain), therapeutic or palliative radiation within 2 weeks prior to the first dose of study drug. Must have recovered from all radiation-related toxicity.

Untreated, uncontrolled CNS metastases.

History of interstitial lung disease or pneumonitis that required treatment with systemic steroids or evidence of active interstitial lung disease or pneumonitis.

Subject requires systemic steroid therapy: prednisone 10 mg daily or equivalent. Inhaled steroids or topical steroid use is permitted.

Systemic anticancer therapy including investigational drugs, within the lesser of 28 days or 5 half-lives of the prior therapy before starting study drug (14 days or 5 half-lives for small molecules). Concurrent hormonal therapy for breast or prostate cancer is permitted.

Previously received cMET-targeted Ab or ADC, bicycle, or small peptide therapies. Note: For Cohort E: Subjects will have previously received and progressed on either cMET-targeted ADC or antibody; however, they must not have been intolerant to the cMET-targeted ADC or antibody therapy.

Myocardial infarction, unstable angina, percutaneous transluminal coronary angioplasty (PTCA) or coronary artery bypass grafting (CABG) or cerebrovascular event (stroke or transient ischemic attack [TIA]) within 6 months of first dose of study drug, symptomatic congestive heart failure (New York Heart Association>Class II), or ventricular arrhythmias requiring treatment.

Elevated QTc>480 msec based on Fridericia's correction formula.

Clinically significant systemic illness that could pose undue risk to the subject or confound the ability to interpret study results.

Active infection requiring IV antibiotics, antivirals, or antifungals within 14 days of Day 1.

Neuropathy>Grade 1 for any reason; history of cirrhosis, hepatic fibrosis, esophageal or gastric varices, or other clinically significant liver disease; or active or chronic corneal disorder.

History of another malignancy within the past 3 years prior to screening except adequately treated basal cell or squamous cell skin cancer, carcinoma in situ of the breast or cervix or organ confined prostate cancer.

Known active COVID-19 infection determined by positive antigen test or PCR test, or has signs and symptoms associated with COVID-19 within 14 days prior to first dose of study drug.

Known hypersensitivity to monoclonal antibodies.

Pregnant (positive pregnancy test at screening) or lactating female.

Known hypersensitivity to MYTX-011 or any of its excipients.

### General Procedures

Whole blood samples will be collected for biomarker research from all subjects. Samples will be tested for circulating nucleic acids (e.g., circulating free DNA [cfDNA]) to evaluate their association with the observed clinical responses to MYTX-011. For Cohort D in Part 2, samples will also be used to confirm MET gene status using central testing.

Tumor samples (if available) and a blood sample will be submitted during the screening period. Samples will be analyzed for expression of cMET by IHC (tumor sample) (prospectively for subjects enrolled in Cohorts A-D for Part 2; and retrospectively for subjects in Part 1 and Cohort E Part 2) and retrospectively for MET amplification/exon 14 skipping (blood) using tests established at a central laboratory. Confirmation of cMET status by IHC at a central laboratory is required for Cohorts A-D before the subject can be enrolled in the study.

Once enrolled in the study, MYTX-011 will be administered intravenously every 21 days until disease progression, unacceptable toxicity, or voluntary withdrawal of consent or up to 2 years, whichever occurs first. Subjects who achieve a confirmed CR by 2 tumor imaging assessments conducted at least 4 weeks apart may continue to receive treatment for up to a total of 1 year from the date of confirmed CR at the Investigator's discretion.

CT scans with contrast (or MRI) will be performed at regular time points until disease progression, death, or withdrawal of consent.

### Primary Outcome Measure

Part 1: Number of patients with dose limiting toxicity (DLT). The DLTs will be based on number and severity of treatment-related adverse events. [Time Frame: Up to Day 21].

Part 2: Number of patients with tumor response. The overall response rate will be based on number of complete responses (CR) and partial responses (PR). [2 years].

### Secondary Outcome Measure

Part 1: Pharmacokinetic values. Total antibody conjugated payload, free payload, time to maximum concentration, last measurable concentration, time to lats measurable concentration, area under the concentration-time curve, half-life, total clearance, volume of distribution at steady state. [Time Frame: multiple timepoints].

Part 1: ADA. Presence of anti-drug antibodies. [multiple timepoints].

Part 1: ORR. Complete response (CR)+partial response (PR). [multiple timepoints].

Part 1: DOR, TTR, DCR. Duration of response (DOR) in patients that achieve CR or PR, time to response, best overall response (OR) and disease control rate (DCR). [2 years].

Part 1: PFS. Progression free survival. [up to 2 years after end of treatment].

Part 1: OS. Overall survival [up to 2 years after end of treatment].

Tumor Assessments. The extent of disease and response assessment will be evaluated by contrast enhanced CT scan/MRI based on RECIST 1.1 criteria. Assessments should include the chest, abdomen, pelvis, brain, and other known sites of disease. Confirmation of Investigator-assessed ORR and DOR may be performed retrospectively via an IRC.

Screening assessments will be done within the screening period (within 28 days prior to enrollment/first dose of MYTX-011) and will be used as the baseline scan. The same method of assessment/imaging modality should be used at subsequent visits for a given subject.

CT scans with contrast (preferred) will be performed during screening, then at Week 6 (±1 week), then every 6 weeks (+1 week) thereafter until disease progression, death, or withdrawal of consent. Imaging may be performed more frequently if clinically indicated. If contrast cannot be used, a non-contrast CT scan of the chest and a contrast enhanced MRI of the abdomen and pelvis may be performed. The same technique should be used throughout the course of the study.

Gadolinium enhanced MRI of the brain will be conducted during screening to assess for brain metastases and every 6 weeks if brain metastases are present. If MRI contrast is contraindicated, MRI without contrast is acceptable. If an MRI is contraindicated, then a CT scan with contrast may be performed.

A bone scan and/or positron emission tomography (PET) scan is allowed for subjects at baseline and during the study if bone metastases are considered and at the discretion of the Investigator as per standard of care; however, the CT/MRI scans should be used for tumor assessments as described above. Response criteria will be assessed using RECIST (version 1.1).

Dose Escalation. In Part 1, subjects will be enrolled to evaluate the safety of escalating doses of MYTX-011 and to establish the RP2D and/or maximum tolerated dose (MTD). The dose escalation scheme is based on a 3-subject cohort minimum Bayesian Optimal Interval (BOIN) design (Yuan et al. 2016), a model-assisted design that employs an escalation/de-escalation procedure, similarly implemented as a classical 3+3 design, but optimized to minimize the probability of making an erroneous decision. Dose-limiting toxicities (DLTs) occurring during Cycle 1 (e.g., 21 days) will be considered when implementing the dosing algorithm and decisions. A Safety Review Committee (SRC) will review and assess data collected during the study to guide dose escalation and the determination of the RP2D.

A sentinel dosing strategy will be used during Part 1 dose escalation for increasing doses (i.e., the dose level is the highest dose level that has been explored). A minimum of 24 hours must elapse between dosing of the first subject and subsequent subjects at each dose level. If no safety concerns are noted in the first subject at each dose level, subsequent subjects may be enrolled.

If a new dose level is opened at a lower/intermediate dose after exploring a higher dose level (i.e., de-escalation), sentinel dosing is not needed unless severe acute toxicities, e.g., Grade 3 or higher infusion-related reaction (IRR), occurred within 24 hours after study drug administration in higher dose levels, or the SRC recommends sentinel dosing for the lower dose level.

The first dose of study drug (this term is used interchangeably with “investigational product”) will be administered over 90 minutes. Pre-medication will not be used for the first infusion. Subjects will be observed closely for at least 2 hours following the initial dose for fever, chills, or other IRRs. Subsequent infusions may be administered over 30-60 minutes if prior infusions were well tolerated.

Part 2. Dose expansion will be initiated once the RP2D has been determined.

Cohort A: Cohort A: Non-squamous NSCLC without actionable EGFR mutations; with high cMET expression by immunohistochemistry (IHC) (3+ with tumor cell positivity of ≥50%). Subjects will be randomized 1:1 to 1 of 2 dose levels. An interim analysis for futility will be performed.

Cohort B: Non-squamous NSCLC without actionable EGFR mutations; with intermediate cMET expression by IHC (3+ with tumor cell positivity of ≥25% to <50%). An interim analysis for futility and efficacy will be performed.

Cohort C: Squamous cell NSCLC without actionable EGFR mutations, with cMET overexpression by IHC (2+ with tumor cell positivity of ≥25%). An interim analysis for futility and efficacy will be performed.

Exploratory Cohort D: NSCLC with cMET expression on tumor biopsy by IHC that does not meet inclusion criteria for Cohorts A, B, or C, and with MET amplification or exon 14 skipping mutation without actionable EGFR mutations.

Exploratory Cohort E: NSCLC with cMET expression by IHC without actionable EGFR mutations and previously received either cMET-targeted ADC or antibody therapy.

Cohorts A-E may be opened for enrollment simultaneously or in a staggered manner at the discretion of the Sponsor. Subjects in Cohort A will be randomized to receive 1 of 2 doses: either the optimal RP2D selected at the end of dose escalation or another dose that is at or below the MTD (or the highest dose tested during dose escalation if the MTD is not determined). Subjects in Cohorts B, C, D, and E will receive MYTX-011 at the RP2D determined at the end of Part 1.

MYTX-011 will be administered by IV intravenous (IV) infusion in 21-day cycles. Dose levels are summarized below. The SRC may recommend changing the frequency of the dosing regimen if the emerging data suggest that the current regimen is not optimal (e.g., to every 2 or 4 weeks).

Dosing of MYTX-011 will be based on a mg/kg basis for subjects weighing up to 100 kg. Subjects weighing >100 kg will receive a fixed dose. The fixed dose for these subjects will be the same dose as subjects who weigh 100 kg. Based on emerging PK, safety, and preliminary anti-tumor activity data during the study, the SRC may recommend changing the fixed dose to a mg/kg dose for subjects who weigh >100 kg. The subject's weight at screening will be used to calculate the subject's dose for the duration of treatment unless during the course of treatment, the subject's weight changes by >10% of the initial weight, the dose will be recalculated based on the updated weight.

Routine premedication should not be administered prior to the first dose of MYTX-011. Subjects will be observed closely for IRRs during the infusion and for at least 2 hours following the initial dose. Subjects will be monitored closely during the infusion and for at least 30 minutes after for subsequent infusions.

Efficacy endpoints will be based on RECIST 1.1 by Investigator assessment for the primary analysis. The following efficacy endpoints will be assessed in both Parts of the study.

Objective Response Rate: ORR is defined as the proportion of subjects with the best response of CR or PR. All response data will also be summarized over time for all subjects using all available scans. Note: CR and PR must be confirmed by 2 tumor imaging assessments conducted at least 4 weeks apart.

Duration of Response (for subjects who achieve CR or PR): DOR is defined as time from the date of first documented evidence of CR or PR until first documented disease progression or death, whichever comes first. For subjects without the PD or death prior to analysis cut-off date, DOR will be censored at the date of last available tumor assessment.

Overall Survival: Overall survival (OS) is defined as the time from the date of first administration of MYTX-011 to the date of death due to any cause. OS will be censored at the last reported date of contact.

Progression free survival (PFS) is defined as the time from the date of first administration of MYTX-011 to the date of documented disease progression per RECIST 1.1 or the date of death (in the absence of progression) whichever comes first. For subjects who have not progressed or died before the analysis cut-off date, PFS will be censored at the date of the last available tumor assessment.

Disease Control Rate: DCR is defined as the proportion of subjects who achieve a best response of CR or PR or SD. SD must occur at least >6 weeks following the first dose of MYTX-011 administration.

Best Objective Response: A single best objective response (BoR) is defined per each subject as the occurrence of CR, PR, SD, PD, or not estimable (NE) as the best achieved response during the study.

Time to Response: Time to response is defined as the time from the first administration of MYTX-011 to the first documentation of CR or PR.

### Example 11. cMET IHC Assay and the cMET Weighted H-Score

cMET protein expression levels are routinely measured by a variety of IHC assays. In this Phase I study, cMET expression levels are evaluated using Ventana Medical System's anti-cMET mAb (SP44 anti-total cMET rabbit mAb, as provided in the CONFIRM® kit, cat no. 790-4430). The ultraView Universal DAB staining kit, as well as negative and positive tissue control slides are also sourced from Ventana. Briefly, tumor biopsies are formalin fixed, paraffinized, sectioned, further processed, stained, and scored for cMET expression by a pathologist. An individual staining value is attributed to each cell: 0=none; 1+=weak; 2+=moderate; 3+=strong.

To obtain a weighted “H-score”, the percentage of tumor cells assigned a given score are multiplied by each respective intensity and then added together. Example calculation: H-score=[1*(% 1+)+2*(% 2+)+3×(% 3+)]. As such, the maximum H-score is 300.

Accordingly, each tumor cell can be assigned an IHC score of 0, 1+, 2+, or 3+ and a plurality of cells (e.g., as present in a tissue section) can be assigned an H-score. In the clinical setting, where human samples are concerned, the IHC and/or H-scores are determined by an appropriately credentialled/qualified pathologist. And while both IHC- and H-scores involve 0, 1+, 2+, and 3+ values, they are not to be confused. For the H-score calculation, 0, 1+, 2+, and 3+ values refer to the intensity of staining of a particular individual cell. For the IHC score, 0, 1+, 2+, and 3+ values refer to the overall staining of a particular area of the tumor sample. FIGS. 5A, 5B, 12, 13B, 14, 15A, and 16 show exemplary staining results for various tumor sections.

If no cells in a fixed field stain positive, the value assigned to the tumor is IHC 0. If the majority of cells stain low, moderate, or high, the value attributed to the tumor is IHC 1+, 2+, or 3+, respectively. The skilled artisan will appreciate that tumors can exhibit heterogeneity.

In other embodiments, if at least 20% of the cells exhibit moderate staining, IHC 2+ is assigned. Likewise, if at least 20% of the cells exhibit strong staining, IHC 3+ is assigned.

### Example 12. Measuring MET Gene Copy Number Amplification

MET amplification can enhance a patient's response to agents that target cMET (see, e.g., Clin Cancer Res 2014; 20:4488-98). The MET/CEP7 MET amplification method is an example of how a skilled artisan may assess a tumor's MET status. Briefly, formalin-fixed, paraffin-embedded tumor blocks, are evaluated using dual-color FISH using a MET/CEP7 probe cocktail prepared with a suitable MET DNA probe labeled with SpectrumRed and the SpectrumGreen CEP7 (Abbott Molecular). For a suitable FISH protocol, see Cappuzzo F, et al. (2005) J Natl Cancer Inst 97:643-655. Another available methods include Biocept Liquid Biopsy MET Detect-R®, MET Amplification Test (Biocept), and Guardant360® (Guardant Health).

### Example 13. Assessing the Presence of Exon 14 Mutation/MET Gene Skipping

MET Exon 14 includes an ubiquitin ligase site on Y1003, providing for increased degradation of the cMET protein. As such, MET Exon 14 yields increased MET protein and cMET activation, leading to oncogenesis. Multiple mutations have been described and any suitable assay can be used to detect any Exon 14 mutation, disclosed herein or otherwise known. For an example method, see Mark M. Awad JCO Mar. 10, 2016:879-881, and methods cited therein, each incorporated herein by reference in their entireties. Relevant FDA-approved companion diagnostics include FoundationOne CDx and Liquid CDx (Foundation Medicine, Inc.), both for detection of MET single nucleotide variants and indels that lead to MET exon 14 skipping in NSCLC (companion diagnostics for TABRECTA® (capmatinib)).

For an example method to detect MET amplification, see Park S, et al. Lung Cancer. 2015 December; 90(3):381-7. This method and methods used in references cited therein for identifying additional Exon 14 mutations are incorporated herein by reference in their entireties.

### Example 14. Assessing Alterations in EGFR Exon 19 and 21 in Cancer Patients

Exon 19 deletions and L858R missense mutations comprise ^{˜}85% of all known NSCLC EGFR alterations and each has exhibited sensitivity the RTK inhibitors, erlotinib and gefitinib.

FDA-approved tests for detecting alternative EGFR variants include the following: Cobas EGFR Mutation Test v2 (Roche Molecular Systems, Inc.), which permits detection of exon 19 deletion or exon 21 L858R substitution mutation and the T790M mutation. Other tests include the following: FoundationOne Liquid CDx (Foundation Medicine, Inc.; exon 19 deletion or exon 21 L858R substitution mutation), Guardant360 CDx (Guardant Health, Inc.; exon 19 deletions, exon 20 insertions, exon 21 L858R and T790M substitutions), Oncomine Dx Target Test (Life Technologies Corporation; exon 19 deletion or exon 21 L858R substitution mutation), and Therascreen EGFR RGQ PCR Kit (Qiagen Manchester, Ltd.; exon 19 deletion or exon 21 L858R substitution mutation). See also, e.g., Yang Y, et al. (2016) Oncol Lett. May; (15):3546-3550.

Patients having NSCLC and EGFR exon 19 deletions enjoy relatively longer survival times when treated with erlotinib or gefitinib as compared to those with the L858R mutation. Jackman D M, et al. (2006) Clin Cancer Res 12:3908-3914, incorporated herein by reference.

All documents cited herein are incorporated herein by reference in their entireties.

And while particular embodiments have been described throughout, skilled artisans will appreciate that modifications can be made without exceeding the scope of the inventions.

### Example 15. Activity of Single and Repeat Dose of MYTX-011 in the cMET Low NCI-H2122 Xenograft Model

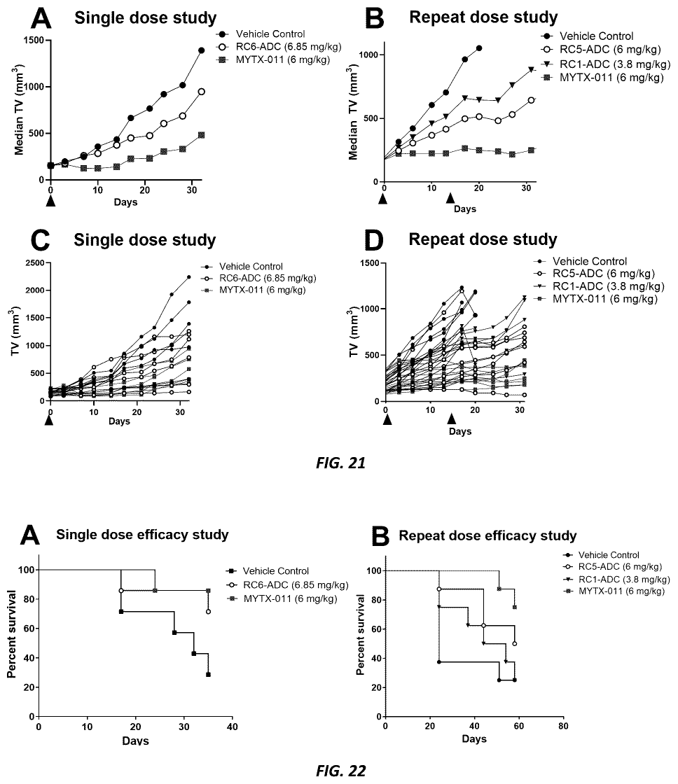

The efficacy of MYTX-011 was assessed in the cMET-low expressing NCI-H2122 xenograft model using female nude mice. The NCI-H2122 model was selected because of its adenocarcinoma non-small cell lung cancer origin, presence of the oncogenic KRAS G12C mutation, and low cMET expression. For the single-dose study, n=7 mice for vehicle and MYTX-011 (6 mg/kg) groups, n=6 mice for the RC6-ADC (6.85 mg/kg) group. For the repeat dose study, n=8 mice per group. Tumor growth curves were plotted until D32 for the single dose study and D31 for the repeat dose study (FIGS. 21B & 21D). Abbreviations: RC1-ADC=RC1-ADC-(DAR3.01); RC5-ADC=RC5-ADC-(DAR1.96); RC6-ADC=RC6-ADC-(DAR3.7); and vehicle=PBS.

Briefly, mice were injected subcutaneously with NCI-H2122 cells and when tumors reached an average size of 175+25 mm³, IV treatment with single doses of MYTX-011 or test articles was initiated. Doses of ADC test article were adjusted to administer equal dosing of the MMAE payload. For the single-dose efficacy study, MYTX-011 was administered at 6 mg/kg, and the non-binding control RC6-ADC-(DAR3.7), was administered at 6.85 mg/kg. And for the repeat-dose study, MYTX-011 and RC-5-ADC (DAR1.96) were administered at 6 mg/kg, and RC1-ADC-(DAR3.1) was administered at 3.8 mg/kg at the Q2WX2 dose regimen. A vehicle control group was included in each study. Tumors were measured with calipers biweekly and body weight was measured biweekly. Animals were euthanized when tumors reached 2000 mm³ or at the end of the study (Day 35 for the single-dose efficacy study and Day 58 for repeat dose efficacy study).

In the single-dose efficacy study, tumor volumes were compared on Day 10. MYTX-011, administered at 6 mg/kg, resulted in significantly decreased tumor volume (FIG. 21A) and increased survival compared to the vehicle control (FIG. 22A). In the repeat dose efficacy study, tumor volumes were compared on Day 17 and Day 20 when all the mice in the compared groups remained alive. MYTX-011, administered at 6 mg/kg, showed significantly reduced tumor volume compared with RC1-ADC-(DAR3.01) and the vehicle control group on D17 (FIG. 21B). MYTX-011, administered at 6 mg/kg, showed significantly reduced tumor volume compared to RC5-ADC-(DAR1.96) group on D20 (FIG. 21B). MYTX-011, administered at 6 mg/kg, also showed significantly increased survival compared with RC1-ADC-(DAR3.01) and the vehicle control group (FIG. 22B). In conclusion, both single and repeated dosing of MYTX-011 was effective against xenograft tumors comprising human cells characterized by low levels of cMET overexpression.

### Example 16. Further Functional Analysis of MYTX-011

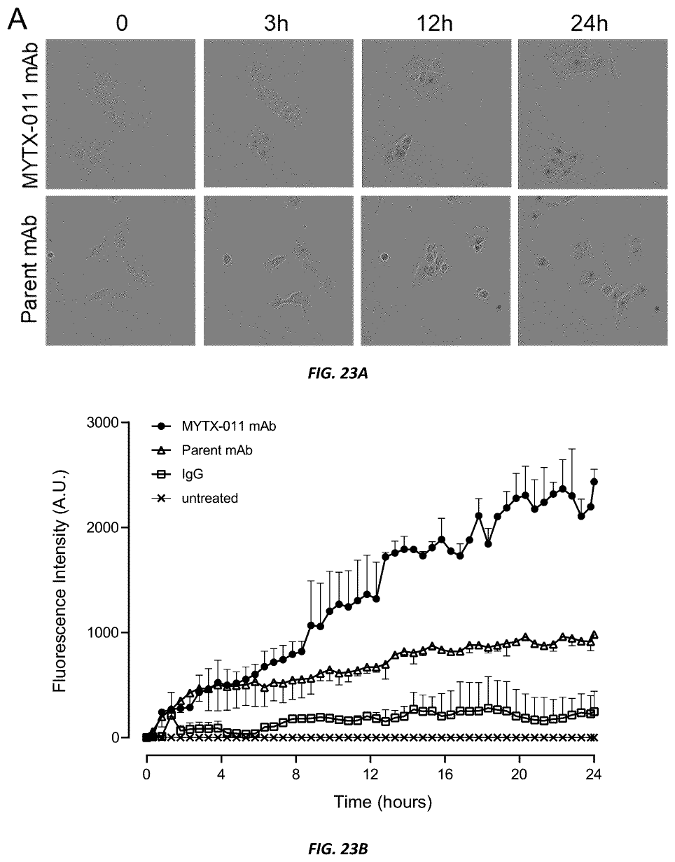

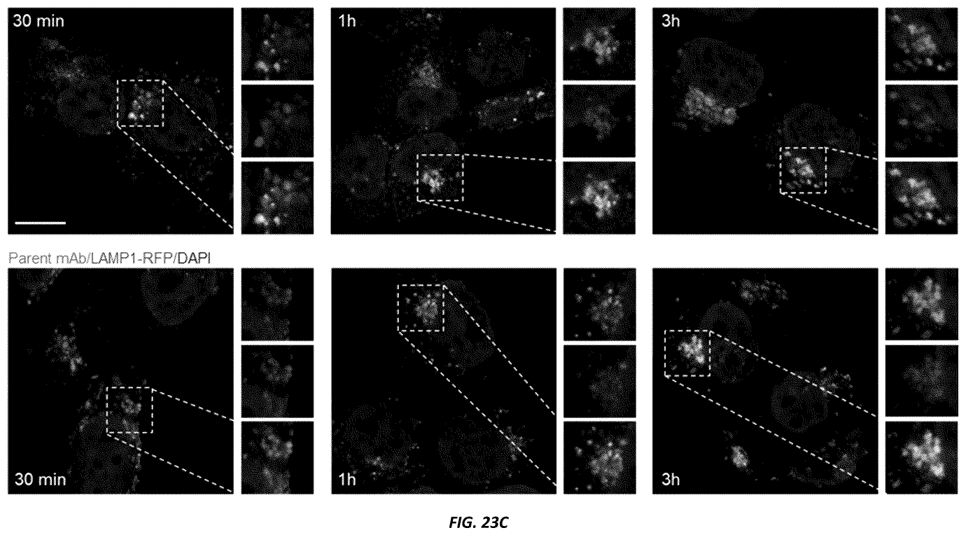

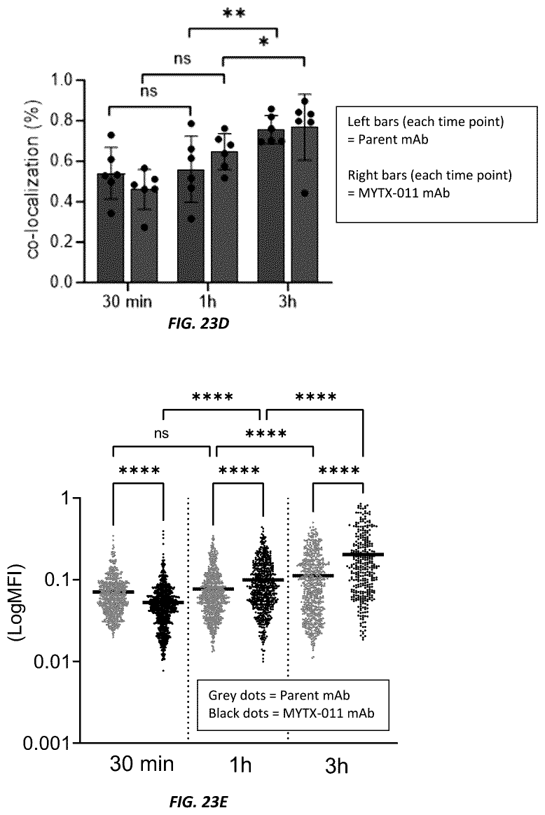

Since pH-dependent MYTX-011 exhibited enhanced internalization and cytotoxicity compared to the non-engineered parent ADC, we sought to resolve the dynamics of internalization over time by treating cells with pHrodo-conjugated mAbs or controls (FIGS. 23A, 23B). At early time points (0-4 hours) MYTX-011 mAb and parent mAb show similar levels of uptake, however, after 6 hours the intracellular levels of MYTX-011 mAb progressively increase reaching the highest intensity at 24 hours corresponding to a threefold increase over the parent mAb. We observed by confocal microscopy that Parent and MYTX-011 mAb are equally trafficked to the lysosomes (FIGS. 23C, 23D), however the latter shows greater accumulation in these organelles (FIG. 23E).

### Example 17. pH-Dependent MYTX-011 ADC Demonstrates Greater cMET-Dependent Cytotoxicity Against a Wide Range of Types of Cancer Cells Vs. Non-pH-Dependent Control ADC

As disclosed above, MYTX-011 exhibited greater efficacy against a wider range of cMET-positive/cMET-overexpressing lung cancer cell types, with or without actionable mutations in EGFR, as compared to an applicable non-pH-dependent control ADCs (Table 7).

TABLE 7
Summary of cMET expression/overexpression levels, histotypes, and mutations for
the wide range of lung cancer xenografts against which MYTX-011 was effective

| | | cMET-expression/ | FACS/IHC | Response to | | MET |
|---|---|---|---|---|---|---|
| CDX/PDX Model | | overexpression | (Example 4) | MYTX-011 | EGFR Status | Status |
| EBC-1 | SqCC | High | 134608/3+ | CR at 0.5 | | MET |
| | | | | mg/kg | | amplified |
| NCI- | KRAS | Moderate | 46450/2+ | PR at 2 | EGFR WT | |
| H1373 | mutant, | | | mg/kg | | |
| | LuAd | | | | | |
| NCI- | LuAd | Moderate | 42360/2+ | PR at 3 | L858R/T790M | |
| H1975 | | | | mg/kg | EGFR-mutant | |
| NCI- | KRAS | Low | 1237/1+ | SD at 6 | EGFR WT | |
| H2122 | mutant | | | mg/kg | | |
| | LuAd | | | | | |
| CTG- | LuAd | High | | SD at 6 | T790M, | MET |
| 3414 | | | | mg/kg | E746_A750del | amplified |
| CTG- | SqCC | Moderate | | SD at 6 | WT | WT |
| 1353 | | | | mg/kg | | |
| CTG- | LuAd | Moderate | | CR at 6 | WT | T301A, |
| 2669 | | | | mg/kg | | D1028N |
| CTG- | LuAd | Moderate | | SD at 6 | WT | WT |
| 2533 | | | | mg/kg | | |
| CTG- | SqCC | Negative | | NR at 6 | WT | WT |
| 2082 | | | | mg/kg | | |

WT: Wild type; SqCC: Lung Squamous Cell Carcinoma; LuAd: Lung adenocarcinoma; CR: Complete Response; PR: Partial response; SD: Stable disease; NR: No response.
Responses were after single dose treatment. All models are insensitive to non-targeting control.

Given that pH engineered anti-cMET ADCs exhibited increased internalization, cytotoxicity, and efficacy against NSCLC cells in vitro and in vivo, we next sought to determine the efficacy of these compounds against a wider range of cMET-positive/cMET-overexpressing cancer cells. Sixty-two (62) cell lines were selected across solid tumor types including NSCLC, head and neck, gastric, pancreatic, esophageal, bladder, kidney and skin cancer, and subjected to a nine-point dose response of MYTX-011, the corresponding parental ADC, or a non-binding control ADC following 96 hours of treatment (DNS).

Interestingly, the parent ADC was active against 15/62 cell lines tested, MYTX-011 was active against an additional 16 cell lines (31/62), while the non-binding control was inactive against all cell lines. This result suggests that pH-dependent binding of ADCs confers more potent cytotoxicity in vitro (FIGS. 24A, 24B). Importantly, sensitivity correlated with cMET expression in the cancer cell lines (FIG. 24C) demonstrating that the increased cytotoxicity of MYTX-011 is on-target. Cell lines that were sensitive to MYTX-011 but not the parental ADC exhibited lower levels of cMET expression demonstrating that MYTX-011 is active against cancer cells with a broader range of cMET expression (FIG. 24D).

cMET surface expression was quantified by flow cytometry using Quantibrite™ beads (Becton Dickinson QUANTBRITE™ Phycoerythrin Fluorescence [PE] Quantification Kit, Cat #340495), with EBC-1 and NCI-H1650 cells included in each experiment to serve as an internal control/reference.

The cMET expression level of each cell line was then divided by the averaged cMET expression of EBC-1 cells, and cell lines were ranked according to the normalized cMET expression (Table 8).

TABLE 8
Level of cMET expression in each of the 62 cancer
cell lines normalized to the average cMET expression
levels for the EBC-1 batch controls.

| | | cMET | Geometric | |
|---|---|---|---|---|
| Cell Line | Tissue | positive | Mean | PE Molecules/ |
| Name | Origin | (%) | (PE) | Bead |
| A549 | Lung | 96.76 | 29,963.42 | 2301.27 |
| Calu-3 | Lung | 92.91 | 19,374.85 | 1436.00 |
| EBC-1 | Lung | 99.95 | 371,959.78 | 30931.59 |
| HCC4006 | Lung | 98.79 | 40,453.43 | 3067.31 |
| HCC827 | Lung | 99.83 | 165,418.75 | 13136.67 |
| NCI-H1155 | Lung | 0.78 | 939.28 | 63.40 |
| NCI-H1299 | Lung | 96.54 | 23,900.47 | 1792.59 |
| NCI-H1581 | Lung | 4.99 | 2,091.44 | 144.70 |
| NCI-H1650 | Lung | 99.24 | 37,329.63 | 2886.98 |
| NCI-H1703 | Lung | 97.37 | 26,053.40 | 1948.75 |
| NCI-H1781 | Lung | 44.67 | 6,487.31 | 464.82 |
| NCI-H1975 | Lung | 99.8 | 119,758.26 | 9420.22 |
| NCI-H1993 | Lung | 99.92 | 365,992.63 | 29755.25 |
| NCI-H2122 | Lung | 38.59 | 16,671.03 | 1237.13 |
| NCI-H2228 | Lung | 84.61 | 19,595.96 | 1461.14 |
| NCI-H292 | Lung | 99.71 | 30,110.27 | 2273.81 |
| NCI-H358 | Lung | 92.55 | 18,965.10 | 1412.74 |
| NCI-H441 | Lung | 90.5 | 39,804.55 | 3030.77 |
| NCI-H460 | Lung | 98.73 | 19,740.91 | 1472.27 |
| NCI-H520 | Lung | 0.2 | 1,077.71 | 73.76 |
| NCI-H727 | Lung | 99.32 | 25,806.49 | 1939.94 |
| NCI-H820 | Lung | 91.96 | 34,670.20 | 2616.30 |
| PC-9 | Lung | 99.93 | 68,439.82 | 5274.41 |
| SK-MES-1 | Lung | 33.85 | 8,434.43 | 1093.29 |
| SNU-2535 | Lung | 46.3 | 9,432.72 | 683.73 |
| 5637 | Bladder | 99.25 | 30,794.83 | 2315.35 |
| HT-1376 | Bladder | 71.91 | 15,378.52 | 1138.50 |
| RT112/84 | Bladder | 68.27 | 10,754.17 | 787.78 |
| RT4 | Bladder | 46.94 | 5,201.50 | 372.94 |
| KYSE-150 | HN/Esophagus | 99.39 | 19,354.28 | 1434.42 |
| KYSE-270 | HN/Esophagus | 83.84 | 5,235.08 | 375.42 |
| KYSE-410 | HN/Esophagus | 99.65 | 41,444.49 | 3144.81 |
| OE19 | HN/Esophagus | 24.87 | 7,687.92 | 553.74 |
| C666-1 | HN/Nose | 29.74 | 3,453.58 | 242.67 |
| Detroit 562 | HN/Pharynx | 95.96 | 36,967.79 | 2858.11 |
| FaDu | HN/Pharynx | 97.26 | 20,271.54 | 1537.82 |
| CAL-27 | HN/Tongue | 92.44 | 21,475.20 | 1596.74 |
| SCC-4 | HN/Tongue | 97.88 | 40,420.16 | 3133.85 |
| 786-O | Kidney | 99.61 | 47,906.25 | 3651.46 |
| A498 | Kidney | 99.86 | 103,396.69 | 8070.75 |
| Caki-1 | Kidney | 98.75 | 69,599.20 | 5489.52 |
| HGC-27 | Stomach | 77.71 | 4,742.48 | 339.10 |
| HS 746.T | Stomach | 99.94 | 229,893.38 | 18393.59 |
| KATO III | Stomach | 99.08 | 75,087.81 | 10363.39 |
| MKN45 | Stomach | 99.6 | 167,930.25 | 13306.09 |
| NCI-N87 | Stomach | 69.56 | 8,952.30 | 647.86 |
| NUGC-4 | Stomach | 99.56 | 80,434.58 | 6229.84 |
| SNU-5 | Stomach | 99.72 | 433,709.78 | 35437.98 |
| YCC-10 | Stomach | 66.17 | 12,971.76 | 949.56 |
| YCC-2 | Stomach | 42.48 | 13,159.42 | 984.77 |
| AsPC-1 | Pancreas | 99.82 | 59,770.35 | 4691.68 |
| BxPC-3 | Pancreas | 99.56 | 42,840.27 | 3254.06 |
| Capan-1 | Pancreas | 96.65 | 45,165.95 | 3436.32 |
| Capan-2 | Pancreas | 80.69 | 9,837.83 | 714.02 |
| HPAF-II | Pancreas | 15.53 | 4,719.27 | 341.91 |
| KP4 | Pancreas | 88.8 | 15,396.60 | 1157.91 |
| Panc 05.04 | Pancreas | 87.67 | 23,537.49 | 1794.02 |
| PANC-1 | Pancreas | 34.15 | 11,507.16 | 857.48 |
| SK-MEL-5 | Skin | 81.88 | 23,012.58 | 1714.71 |
| LN-229 | Brain & Nerves | 90.37 | 20,614.71 | 1539.41 |
| Calu-6 | Lung | 99.34 | 49,788.75 | 3799.47 |
| NCI-H23 | Lung | 75.82 | 16,717.30 | 1233.39 |
| EBC-1 | Lung | 99.91 | 215,947.03 | 30721.82 |
| (Control) | | | | |
| EBC-1 | Lung | 99.76 | 211,315.14 | 16863.15 |
| (Control) | | | | |
| EBC-1 | Lung | 99.71 | 116,310.04 | 9141.09 |
| (Control) | | | | |
| NCI-H1650 | Lung | 95.72 | 27,433.95 | 3678.50 |
| (Control) | | | | |
| NCI-H1650 | Lung | 95.77 | 27,019.53 | 2023.30 |
| (Control) | | | | |
| NCI-H1650 | Lung | 95.38 | 25,183.69 | 1891.76 |
| (Control) | | | | |

PE = Phycoerythrin Fluorescence

Notably, cell lines that were insensitive to MYTX-011 were largely resistant to free MMAE, indicating that payload sensitivity is an independent determinant of MYTX-011 cytotoxicity (FIG. 24E). And as illustrated by the IC50 values shown in FIG. 24F, MYTX-011 demonstrated markedly increased cytotoxicity against cell lines derived from gastric, head & neck, lung, bladder, esophageal, and pancreatic cancers. Taken together, these results show that MYTX-011 cytotoxicity correlates with cMET expression levels, and MYTX-011 demonstrates cytotoxicity to cells that were otherwise resistant to non-pH-dependent parent ADC.

### Example 18. Efficacy of MYTX-011 Against Gastric and Head & Neck Cancers In Vivo

The efficacy of MYTX-011 was next evaluated in several mouse models of cancer, including gastric, head & neck, and esophageal cancers. Briefly, non-MET amplified, moderately cMET-overexpressing cancer models (i.e., similar to the lung cancer lines NCI-H1975 and NCI-H1373) were identified based on available cMET RNA/protein expression data. Cell-derived xenograft (CDX) models included two (2) for head & neck cancer (Detroit-562 and FaDu), one (1) for esophageal cancer (KYSE-150), and two (2) for gastric cancer (NUGC-4 and SNU-16).

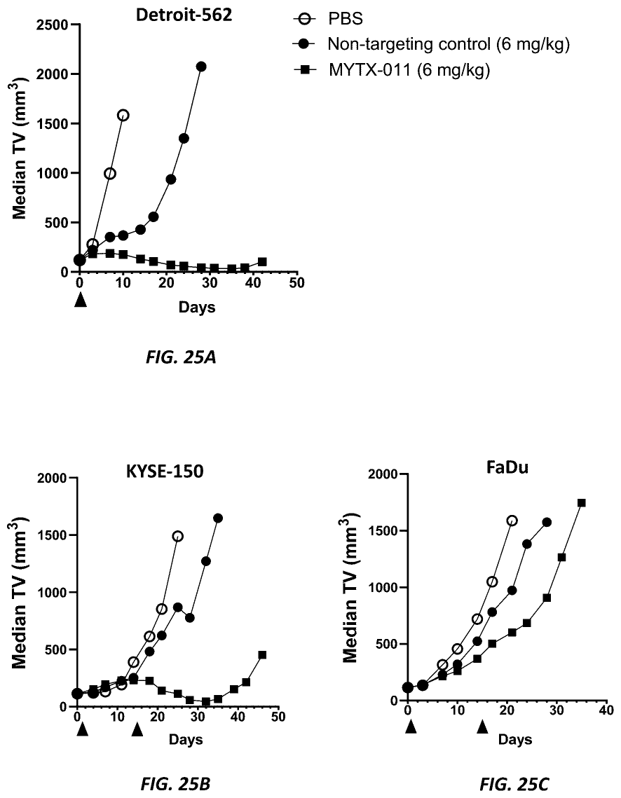

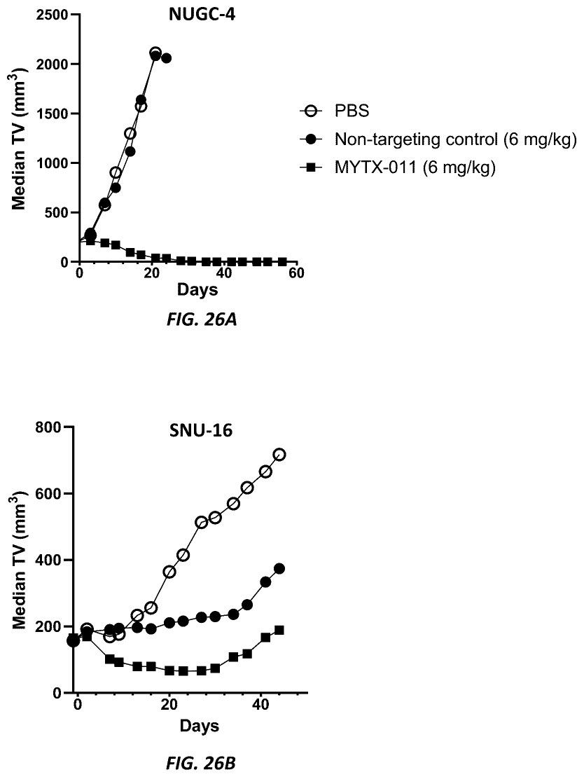

The cMET-overexpressing head & neck cancer cell lines, Detroit 562 and FaDu, and the esophageal cancer cell line, KYSE-150, were propagated in vivo in NOD/SCID mice. Briefly, animals were subcutaneously inoculated with 5×10⁶ cells in PBS for tumor development. Measurements of the tumors' length (L) and width (W) were taken via an electronic caliper. The volume was calculated using the following equation: Tumor volume=(length×width×width)/2. Once the tumors reached a volume of 100-200 mm³, mice were randomized and human IgG1 (huIgG1) treatment was initiated on Day 0. On Day 1, a bolus (6 mg/kg) of either cMET-specific MYTX-011 ADC or non-binding control ADC was administered to each animal via the tail vein. FaDu and KYSE-150 models were also dosed on D15 with either MYTX-011 or the non-binding control ADC. Mice injected with PBS served as vehicle controls. Body weight and tumor volumes were measured bi-weekly. An individual mouse was euthanized if its tumor volume exceeded 1,500 mm³. Detroit 562 and FaDu models were terminated on D42 and the KYSE-150 model study was terminated on day 46. As indicated in FIGS. 25A and 25B, MYTX-011 was highly effective against Detroit-562- and KYSE-150-cell derived tumors in vivo.

Next, the cMET-overexpressing gastric cancer cell lines, NUGC-4 and SNU-16, were propagated in vivo in nude mice. Briefly, animals were inoculated subcutaneously with 5×10⁶ cells in PBS for tumor development. Measurements of the tumors' length (L) and width (W) were taken via an electronic caliper. The volume was calculated using the following equation: Tumor volume=(length×width×width)/2. Once the tumors reached a volume of 100-200 mm³, mice were randomized and mice were dosed with a single bolus dose (6 mg/kg) of either MYTX-011 or non-binding control ADC, administered via the tail vein. Mice injected with PBS served as vehicle controls. Body weight and tumor volumes were measured bi-weekly. The individual mouse was euthanized if its tumor volume exceeded 1,500 mm³. NUGC-4 model was terminated on D56, and SNU-16 model was terminated on day 51. The data showed that a single dose of MYTX-011 was highly active in NUGC-4 and SNU-16 xenografts. SP44-based cMET IHC will be used to evaluate the expression in both the head & neck and gastric cancer xenografts. Additional SP44 cMET IHC is planned to be conducted on the TMAs of human patients suffering head & neck or gastric cancer, along with additional markers (e.g., p16 and HER2).

Overall conclusions. The data of this disclosure provide evidence that pH-dependent, anti-cMET antibody drug conjugates (“anti-cMET-pH-ADCs”, including MYTX-011) are more effective against cancer cells (both in vitro and in vivo) than corresponding non-pH-dependent, anti-cMET-ADCs. For example, MYTX-011 shows activity in moderate cMET overexpressing squamous cell carcinoma and EGFR mutant PDX models. MYTX-011 is also active in highly cMET heterogeneous tumors, implicating in vivo bystander activity as has been described for other MMAE-based ADCs. Further, MYTX-011 demonstrated efficacy in the H2122 CDX model, which is characterized by low cMET expression (i.e., cMET-positive/expressing/borderline overexpression). And as indicated in Example 18, MYTX-011 also exhibited efficacy in CDX models of gastric and head & neck cancer. Taken together, these findings highlight the potential of MYTX-011 as a therapeutic candidate for treating a broader range of cMET+/cMET-expressing/cMET-overexpressing malignancies than non-pH-engineered cMET-ADCs.

### Example 19. Clinical Trial Results

Human subjects having a cMET-positive/cMET-expressing or cMET-overexpressing tumor are enrolled and treated with MYTX-011 as described herein. In some embodiments, MYTX-011 provides one or more benefit to the human subjects. In some embodiments, treatment with MYTX-011 at 1 mg/kg produces stable disease in a human subject. In some embodiments, treatment with MYTX-011 at 2 mg/kg produces stable disease and/or a Partial Response (PR) in a human subject. In some embodiments, treatment with MYTX-011 at 3 mg/kg produces stable disease and/or a PR in a human subject. In some embodiments, treatment with MYTX-011 at 4 mg/kg produces stable disease and/or a PR in a human subject. In some embodiments, treatment with MYTX-011 at 5 mg/kg produces stable disease and/or a PR in a human subject.

In some embodiments, the PR is achieved despite progression of the human subject's tumor in 1 or 2 prior treatment regimens. In some embodiments, the prior treatment regimen(s) included one that targeted a MET Exon 14 mutation.

In some embodiments, MYTX-011 at 1, 2, 3, 4, 5, 6, 7, or 8 mg/kg provides a benefit selected from one or more of the following: increased Overall Survival, increased Progression-Free Survival, Stable Disease, Partial Response and tumor shrinkage. In some embodiments, as little as 1, 2, 3, or 4 mg/kg of MYTX-011 causes at least a 20% reduction in tumor volume within as little as 2, 3, 4, or 5 treatment cycles.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

SEQUENCE APPENDIX
>Mature Human MET
(SEQ ID NO: 1)
ECKEALAKSEMNVNMKYQLPNFTAETPIQNVILHEHHIFLGATNYIYVLNEEDLQKVAEYKTGPVLEHPDCFPCQDCSSKANLSGGV
WKDNINMALVVDTYYDDQLISCGSVNRGTCQRHVFPHNHTADIQSEVHCIFSPQIEEPSQCPDCVVSALGAKVLSSVKDRFINFFVG
NTINSSYFPDHPLHSISVRRLKETKDGFMFLTDQSYIDVLPEFRDSYPIKYVHAFESNNFIYFLTVQRETLDAQTFHTRIIRFCSINSGLHS
YMEMPLECILTEKRKKRSTKKEVFNILQAAYVSKPGAQLARQIGASLNDDILFGVFAQSKPDSAEPMDRSAMCAFPIKYVNDFFNKIV
NKNNVRCLQHFYGPNHEHCFNRTLLRNSSGCEARRDEYRTEFTTALQRVDLFMGQFSEVLLTSISTFIKGDLTIANLGTSEGRFMQV
VVSRSGPSTPHVNFLLDSHPVSPEVIVEHTLNQNGYTLVITGKKITKIPLNGLGCRHFQSCSQCLSAPPFVQCGWCHDKCVRSEECLS
GTWTQQICLPAIYKVFPNSAPLEGGTRLTICGWDFGFRRNNKFDLKKTRVLLGNESCTLTLSESTMNTLKCTVGPAMNKHFNMSIIIS
NGHGTTQYSTFSYVDPVITSISPKYGPMAGGTLLTLTGNYLNSGNSRHISIGGKTCTLKSVSNSILECYTPAQTISTEFAVKLKIDLANRE
TSIFSYREDPIVYEIHPTKSFISGGSTITGVGKNLNSVSVPRMVINVHEAGRNFTVACQHRSNSEIICCTTPSLQQLNLQLPLKTKAFFML
DGILSKYFDLIYVHNPVFKPFEKPVMISMGNENVLEIKGNDIDPEAVKGEVLKVGNKSCENIHLHSEAVLCTVPNDLLKLNSELNIEWK
QAISSTVLGKVIVQPDQNFTGLIAGVVSISTALLLLLGFFLWLKKRKQIKDLGSELVRYDARVHTPHLDRLVSARSVSPTTEMVSNESVD
YRATFPEDQFPNSSQNGSCRQVQYPLTDMSPILTSGDSDISSPLLQNTVHIDLSALNPELVQAVQHVVIGPSSLIVHFNEVIGRGHFG
CVYHGTLLDNDGKKIHCAVKSLNRITDIGEVSQFLTEGIIMKDFSHPNVLSLLGICLRSEGSPLVVLPYMKHGDLRNFIRNETHNPTVK
DLIGFGLQVAKGMKYLASKKFVHRDLAARNCMLDEKFTVKVADFGLARDMYDKEYYSVHNKTGAKLPVKWMALESLQTQKFTTKS
DVWSFGVLLWELMTRGAPPYPDVNTFDITVYLLQGRRLLQPEYCPDPLYEVMLKCWHPKAEMRPSFSELVSRISAIFSTFIGEHYVH
VNATYVNVKCVAPYPSLLSSEDNADDEVDTRPASFWETS
>cDNA Encoding Mature Human MET
(SEQ ID NO: 2)
GAGTGTAAAGAGGCACTAGCAAAGTCCGAGATGAATGTGAATATGAAGTATCAGCTTCCCAACTTCACCGCGGAAACACCCAT
CCAGAATGTCATTCTACATGAGCATCACATTTTCCTTGGTGCCACTAACTACATTTATGTTTTAAATGAGGAAGACCTTCAGAAG
GTTGCTGAGTACAAGACTGGGCCTGTGCTGGAACACCCAGATTGTTTCCCATGTCAGGACTGCAGCAGCAAAGCCAATTTATC
AGGAGGTGTTTGGAAAGATAACATCAACATGGCTCTAGTTGTCGACACCTACTATGATGATCAACTCATTAGCTGTGGCAGCG
TCAACAGAGGGACCTGCCAGCGACATGTCTTTCCCCACAATCATACTGCTGACATACAGTCGGAGGTTCACTGCATATTCTCCC
CACAGATAGAAGAGCCCAGCCAGTGTCCTGACTGTGTGGTGAGCGCCCTGGGAGCCAAAGTCCTTTCATCTGTAAAGGACCG
GTTCATCAACTTCTTTGTAGGCAATACCATAAATTCTTCTTATTTCCCAGATCATCCATTGCATTCGATATCAGTGAGAAGGCTAA
AGGAAACGAAAGATGGTTTTATGTTTTTGACGGACCAGTCCTACATTGATGTTTTACCTGAGTTCAGAGATTCTTACCCCATTAA
GTATGTCCATGCCTTTGAAAGCAACAATTTTATTTACTTCTTGACGGTCCAAAGGGAAACTCTAGATGCTCAGACTTTTCACACA
AGAATAATCAGGTTCTGTTCCATAAACTCTGGATTGCATTCCTACATGGAAATGCCTCTGGAGTGTATTCTCACAGAAAAGAGA
AAAAAGAGATCCACAAAGAAGGAAGTGTTTAATATACTTCAGGCTGCGTATGTCAGCAAGCCTGGGGCCCAGCTTGCTAGACA
AATAGGAGCCAGCCTGAATGATGACATTCTTTTCGGGGTGTTCGCACAAAGCAAGCCAGATTCTGCCGAACCAATGGATCGAT
CTGCCATGTGTGCATTCCCTATCAAATATGTCAACGACTTCTTCAACAAGATCGTCAACAAAAACAATGTGAGATGTCTCCAGC
ATTTTTACGGACCCAATCATGAGCACTGCTTTAATAGGACACTTCTGAGAAATTCATCAGGCTGTGAAGCGCGCCGTGATGAAT
ATCGAACAGAGTTTACCACAGCTTTGCAGCGCGTTGACTTATTCATGGGTCAATTCAGCGAAGTCCTCTTAACATCTATATCCAC
CTTCATTAAAGGAGACCTCACCATAGCTAATCTTGGGACATCAGAGGGTCGCTTCATGCAGGTTGTGGTTTCTCGATCAGGACC
ATCAACCCCTCATGTGAATTTTCTCCTGGACTCCCATCCAGTGTCTCCAGAAGTGATTGTGGAGCATACATTAAACCAAAATGG
CTACACACTGGTTATCACTGGGAAGAAGATCACGAAGATCCCATTGAATGGCTTGGGCTGCAGACATTTCCAGTCCTGCAGTC
AATGCCTCTCTGCCCCACCCTTTGTTCAGTGTGGCTGGTGCCACGACAAATGTGTGCGATCGGAGGAATGCCTGAGCGGGACA
TGGACTCAACAGATCTGTCTGCCTGCAATCTACAAGGTTTTCCCAAATAGTGCACCCCTTGAAGGAGGGACAAGGCTGACCAT
ATGTGGCTGGGACTTTGGATTTCGGAGGAATAATAAATTTGATTTAAAGAAAACTAGAGTTCTCCTTGGAAATGAGAGCTGCA
CCTTGACTTTAAGTGAGAGCACGATGAATACATTGAAATGCACAGTTGGTCCTGCCATGAATAAGCATTTCAATATGTCCATAA
TTATTTCAAATGGCCACGGGACAACACAATACAGTACATTCTCCTATGTGGATCCTGTAATAACAAGTATTTCGCCGAAATACG
GTCCTATGGCTGGTGGCACTTTACTTACTTTAACTGGAAATTACCTAAACAGTGGGAATTCTAGACACATTTCAATTGGTGGAA
AAACATGTACTTTAAAAAGTGTGTCAAACAGTATTCTTGAATGTTATACCCCAGCCCAAACCATTTCAACTGAGTTTGCTGTTAA
ATTGAAAATTGACTTAGCCAACCGAGAGACAAGCATCTTCAGTTACCGTGAAGATCCCATTGTCTATGAAATTCATCCAACCAA
ATCTTTTATTAGTGGTGGGAGCACAATAACAGGTGTTGGGAAAAACCTGAATTCAGTTAGTGTCCCGAGAATGGTCATAAATG
TGCATGAAGCAGGAAGGAACTTTACAGTGGCATGTCAACATCGCTCTAATTCAGAGATAATCTGTTGTACCACTCCTTCCCTGC
AACAGCTGAATCTGCAACTCCCCCTGAAAACCAAAGCCTTTTTCATGTTAGATGGGATCCTTTCCAAATACTTTGATCTCATTTAT
GTACATAATCCTGTGTTTAAGCCTTTTGAAAAGCCAGTGATGATCTCAATGGGCAATGAAAATGTACTGGAAATTAAGGGAAA
TGATATTGACCCTGAAGCAGTTAAAGGTGAAGTGTTAAAAGTTGGAAATAAGAGCTGTGAGAATATACACTTACATTCTGAAG
CCGTTTTATGCACGGTCCCCAATGACCTGCTGAAATTGAACAGCGAGCTAAATATAGAGTGGAAGCAAGCAATTTCTTCAACC
GTCCTTGGAAAAGTAATAGTTCAACCAGATCAGAATTTCACAGGATTGATTGCTGGTGTTGTCTCAATATCAACAGCACTGTTA
TTACTACTTGGGTTTTTCCTGTGGCTGAAAAAGAGAAAGCAAATTAAAGATCTGGGCAGTGAATTAGTTCGCTACGATGCAAG
AGTACACACTCCTCATTTGGATAGGCTTGTAAGTGCCCGAAGTGTAAGCCCAACTACAGAAATGGTTTCAAATGAATCTGTAGA
CTACCGAGCTACTTTTCCAGAAGATCAGTTTCCTAATTCATCTCAGAACGGTTCATGCCGACAAGTGCAGTATCCTCTGACAGAC
ATGTCCCCCATCCTAACTAGTGGGGACTCTGATATATCCAGTCCATTACTGCAAAATACTGTCCACATTGACCTCAGTGCTCTAA
ATCCAGAGCTGGTCCAGGCAGTGCAGCATGTAGTGATTGGGCCCAGTAGCCTGATTGTGCATTTCAATGAAGTCATAGGAAGA
GGGCATTTTGGTTGTGTATATCATGGGACTTTGTTGGACAATGATGGCAAGAAAATTCACTGTGCTGTGAAATCCTTGAACAG
AATCACTGACATAGGAGAAGTTTCCCAATTTCTGACCGAGGGAATCATCATGAAAGATTTTAGTCATCCCAATGTCCTCTCGCT
CCTGGGAATCTGCCTGCGAAGTGAAGGGTCTCCGCTGGTGGTCCTACCATACATGAAACATGGAGATCTTCGAAATTTCATTC
GAAATGAGACTCATAATCCAACTGTAAAAGATCTTATTGGCTTTGGTCTTCAAGTAGCCAAAGGCATGAAATATCTTGCAAGCA
AAAAGTTTGTCCACAGAGACTTGGCTGCAAGAAACTGTATGCTGGATGAAAAATTCACAGTCAAGGTTGCTGATTTTGGTCTT
GCCAGAGACATGTATGATAAAGAATACTATAGTGTACACAACAAAACAGGTGCAAAGCTGCCAGTGAAGTGGATGGCTTTGG
AAAGTCTGCAAACTCAAAAGTTTACCACCAAGTCAGATGTGTGGTCCTTTGGCGTGCTCCTCTGGGAGCTGATGACAAGAGGA
GCCCCACCTTATCCTGACGTAAACACCTTTGATATAACTGTTTACTTGTTGCAAGGGAGAAGACTCCTACAACCCGAATACTGCC
CAGACCCCTTATATGAAGTAATGCTAAAATGCTGGCACCCTAAAGCCGAAATGCGCCCATCCTTTTCTGAACTGGTGTCCCGGA
TATCAGCGATCTTCTCTACTTTCATTGGGGAGCACTATGTCCATGTGAACGCTACTTATGTGAACGTAAAATGTGTCGCTCCGTA
TCCTTCTCTGTTGTCATCAGAAGATAACGCTGATGATGAGGTGGACACACGACCAGCCTCCTTCTGGGAGACATCA
>Extracellular Domain of MET
(SEQ ID NO: 3)
ECKEALAKSEMNVNMKYQLPNFTAETPIQNVILHEHHIFLGATNYIYVLNEEDLQKVAEYKTGPVLEHPDCFPCQDCSSKANLSGGV
WKDNINMALVVDTYYDDQLISCGSVNRGTCQRHVFPHNHTADIQSEVHCIFSPQIEEPSQCPDCVVSALGAKVLSSVKDRFINFFVG
NTINSSYFPDHPLHSISVRRLKETKDGFMFLTDQSYIDVLPEFRDSYPIKYVHAFESNNFIYFLTVQRETLDAQTFHTRIIRFCSINSGLHS
YMEMPLECILTEKRKKRSTKKEVFNILQAAYVSKPGAQLARQIGASLNDDILFGVFAQSKPDSAEPMDRSAMCAFPIKYVNDFFNKIV
NKNNVRCLQHFYGPNHEHCFNRTLLRNSSGCEARRDEYRTEFTTALQRVDLFMGQFSEVLLTSISTFIKGDLTIANLGTSEGRFMQV
VVSRSGPSTPHVNFLLDSHPVSPEVIVEHTLNQNGYTLVITGKKITKIPLNGLGCRHFQSCSQCLSAPPFVQCGWCHDKCVRSEECLS
GTWTQQICLPAIYKVFPNSAPLEGGTRLTICGWDFGFRRNNKFDLKKTRVLLGNESCTLTLSESTMNTLKCTVGPAMNKHFNMSIIIS
NGHGTTQYSTFSYVDPVITSISPKYGPMAGGTLLTLTGNYLNSGNSRHISIGGKTCTLKSVSNSILECYTPAQTISTEFAVKLKIDLANRE
TSIFSYREDPIVYEIHPTKSFISGGSTITGVGKNLNSVSVPRMVINVHEAGRNFTVACQHRSNSEIICCTTPSLQQLNLQLPLKTKAFFML
DGILSKYFDLIYVHNPVFKPFEKPVMISMGNENVLEIKGNDIDPEAVKGEVLKVGNKSCENIHLHSEAVLCTVPNDLLKLNSELNIEWK
QAISSTVLGKVIVQPDQNFT
>cDNA Encoding Extracellular Domain of MET
(SEQ ID NO: 4)
GAGTGTAAAGAGGCACTAGCAAAGTCCGAGATGAATGTGAATATGAAGTATCAGCTTCCCAACTTCACCGCGGAAACACCCAT
CCAGAATGTCATTCTACATGAGCATCACATTTTCCTTGGTGCCACTAACTACATTTATGTTTTAAATGAGGAAGACCTTCAGAAG
GTTGCTGAGTACAAGACTGGGCCTGTGCTGGAACACCCAGATTGTTTCCCATGTCAGGACTGCAGCAGCAAAGCCAATTTATC
AGGAGGTGTTTGGAAAGATAACATCAACATGGCTCTAGTTGTCGACACCTACTATGATGATCAACTCATTAGCTGTGGCAGCG
TCAACAGAGGGACCTGCCAGCGACATGTCTTTCCCCACAATCATACTGCTGACATACAGTCGGAGGTTCACTGCATATTCTCCC
CACAGATAGAAGAGCCCAGCCAGTGTCCTGACTGTGTGGTGAGCGCCCTGGGAGCCAAAGTCCTTTCATCTGTAAAGGACCG
GTTCATCAACTTCTTTGTAGGCAATACCATAAATTCTTCTTATTTCCCAGATCATCCATTGCATTCGATATCAGTGAGAAGGCTAA
AGGAAACGAAAGATGGTTTTATGTTTTTGACGGACCAGTCCTACATTGATGTTTTACCTGAGTTCAGAGATTCTTACCCCATTAA
GTATGTCCATGCCTTTGAAAGCAACAATTTTATTTACTTCTTGACGGTCCAAAGGGAAACTCTAGATGCTCAGACTTTTCACACA
AGAATAATCAGGTTCTGTTCCATAAACTCTGGATTGCATTCCTACATGGAAATGCCTCTGGAGTGTATTCTCACAGAAAAGAGA
AAAAAGAGATCCACAAAGAAGGAAGTGTTTAATATACTTCAGGCTGCGTATGTCAGCAAGCCTGGGGCCCAGCTTGCTAGACA
AATAGGAGCCAGCCTGAATGATGACATTCTTTTCGGGGTGTTCGCACAAAGCAAGCCAGATTCTGCCGAACCAATGGATCGAT
CTGCCATGTGTGCATTCCCTATCAAATATGTCAACGACTTCTTCAACAAGATCGTCAACAAAAACAATGTGAGATGTCTCCAGC
ATTTTTACGGACCCAATCATGAGCACTGCTTTAATAGGACACTTCTGAGAAATTCATCAGGCTGTGAAGCGCGCCGTGATGAAT
ATCGAACAGAGTTTACCACAGCTTTGCAGCGCGTTGACTTATTCATGGGTCAATTCAGCGAAGTCCTCTTAACATCTATATCCAC
CTTCATTAAAGGAGACCTCACCATAGCTAATCTTGGGACATCAGAGGGTCGCTTCATGCAGGTTGTGGTTTCTCGATCAGGACC
ATCAACCCCTCATGTGAATTTTCTCCTGGACTCCCATCCAGTGTCTCCAGAAGTGATTGTGGAGCATACATTAAACCAAAATGG
CTACACACTGGTTATCACTGGGAAGAAGATCACGAAGATCCCATTGAATGGCTTGGGCTGCAGACATTTCCAGTCCTGCAGTC
AATGCCTCTCTGCCCCACCCTTTGTTCAGTGTGGCTGGTGCCACGACAAATGTGTGCGATCGGAGGAATGCCTGAGCGGGACA
TGGACTCAACAGATCTGTCTGCCTGCAATCTACAAGGTTTTCCCAAATAGTGCACCCCTTGAAGGAGGGACAAGGCTGACCAT
ATGTGGCTGGGACTTTGGATTTCGGAGGAATAATAAATTTGATTTAAAGAAAACTAGAGTTCTCCTTGGAAATGAGAGCTGCA
CCTTGACTTTAAGTGAGAGCACGATGAATACATTGAAATGCACAGTTGGTCCTGCCATGAATAAGCATTTCAATATGTCCATAA
TTATTTCAAATGGCCACGGGACAACACAATACAGTACATTCTCCTATGTGGATCCTGTAATAACAAGTATTTCGCCGAAATACG
GTCCTATGGCTGGTGGCACTTTACTTACTTTAACTGGAAATTACCTAAACAGTGGGAATTCTAGACACATTTCAATTGGTGGAA
AAACATGTACTTTAAAAAGTGTGTCAAACAGTATTCTTGAATGTTATACCCCAGCCCAAACCATTTCAACTGAGTTTGCTGTTAA
ATTGAAAATTGACTTAGCCAACCGAGAGACAAGCATCTTCAGTTACCGTGAAGATCCCATTGTCTATGAAATTCATCCAACCAA
ATCTTTTATTAGTGGTGGGAGCACAATAACAGGTGTTGGGAAAAACCTGAATTCAGTTAGTGTCCCGAGAATGGTCATAAATG
TGCATGAAGCAGGAAGGAACTTTACAGTGGCATGTCAACATCGCTCTAATTCAGAGATAATCTGTTGTACCACTCCTTCCCTGC
AACAGCTGAATCTGCAACTCCCCCTGAAAACCAAAGCCTTTTTCATGTTAGATGGGATCCTTTCCAAATACTTTGATCTCATTTAT
GTACATAATCCTGTGTTTAAGCCTTTTGAAAAGCCAGTGATGATCTCAATGGGCAATGAAAATGTACTGGAAATTAAGGGAAA
TGATATTGACCCTGAAGCAGTTAAAGGTGAAGTGTTAAAAGTTGGAAATAAGAGCTGTGAGAATATACACTTACATTCTGAAG
CCGTTTTATGCACGGTCCCCAATGACCTGCTGAAATTGAACAGCGAGCTAAATATAGAGTGGAAGCAAGCAATTTCTTCAACC
GTCCTTGGAAAAGTAATAGTTCAACCAGATCAGAATTTCACA
>MYT5351 Heavy Chain Variable Region
(SEQ ID NO: 5)
QVQLVQSGAEVKKPGASVKVSCKASGYTFTSYWMHWVKQAPGQGLDWIGHIKPSTDNTEYNQKFKDKATLTADKSTSTAYMELS
SLRSEDTAVYYCARSYGNYPLMDYWGQGTTVTVSS
>MYT5351 Light Chain Variable Region
(SEQ ID NO: 6)
QIVLTQSPAILSLSPGERATLSCSASSSVTSNYLYWYQQKPGSSPKLLIYSHSNLASGVPARFSGSGSGTSYTLTISSLEAEDAASYFCHQ
WSSYPPTFGSGTKLEIK
>MYT4313 Heavy Chain Variable Region
(SEQ ID NO: 7)
QVQLVQSGAEVKKPGASVKVSCKASGYTFTSYWMHWVKQAPGQGLDWIGYIKPSTDNTHYNQKFKDKATLTADKSTSTAYMELS
SLRSEDTAVYYCARSYGHYPLMDYWGQGTTVTVSS
>MYT4313 Light Chain Variable Region
(SEQ ID NO: 8)
QIVLTQSPAILSLSPGERATLSCSASSSVTSNYLYWYQQKPGSSPKLLIYSTSNLASGVPARFSGSGSGTSYTLTISSLEAEDAASYFCHQ
WSSYPPTFGSGTKLEIK
>MYT4325 Heavy Chain Variable Region
(SEQ ID NO: 9)
QVQLVQSGAEVKKPGASVKVSCKASGYTFTSYWMHWVKQAPGQGLDWIGYIKPSTDNTHYNQKFKDKATLTADKSTSTAYMELS
SLRSEDTAVYYCARSYGHYPLMHYWGQGTTVTVSS
>MYT4325 Light Chain Variable Region
(SEQ ID NO: 10)
QIVLTQSPAILSLSPGERATLSCSASSSVTSNYLYWYQQKPGSSPKLLIYSTSNLASGVPARFSGSGSGTSYTLTISSLEAEDAASYFCHQ
WSSYPPTFGSGTKLEIK
>MYT4826 Heavy Chain Variable Region
(SEQ ID NO: 11)
QVQLVQSGAEVKKPGASVKVSCKASGYTFTHYYMHWVRQAPGQGLEWMGRVNPNRRHTTYNQKFEGRVTMTTDTSTSTAYME
LRSLRSDDTAVYYCARANWLDYWGQGTTVTVSS
>MYT4826 Light Chain Variable Region
(SEQ ID NO: 12)
DIQMTQSPSSLSASVGDRVTITCSVSSSVSSIYLHWYQQKPGKAPKLLIYSTSNLASGVPSRFSGSGSGTDFTLTISSLQPEDFATYYCQ
VYSGYPLTFGGGTKVEIK
>MYT4837 Heavy Chain Variable Region
(SEQ ID NO: 13)
QVQLVQSGAEVKKPGASVKVSCKASGYTFTHYYMHWVRQAPGQGLEWMGRVNPHRRHTTYNQKFEGRVTMTTDTSTSTAYME
LRSLRSDDTAVYYCARANWLDYWGQGTTVTVSS
>MYT4837 Light Chain Variable Region
(SEQ ID NO: 14)
DIQMTQSPSSLSASVGDRVTITCSVSSSVSSIYLHWYQQKPGKAPKLLIYSTSNLASGVPSRFSGSGSGTDFTLTISSLQPEDFATYYCQ
VYSGYPLTFGGGTKVEIK
>MYT4849 Heavy Chain Variable Region
(SEQ ID NO: 15)
QVQLVQSGAEVKKPGASVKVSCKASGYTFTDYYMHWVRQAPGQGLEWMGRVNPNRRGTTYNQKFEGRVTMTTDTSTSTAYME
LRSLRSDDTAVYYCARANWLDYWGQGTTVTVSS
>MYT4849 Light Chain Variable Region
(SEQ ID NO: 16)
DIQMTQSPSSLSASVGDRVTITCSVSSSVSSIHLHWYQQKPGKAPKLLIYHTSNLASGVPSRFSGSGSGTDFTLTISSLQPEDFATYYCQ
VYSGYPLTFGGGTKVEIK
>MYT4942 Heavy Chain Variable Region
(SEQ ID NO: 17)
QVQLVQSGAEVKKPGASVKVSCKASGHIFTAYTMHWVRQAPGQGLEWMGWIKPNNGLANYAQKFQGRVTMTRDTSISTAYME
LSRLRSDDTAVYYCARSEITHEFDHWGQGTLVTVSS
>MYT4942 Light Chain Variable Region
(SEQ ID NO: 18)
DIVMTQSPDSLAVSLGERATINCKSSESVDSYANSHLHWYQQKPGQPPKLLIYRASTRESGVPDRFSGSGSGTDFTLTISSLQAEDVA
VYYCQQSKEDPLTFGGGTKVEIK
>MYT5309 Heavy Chain Variable Region
(SEQ ID NO: 19)
QVQLVQSGAEVKKPGASVKVSCKASGYTFTHYYMHWVRQAPGQGLEWMGRVNPNRRGTTYNQKFEGRVTMTTDTSTSTAYME
LRSLRSDDTAVYYCARANWLDYWGQGTTVTVSS
>MYT5309 Light Chain Variable Region
(SEQ ID NO: 20)
DIQMTQSPSSLSASVGDRVTITCSVSSSVSSIYLHWYQQKPGKAPKLLIYSTSNLASGVPSRFSGSGSGTDFTLTISSLQPEDFATYYCQ
HYSGYPLTFGGGTKVEIK
>MYT5344 Heavy Chain Variable Region
(SEQ ID NO: 21)
QVQLVQSGAEVKKPGASVKVSCKASGYTFTSYWMHWVKQAPGQGLDWIGYIKHSTDNTEYNQKFKDKATLTADKSTSTAYMELS
SLRSEDTAVYYCARSYGNYPLMDYWGQGTTVTVSS
>MYT5344 Light Chain Variable Region
(SEQ ID NO: 22)
QIVLTQSPAILSLSPGERATLSCSASSSVTSHYLYWYQQKPGSSPKLLIYSTSNLASGVPARFSGSGSGTSYTLTISSLEAEDAASYFCHQ
WSSYPPTFGSGTKLEIK
>MYT5367 Heavy Chain Variable Region
(SEQ ID NO: 23)
QVQLVQSGAEVKKPGASVKVSCKASGYTFTSYWMHWVKQAPGQGLDWIGHIKPSTDNTEYNQKFKDKATLTADKSTSTAYMELS
SLRSEDTAVYYCARSYGNYPLMDYWGQGTTVTVSS
>MYT5367 Light Chain Variable Region
(SEQ ID NO: 24)
QIVLTQSPAILSLSPGERATLSCSASSSVTSNYLYWYQQKPGSSPKLLIYSTSNLASGVPARFSGSGSGTSYTLTISSLEAEDAASYFCHQ
WSHYHPTFGSGTKLEIK
>MYT4827 Heavy Chain Variable Region
(SEQ ID NO: 25)
QVQLVQSGAEVKKPGASVKVSCKASGYTFTHYYMHWVRQAPGQGLEWMGRVNPNRRGTTYNQKFEGRVTMTTDTSTSTAYME
LRSLRSDDTAVYYCARAHWLDYWGQGTTVTVSS
>MYT4827 Light Chain Variable Region
(SEQ ID NO: 26)
DIQMTQSPSSLSASVGDRVTITCSVSSSVSSIYLHWYQQKPGKAPKLLIYSTSNLASGVPSRFSGSGSGTDFTLTISSLQPEDFATYYCQ
VYSGYPLTFGGGTKVEIK
>MYT4312 Heavy Chain Variable Region
(SEQ ID NO: 27)
QVQLVQSGAEVKKPGASVKVSCKASGYTFTSYWMHWVKQAPGQGLDWIGHIKPSTDNTEYNQKFKDKATLTADKSTSTAYMELS
SLRSEDTAVYYCARSYGNYPLMHYWGQGTTVTVSS
>MYT4312 Light Chain Variable Region
(SEQ ID NO: 28)
QIVLTQSPAILSLSPGERATLSCSASSSVTSNYLYWYQQKPGSSPKLLIYSTSNLASGVPARFSGSGSGTSYTLTISSLEAEDAASYFCHQ
WSSYPPTFGSGTKLEIK
>MYT4953 Heavy Chain Variable Region
(SEQ ID NO: 29)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCHRSEITHEFDYWGQGTLVTVSS
>MYT4953 Light Chain Variable Region
(SEQ ID NO: 30)
DIVMTQSPDSLAVSLGERATINCKSSESVDSYANSFLHWYQQKPGQPPKLLIYRASTRESGVPDRFSGSGSGTDFTLTISSLQAEDVAV
YYCQQSKEDPLTFGGGTKVEIK
>MYT4940 Heavy Chain Variable Region
(SEQ ID NO: 31)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCARHEITTEFDHWGQGTLVTVSS
>MYT4940 Light Chain Variable Region
(SEQ ID NO: 32)
DIVMTQSPDSLAVSLGERATINCKSSESVDSYANSHLHWYQQKPGQPPKLLIYRASTRESGVPDRFSGSGSGTDFTLTISSLQAEDVA
VYYCQQSKEDPLTFGGGTKVEIK
>MYT4888 Heavy Chain Variable Region
(SEQ ID NO: 33)
QVQLVQSGAEVKKPGASVKVSCKASGYTFTSYWMHWVKQAPGQGLDWIGYIKPSTDNTEYNQKFKDKATLTADKSTSTAYMELS
SLRSEDTAVYYCARSYGNYPLHDYWGQGTTVTVSS
>MYT4888 Light Chain Variable Region
(SEQ ID NO: 34)
QIVLTQSPAILSLSPGERATLSCSASSSVTSNYLYWYQQKPGSSPKLLIYSTSNLASGVPARFSGSGSGTSYTLTISSLEAEDAASYFCHQ
WSSYPPTFGSGTKLEIK
>MYT5351 Heavy Chain Triple Hinge
(SEQ ID NO: 35)
QVQLVQSGAEVKKPGASVKVSCKASGYTFTSYWMHWVKQAPGQGLDWIGHIKPSTDNTEYNQKFKDKATLTADKSTSTAYMELS
SLRSEDTAVYYCARSYGNYPLMDYWGQGTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT5351 Heavy Chain Triple Hinge + “LS” mutation
(SEQ ID NO: 36)
QVQLVQSGAEVKKPGASVKVSCKASGYTFTSYWMHWVKQAPGQGLDWIGHIKPSTDNTEYNQKFKDKATLTADKSTSTAYMELS
SLRSEDTAVYYCARSYGNYPLMDYWGQGTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT5351 Heavy Chain Triple Hinge + “YTE” mutation
(SEQ ID NO: 37)
QVQLVQSGAEVKKPGASVKVSCKASGYTFTSYWMHWVKQAPGQGLDWIGHIKPSTDNTEYNQKFKDKATLTADKSTSTAYMELS
SLRSEDTAVYYCARSYGNYPLMDYWGQGTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLYITREPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT5351 Heavy Chain Triple Hinge and “A118C”
(SEQ ID NO: 38)
QVQLVQSGAEVKKPGASVKVSCKASGYTFTSYWMHWVKQAPGQGLDWIGHIKPSTDNTEYNQKFKDKATLTADKSTSTAYMELS
SLRSEDTAVYYCARSYGNYPLMDYWGQGTTVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT5351 Heavy Chain Triple Hinge + “LS” mutation and “A118C”
(SEQ ID NO: 39)
QVQLVQSGAEVKKPGASVKVSCKASGYTFTSYWMHWVKQAPGQGLDWIGHIKPSTDNTEYNQKFKDKATLTADKSTSTAYMELS
SLRSEDTAVYYCARSYGNYPLMDYWGQGTTVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT5351 Heavy Chain Triple Hinge + “YTE” mutation and “A118C”
(SEQ ID NO: 40)
QVQLVQSGAEVKKPGASVKVSCKASGYTFTSYWMHWVKQAPGQGLDWIGHIKPSTDNTEYNQKFKDKATLTADKSTSTAYMELS
SLRSEDTAVYYCARSYGNYPLMDYWGQGTTVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLYITREPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT5351 Light Chain
(SEQ ID NO: 41)
QIVLTQSPAILSLSPGERATLSCSASSSVTSNYLYWYQQKPGSSPKLLIYSHSNLASGVPARFSGSGSGTSYTLTISSLEAEDAASYFCHQ
WSSYPPTFGSGTKLEIKRTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC**
>MYT5351 Light Chain (“V205C”)
(SEQ ID NO: 42)
QIVLTQSPAILSLSPGERATLSCSASSSVTSNYLYWYQQKPGSSPKLLIYSHSNLASGVPARFSGSGSGTSYTLTISSLEAEDAASYFCHQ
WSSYPPTFGSGTKLEIKRTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPCTKSFNRGEC**
>MYT4313 Heavy Chain Triple Hinge
(SEQ ID NO: 43)
QVQLVQSGAEVKKPGASVKVSCKASGYTFTSYWMHWVKQAPGQGLDWIGYIKPSTDNTHYNQKFKDKATLTADKSTSTAYMELS
SLRSEDTAVYYCARSYGHYPLMDYWGQGTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4313 Heavy Chain Triple Hinge + “LS” mutation
(SEQ ID NO: 44)
QVQLVQSGAEVKKPGASVKVSCKASGYTFTSYWMHWVKQAPGQGLDWIGYIKPSTDNTHYNQKFKDKATLTADKSTSTAYMELS
SLRSEDTAVYYCARSYGHYPLMDYWGQGTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT4313 Heavy Chain Triple Hinge + “YTE” mutation
(SEQ ID NO: 45)
QVQLVQSGAEVKKPGASVKVSCKASGYTFTSYWMHWVKQAPGQGLDWIGYIKPSTDNTHYNQKFKDKATLTADKSTSTAYMELS
SLRSEDTAVYYCARSYGHYPLMDYWGQGTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLYITREPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4313 Heavy Chain Triple Hinge and “A118C”
(SEQ ID NO: 46)
QVQLVQSGAEVKKPGASVKVSCKASGYTFTSYWMHWVKQAPGQGLDWIGYIKPSTDNTHYNQKFKDKATLTADKSTSTAYMELS
SLRSEDTAVYYCARSYGHYPLMDYWGQGTTVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4313 Heavy Chain Triple Hinge + “LS” mutation and “A118C”
(SEQ ID NO: 47)
QVQLVQSGAEVKKPGASVKVSCKASGYTFTSYWMHWVKQAPGQGLDWIGYIKPSTDNTHYNQKFKDKATLTADKSTSTAYMELS
SLRSEDTAVYYCARSYGHYPLMDYWGQGTTVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT4313 Heavy Chain Triple Hinge + “YTE” mutation and “A118C”
(SEQ ID NO: 48)
QVQLVQSGAEVKKPGASVKVSCKASGYTFTSYWMHWVKQAPGQGLDWIGYIKPSTDNTHYNQKFKDKATLTADKSTSTAYMELS
SLRSEDTAVYYCARSYGHYPLMDYWGQGTTVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLYITREPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4313 Light Chain
(SEQ ID NO: 49)
QIVLTQSPAILSLSPGERATLSCSASSSVTSNYLYWYQQKPGSSPKLLIYSTSNLASGVPARFSGSGSGTSYTLTISSLEAEDAASYFCHQ
WSSYPPTFGSGTKLEIKRTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC**
>MYT4313 Light Chain “V205C”
(SEQ ID NO: 50)
QIVLTQSPAILSLSPGERATLSCSASSSVTSNYLYWYQQKPGSSPKLLIYSTSNLASGVPARFSGSGSGTSYTLTISSLEAEDAASYFCHQ
WSSYPPTFGSGTKLEIKRTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPCTKSFNRGEC**
>MYT4325 Heavy Chain Triple Hinge
(SEQ ID NO: 51)
QVQLVQSGAEVKKPGASVKVSCKASGYTFTSYWMHWVKQAPGQGLDWIGYIKPSTDNTHYNQKFKDKATLTADKSTSTAYMELS
SLRSEDTAVYYCARSYGHYPLMHYWGQGTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4325 Heavy Chain Triple Hinge + “LS” mutation
(SEQ ID NO: 52)
QVQLVQSGAEVKKPGASVKVSCKASGYTFTSYWMHWVKQAPGQGLDWIGYIKPSTDNTHYNQKFKDKATLTADKSTSTAYMELS
SLRSEDTAVYYCARSYGHYPLMHYWGQGTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT4325 Heavy Chain Triple Hinge + “YTE” mutation
(SEQ ID NO: 53)
QVQLVQSGAEVKKPGASVKVSCKASGYTFTSYWMHWVKQAPGQGLDWIGYIKPSTDNTHYNQKFKDKATLTADKSTSTAYMELS
SLRSEDTAVYYCARSYGHYPLMHYWGQGTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLYITREPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4325 Heavy Chain Triple Hinge and “A118C”
(SEQ ID NO: 54)
QVQLVQSGAEVKKPGASVKVSCKASGYTFTSYWMHWVKQAPGQGLDWIGYIKPSTDNTHYNQKFKDKATLTADKSTSTAYMELS
SLRSEDTAVYYCARSYGHYPLMHYWGQGTTVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4325 Heavy Chain (Triple Hinge + “LS” mutation and A118C)
(SEQ ID NO: 55)
QVQLVQSGAEVKKPGASVKVSCKASGYTFTSYWMHWVKQAPGQGLDWIGYIKPSTDNTHYNQKFKDKATLTADKSTSTAYMELS
SLRSEDTAVYYCARSYGHYPLMHYWGQGTTVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT4325 Heavy Chain (Triple Hinge + “YTE” mutation and A118C)
(SEQ ID NO: 56)
QVQLVQSGAEVKKPGASVKVSCKASGYTFTSYWMHWVKQAPGQGLDWIGYIKPSTDNTHYNQKFKDKATLTADKSTSTAYMELS
SLRSEDTAVYYCARSYGHYPLMHYWGQGTTVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLYITREPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4325 Light Chain
(SEQ ID NO: 57)
QIVLTQSPAILSLSPGERATLSCSASSSVTSNYLYWYQQKPGSSPKLLIYSTSNLASGVPARFSGSGSGTSYTLTISSLEAEDAASYFCHQ
WSSYPPTFGSGTKLEIKRTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC**
>MYT4325 Light Chain “V205C”
(SEQ ID NO: 58)
QIVLTQSPAILSLSPGERATLSCSASSSVTSNYLYWYQQKPGSSPKLLIYSTSNLASGVPARFSGSGSGTSYTLTISSLEAEDAASYFCHQ
WSSYPPTFGSGTKLEIKRTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPCTKSFNRGEC**
>MYT4826 Heavy Chain Triple Hinge
(SEQ ID NO: 59)
QVQLVQSGAEVKKPGASVKVSCKASGYTFTHYYMHWVRQAPGQGLEWMGRVNPNRRHTTYNQKFEGRVTMTTDTSTSTAYME
LRSLRSDDTAVYYCARANWLDYWGQGTTVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4826 Heavy Chain (Triple Hinge + “LS” mutation)
(SEQ ID NO: 60)
QVQLVQSGAEVKKPGASVKVSCKASGYTFTHYYMHWVRQAPGQGLEWMGRVNPNRRHTTYNQKFEGRVTMTTDTSTSTAYME
LRSLRSDDTAVYYCARANWLDYWGQGTTVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT4826 Heavy Chain Triple Hinge + “YTE” mutation
(SEQ ID NO: 61)
QVQLVQSGAEVKKPGASVKVSCKASGYTFTHYYMHWVRQAPGQGLEWMGRVNPNRRHTTYNQKFEGRVTMTTDTSTSTAYME
LRSLRSDDTAVYYCARANWLDYWGQGTTVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLYITREPEVTCV
VVDVSHEDPEVKFNWYVDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSRWQQGNVFSCSVMHEALH
NHYTQKSLSLSPG**
>MYT4826 Heavy Chain Triple Hinge and “A118C”
(SEQ ID NO: 62)
QVQLVQSGAEVKKPGASVKVSCKASGYTFTHYYMHWVRQAPGQGLEWMGRVNPNRRHTTYNQKFEGRVTMTTDTSTSTAYME
LRSLRSDDTAVYYCARANWLDYWGQGTTVTVSSCSTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4826 Heavy Chain Triple Hinge + “LS” mutation and “A118C”
(SEQ ID NO: 63)
QVQLVQSGAEVKKPGASVKVSCKASGYTFTHYYMHWVRQAPGQGLEWMGRVNPNRRHTTYNQKFEGRVTMTTDTSTSTAYME
LRSLRSDDTAVYYCARANWLDYWGQGTTVTVSSCSTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT4826 Heavy Chain Triple Hinge + “YTE” mutation and “A118C”
(SEQ ID NO: 64)
QVQLVQSGAEVKKPGASVKVSCKASGYTFTHYYMHWVRQAPGQGLEWMGRVNPNRRHTTYNQKFEGRVTMTTDTSTSTAYME
LRSLRSDDTAVYYCARANWLDYWGQGTTVTVSSCSTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLYITREPEVTCV
VVDVSHEDPEVKFNWYVDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSRWQQGNVFSCSVMHEALH
NHYTQKSLSLSPG**
>MYT4826 Light Chain
(SEQ ID NO: 65)
DIQMTQSPSSLSASVGDRVTITCSVSSSVSSIYLHWYQQKPGKAPKLLIYSTSNLASGVPSRFSGSGSGTDFTLTISSLQPEDFATYYCQ
VYSGYPLTFGGGTKVEIKRTVAAPSVFIFPPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC**
>MYT4826 Light Chain “V205C”
(SEQ ID NO: 66)
DIQMTQSPSSLSASVGDRVTITCSVSSSVSSIYLHWYQQKPGKAPKLLIYSTSNLASGVPSRFSGSGSGTDFTLTISSLQPEDFATYYCQ
VYSGYPLTFGGGTKVEIKRTVAAPSVFIFPPSDEQLKSGTASVVCLLNNFYPREAKVQWKVDNALQSGNSQESVTEQDSKDSTYSLSS
TLTLSKADYEKHKVYACEVTHQGLSSPCTKSFNRGEC**
>MYT4837 Heavy Chain Triple Hinge
(SEQ ID NO: 67)
QVQLVQSGAEVKKPGASVKVSCKASGYTFTHYYMHWVRQAPGQGLEWMGRVNPHRRHTTYNQKFEGRVTMTTDTSTSTAYME
LRSLRSDDTAVYYCARANWLDYWGQGTTVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4837 Heavy Chain Triple Hinge + “LS” mutation
(SEQ ID NO: 68)
QVQLVQSGAEVKKPGASVKVSCKASGYTFTHYYMHWVRQAPGQGLEWMGRVNPHRRHTTYNQKFEGRVTMTTDTSTSTAYME
LRSLRSDDTAVYYCARANWLDYWGQGTTVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT4837 Heavy Chain Triple Hinge + “YTE” mutation
(SEQ ID NO: 69)
QVQLVQSGAEVKKPGASVKVSCKASGYTFTHYYMHWVRQAPGQGLEWMGRVNPHRRHTTYNQKFEGRVTMTTDTSTSTAYME
LRSLRSDDTAVYYCARANWLDYWGQGTTVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLYITREPEVTCV
VVDVSHEDPEVKFNWYVDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSRWQQGNVFSCSVMHEALH
NHYTQKSLSLSPG**
>MYT4837 Heavy Chain Triple Hinge and “A118C”
(SEQ ID NO: 70)
QVQLVQSGAEVKKPGASVKVSCKASGYTFTHYYMHWVRQAPGQGLEWMGRVNPHRRHTTYNQKFEGRVTMTTDTSTSTAYME
LRSLRSDDTAVYYCARANWLDYWGQGTTVTVSSCSTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4837 Heavy Chain Triple Hinge + “LS” mutation and “A118C”
(SEQ ID NO: 71)
QVQLVQSGAEVKKPGASVKVSCKASGYTFTHYYMHWVRQAPGQGLEWMGRVNPHRRHTTYNQKFEGRVTMTTDTSTSTAYME
LRSLRSDDTAVYYCARANWLDYWGQGTTVTVSSCSTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT4837 Heavy Chain Triple Hinge + “YTE” mutation and “A118C”
(SEQ ID NO: 72)
QVQLVQSGAEVKKPGASVKVSCKASGYTFTHYYMHWVRQAPGQGLEWMGRVNPHRRHTTYNQKFEGRVTMTTDTSTSTAYME
LRSLRSDDTAVYYCARANWLDYWGQGTTVTVSSCSTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLYITREPEVTCV
VVDVSHEDPEVKFNWYVDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSRWQQGNVFSCSVMHEALH
NHYTQKSLSLSPG**
>MYT4837 Light Chain
(SEQ ID NO: 73)
DIQMTQSPSSLSASVGDRVTITCSVSSSVSSIYLHWYQQKPGKAPKLLIYSTSNLASGVPSRFSGSGSGTDFTLTISSLQPEDFATYYCQ
VYSGYPLTFGGGTKVEIKRTVAAPSVFIFPPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC**
>MYT4837 Light Chain “V205C”
(SEQ ID NO: 74)
DIQMTQSPSSLSASVGDRVTITCSVSSSVSSIYLHWYQQKPGKAPKLLIYSTSNLASGVPSRFSGSGSGTDFTLTISSLQPEDFATYYCQ
VYSGYPLTFGGGTKVEIKRTVAAPSVFIFPPSDEQLKSGTASVVCLLNNFYPREAKVQWKVDNALQSGNSQESVTEQDSKDSTYSLSS
TLTLSKADYEKHKVYACEVTHQGLSSPCTKSFNRGEC**
>MYT4849 Heavy Chain Triple Hinge
(SEQ ID NO: 75)
QVQLVQSGAEVKKPGASVKVSCKASGYTFTDYYMHWVRQAPGQGLEWMGRVNPNRRGTTYNQKFEGRVTMTTDTSTSTAYME
LRSLRSDDTAVYYCARANWLDYWGQGTTVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4849 Heavy Chain Triple Hinge + “LS” mutation
(SEQ ID NO: 76)
QVQLVQSGAEVKKPGASVKVSCKASGYTFTDYYMHWVRQAPGQGLEWMGRVNPNRRGTTYNQKFEGRVTMTTDTSTSTAYME
LRSLRSDDTAVYYCARANWLDYWGQGTTVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT4849 Heavy Chain Triple Hinge + “YTE” mutation
(SEQ ID NO: 77)
QVQLVQSGAEVKKPGASVKVSCKASGYTFTDYYMHWVRQAPGQGLEWMGRVNPNRRGTTYNQKFEGRVTMTTDTSTSTAYME
LRSLRSDDTAVYYCARANWLDYWGQGTTVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLYITREPEVTCV
VVDVSHEDPEVKFNWYVDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSRWQQGNVFSCSVMHEALH
NHYTQKSLSLSPG**
>MYT4849 Heavy Chain Triple Hinge and “A118C”
(SEQ ID NO: 78)
QVQLVQSGAEVKKPGASVKVSCKASGYTFTDYYMHWVRQAPGQGLEWMGRVNPNRRGTTYNQKFEGRVTMTTDTSTSTAYME
LRSLRSDDTAVYYCARANWLDYWGQGTTVTVSSCSTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4849 Heavy Chain Triple Hinge + “LS” mutation and “A118C”
(SEQ ID NO: 79)
QVQLVQSGAEVKKPGASVKVSCKASGYTFTDYYMHWVRQAPGQGLEWMGRVNPNRRGTTYNQKFEGRVTMTTDTSTSTAYME
LRSLRSDDTAVYYCARANWLDYWGQGTTVTVSSCSTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT4849 Heavy Chain Triple Hinge + “YTE” mutation and “A118C”
(SEQ ID NO: 80)
QVQLVQSGAEVKKPGASVKVSCKASGYTFTDYYMHWVRQAPGQGLEWMGRVNPNRRGTTYNQKFEGRVTMTTDTSTSTAYME
LRSLRSDDTAVYYCARANWLDYWGQGTTVTVSSCSTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLYITREPEVTCV
VVDVSHEDPEVKFNWYVDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSRWQQGNVFSCSVMHEALH
NHYTQKSLSLSPG**
>MYT4849 Light Chain
(SEQ ID NO: 81)
DIQMTQSPSSLSASVGDRVTITCSVSSSVSSIHLHWYQQKPGKAPKLLIYHTSNLASGVPSRFSGSGSGTDFTLTISSLQPEDFATYYCQ
VYSGYPLTFGGGTKVEIKRTVAAPSVFIFPPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC**
>MYT4849 Light Chain “V205C”
(SEQ ID NO: 82)
DIQMTQSPSSLSASVGDRVTITCSVSSSVSSIHLHWYQQKPGKAPKLLIYHTSNLASGVPSRFSGSGSGTDFTLTISSLQPEDFATYYCQ
VYSGYPLTFGGGTKVEIKRTVAAPSVFIFPPSDEQLKSGTASVVCLLNNFYPREAKVQWKVDNALQSGNSQESVTEQDSKDSTYSLSS
TLTLSKADYEKHKVYACEVTHQGLSSPCTKSFNRGEC**
>MYT4942 Heavy Chain Triple Hinge
(SEQ ID NO: 83)
QVQLVQSGAEVKKPGASVKVSCKASGHIFTAYTMHWVRQAPGQGLEWMGWIKPNNGLANYAQKFQGRVTMTRDTSISTAYME
LSRLRSDDTAVYYCARSEITHEFDHWGQGTLVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4942 Heavy Chain Triple Hinge + “LS” mutation
(SEQ ID NO: 84)
QVQLVQSGAEVKKPGASVKVSCKASGHIFTAYTMHWVRQAPGQGLEWMGWIKPNNGLANYAQKFQGRVTMTRDTSISTAYME
LSRLRSDDTAVYYCARSEITHEFDHWGQGTLVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT4942 Heavy Chain Triple Hinge + “YTE” mutation
(SEQ ID NO: 85)
QVQLVQSGAEVKKPGASVKVSCKASGHIFTAYTMHWVRQAPGQGLEWMGWIKPNNGLANYAQKFQGRVTMTRDTSISTAYME
LSRLRSDDTAVYYCARSEITHEFDHWGQGTLVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLYITREPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4942 Heavy Chain Triple Hinge and “A118C”
(SEQ ID NO: 86)
QVQLVQSGAEVKKPGASVKVSCKASGHIFTAYTMHWVRQAPGQGLEWMGWIKPNNGLANYAQKFQGRVTMTRDTSISTAYME
LSRLRSDDTAVYYCARSEITHEFDHWGQGTLVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4942 Heavy Chain Triple Hinge + “LS” mutation and “A118C”
(SEQ ID NO: 87)
QVQLVQSGAEVKKPGASVKVSCKASGHIFTAYTMHWVRQAPGQGLEWMGWIKPNNGLANYAQKFQGRVTMTRDTSISTAYME
LSRLRSDDTAVYYCARSEITHEFDHWGQGTLVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT4942 Heavy Chain Triple Hinge + “YTE” mutation and “A118C”
(SEQ ID NO: 88)
QVQLVQSGAEVKKPGASVKVSCKASGHIFTAYTMHWVRQAPGQGLEWMGWIKPNNGLANYAQKFQGRVTMTRDTSISTAYME
LSRLRSDDTAVYYCARSEITHEFDHWGQGTLVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLYITREPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4942 Light Chain
(SEQ ID NO: 89)
DIVMTQSPDSLAVSLGERATINCKSSESVDSYANSHLHWYQQKPGQPPKLLIYRASTRESGVPDRFSGSGSGTDFTLTISSLQAEDVA
VYYCQQSKEDPLTFGGGTKVEIKRTVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC**
>MYT4942 Light Chain “V205C”
(SEQ ID NO: 90)
DIVMTQSPDSLAVSLGERATINCKSSESVDSYANSHLHWYQQKPGQPPKLLIYRASTRESGVPDRFSGSGSGTDFTLTISSLQAEDVA
VYYCQQSKEDPLTFGGGTKVEIKRTVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPCTKSFNRGEC**
>MYT5309 Heavy Chain Triple Hinge
(SEQ ID NO: 91)
QVQLVQSGAEVKKPGASVKVSCKASGYTFTHYYMHWVRQAPGQGLEWMGRVNPNRRGTTYNQKFEGRVTMTTDTSTSTAYME
LRSLRSDDTAVYYCARANWLDYWGQGTTVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT5309 Heavy Chain Triple Hinge + “LS” mutation
(SEQ ID NO: 92)
QVQLVQSGAEVKKPGASVKVSCKASGYTFTHYYMHWVRQAPGQGLEWMGRVNPNRRGTTYNQKFEGRVTMTTDTSTSTAYME
LRSLRSDDTAVYYCARANWLDYWGQGTTVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT5309 Heavy Chain Triple Hinge + “YTE” mutation
(SEQ ID NO: 93)
QVQLVQSGAEVKKPGASVKVSCKASGYTFTHYYMHWVRQAPGQGLEWMGRVNPNRRGTTYNQKFEGRVTMTTDTSTSTAYME
LRSLRSDDTAVYYCARANWLDYWGQGTTVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLYITREPEVTCV
VVDVSHEDPEVKFNWYVDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSRWQQGNVFSCSVMHEALH
NHYTQKSLSLSPG**
>MYT5309 Heavy Chain Triple Hinge and “A118C”
(SEQ ID NO: 94)
QVQLVQSGAEVKKPGASVKVSCKASGYTFTHYYMHWVRQAPGQGLEWMGRVNPNRRGTTYNQKFEGRVTMTTDTSTSTAYME
LRSLRSDDTAVYYCARANWLDYWGQGTTVTVSSCSTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT5309 Heavy Chain Triple Hinge + “LS” mutation and “A118C”
(SEQ ID NO: 95)
QVQLVQSGAEVKKPGASVKVSCKASGYTFTHYYMHWVRQAPGQGLEWMGRVNPNRRGTTYNQKFEGRVTMTTDTSTSTAYME
LRSLRSDDTAVYYCARANWLDYWGQGTTVTVSSCSTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT5309 Heavy Chain Triple Hinge + “YTE” mutation and “A118C”
(SEQ ID NO: 96)
QVQLVQSGAEVKKPGASVKVSCKASGYTFTHYYMHWVRQAPGQGLEWMGRVNPNRRGTTYNQKFEGRVTMTTDTSTSTAYME
LRSLRSDDTAVYYCARANWLDYWGQGTTVTVSSCSTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLYITREPEVTCV
VVDVSHEDPEVKFNWYVDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSRWQQGNVFSCSVMHEALH
NHYTQKSLSLSPG**
>MYT5309 Light Chain
(SEQ ID NO: 97)
DIQMTQSPSSLSASVGDRVTITCSVSSSVSSIYLHWYQQKPGKAPKLLIYSTSNLASGVPSRFSGSGSGTDFTLTISSLQPEDFATYYCQ
HYSGYPLTFGGGTKVEIKRTVAAPSVFIFPPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC**
>MYT5309 Light Chain “V205C”
(SEQ ID NO: 98)
DIQMTQSPSSLSASVGDRVTITCSVSSSVSSIYLHWYQQKPGKAPKLLIYSTSNLASGVPSRFSGSGSGTDFTLTISSLQPEDFATYYCQ
HYSGYPLTFGGGTKVEIKRTVAAPSVFIFPPSDEQLKSGTASVVCLLNNFYPREAKVQWKVDNALQSGNSQESVTEQDSKDSTYSLSS
TLTLSKADYEKHKVYACEVTHQGLSSPCTKSFNRGEC**
>MYT5344 Heavy Chain Triple Hinge
(SEQ ID NO: 99)
QVQLVQSGAEVKKPGASVKVSCKASGYTFTSYWMHWVKQAPGQGLDWIGYIKHSTDNTEYNQKFKDKATLTADKSTSTAYMELS
SLRSEDTAVYYCARSYGNYPLMDYWGQGTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT5344 Heavy Chain Triple Hinge + “LS” mutation
(SEQ ID NO: 100)
QVQLVQSGAEVKKPGASVKVSCKASGYTFTSYWMHWVKQAPGQGLDWIGYIKHSTDNTEYNQKFKDKATLTADKSTSTAYMELS
SLRSEDTAVYYCARSYGNYPLMDYWGQGTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT5344 Heavy Chain Triple Hinge + “YTE” mutation
(SEQ ID NO: 101)
QVQLVQSGAEVKKPGASVKVSCKASGYTFTSYWMHWVKQAPGQGLDWIGYIKHSTDNTEYNQKFKDKATLTADKSTSTAYMELS
SLRSEDTAVYYCARSYGNYPLMDYWGQGTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLYITREPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT5344 Heavy Chain Triple Hinge and “A118C”
(SEQ ID NO: 102)
QVQLVQSGAEVKKPGASVKVSCKASGYTFTSYWMHWVKQAPGQGLDWIGYIKHSTDNTEYNQKFKDKATLTADKSTSTAYMELS
SLRSEDTAVYYCARSYGNYPLMDYWGQGTTVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT5344 Heavy Chain Triple Hinge + “LS” mutation and “A118C”
(SEQ ID NO: 103)
QVQLVQSGAEVKKPGASVKVSCKASGYTFTSYWMHWVKQAPGQGLDWIGYIKHSTDNTEYNQKFKDKATLTADKSTSTAYMELS
SLRSEDTAVYYCARSYGNYPLMDYWGQGTTVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT5344 Heavy Chain Triple Hinge + “YTE” mutation and “A118C”
(SEQ ID NO: 104)
QVQLVQSGAEVKKPGASVKVSCKASGYTFTSYWMHWVKQAPGQGLDWIGYIKHSTDNTEYNQKFKDKATLTADKSTSTAYMELS
SLRSEDTAVYYCARSYGNYPLMDYWGQGTTVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLYITREPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT5344 Light Chain
(SEQ ID NO: 105)
QIVLTQSPAILSLSPGERATLSCSASSSVTSHYLYWYQQKPGSSPKLLIYSTSNLASGVPARFSGSGSGTSYTLTISSLEAEDAASYFCHQ
WSSYPPTFGSGTKLEIKRTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC**
>MYT5344 Light Chain “V205C”
(SEQ ID NO: 106)
QIVLTQSPAILSLSPGERATLSCSASSSVTSHYLYWYQQKPGSSPKLLIYSTSNLASGVPARFSGSGSGTSYTLTISSLEAEDAASYFCHQ
WSSYPPTFGSGTKLEIKRTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPCTKSFNRGEC**
>MYT5367 Heavy Chain Triple Hinge
(SEQ ID NO: 107)
QVQLVQSGAEVKKPGASVKVSCKASGYTFTSYWMHWVKQAPGQGLDWIGHIKPSTDNTEYNQKFKDKATLTADKSTSTAYMELS
SLRSEDTAVYYCARSYGNYPLMDYWGQGTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT5367 Heavy Chain Triple Hinge + “LS” mutation
(SEQ ID NO: 108)
QVQLVQSGAEVKKPGASVKVSCKASGYTFTSYWMHWVKQAPGQGLDWIGHIKPSTDNTEYNQKFKDKATLTADKSTSTAYMELS
SLRSEDTAVYYCARSYGNYPLMDYWGQGTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT5367 Heavy Chain Triple Hinge + “YTE” mutation
(SEQ ID NO: 109)
QVQLVQSGAEVKKPGASVKVSCKASGYTFTSYWMHWVKQAPGQGLDWIGHIKPSTDNTEYNQKFKDKATLTADKSTSTAYMELS
SLRSEDTAVYYCARSYGNYPLMDYWGQGTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLYITREPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT5367 Heavy Chain Triple Hinge and “A118C”
(SEQ ID NO: 110)
QVQLVQSGAEVKKPGASVKVSCKASGYTFTSYWMHWVKQAPGQGLDWIGHIKPSTDNTEYNQKFKDKATLTADKSTSTAYMELS
SLRSEDTAVYYCARSYGNYPLMDYWGQGTTVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT5367 Heavy Chain Triple Hinge + “LS” mutation and “A118C”
(SEQ ID NO: 111)
QVQLVQSGAEVKKPGASVKVSCKASGYTFTSYWMHWVKQAPGQGLDWIGHIKPSTDNTEYNQKFKDKATLTADKSTSTAYMELS
SLRSEDTAVYYCARSYGNYPLMDYWGQGTTVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT5367 Heavy Chain Triple Hinge + “YTE” mutation and “A118C”
(SEQ ID NO: 112)
QVQLVQSGAEVKKPGASVKVSCKASGYTFTSYWMHWVKQAPGQGLDWIGHIKPSTDNTEYNQKFKDKATLTADKSTSTAYMELS
SLRSEDTAVYYCARSYGNYPLMDYWGQGTTVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLYITREPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT5367 Light Chain
(SEQ ID NO: 113)
QIVLTQSPAILSLSPGERATLSCSASSSVTSNYLYWYQQKPGSSPKLLIYSTSNLASGVPARFSGSGSGTSYTLTISSLEAEDAASYFCHQ
WSHYHPTFGSGTKLEIKRTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC**
>MYT5367 Light Chain “V205C”
(SEQ ID NO: 114)
QIVLTQSPAILSLSPGERATLSCSASSSVTSNYLYWYQQKPGSSPKLLIYSTSNLASGVPARFSGSGSGTSYTLTISSLEAEDAASYFCHQ
WSHYHPTFGSGTKLEIKRTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPCTKSFNRGEC**
>MYT4827 Heavy Chain Triple Hinge
(SEQ ID NO: 115)
QVQLVQSGAEVKKPGASVKVSCKASGYTFTHYYMHWVRQAPGQGLEWMGRVNPNRRGTTYNQKFEGRVTMTTDTSTSTAYME
LRSLRSDDTAVYYCARAHWLDYWGQGTTVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4827 Heavy Chain Triple Hinge + “LS” mutation
(SEQ ID NO: 116)
QVQLVQSGAEVKKPGASVKVSCKASGYTFTHYYMHWVRQAPGQGLEWMGRVNPNRRGTTYNQKFEGRVTMTTDTSTSTAYME
LRSLRSDDTAVYYCARAHWLDYWGQGTTVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT4827 Heavy Chain Triple Hinge + “YTE” mutation
(SEQ ID NO: 117)
QVQLVQSGAEVKKPGASVKVSCKASGYTFTHYYMHWVRQAPGQGLEWMGRVNPNRRGTTYNQKFEGRVTMTTDTSTSTAYME
LRSLRSDDTAVYYCARAHWLDYWGQGTTVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLYITREPEVTCV
VVDVSHEDPEVKFNWYVDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSRWQQGNVFSCSVMHEALH
NHYTQKSLSLSPG**
>MYT4827 Heavy Chain Triple Hinge and “A118C”
(SEQ ID NO: 118)
QVQLVQSGAEVKKPGASVKVSCKASGYTFTHYYMHWVRQAPGQGLEWMGRVNPNRRGTTYNQKFEGRVTMTTDTSTSTAYME
LRSLRSDDTAVYYCARAHWLDYWGQGTTVTVSSCSTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4827 Heavy Chain Triple Hinge + “LS” mutation and “A118C”
(SEQ ID NO: 119)
QVQLVQSGAEVKKPGASVKVSCKASGYTFTHYYMHWVRQAPGQGLEWMGRVNPNRRGTTYNQKFEGRVTMTTDTSTSTAYME
LRSLRSDDTAVYYCARAHWLDYWGQGTTVTVSSCSTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT4827 Heavy Chain Triple Hinge + “YTE” mutation and “A118C”
(SEQ ID NO: 120)
QVQLVQSGAEVKKPGASVKVSCKASGYTFTHYYMHWVRQAPGQGLEWMGRVNPNRRGTTYNQKFEGRVTMTTDTSTSTAYME
LRSLRSDDTAVYYCARAHWLDYWGQGTTVTVSSCSTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLYITREPEVTCV
VVDVSHEDPEVKFNWYVDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSRWQQGNVFSCSVMHEALH
NHYTQKSLSLSPG**
>MYT4827 Light Chain
(SEQ ID NO: 121)
DIQMTQSPSSLSASVGDRVTITCSVSSSVSSIYLHWYQQKPGKAPKLLIYSTSNLASGVPSRFSGSGSGTDFTLTISSLQPEDFATYYCQ
VYSGYPLTFGGGTKVEIKRTVAAPSVFIFPPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC**
>MYT4827 Light Chain “V205C”
(SEQ ID NO: 122)
DIQMTQSPSSLSASVGDRVTITCSVSSSVSSIYLHWYQQKPGKAPKLLIYSTSNLASGVPSRFSGSGSGTDFTLTISSLQPEDFATYYCQ
VYSGYPLTFGGGTKVEIKRTVAAPSVFIFPPSDEQLKSGTASVVCLLNNFYPREAKVQWKVDNALQSGNSQESVTEQDSKDSTYSLSS
TLTLSKADYEKHKVYACEVTHQGLSSPCTKSFNRGEC**
>MYT4312 Heavy Chain Triple Hinge
(SEQ ID NO: 123)
QVQLVQSGAEVKKPGASVKVSCKASGYTFTSYWMHWVKQAPGQGLDWIGHIKPSTDNTEYNQKFKDKATLTADKSTSTAYMELS
SLRSEDTAVYYCARSYGNYPLMHYWGQGTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4312 Heavy Chain Triple Hinge + “LS” mutation
(SEQ ID NO: 124)
QVQLVQSGAEVKKPGASVKVSCKASGYTFTSYWMHWVKQAPGQGLDWIGHIKPSTDNTEYNQKFKDKATLTADKSTSTAYMELS
SLRSEDTAVYYCARSYGNYPLMHYWGQGTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT4312 Heavy Chain Triple Hinge + “YTE” mutation
(SEQ ID NO: 125)
QVQLVQSGAEVKKPGASVKVSCKASGYTFTSYWMHWVKQAPGQGLDWIGHIKPSTDNTEYNQKFKDKATLTADKSTSTAYMELS
SLRSEDTAVYYCARSYGNYPLMHYWGQGTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLYITREPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4312 Heavy Chain Triple Hinge and “A118C”
(SEQ ID NO: 126)
QVQLVQSGAEVKKPGASVKVSCKASGYTFTSYWMHWVKQAPGQGLDWIGHIKPSTDNTEYNQKFKDKATLTADKSTSTAYMELS
SLRSEDTAVYYCARSYGNYPLMHYWGQGTTVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4312 Heavy Chain Triple Hinge + “LS” mutation and “A118C”
(SEQ ID NO: 127)
QVQLVQSGAEVKKPGASVKVSCKASGYTFTSYWMHWVKQAPGQGLDWIGHIKPSTDNTEYNQKFKDKATLTADKSTSTAYMELS
SLRSEDTAVYYCARSYGNYPLMHYWGQGTTVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT4312 Heavy Chain (Triple Hinge + “YTE” mutation and “A118C”
(SEQ ID NO: 128)
QVQLVQSGAEVKKPGASVKVSCKASGYTFTSYWMHWVKQAPGQGLDWIGHIKPSTDNTEYNQKFKDKATLTADKSTSTAYMELS
SLRSEDTAVYYCARSYGNYPLMHYWGQGTTVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLYITREPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4312 Light Chain
(SEQ ID NO: 129)
QIVLTQSPAILSLSPGERATLSCSASSSVTSNYLYWYQQKPGSSPKLLIYSTSNLASGVPARFSGSGSGTSYTLTISSLEAEDAASYFCHQ
WSSYPPTFGSGTKLEIKRTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC**
>MYT4312 Light Chain “V205C”
(SEQ ID NO: 130)
QIVLTQSPAILSLSPGERATLSCSASSSVTSNYLYWYQQKPGSSPKLLIYSTSNLASGVPARFSGSGSGTSYTLTISSLEAEDAASYFCHQ
WSSYPPTFGSGTKLEIKRTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPCTKSFNRGEC**
>MYT4953 Heavy Chain Triple Hinge
(SEQ ID NO: 131)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCHRSEITHEFDYWGQGTLVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4953 Heavy Chain Triple Hinge + “LS” mutation
(SEQ ID NO: 132)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCHRSEITHEFDYWGQGTLVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT4953 Heavy Chain Triple Hinge + “YTE” mutation
(SEQ ID NO: 133)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCHRSEITHEFDYWGQGTLVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLYITREPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4953 Heavy Chain Triple Hinge and “A118C”
(SEQ ID NO: 134)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCHRSEITHEFDYWGQGTLVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4953 Heavy Chain Triple Hinge + “LS” mutation and “A118C”
(SEQ ID NO: 135)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCHRSEITHEFDYWGQGTLVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT4953 Heavy Chain Triple Hinge + “YTE” mutation and “A118C”
(SEQ ID NO: 136)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCHRSEITHEFDYWGQGTLVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLYITREPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4953 Light Chain
(SEQ ID NO: 137)
DIVMTQSPDSLAVSLGERATINCKSSESVDSYANSFLHWYQQKPGQPPKLLIYRASTRESGVPDRFSGSGSGTDFTLTISSLQAEDVAV
YYCQQSKEDPLTFGGGTKVEIKRTVAAPSVFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC**
>MYT4953 Light Chain “V205C”
(SEQ ID NO: 138)
DIVMTQSPDSLAVSLGERATINCKSSESVDSYANSFLHWYQQKPGQPPKLLIYRASTRESGVPDRFSGSGSGTDFTLTISSLQAEDVAV
YYCQQSKEDPLTFGGGTKVEIKRTVAAPSVFIFPPSDEQLKSGTASVVCLLNNFYPREAKVQWKVDNALQSGNSQESVTEQDSKDST
YSLSSTLTLSKADYEKHKVYACEVTHQGLSSPCTKSFNRGEC**
>MYT4940 Heavy Chain Triple Hinge
(SEQ ID NO: 139)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCARHEITTEFDHWGQGTLVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4940 Heavy Chain (Triple Hinge + “LS” mutation
(SEQ ID NO: 140)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCARHEITTEFDHWGQGTLVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT4940 Heavy Chain Triple Hinge + “YTE” mutation
(SEQ ID NO: 141)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCARHEITTEFDHWGQGTLVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLYITREPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4940 Heavy Chain Triple Hinge and “A118C”
(SEQ ID NO: 142)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCARHEITTEFDHWGQGTLVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4940 Heavy Chain Triple Hinge + “LS” mutation and “A118C”
(SEQ ID NO: 143)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCARHEITTEFDHWGQGTLVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT4940 Heavy Chain Triple Hinge + “YTE” mutation and “A118C”
(SEQ ID NO: 144)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCARHEITTEFDHWGQGTLVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLYITREPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4940 Light Chain
(SEQ ID NO: 145)
DIVMTQSPDSLAVSLGERATINCKSSESVDSYANSHLHWYQQKPGQPPKLLIYRASTRESGVPDRFSGSGSGTDFTLTISSLQAEDVA
VYYCQQSKEDPLTFGGGTKVEIKRTVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC**
>MYT4940 Light Chain “V205C”
(SEQ ID NO: 146)
DIVMTQSPDSLAVSLGERATINCKSSESVDSYANSHLHWYQQKPGQPPKLLIYRASTRESGVPDRFSGSGSGTDFTLTISSLQAEDVA
VYYCQQSKEDPLTFGGGTKVEIKRTVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPCTKSFNRGEC**
>MYT4888 Heavy Chain Triple Hinge
(SEQ ID NO: 147)
QVQLVQSGAEVKKPGASVKVSCKASGYTFTSYWMHWVKQAPGQGLDWIGYIKPSTDNTEYNQKFKDKATLTADKSTSTAYMELS
SLRSEDTAVYYCARSYGNYPLHDYWGQGTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4888 Heavy Chain Triple Hinge + “LS” mutation
(SEQ ID NO: 148)
QVQLVQSGAEVKKPGASVKVSCKASGYTFTSYWMHWVKQAPGQGLDWIGYIKPSTDNTEYNQKFKDKATLTADKSTSTAYMELS
SLRSEDTAVYYCARSYGNYPLHDYWGQGTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT4888 Heavy Chain Triple Hinge + “YTE” mutation
(SEQ ID NO: 149)
QVQLVQSGAEVKKPGASVKVSCKASGYTFTSYWMHWVKQAPGQGLDWIGYIKPSTDNTEYNQKFKDKATLTADKSTSTAYMELS
SLRSEDTAVYYCARSYGNYPLHDYWGQGTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLYITREPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4888 Heavy Chain Triple Hinge and “A118C”
(SEQ ID NO: 150)
QVQLVQSGAEVKKPGASVKVSCKASGYTFTSYWMHWVKQAPGQGLDWIGYIKPSTDNTEYNQKFKDKATLTADKSTSTAYMELS
SLRSEDTAVYYCARSYGNYPLHDYWGQGTTVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4888 Heavy Chain Triple Hinge + “LS” mutation and “A118C”
(SEQ ID NO: 151)
QVQLVQSGAEVKKPGASVKVSCKASGYTFTSYWMHWVKQAPGQGLDWIGYIKPSTDNTEYNQKFKDKATLTADKSTSTAYMELS
SLRSEDTAVYYCARSYGNYPLHDYWGQGTTVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>MYT4888 Heavy Chain Triple Hinge + “YTE” mutation and “A118C”
(SEQ ID NO: 152)
QVQLVQSGAEVKKPGASVKVSCKASGYTFTSYWMHWVKQAPGQGLDWIGYIKPSTDNTEYNQKFKDKATLTADKSTSTAYMELS
SLRSEDTAVYYCARSYGNYPLHDYWGQGTTVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLYITREPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>MYT4888 Light Chain
(SEQ ID NO: 153)
QIVLTQSPAILSLSPGERATLSCSASSSVTSNYLYWYQQKPGSSPKLLIYSTSNLASGVPARFSGSGSGTSYTLTISSLEAEDAASYFCHQ
WSSYPPTFGSGTKLEIKRTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC**
>MYT4888 Light Chain “V205C”
(SEQ ID NO: 154)
QIVLTQSPAILSLSPGERATLSCSASSSVTSNYLYWYQQKPGSSPKLLIYSTSNLASGVPARFSGSGSGTSYTLTISSLEAEDAASYFCHQ
WSSYPPTFGSGTKLEIKRTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPCTKSFNRGEC**
>Heavy Chain Constant Domain
(SEQ ID NO: 155)
ASTKGPSVFPLAPSSKSTSGGTAALGCLVKDYFPEPVTVSWNSGALTSGVHTFPAVLQSSGLYSLSSVVTVPSSSLGTQTYICNVNHKP
SNTKVDKRVEPKSCDKTHTCPPCPAPELLGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYTQKSLSLSPG**
>Heavy Chain Constant Domain “A118C”
(SEQ ID NO: 156)
CSTKGPSVFPLAPSSKSTSGGTAALGCLVKDYFPEPVTVSWNSGALTSGVHTFPAVLQSSGLYSLSSVVTVPSSSLGTQTYICNVNHKP
SNTKVDKRVEPKSCDCHCPPCPAPELLGGPSVFLFPPKPKDTLYITREPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSREEMTKNQVSLTCLVKGFYPSDIAVEWES
NGQPENNYKTTPPVLDSDGSFFLYSKLTVDKSRWQQGNVFSCSVLHEALHSHYTQKSLSLSPG**
>Light Chain Constant Domain
(SEQ ID NO: 157)
RTVAAPSVFIFPPSDEQLKSGTASVVCLLNNFYPREAKVQWKVDNALQSGNSQESVTEQDSKDSTYSLSSTLTLSKADYEKHKVYACE
VTHQGLSSPVTKSFNRGEC**
>Light Chain Constant Domain “V205C”
(SEQ ID NO: 158)
RTVAAPSVFIFPPSDEQLKSGTASVVCLLNNFYPREAKVQWKVDNALQSGNSQESVTEQDSKDSTYSLSSTLTLSKADYEKHKVYACE
VTHQGLSSPCTKSFNRGEC**
>Telisotuzumab Heavy Chain variable region
(SEQ ID NO: 159)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCARSEITTEFDYWGQGTLVTVSS
>Telisotuzumab Light Chain variable region
(SEQ ID NO: 160)
DIVMTQSPDSLAVSLGERATINCKSSESVDSYANSFLHWYQQKPGQPPKLLIYRASTRESGVPDRFSGSGSGTDFTLTISSLQAEDVAV
YYCQQSKEDPLTFGGGTKVEIK
>Emibetuzumab Heavy Chain variable region
(SEQ ID NO: 161)
QVQLVQSGAEVKKPGASVKVSCKASGYTFTDYYMHWVRQAPGQGLEWMGRVNPNRRGTTYNQKFEGRVTMTTDTSTSTAYME
LRSLRSDDTAVYYCARANWLDYWGQGTTVTVSS
>Emibetuzumab Light Chain variable region
(SEQ ID NO: 162)
DIQMTQSPSSLSASVGDRVTITCSVSSSVSSIYLHWYQQKPGKAPKLLIYSTSNLASGVPSRFSGSGSGTDFTLTISSLQPEDFATYYCQ
VYSGYPLTFGGGTKVEIK
>P3D12 anti-cMET Heavy Chain variable region
(SEQ ID NO: 163)
QVQLVQSGAEVKKPGASVKVSCKASGYTFTSYWMHWVKQAPGQGLDWIGYIKPSTDNTEYNQKFKDKATLTADKSTSTAYMELS
SLRSEDTAVYYCARSYGNYPLMDYWGQGTTVTVSS
>P3D12 anti-cMET Light Chain variable region
(SEQ ID NO: 164)
QIVLTQSPAILSLSPGERATLSCSASSSVTSNYLYWYQQKPGSSPKLLIYSTSNLASGVPARFSGSGSGTSYTLTISSLEAEDAASYFCHQ
WSSYPPTFGSGTKLEIK
>Telisotuzumab Heavy Chain Triple Hinge
(SEQ ID NO: 165)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCARSEITTEFDYWGQGTLVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>Telisotuzumab Heavy Chain Triple Hinge + “LS”
(SEQ ID NO: 166)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCARSEITTEFDYWGQGTLVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>Telisotuzumab Heavy Chain Triple Hinge + “YTE”
(SEQ ID NO: 167)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCARSEITTEFDYWGQGTLVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLYITREPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>Telisotuzumab Heavy Chain Triple Hinge and “A118C”
(SEQ ID NO: 168)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCARSEITTEFDYWGQGTLVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>Telisotuzumab Heavy Chain Triple Hinge + “LS” and “A118C”
(SEQ ID NO: 169)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCARSEITTEFDYWGQGTLVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>Telisotuzumab Heavy Chain Triple Hinge + “YTE” and “A118C”
(SEQ ID NO: 170)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCARSEITTEFDYWGQGTLVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLYITREPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>Telisotuzumab Light Chain
(SEQ ID NO: 171)
DIVMTQSPDSLAVSLGERATINCKSSESVDSYANSFLHWYQQKPGQPPKLLIYRASTRESGVPDRFSGSGSGTDFTLTISSLQAEDVAV
YYCQQSKEDPLTFGGGTKVEIKRTVAAPSVFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC**
>Telisotuzumab Light Chain “V205C”
(SEQ ID NO: 172)
DIVMTQSPDSLAVSLGERATINCKSSESVDSYANSFLHWYQQKPGQPPKLLIYRASTRESGVPDRFSGSGSGTDFTLTISSLQAEDVAV
YYCQQSKEDPLTFGGGTKVEIKRTVAAPSVFIFPPSDEQLKSGTASVVCLLNNFYPREAKVQWKVDNALQSGNSQESVTEQDSKDST
YSLSSTLTLSKADYEKHKVYACEVTHQGLSSPCTKSFNRGEC**
>Emibetuzumab Heavy Chain with Triple Hinge
(SEQ ID NO: 173)
QVQLVQSGAEVKKPGASVKVSCKASGYTFTDYYMHWVRQAPGQGLEWMGRVNPNRRGTTYNQKFEGRVTMTTDTSTSTAYME
LRSLRSDDTAVYYCARANWLDYWGQGTTVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>Emibetuzumab Heavy Chain with Triple Hinge and “LS”
(SEQ ID NO: 174)
QVQLVQSGAEVKKPGASVKVSCKASGYTFTDYYMHWVRQAPGQGLEWMGRVNPNRRGTTYNQKFEGRVTMTTDTSTSTAYME
LRSLRSDDTAVYYCARANWLDYWGQGTTVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>Emibetuzumab Heavy Chain with Triple Hinge and “YTE”
(SEQ ID NO: 175)
QVQLVQSGAEVKKPGASVKVSCKASGYTFTDYYMHWVRQAPGQGLEWMGRVNPNRRGTTYNQKFEGRVTMTTDTSTSTAYME
LRSLRSDDTAVYYCARANWLDYWGQGTTVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLYITREPEVTCV
VVDVSHEDPEVKFNWYVDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSRWQQGNVFSCSVMHEALH
NHYTQKSLSLSPG**
>Emibetuzumab Heavy Chain with Triple Hinge and “A118C”
(SEQ ID NO: 176)
QVQLVQSGAEVKKPGASVKVSCKASGYTFTDYYMHWVRQAPGQGLEWMGRVNPNRRGTTYNQKFEGRVTMTTDTSTSTAYME
LRSLRSDDTAVYYCARANWLDYWGQGTTVTVSSCSTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>Emibetuzumab Heavy Chain with Triple Hinge and “LS” and “A118C”
(SEQ ID NO: 177)
QVQLVQSGAEVKKPGASVKVSCKASGYTFTDYYMHWVRQAPGQGLEWMGRVNPNRRGTTYNQKFEGRVTMTTDTSTSTAYME
LRSLRSDDTAVYYCARANWLDYWGQGTTVTVSSCSTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>Emibetuzumab Heavy Chain with Triple Hinge and “YTE” and “A118C”
(SEQ ID NO: 178)
QVQLVQSGAEVKKPGASVKVSCKASGYTFTDYYMHWVRQAPGQGLEWMGRVNPNRRGTTYNQKFEGRVTMTTDTSTSTAYME
LRSLRSDDTAVYYCARANWLDYWGQGTTVTVSSCSTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDCHCPPCPAPELLGGPSVFLFPPKPKDTLYITREPEVTCV
VVDVSHEDPEVKFNWYVDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSRWQQGNVFSCSVMHEALH
NHYTQKSLSLSPG**
>Emibetuzumab Light Chain
(SEQ ID NO: 179)
DIQMTQSPSSLSASVGDRVTITCSVSSSVSSIYLHWYQQKPGKAPKLLIYSTSNLASGVPSRFSGSGSGTDFTLTISSLQPEDFATYYCQ
VYSGYPLTFGGGTKVEIKRTVAAPSVFIFPPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC**
>Emibetuzumab Light Chain “V205C”
(SEQ ID NO: 180)
DIQMTQSPSSLSASVGDRVTITCSVSSSVSSIYLHWYQQKPGKAPKLLIYSTSNLASGVPSRFSGSGSGTDFTLTISSLQPEDFATYYCQ
VYSGYPLTFGGGTKVEIKRTVAAPSVFIFPPSDEQLKSGTASVVCLLNNFYPREAKVQWKVDNALQSGNSQESVTEQDSKDSTYSLSS
TLTLSKADYEKHKVYACEVTHQGLSSPCTKSFNRGEC**
>P3D12 anti-cMET Heavy Chain with Triple Hinge
(SEQ ID NO: 181)
QVQLVQSGAEVKKPGASVKVSCKASGYTFTSYWMHWVKQAPGQGLDWIGYIKPSTDNTEYNQKFKDKATLTADKSTSTAYMELS
SLRSEDTAVYYCARSYGNYPLMDYWGQGTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>P3D12 anti-cMET Heavy Chain with Triple Hinge and “LS”
(SEQ ID NO: 182)
QVQLVQSGAEVKKPGASVKVSCKASGYTFTSYWMHWVKQAPGQGLDWIGYIKPSTDNTEYNQKFKDKATLTADKSTSTAYMELS
SLRSEDTAVYYCARSYGNYPLMDYWGQGTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>P3D12 anti-cMET Heavy Chain with Triple Hinge and “YTE”
(SEQ ID NO: 183)
QVQLVQSGAEVKKPGASVKVSCKASGYTFTSYWMHWVKQAPGQGLDWIGYIKPSTDNTEYNQKFKDKATLTADKSTSTAYMELS
SLRSEDTAVYYCARSYGNYPLMDYWGQGTTVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLYITREPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>P3D12 anti-cMET Heavy Chain with Triple Hinge and “A118C”
(SEQ ID NO: 184)
QVQLVQSGAEVKKPGASVKVSCKASGYTFTSYWMHWVKQAPGQGLDWIGYIKPSTDNTEYNQKFKDKATLTADKSTSTAYMELS
SLRSEDTAVYYCARSYGNYPLMDYWGQGTTVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>P3D12 anti-cMET Heavy Chain with Triple Hinge and “LS” and “A118C”
(SEQ ID NO: 185)
QVQLVQSGAEVKKPGASVKVSCKASGYTFTSYWMHWVKQAPGQGLDWIGYIKPSTDNTEYNQKFKDKATLTADKSTSTAYMELS
SLRSEDTAVYYCARSYGNYPLMDYWGQGTTVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQGNVFSCSVLHEALH
SHYTQKSLSLSPG**
>P3D12 anti-cMET Heavy Chain with Triple Hinge and “YTE” and “A118C”
(SEQ ID NO: 186)
QVQLVQSGAEVKKPGASVKVSCKASGYTFTSYWMHWVKQAPGQGLDWIGYIKPSTDNTEYNQKFKDKATLTADKSTSTAYMELS
SLRSEDTAVYYCARSYGNYPLMDYWGQGTTVTVSSCSTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDCHCPPCPAPELLGGPSVFLFPPKPKDTLYITREPEVTC
VVVDVSHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCSVMHEAL
HNHYTQKSLSLSPG**
>P3D12 anti-cMET Light Chain
(SEQ ID NO: 187)
QIVLTQSPAILSLSPGERATLSCSASSSVTSNYLYWYQQKPGSSPKLLIYSTSNLASGVPARFSGSGSGTSYTLTISSLEAEDAASYFCHQ
WSSYPPTFGSGTKLEIKRTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC**
>P3D12 anti-cMET Light Chain “V205C”
(SEQ ID NO: 188)
QIVLTQSPAILSLSPGERATLSCSASSSVTSNYLYWYQQKPGSSPKLLIYSTSNLASGVPARFSGSGSGTSYTLTISSLEAEDAASYFCHQ
WSSYPPTFGSGTKLEIKRTVAAPSVFIFPPSDEQLKSGTASVVCLLNNFYPREAKVQWKVDNALQSGNSQESVTEQDSKDSTYSLSST
LTLSKADYEKHKVYACEVTHQGLSSPCTKSFNRGEC**
>Heavy Chain Constant Domain
(SEQ ID NO: 189)
ASTKGPSVFPLAPSSKSTSGGTAALGCLVKDYFPEPVTVSWNSGALTSGVHTFPAVLQSSGLYSLSSVVTVPSSSLGTQTYICNVNHKP
SNTKVDKRVEPKSCDKTHTCPPCPAPELLGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNHYTQKSLSLSPG**
>Light Chain of Telisotuzumab IgG histidine scanning variant #1
(SEQ ID NO: 190)
DIVMTQSPDSLAVSLGERATINCKSSESVDSYANSFLHWYQQKPGQPPKLLIYRASTREHGVPDRFSGSGSGTDFTLTISSLQAEDVA
VYYCQQSKEDPLTFGGGTKVEIK
>Light Chain of Telisotuzumab IgG histidine scanning variant #2
(SEQ ID NO: 191)
DIVMTQSPDSLAVSLGERATINCKSSESVDSYAHSFLHWYQQKPGQPPKLLIYRASTRESGVPDRFSGSGSGTDFTLTISSLQAEDVAV
YYCQQSKEDPLTFGGGTKVEIK
>Light Chain of Telisotuzumab IgG histidine scanning variant #3
(SEQ ID NO: 192)
DIVMTQSPDSLAVSLGERATINCKSSESVDSYANSFLHWYQQKPGQPPKLLIYHASTRESGVPDRFSGSGSGTDFTLTISSLQAEDVA
VYYCQQSKEDPLTFGGGTKVEIK
>Heavy Chain of Telisotuzumab IgG histidine scanning variant #1
(SEQ ID NO: 193)
QVQLVQSGAEVKKPGASVKVSCKASGHIFTAYTMHWVRQAPGQGLEWMGWIKPNNGLANYAQKFQGRVTMTRDTSISTAYME
LSRLRSDDTAVYYCARSEITTEFDYWGQGTLVTVSS
>Heavy Chain of Telisotuzumab IgG histidine scanning variant #2
(SEQ ID NO: 194)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCARHEITTEFDYWGQGTLVTVSS
>Heavy Chain of Telisotuzumab IgG histidine scanning variant #3
(SEQ ID NO: 195)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCARSEITHEFDYWGQGTLVTVSS
>Heavy Chain of Telisotuzumab IgG histidine scanning variant #4
(SEQ ID NO: 196)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCARSEITTEFDHWGQGTLVTVSS
>Heavy Chain Combinations of Telisotuzumab IgG histidine scanning variant #5
(SEQ ID NO: 197)
QVQLVQSGAEVKKPGASVKVSCKASGHIFTAYTMHWVRQAPGQGLEWMGWIKPNNGLANYAQKFQGRVTMTRDTSISTAYME
LSRLRSDDTAVYYCARHEITTEFDYWGQGTLVTVSS
>Heavy Chain Combinations of Telisotuzumab IgG histidine scanning variant #6
(SEQ ID NO: 198)
QVQLVQSGAEVKKPGASVKVSCKASGHIFTAYTMHWVRQAPGQGLEWMGWIKPNNGLANYAQKFQGRVTMTRDTSISTAYME
LSRLRSDDTAVYYCARSEITHEFDYWGQGTLVTVSS
>Heavy Chain Combinations of Telisotuzumab IgG histidine scanning variant #7
(SEQ ID NO: 199)
QVQLVQSGAEVKKPGASVKVSCKASGHIFTAYTMHWVRQAPGQGLEWMGWIKPNNGLANYAQKFQGRVTMTRDTSISTAYME
LSRLRSDDTAVYYCARSEITTEFDHWGQGTLVTVSS
>Heavy Chain Combinations of Telisotuzumab IgG histidine scanning variant #8
(SEQ ID NO: 200)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCARHEITHEFDYWGQGTLVTVSS
>Heavy Chain Combinations of Telisotuzumab IgG histidine scanning variant #9
(SEQ ID NO: 201)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCARSEITHEFDHWGQGTLVTVSS
>Heavy Chain Combinations of Telisotuzumab IgG histidine scanning variant #10
(SEQ ID NO: 202)
QVQLVQSGAEVKKPGASVKVSCKASGHIFTAYTMHWVRQAPGQGLEWMGWIKPNNGLANYAQKFQGRVTMTRDTSISTAYME
LSRLRSDDTAVYYCARHEITHEFDYWGQGTLVTVSS
>Heavy Chain Combinations of Telisotuzumab IgG histidine scanning variant #11
(SEQ ID NO: 203)
QVQLVQSGAEVKKPGASVKVSCKASGHIFTAYTMHWVRQAPGQGLEWMGWIKPNNGLANYAQKFQGRVTMTRDTSISTAYME
LSRLRSDDTAVYYCARHEITTEFDHWGQGTLVTVSS
>Heavy Chain Combinations of Telisotuzumab IgG histidine scanning variant #12
(SEQ ID NO: 204)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCARHEITHEFDHWGQGTLVTVSS
>Light Chain Combinations of Telisotuzumab IgG histidine scanning variant #1
(SEQ ID NO: 205)
DIVMTQSPDSLAVSLGERATINCKSSESVDSYAHSHLHWYQQKPGQPPKLLIYRASTRESGVPDRFSGSGSGTDFTLTISSLQAEDVA
VYYCQQSKEDPLTFGGGTKVEIK
>Light Chain Combinations of Telisotuzumab IgG histidine scanning variant #2
(SEQ ID NO: 206)
DIVMTQSPDSLAVSLGERATINCKSSESVDSYAHSFLHWYQQKPGQPPKLLIYHASTRESGVPDRFSGSGSGTDFTLTISSLQAEDVA
VYYCQQSKEDPLTFGGGTKVEIK
>Light Chain Combinations of Telisotuzumab IgG histidine scanning variant #3
(SEQ ID NO: 207)
DIVMTQSPDSLAVSLGERATINCKSSESVDSYAHSFLHWYQQKPGQPPKLLIYRASTREHGVPDRFSGSGSGTDFTLTISSLQAEDVA
VYYCQQSKEDPLTFGGGTKVEIK
>Light Chain Combinations of Telisotuzumab IgG histidine scanning variant #4
(SEQ ID NO: 208)
DIVMTQSPDSLAVSLGERATINCKSSESVDSYANSHLHWYQQKPGQPPKLLIYHASTRESGVPDRFSGSGSGTDFTLTISSLQAEDVA
VYYCQQSKEDPLTFGGGTKVEIK
>Light Chain Combinations of Telisotuzumab IgG histidine scanning variant #5
(SEQ ID NO: 209)
DIVMTQSPDSLAVSLGERATINCKSSESVDSYANSHLHWYQQKPGQPPKLLIYRASTREHGVPDRFSGSGSGTDFTLTISSLQAEDVA
VYYCQQSKEDPLTFGGGTKVEIK
>Light Chain Combinations of Telisotuzumab IgG histidine scanning variant #6
(SEQ ID NO: 210)
DIVMTQSPDSLAVSLGERATINCKSSESVDSYANSFLHWYQQKPGQPPKLLIYHASTREHGVPDRFSGSGSGTDFTLTISSLQAEDVA
VYYCQQSKEDPLTFGGGTKVEIK
>Light Chain Combinations of Telisotuzumab IgG histidine scanning variant #7
(SEQ ID NO: 211)
DIVMTQSPDSLAVSLGERATINCKSSESVDSYAHSHLHWYQQKPGQPPKLLIYHASTRESGVPDRFSGSGSGTDFTLTISSLQAEDVA
VYYCQQSKEDPLTFGGGTKVEIK
>Light Chain Combinations of Telisotuzumab IgG histidine scanning variant #8
(SEQ ID NO: 212)
DIVMTQSPDSLAVSLGERATINCKSSESVDSYAHSHLHWYQQKPGQPPKLLIYRASTREHGVPDRFSGSGSGTDFTLTISSLQAEDVA
VYYCQQSKEDPLTFGGGTKVEIK
>Light Chain Combinations of Telisotuzumab IgG histidine scanning variant #9
(SEQ ID NO: 213)
DIVMTQSPDSLAVSLGERATINCKSSESVDSYAHSFLHWYQQKPGQPPKLLIYHASTREHGVPDRFSGSGSGTDFTLTISSLQAEDVA
VYYCQQSKEDPLTFGGGTKVEIK
>Light Chain Combinations of Telisotuzumab IgG histidine scanning variant #10
(SEQ ID NO: 214)
DIVMTQSPDSLAVSLGERATINCKSSESVDSYANSHLHWYQQKPGQPPKLLIYHASTREHGVPDRFSGSGSGTDFTLTISSLQAEDVA
VYYCQQSKEDPLTFGGGTKVEIK
>Light Chain of Emibetuzumab IgG histidine scanning variant #1
(SEQ ID NO: 215)
DIQMTQSPSSLSASVGDRVTITCSVSSSVSSIHLHWYQQKPGKAPKLLIYSTSNLASGVPSRFSGSGSGTDFTLTISSLQPEDFATYYCQ
VYSGYPLTFGGGTKVEIK
>Heavy Chain of Emibetuzumab IgG histidine scanning variant #1
(SEQ ID NO: 216)
QVQLVQSGAEVKKPGASVKVSCKASGYTFTDYYMHWVRQAPGQGLEWMGRVHPNRRGTTYNQKFEGRVTMTTDTSTSTAYME
LRSLRSDDTAVYYCARANWLDYWGQGTTVTVSS
>Heavy Chain Combinations of Emibetuzumab IgG histidine scanning variant #2
(SEQ ID NO: 217)
QVQLVQSGAEVKKPGASVKVSCKASGYTFTDYYMHWVRQAPGQGLEWMGRVHPHRRGTTYNQKFEGRVTMTTDTSTSTAYME
LRSLRSDDTAVYYCARANWLDYWGQGTTVTVSS
>Heavy Chain Combinations of Emibetuzumab IgG histidine scanning variant #3
(SEQ ID NO: 218)
QVQLVQSGAEVKKPGASVKVSCKASGYTFTDYYMHWVRQAPGQGLEWMGRVHPNRRHTTYNQKFEGRVTMTTDTSTSTAYME
LRSLRSDDTAVYYCARANWLDYWGQGTTVTVSS
>Heavy Chain Combinations of Emibetuzumab IgG histidine scanning variant #4
(SEQ ID NO: 219)
QVQLVQSGAEVKKPGASVKVSCKASGYTFTDYYMHWVRQAPGQGLEWMGRVNPHRRHTTYNQKFEGRVTMTTDTSTSTAYME
LRSLRSDDTAVYYCARANWLDYWGQGTTVTVSS
>Heavy Chain Combinations of Emibetuzumab IgG histidine scanning variant #5
(SEQ ID NO: 220)
QVQLVQSGAEVKKPGASVKVSCKASGYTFTDYYMHWVRQAPGQGLEWMGRVNPHRRGTTYNQKFEGRVTMTTDTSTSTAYME
LRSLRSDDTAVYYCARAHWLDYWGQGTTVTVSS
>Heavy Chain Combinations of Emibetuzumab IgG histidine scanning variant #6
(SEQ ID NO: 221)
QVQLVQSGAEVKKPGASVKVSCKASGYTFTDYYMHWVRQAPGQGLEWMGRVHPHRRHTTYNQKFEGRVTMTTDTSTSTAYME
LRSLRSDDTAVYYCARANWLDYWGQGTTVTVSS
>Heavy Chain Combinations of Emibetuzumab IgG histidine scanning variant #7
(SEQ ID NO: 222)
QVQLVQSGAEVKKPGASVKVSCKASGYTFTDYYMHWVRQAPGQGLEWMGRVHPHRRGTTYNQKFEGRVTMTTDTSTSTAYME
LRSLRSDDTAVYYCARAHWLDYWGQGTTVTVSS
>Light Chain of Emibetuzumab IgG histidine scanning variant #1
(SEQ ID NO: 223)
DIQMTQSPSSLSASVGDRVTITCSVSSSVSSIYLHWYQQKPGKAPKLLIYSTSNLASGVPSRFSGSGSGTDFTLTISSLQPEDFATYYCQ
VHSGYPLTFGGGTKVEIK
>Light Chain of Emibetuzumab IgG histidine scanning variant #2
(SEQ ID NO: 224)
DIQMTQSPSSLSASVGDRVTITCSVSSSVSSIYLHWYQQKPGKAPKLLIYSTSNLASGVPSRFSGSGSGTDFTLTISSLQPEDFATYYCQ
VYSGHPLTFGGGTKVEIK
>Heavy Chain of hucMET27Gv1.3 IgG
(SEQ ID NO: 225)
EVQLVESGGGLVQPGGSLRLSCAASGFTFSSYDMSWVRQAPGKGLEWVATINSNGVSIYYPDSVKGRFTISRDNAKNSLYLQMNSL
RAEDTAVYYCAREEITTEMDYWGQGTLVTVSS
>Light Chain Combinations of hucMET27Gv1.3 IgG histidine scanning variant #1
(SEQ ID NO: 226)
EIVLTQSPATLSLSPGERATLSCRASESVDSYGNSHIHWYQQKPGQAPRLLIYRASNLESGIPARFSGSGSGTDFTLTISSLEPEDFAVYY
CQQSNEEHLTFGQGTKVELK
>Heavy Chain of P3D12 IgG histidine scanning variant #1
(SEQ ID NO: 227)
QVQLVQSGAEVKKPGASVKVSCKASGYTFTSYWMAWVKQAPGQGLDWIGYIKPSTDNTEYNQKFKDKATLTADKSTSTAYMELSS
LRSEDTAVYYCARSYGNYPLMDYWGQGTTVTVSS
>Heavy Chain of P3D12 IgG histidine scanning variant #2
(SEQ ID NO: 228)
QVQLVQSGAEVKKPGASVKVSCKASGYTFTSYWMHWVKQAPGQGLDWIGYIKPSTDNTEYNQKFKDKATLTADKSTSTAYMELS
SLRSEDTAVYYCARSHGNYPLMDYWGQGTTVTVSS
>Heavy Chain Combinations of P3D12 IgG histidine scanning variant #1
(SEQ ID NO: 229)
QVQLVQSGAEVKKPGASVKVSCKASGYTHTSYWMHWVKQAPGQGLDWIGYIKPSTDNTHYNQKFKDKATLTADKSTSTAYMELS
SLRSEDTAVYYCARSYGHYPLMDYWGQGTTVTVSS
>Heavy Chain Combinations of P3D12 IgG histidine scanning variant #2
(SEQ ID NO: 230)
QVQLVQSGAEVKKPGASVKVSCKASGYTHTSYWMHWVKQAPGQGLDWIGYIKPSTDNTHYNQKFKDKATLTADKSTSTAYMELS
SLRSEDTAVYYCARSYGNYPLMHYWGQGTTVTVSS
>Heavy Chain Combinations of P3D12 IgG histidine scanning variant #3
(SEQ ID NO: 231)
QVQLVQSGAEVKKPGASVKVSCKASGYTFTSYWMHWVKQAPGQGLDWIGHIKPSTDNTEYNQKFKDKATLTADKSTSTAYMELS
SLRSEDTAVYYCARSYGHYPLMHYWGQGTTVTVSS
>Light Chain of P3D12 IgG histidine scanning variant #1
(SEQ ID NO: 232)
QIVLTQSPAILSLSPGERATLSCSASSSVTSNYLYWYQQKPGSSPKLLIYSTSNLASGVPARFSGSGSGTSYTLTISSLEAEDAASYFCHQ
WSSYPHTFGSGTKLEIK
>Light Chain Combinations of P3D12 IgG histidine scanning variant #1
(SEQ ID NO: 233)
QIVLTQSPAILSLSPGERATLSCSASSSVTSNYLYWYQQKPGSSPKLLIYSTSNLASGVPARFSGSGSGTSYTLTISSLEAEDAASYFCHQ
WSHHHPTFGSGTKLEIK
>Heavy Chain Combinations of Telisotuzumab IgG histidine scanning variant #13
(SEQ ID NO: 234)
QVQLVQSGAEVKKPGASVKVSCKASGYIFTAYTMHWVRQAPGQGLEWMGWIKPNNGLANYAQKFQGRVTMTRDTSISTAYME
LSRLRSDDTAVYYCAHSEITHEFDHWGQGTLVTVSS
>Light Chain of hucMET27Gv1.3 IgG
(SEQ ID NO: 235)
EIVLTQSPATLSLSPGERATLSCRASESVDSYGNSFIHWYQQKPGQAPRLLIYRASNLESGIPARFSGSGSGTDFTLTISSLEPEDFAVYY
CQQSNEEPLTFGQGTKVELK
>Heavy Chain CDR-H1
(SEQ ID NO: 236)
GYTFTDYYMH
>Heavy Chain CDR-H2
(SEQ ID NO: 237)
RVNPNRRGTTYNQKFEG
>Heavy Chain CDR-H3
(SEQ ID NO: 238)
ARANWLDY
>Light Chain CDR-L1
(SEQ ID NO: 239)
SVSSSVSSIHLH
>Light Chain CDR-L2
(SEQ ID NO: 240)
HTSNLAS
>Light Chain CDR-L3
(SEQ ID NO: 241)
QVYSGYPLT
>MYT4309 Heavy Chain Variable Domain
(SEQ ID NO: 242)
QVQLVQSGAEVKKPGASVKVSCKASGYTHTSYWMHWVKQAPGQGLDWIGYIKPSTDNTEYNQKFKDKATLTADKSTSTAYMELS
SLRSEDTAVYYCARSYGNYPLMHYWGQGTTVTVSS
>MYT4310 Heavy Chain Variable Domain
(SEQ ID NO: 243)
QVQLVQSGAEVKKPGASVKVSCKASGYTFTSYWMHWVKQAPGQGLDWIGHIKPSTDNTHYNQKFKDKATLTADKSTSTAYMELS
SLRSEDTAVYYCARSYGNYPLMDYWGQGTTVTVSS
>MYT4311 Heavy Chain Variable Domain
(SEQ ID NO: 244)
QVQLVQSGAEVKKPGASVKVSCKASGYTFTSYWMHWVKQAPGQGLDWIGHIKPSTDNTEYNQKFKDKATLTADKSTSTAYMELS
SLRSEDTAVYYCARSYGHYPLMDYWGQGTTVTVSS
>MYT4318 Heavy Chain Variable Domain
(SEQ ID NO: 245)
QVQLVQSGAEVKKPGASVKVSCKASGYTHTSYWMHWVKQAPGQGLDWIGHIKPSTDNTEYNQKFKDKATLTADKSTSTAYMELS
SLRSEDTAVYYCARSYGNYPLMHYWGQGTTVTVSS
>MYT4322 Heavy Chain Variable Domain
(SEQ ID NO: 246)
QVQLVQSGAEVKKPGASVKVSCKASGYTFTSYWMHWVKQAPGQGLDWIGHIKPSTDNTHYNQKFKDKATLTADKSTSTAYMELS
SLRSEDTAVYYCARSYGHYPLMDYWGQGTTVTVSS
>MYT4323 Heavy Chain Variable Domain
(SEQ ID NO: 247)
QVQLVQSGAEVKKPGASVKVSCKASGYTFTSYWMHWVKQAPGQGLDWIGHIKPSTDNTHYNQKFKDKATLTADKSTSTAYMELS
SLRSEDTAVYYCARSYGNYPLMHYWGQGTTVTVSS
>MYT4327 Light Chain Variable Domain
(SEQ ID NO: 248)
DIQMTQSPSSLSASVGDRVTITCSVSSSVSSIHLHWYQQKPGKAPKLLIYSTSNLASGVPSRFSGSGSGTDFTLTISSLQPEDFATYYCQ
HYSGYPLTFGGGTKVEIK
>MYT4332 Light Chain Variable Domain
(SEQ ID NO: 249)
DIQMTQSPSSLSASVGDRVTITCSVSSSVSSIYLHWYQQKPGKAPKLLIYHTSNLASGVPSRFSGSGSGTDFTLTISSLQPEDFATYYCQ
VYSGHPLTFGGGTKVEIK
>MYT4334 Light Chain Variable Domain
(SEQ ID NO: 250)
DIQMTQSPSSLSASVGDRVTITCSVSSSVSSIYLHWYQQKPGKAPKLLIYSTSNLASGVPSRFSGSGSGTDFTLTISSLQPEDFATYYCQ
HYSGHPLTFGGGTKVEIK
>MYT4336 Light Chain Variable Domain
(SEQ ID NO: 251)
DIQMTQSPSSLSASVGDRVTITCSVSSSVSSIHLHWYQQKPGKAPKLLIYHTSNLASGVPSRFSGSGSGTDFTLTISSLQPEDFATYYCQ
HYSGYPLTFGGGTKVEIK

1. A method of treating a cMET-positive, cMET-overexpressing, and/or MET-amplified solid tumor cancer, comprising administering an effective amount of an anti-cMET antibody drug conjugate (ADC) to a human subject having said cancer, over a sufficient period of time to provide a therapeutic benefit, wherein the antibody component of the ADC exhibits cMET-specific pH-dependent binding, wherein the antibody comprises heavy and light chain CDRs present in the amino acid sequences as set forth in one of the following pairs: SEQ ID NOs: 15 & 16; SEQ ID NOs: 5 & 6; SEQ ID NOs: 7 & 8; SEQ ID NOs: 9 & 10; SEQ ID NOs: 11 & 12; SEQ ID NOs: 13 & 14; SEQ ID NOs: 17 & 18; SEQ ID NOs: 19 & 20; SEQ ID NOs: 21 & 22; SEQ ID NOs: 23 & 24; SEQ ID NOs: 25 & 26; SEQ ID NOs: 27 & 28; SEQ ID NOs: 29 & 30; SEQ ID NOs: 31 & 32; and SEQ ID NOs: 33 & 34; optionally wherein the CDRs are determined using the Kabat or the IMGT system.

2. A method of treating a cMET-positive solid tumor cancer having low or intermediate expression of cMET, comprising administering an effective amount of an anti-cMET antibody drug conjugate (ADC) to a human subject previously identified or selected as having said cancer, over a sufficient period of time to provide a therapeutic benefit, wherein the antibody component of the ADC exhibits cMET-specific pH-dependent binding, and optionally wherein the antibody comprises heavy and light chain CDRs present in the amino acid sequences as set forth in one of the following pairs: SEQ ID NOs: 15 & 16; SEQ ID NOs: 5 & 6; SEQ ID NOs: 7 & 8; SEQ ID NOs: 9 & 10; SEQ ID NOs: 11 & 12; SEQ ID NOs: 13 & 14; SEQ ID NOs: 17 & 18; SEQ ID NOs: 19 & 20; SEQ ID NOs: 21 & 22; SEQ ID NOs: 23 & 24; SEQ ID NOs: 25 & 26; SEQ ID NOs: 27 & 28; SEQ ID NOs: 29 & 30; SEQ ID NOs: 31 & 32; and SEQ ID NOs: 33 & 34; optionally wherein the CDRs are determined using the Kabat or the IMGT system.

3. A method of treating a cMET-positive solid tumor cancer having a cMET immunohistochemistry score of 1+, 2+, or 3+, and/or an H-score of at least about 50 to 300, comprising administering an effective amount of an anti-cMET antibody drug conjugate (ADC) to a human subject previously identified or selected as having said cancer, over a sufficient period of time to provide a therapeutic benefit, wherein the antibody component of the ADC exhibits cMET-specific pH-dependent binding, wherein the antibody comprises heavy and light chain CDRs present in the amino acid sequences as set forth in one of the following pairs: SEQ ID NOs: 15 & 16; SEQ ID NOs: 5 & 6; SEQ ID NOs: 7 & 8; SEQ ID NOs: 9 & 10; SEQ ID NOs: 11 & 12; SEQ ID NOs: 13 & 14; SEQ ID NOs: 17 & 18; SEQ ID NOs: 19 & 20; SEQ ID NOs: 21 & 22; SEQ ID NOs: 23 & 24; SEQ ID NOs: 25 & 26; SEQ ID NOs: 27 & 28; SEQ ID NOs: 29 & 30; SEQ ID NOs: 31 & 32; and SEQ ID NOs: 33 & 34; optionally wherein the CDRs are determined using the Kabat or the IMGT system.

4. The method of claim 3, wherein the heavy chain and the light chain of the antibody component of the ADC comprises the heavy chain CDRs and the light chain CDRs present in SEQ ID NO: 15 and SEQ ID NO: 16, respectively, and/or wherein the heavy chain comprises the sequence set forth in SEQ ID NO: 15 and the light chain comprises the sequence set forth in SEQ ID NO: 16, and wherein the cancer is non-small cell lung cancer (“NSCLC”), optionally selected from non-squamous NSCLC, squamous NSCLC, and not otherwise specified NSCLC.

5. The method of claim 3 or 4, wherein a biopsy from a tumor of said cancer and/or the entire tumor itself, comprises at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100% of cancer cells having a cMET expression level of at least a 1+, a 2+, or a 3+, as scored by an applicable and/or regulatory-agency approved immunohistochemistry (IHC) assay.

6. The method of claim 5, wherein:
(a) at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100% of the tumor cells have an IHC score of 1+ or 2+;
(b) at least about 1%, 2%, 3%, 4%, 5%, 6%, 7%, 8%, 9%, 10%, 15%, 20%, 25%, 30%, 35%, 40%, 45%, 50%, , 60%, 70%, 80%, 90%, or 100% of the tumor cells have an IHC score of 2+;
(c) at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100% of the tumor cells have an IHC score of 1+;
(d) at least about 1%, 2%, 3%, 4%, 5%, 6%, 7%, 8%, 9%, 10%, 15%, or about 20% of the tumor cells have an IHC score of 1+ and wherein no more than about 1%, 2%, 3%, 4% or 5% of the tumor cells have an IHC score of 2+ or 3+; and/or
(e) at least about 1%, 2%, 3%, 4%, 5%, 6%, 7%, 8%, 9%, 10%, 15%, or about 20% of the tumor cells have an IHC score of 1+ and wherein no more than about 1%, 2%, 3%, 4% or 5% of the tumor cells have an IHC score of 2+ or 3+.

7. The method of claim 6, wherein:
(a) no more than about 1%, 2%, 3%, 4%, 5%, 6%, 7%, 8%, 9%, 10%, 15%, or about 20% of the tumor cells have an IHC score of 2+ and wherein no more than about 1% have an IHC score of 3+;
(b) no tumor cells have an IHC score of greater than 1+; or
(c) no tumor cells have an IHC score of greater than 2+.

8. The method of claim 6, further comprising the steps of determining an H-score for the tumor, wherein the H-score is between about 10 and 300, about 10 and about 250, between about 20 and about 225, between about 20 and about 200, between about 20 and about 175, or between about 20 and about 150.

9. The method of claim 8, wherein the H-score is no more than about 100, about 90, about 80, about 70, or about 50.

10. The method of claim 6, wherein the tumor is homogeneous or heterogeneous for cMET expression and/or MET amplification.

11. The method of claim 10, wherein at least about 10%, 20%, or 30% of the tumor cells have a first frequently occurring IHC score selected from one of 0, 1+, 2+, and 3+ and at least about 10%, 20%, or 30% of the tumor cells have a second frequently occurring IHC score selected from one of 0, 1+, 2+, and 3+, wherein the first and second frequently occurring IHC scores are different.

12. The method of claim 11, wherein the first and second IHC scores are selected from the following pairs of scores: (0, +1), (0, +2), (0, +3), (+1, +2), (+1, +3), and (+2, +3).

13. The method of claim 12, wherein at least about 10%, 20%, or 30% of the tumor cells have a third frequently occurring IHC score, wherein the third score is distinct from the first and second frequently occurring IHC scores.

14. The method of claim 6, wherein the cancer has developed resistance to targeted therapies against one or more actionable mutation present in one or more gene selected from the group consisting of EGFR, ALK, KRAS, ROS, BRAF, NTRK1/2/3, MET, RET, ERBB2, and any other gene known to have an actionable mutation associated with the cancer.

15. The method of claim 14, wherein the actionable mutation is selected from one or more of the following:
a) an EGFR gene mutation selected from an exon 20 T790M substitution, an exon 20 C797X substitution, an exon 21 L858R substitution, and an exon 19 deletion, optionally wherein the exon 19 deletion is the E746_A750 deletion as determined by a regulatory agency approved test;
b) an ALK gene rearrangement;
c) a KRAS gene mutation, optionally wherein the mutation is an exon 1 G12C substitution;
d) a BRAF gene mutation, optionally wherein the mutation is in Exon 15 V600E substitution;
e) a MET gene mutation, optionally wherein the mutation is an ex14 skipping mutation;
f) a ROS1 gene rearrangement, optionally where the ROS1 gene is fused with a gene or portion of a gene selected from one of the following: CD74, EZR, SDC4, SLC34A2, CCCKC6, TFG, SLMAP, MYO5C, FIG, LIMA1, CLTC, GOPC, ZZCCHC8, CEP72, MLL3, KDELR2, LRIG3, MSN, MPRIP, WNK1, SLC6A17, TMEM106B, FAM135B, TPM3, and TDP52L1;
g) a fusion of the NTRK1, 2, or 3 gene; and
h) a RET1 gene rearrangement, optionally where the rearrangement is a fusion with KIF5B or CCDC6.

16. The method of claim 15, wherein the cancer has an amplification, mutation, or overexpression of the ERBB2 gene, optionally wherein the ERBB2 gene has insertions in exon 20 and/or nucleotide substitutions encoding amino acid substitutions selected from one or more of the following: L755S, G776C, G660D, R678Q, E693K, and Q709L.

17. The method of claim 15, wherein the anti-cMET ADC is administered as a monotherapy or adjunctive to an additional anticancer agent, wherein the additional agent is administered according to its regulatory agency-approved dosing regimen, optionally wherein the additional anticancer agent is an inhibitor and/or targeting agent of EGFR, ALK, KRAS, ROS, BRAF, NTRK1/2/3, MET, RET, or ERBB2.

18. The method of claim 17, wherein the additional anticancer agent is selected from osimertinib (TAGRISSO®), afatinib (GIOTRIF®), axitinib (INLYTA®), bosutinib (BOSULIF®), crizotinib (XALKORI®), dasatinib (SPRYCE®), erlotinib (TARCEVA®), gefitinib (IRESSA®), imatinib (GLEEVEC®), lapatinib (TYVERB®), nilotinib (TASIGNA®), pazopanib (VOTRIENT®), ponatinib (ICLUSIG®), radotinib (SUPECT®), regorafenib (STIVARGA®), sorafenib (NEXAVAR®), sunitinib (SUTENT®), toceranib (PALLADIA®), and vatalanib; and/or wherein the additional anticancer agent is capable of inhibiting EGFR comprising an exon 21 L858R substitution or an exon 19 E746_A750 deletion.

19. The method of claim 17, wherein the ALK inhibitor is selected from alectinib, brigatinib, lorlatinib, ceritinib, and crizotinib; the KRAS inhibitor is selected from sotorasib and adagrasib; the BRAF inhibitor is selected from dabrafinib, vemurafenib, and trametinib; the MET inhibitor is selected from tepotinib, crizotinib, and capmatinib; the ROS1 inhibitor is selected from entrectinib, crizotinib, ceritinib, and lorlatinib; the NTRK1/2/3 inhibitor is selected from larotrectinib and entrecteinib; the RET inhibitor is selected from selpercatinib, pralsetinib, and cabozantinib; the ERBB2 targeting agent is selected from transtuzumab-deruxtecan and trastuzumab-emtansine.

20. The method of claim 17, wherein the additional anticancer agent comprises an inhibitor of PD1, optionally an anti-PD1 antibody, optionally wherein the anti-PD1 antibody is pembrolizumab (Keytruda), nivolumab (Opdivo), or cemiplimab (Libtayo); or an inhibitor of PD-L1, optionally an anti-PD-L1 antibody such as durvalumab, or azetolizumab.

21. The method of claim 1, 6, or 15, wherein the cancer is resistant to prior treatment with an anti-cMET antibody, an anti-cMET ADC, a chemotherapy, a small molecule directed against cMET, and/or a radiation therapy.

22. The method of claim 1, 6, or 15, wherein the anti-cMET ADC is administered in an amount ranging from about 0.5 mg/kg to about 6.0 mg/kg, about 2.0 mg/kg to about 5.0 mg/kg, or about 4.5 mg/kg once every three weeks (Q3W).

23. The method of claim 1, 6, or 15, wherein the anti-cMET ADC comprises an anti-cMET antibody linked the drug component of the ADC by way of a linker.

24. The method of claim 23, wherein the anti-cMET antibody is a full-length antibody, optionally wherein the anti-cMET antibody is an IgG1 antibody.

25. The method of claim 24, wherein the anti-cMET antibody binds to cMET in vitro or in vivo with at least a 5-fold, 6-fold, 7-fold, 8-fold, 9-fold, 10-fold, 30-fold, 100-fold, 300-fold, or 1000-fold greater affinity at physiologic pH than it does at pH 5.4.

26. The method of claim 24, wherein the anti-cMET antibody comprises a VH chain comprising the amino acid sequences as set forth in SEQ ID NO: 236, SEQ ID NO: 237, and SEQ ID NO: 238 and a VL chain comprising the amino acid sequences as set forth in SEQ ID NO: 239, SEQ ID NO: 240, and SEQ ID NO: 241.

27. The method of claim 26, wherein the anti-cMET antibody comprises a VH chain comprising the amino acid sequence of SEQ ID NO: 15 and a VL chain comprising the amino acid sequence of SEQ ID NO: 16.

28. The method of claim 27, wherein the anti-cMET antibody comprises a heavy chain comprising the amino acid sequence of SEQ ID NO: 75 and a light chain comprising the amino acid sequence of SEQ ID NO: 82.

29. The method of claim 28, wherein the linker is cleavable by a lysosomal enzyme, optionally wherein the enzyme is Cathepsin B.

30. The method of claim 29, wherein the linker comprises a peptide selected from the group consisting of Cit-Cit; Cit-Val; Val-Cit; Cit-Ala; Ala-Cit; Cit-Asn; Asn-Cit; Cit-Ser; Ser-Cit; Cit-Lys; Lys-Cit; Cit-Asp; Asp-Cit; Ala-Ala; Glu-Val; Val-Glu; Ala-Val; and Val-Ala; and salts thereof.

31. The method of claim 1, 6, or 15, wherein the drug component of the ADC is a microtubule inhibitor, wherein the inhibitor is an auristatin.

32. The method of claim 31, wherein the ADC comprises a compound having a structure of formula (I): [D-L-XY]ₙ-Ab or salts thereof, where each “D” represents a cytotoxic and/or cytostatic agent (“drug”); each “L” represents a linker; “Ab” represents a pH-dependent anti-cMET antigen binding moiety; each “XY” represents a linkage formed between a functional group R^{x} on the linker and a “complementary” functional group R^{y} on the antigen binding moiety; and n represents the number of drugs linked to Ab of the ADC; optionally wherein the
compound has the following structure (II): 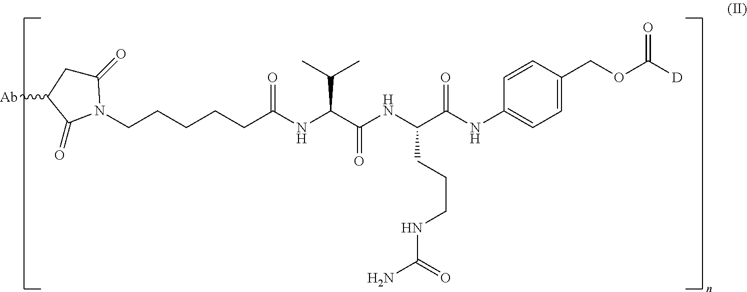
and the anti-cMET antibody is Q397.

33. The method of claim 32, wherein the compound has the following structure (III): 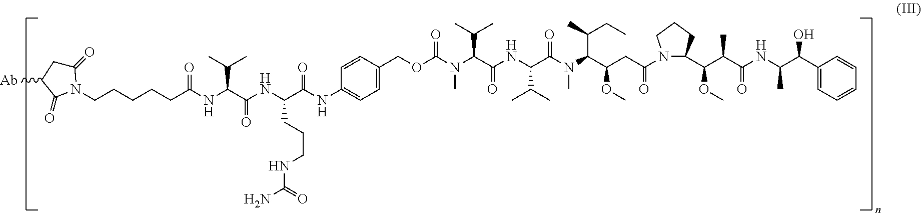
or a pharmaceutically acceptable salt thereof, wherein n has an average value of 2, or is equal to 2, and the Ab is the full length anti-cMET pH-antibody Q397.

34. The method of claim 1, wherein the human subject is a patient diagnosed with cMET-overexpressing, MET amplified and/or MET ex14 skipping mutation non-small cell lung cancer (“NSCLC”), comprising the step of administering to the patient an effective amount of an anti-cMET antibody drug conjugate (“ADC”) for a period of time sufficient to provide one or more therapeutic benefit(s), wherein the antibody component of the ADC exhibits cMET-specific pH-dependent binding, wherein the antibody comprises heavy and light chain CDRs present in the amino acid sequences as set forth in one of the following pairs: SEQ ID NOs: 15 & 16; SEQ ID NOs: 5 & 6; SEQ ID NOs: 7 & 8; SEQ ID NOs: 9 & 10; SEQ ID NOs: 11 & 12; SEQ ID NOs: 13 & 14; SEQ ID NOs: 17 & 18; SEQ ID NOs: 19 & 20; SEQ ID NOs: 21 & 22; SEQ ID NOs: 23 & 24; SEQ ID NOs: 25 & 26; SEQ ID NOs: 27 & 28; SEQ ID NOs: 29 & 30; SEQ ID NOs: 31 & 32; and SEQ ID NOs: 33 & 34.

35. The method of claim 34, wherein the cancer has resisted prior treatment with:
(a) a microtubule inhibitor, optionally wherein the microtubule inhibitor is an auristatin; and/or
(b) an anti-cMET ADC, an anti-cMET antibody, or a small molecule targeting cMET; and
wherein the anti-cMET ADC is administered as monotherapy or adjunctive to an additional anticancer agent administered according to its regulatory agency-approved dosing regimen.

36. The method of claim 35, wherein the anti-cMET ADC is effective against non-squamous cell and/or squamous cell NSCLC; and/or wherein the NSCLC is wildtype for human EGFR or wherein the NSCLC is mutated for human EGFR;
optionally wherein the NSCLC has resisted at least 1, 2, 3, 4, 5, 6, 7, 8, or more prior therapeutic regimen(s).

37. (canceled)

38. A method of treating a non-squamous non-small cell lung cancer (“nsNSCLC”) tumor that expresses or overexpress c-Met, comprising administering to a human subject having said NSCLC tumor an effective amount of a pharmaceutical composition comprising MYTX-011 and a pharmaceutically acceptable carrier, wherein ≥25% of neoplastic cells from tumor tissue of the c-Met expressing or overexpressing nsNSCLC from the subject have 2+ membrane or membrane plus cytoplasmic staining when assessed by c-Met immunohistochemistry (IHC);
optionally wherein the composition remains effective for treating cancer when frozen and rethawed up to at least 2 times; and
optionally wherein the carrier comprises L-Histidine; L-Histidine HCl, monohydrate; D (+)-Trehalose dihydrate; and Polysorbate 80.

39. The method of claim 1, wherein the tumor comprises cells having an FGFR3 amplification, a MET Exon 14 mutation, or an EML4-ALK fusion; optionally wherein the subject has received at least 2 prior treatment regimens for the NSCLC, and/or wherein the subject's tumor is positive for a MET Exon 14 mutation, optionally wherein the tumor has progressed after prior treatment with tyrosine kinase inhibitor.

40. (canceled)


SEQUENCE LISTING (251 sequences)

SEQ ID NO: 1 - protein, 1366 aa, Homo sapiens
ECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 2 - DNA, 4098 bp, Homo sapiens
  source 1..4098: mol_type = unassigned DNA
gagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatca

SEQ ID NO: 3 - protein, 908 aa, Homo sapiens
ECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFT

SEQ ID NO: 4 - DNA, 2724 bp, Homo sapiens
  source 1..2724: mol_type = unassigned DNA
gagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcaca

SEQ ID NO: 5 - protein, 119 aa, synthetic construct
  REGION 1..119: note = MYT5351 Heavy Chain Variable Region
QVQLVQSGAEVKKPGASVKVSCKASGYTFTSYWMHWVKQAPGQGLDWIGHIKPSTDNTEYNQKFKDKATLTADKSTSTAYMELSSLRSEDTAVYYCARSYGNYPLMDYWGQGTTVTVSS

SEQ ID NO: 6 - protein, 108 aa, synthetic construct
  REGION 1..108: note = MYT5351 Light Variable Region
QIVLTQSPAILSLSPGERATLSCSASSSVTSNYLYWYQQKPGSSPKLLIYSHSNLASGVPARFSGSGSGTSYTLTISSLEAEDAASYFCHQWSSYPPTFGSGTKLEIK

SEQ ID NO: 7 - protein, 119 aa, synthetic construct
  REGION 1..119: note = MYT4313 Heavy Chain Variable Region
QVQLVQSGAEVKKPGASVKVSCKASGYTFTSYWMHWVKQAPGQGLDWIGYIKPSTDNTHYNQKFKDKATLTADKSTSTAYMELSSLRSEDTAVYYCARSYGHYPLMDYWGQGTTVTVSS

SEQ ID NO: 8 - protein, 108 aa, synthetic construct
  REGION 1..108: note = MYT4313 Light Chain Variable Region
QIVLTQSPAILSLSPGERATLSCSASSSVTSNYLYWYQQKPGSSPKLLIYSTSNLASGVPARFSGSGSGTSYTLTISSLEAEDAASYFCHQWSSYPPTFGSGTKLEIK

SEQ ID NO: 9 - protein, 119 aa, synthetic construct
  REGION 1..119: note = MYT4325 Heavy Chain Variable Region
QVQLVQSGAEVKKPGASVKVSCKASGYTFTSYWMHWVKQAPGQGLDWIGYIKPSTDNTHYNQKFKDKATLTADKSTSTAYMELSSLRSEDTAVYYCARSYGHYPLMHYWGQGTTVTVSS

SEQ ID NO: 10 - protein, 108 aa, synthetic construct; identical to SEQ ID NO: 8
  REGION 1..108: note = MYT4325 Light Chain Variable Region

SEQ ID NO: 11 - protein, 115 aa, synthetic construct
  REGION 1..115: note = MYT4826 Heavy Chain Variable Region
QVQLVQSGAEVKKPGASVKVSCKASGYTFTHYYMHWVRQAPGQGLEWMGRVNPNRRHTTYNQKFEGRVTMTTDTSTSTAYMELRSLRSDDTAVYYCARANWLDYWGQGTTVTVSS

SEQ ID NO: 12 - protein, 108 aa, synthetic construct [sequence omitted]

SEQ ID NO: 13 - protein, 115 aa, synthetic construct
  REGION 1..115: note = MYT4837 Heavy Chain Variable Region
QVQLVQSGAEVKKPGASVKVSCKASGYTFTHYYMHWVRQAPGQGLEWMGRVNPHRRHTTYNQKFEGRVTMTTDTSTSTAYMELRSLRSDDTAVYYCARANWLDYWGQGTTVTVSS

SEQ ID NO: 14 - protein, 108 aa, synthetic construct [sequence omitted]

SEQ ID NO: 15 - protein, 115 aa, synthetic construct [sequence omitted]

SEQ ID NO: 16 - protein, 108 aa, synthetic construct
  REGION 1..108: note = MYT4849 Light Chain Variable Region
DIQMTQSPSSLSASVGDRVTITCSVSSSVSSIHLHWYQQKPGKAPKLLIYHTSNLASGVPSRFSGSGSGTDFTLTISSLQPEDFATYYCQVYSGYPLTFGGGTKVEIK

SEQ ID NO: 17 - protein, 118 aa, synthetic construct
  REGION 1..118: note = MYT4942 Heavy Chain Variable Region
QVQLVQSGAEVKKPGASVKVSCKASGHIFTAYTMHWVRQAPGQGLEWMGWIKPNNGLANYAQKFQGRVTMTRDTSISTAYMELSRLRSDDTAVYYCARSEITHEFDHWGQGTLVTVSS

SEQ ID NO: 18 - protein, 111 aa, synthetic construct
  REGION 1..111: note = MYT4942 Light Chain Variable Region
DIVMTQSPDSLAVSLGERATINCKSSESVDSYANSHLHWYQQKPGQPPKLLIYRASTRESGVPDRFSGSGSGTDFTLTISSLQAEDVAVYYCQQSKEDPLTFGGGTKVEIK

SEQ ID NO: 19 - protein, 115 aa, synthetic construct
  REGION 1..115: note = MYT5309 Heavy Chain Variable Region
QVQLVQSGAEVKKPGASVKVSCKASGYTFTHYYMHWVRQAPGQGLEWMGRVNPNRRGTTYNQKFEGRVTMTTDTSTSTAYMELRSLRSDDTAVYYCARANWLDYWGQGTTVTVSS

SEQ ID NO: 20 - protein, 108 aa, synthetic construct
  REGION 1..108: note = MYT5309 Light Chain Variable Region
DIQMTQSPSSLSASVGDRVTITCSVSSSVSSIYLHWYQQKPGKAPKLLIYSTSNLASGVPSRFSGSGSGTDFTLTISSLQPEDFATYYCQHYSGYPLTFGGGTKVEIK

SEQ ID NO: 21 - protein, 119 aa, synthetic construct
  REGION 1..119: note = MYT5344 Heavy Chain Variable Region
QVQLVQSGAEVKKPGASVKVSCKASGYTFTSYWMHWVKQAPGQGLDWIGYIKHSTDNTEYNQKFKDKATLTADKSTSTAYMELSSLRSEDTAVYYCARSYGNYPLMDYWGQGTTVTVSS

SEQ ID NO: 22 - protein, 108 aa, synthetic construct
  REGION 1..108: note = MYT5344 Light Chain Variable Region
QIVLTQSPAILSLSPGERATLSCSASSSVTSHYLYWYQQKPGSSPKLLIYSTSNLASGVPARFSGSGSGTSYTLTISSLEAEDAASYFCHQWSSYPPTFGSGTKLEIK

SEQ ID NO: 23 - protein, 119 aa, synthetic construct; identical to SEQ ID NO: 5
  REGION 1..119: note = MYT5367 Heavy Chain Variable Region

SEQ ID NO: 24 - protein, 108 aa, synthetic construct
  REGION 1..108: note = MYT5367 Light Chain Variable Region
QIVLTQSPAILSLSPGERATLSCSASSSVTSNYLYWYQQKPGSSPKLLIYSTSNLASGVPARFSGSGSGTSYTLTISSLEAEDAASYFCHQWSHYHPTFGSGTKLEIK

SEQ ID NO: 25 - protein, 115 aa, synthetic construct
  REGION 1..115: note = MYT4827 Heavy Chain Variable Region
QVQLVQSGAEVKKPGASVKVSCKASGYTFTHYYMHWVRQAPGQGLEWMGRVNPNRRGTTYNQKFEGRVTMTTDTSTSTAYMELRSLRSDDTAVYYCARAHWLDYWGQGTTVTVSS

SEQ ID NO: 26 - protein, 108 aa, synthetic construct [sequence omitted]

SEQ ID NO: 27 - protein, 119 aa, synthetic construct
  REGION 1..119: note = MYT4312 Heavy Chain Variable Region
QVQLVQSGAEVKKPGASVKVSCKASGYTFTSYWMHWVKQAPGQGLDWIGHIKPSTDNTEYNQKFKDKATLTADKSTSTAYMELSSLRSEDTAVYYCARSYGNYPLMHYWGQGTTVTVSS

SEQ ID NO: 28 - protein, 108 aa, synthetic construct; identical to SEQ ID NO: 8
  REGION 1..108: note = MYT4312 Light Chain Variable Region

SEQ ID NO: 29 - protein, 118 aa, synthetic construct
  REGION 1..118: note = MYT4953 Heavy Chain Variable Region
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCHRSEITHEFDYWGQGTLVTVSS

SEQ ID NO: 30 - protein, 111 aa, synthetic construct
  REGION 1..111: note = MYT4953 Light Chain Variable Region
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIK

SEQ ID NO: 31 - protein, 118 aa, synthetic construct
  REGION 1..118: note = MYT4940 Heavy Chain Variable Region
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARHEITTEFDHWGQGTLVTVSS

SEQ ID NO: 32 - protein, 111 aa, synthetic construct; identical to SEQ ID NO: 18
  REGION 1..111: note = MYT4940 Light Chain Variable Region

SEQ ID NO: 33 - protein, 119 aa, synthetic construct
  REGION 1..119: note = MYT4888 Heavy Chain Variable Region
QVQLVQSGAEVKKPGASVKVSCKASGYTFTSYWMHWVKQAPGQGLDWIGYIKPSTDNTEYNQKFKDKATLTADKSTSTAYMELSSLRSEDTAVYYCARSYGNYPLHDYWGQGTTVTVSS

SEQ ID NO: 34 - protein, 108 aa, synthetic construct; identical to SEQ ID NO: 8
  REGION 1..108: note = MYT4888 Light Chain Variable Region

SEQ ID NO: 35 - protein, 446 aa, synthetic construct
  REGION 1..446: note = MYT5351 Heavy chain Triple hinge
QVQLVQSGAEVKKPGASVKVSCKASGYTFTSYWMHWVKQAPGQGLDWIGHIKPSTDNTEYNQKFKDKATLTADKSTSTAYMELSSLRSEDTAVYYCARSYGNYPLMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 446 aa, synthetic construct
  REGION 1..446: note = MYT5351 Heavy chain Triple hinge + LS mutation
QVQLVQSGAEVKKPGASVKVSCKASGYTFTSYWMHWVKQAPGQGLDWIGHIKPSTDNTEYNQKFKDKATLTADKSTSTAYMELSSLRSEDTAVYYCARSYGNYPLMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 37 - protein, 446 aa, synthetic construct
  REGION 1..446: note = MYT5351 Heavy chain Triple hinge + YTE mutation
QVQLVQSGAEVKKPGASVKVSCKASGYTFTSYWMHWVKQAPGQGLDWIGHIKPSTDNTEYNQKFKDKATLTADKSTSTAYMELSSLRSEDTAVYYCARSYGNYPLMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 446 aa, synthetic construct
  REGION 1..446: note = MYT5351 Heavy chain Triple hinge and A118C
QVQLVQSGAEVKKPGASVKVSCKASGYTFTSYWMHWVKQAPGQGLDWIGHIKPSTDNTEYNQKFKDKATLTADKSTSTAYMELSSLRSEDTAVYYCARSYGNYPLMDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 446 aa, synthetic construct
  REGION 1..446: note = MYT5351 Heavy chain Triple hinge + LS mutation and A118C
QVQLVQSGAEVKKPGASVKVSCKASGYTFTSYWMHWVKQAPGQGLDWIGHIKPSTDNTEYNQKFKDKATLTADKSTSTAYMELSSLRSEDTAVYYCARSYGNYPLMDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 40 - protein, 446 aa, synthetic construct
  REGION 1..446: note = MYT5351 Heavy chain Triple hinge + YTE mutation and A118C
QVQLVQSGAEVKKPGASVKVSCKASGYTFTSYWMHWVKQAPGQGLDWIGHIKPSTDNTEYNQKFKDKATLTADKSTSTAYMELSSLRSEDTAVYYCARSYGNYPLMDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 215 aa, synthetic construct
  REGION 1..215: note = MYT5351 Light Chain
QIVLTQSPAILSLSPGERATLSCSASSSVTSNYLYWYQQKPGSSPKLLIYSHSNLASGVPARFSGSGSGTSYTLTISSLEAEDAASYFCHQWSSYPPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 215 aa, synthetic construct
  REGION 1..215: note = MYT5351 Light Chain (V205C)
QIVLTQSPAILSLSPGERATLSCSASSSVTSNYLYWYQQKPGSSPKLLIYSHSNLASGVPARFSGSGSGTSYTLTISSLEAEDAASYFCHQWSSYPPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 43 - protein, 446 aa, synthetic construct
  REGION 1..446: note = MYT4313 Heavy chain Triple hinge
QVQLVQSGAEVKKPGASVKVSCKASGYTFTSYWMHWVKQAPGQGLDWIGYIKPSTDNTHYNQKFKDKATLTADKSTSTAYMELSSLRSEDTAVYYCARSYGHYPLMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 446 aa, synthetic construct
  REGION 1..446: note = MYT4313 Heavy chain Triple hinge + LS mutation
QVQLVQSGAEVKKPGASVKVSCKASGYTFTSYWMHWVKQAPGQGLDWIGYIKPSTDNTHYNQKFKDKATLTADKSTSTAYMELSSLRSEDTAVYYCARSYGHYPLMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 45 - protein, 446 aa, synthetic construct
  REGION 1..446: note = MYT4313 Heavy chain Triple hinge + YTE mutation
QVQLVQSGAEVKKPGASVKVSCKASGYTFTSYWMHWVKQAPGQGLDWIGYIKPSTDNTHYNQKFKDKATLTADKSTSTAYMELSSLRSEDTAVYYCARSYGHYPLMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 46 - protein, 446 aa, synthetic construct
  REGION 1..446: note = MYT4313 Heavy chain Triple hinge and A118C
QVQLVQSGAEVKKPGASVKVSCKASGYTFTSYWMHWVKQAPGQGLDWIGYIKPSTDNTHYNQKFKDKATLTADKSTSTAYMELSSLRSEDTAVYYCARSYGHYPLMDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 446 aa, synthetic construct
  REGION 1..446: note = MYT4313 Heavy chain Triple hinge + LS mutation and A118C
QVQLVQSGAEVKKPGASVKVSCKASGYTFTSYWMHWVKQAPGQGLDWIGYIKPSTDNTHYNQKFKDKATLTADKSTSTAYMELSSLRSEDTAVYYCARSYGHYPLMDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 48 - protein, 446 aa, synthetic construct
  REGION 1..446: note = MYT4313 Heavy chain Triple hinge + YTE mutation and A118C
QVQLVQSGAEVKKPGASVKVSCKASGYTFTSYWMHWVKQAPGQGLDWIGYIKPSTDNTHYNQKFKDKATLTADKSTSTAYMELSSLRSEDTAVYYCARSYGHYPLMDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 215 aa, synthetic construct
  REGION 1..215: note = MYT4313 Light Chain
QIVLTQSPAILSLSPGERATLSCSASSSVTSNYLYWYQQKPGSSPKLLIYSTSNLASGVPARFSGSGSGTSYTLTISSLEAEDAASYFCHQWSSYPPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 215 aa, synthetic construct
  REGION 1..215: note = MYT4313 Light Chain V205C
QIVLTQSPAILSLSPGERATLSCSASSSVTSNYLYWYQQKPGSSPKLLIYSTSNLASGVPARFSGSGSGTSYTLTISSLEAEDAASYFCHQWSSYPPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 51 - protein, 446 aa, synthetic construct
  REGION 1..446: note = MYT4325 Heavy chain Triple hinge
QVQLVQSGAEVKKPGASVKVSCKASGYTFTSYWMHWVKQAPGQGLDWIGYIKPSTDNTHYNQKFKDKATLTADKSTSTAYMELSSLRSEDTAVYYCARSYGHYPLMHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - protein, 446 aa, synthetic construct
  REGION 1..446: note = MYT4325 Heavy chain Triple hinge + LS mutation
QVQLVQSGAEVKKPGASVKVSCKASGYTFTSYWMHWVKQAPGQGLDWIGYIKPSTDNTHYNQKFKDKATLTADKSTSTAYMELSSLRSEDTAVYYCARSYGHYPLMHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 53 - protein, 446 aa, synthetic construct
  REGION 1..446: note = MYT4325 Heavy chain Triple hinge + YTE mutation
QVQLVQSGAEVKKPGASVKVSCKASGYTFTSYWMHWVKQAPGQGLDWIGYIKPSTDNTHYNQKFKDKATLTADKSTSTAYMELSSLRSEDTAVYYCARSYGHYPLMHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 446 aa, synthetic construct
  REGION 1..446: note = MYT4325 Heavy chain Triple hinge and A118C
QVQLVQSGAEVKKPGASVKVSCKASGYTFTSYWMHWVKQAPGQGLDWIGYIKPSTDNTHYNQKFKDKATLTADKSTSTAYMELSSLRSEDTAVYYCARSYGHYPLMHYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 55 - protein, 446 aa, synthetic construct
  REGION 1..446: note = MYT4325 Heavy chain (triple hinge + LS mutation and A118C)
QVQLVQSGAEVKKPGASVKVSCKASGYTFTSYWMHWVKQAPGQGLDWIGYIKPSTDNTHYNQKFKDKATLTADKSTSTAYMELSSLRSEDTAVYYCARSYGHYPLMHYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 56 - protein, 446 aa, synthetic construct
  REGION 1..446: note = MYT4325 Heavy chain (triple hinge + YTE mutation and A118C)
QVQLVQSGAEVKKPGASVKVSCKASGYTFTSYWMHWVKQAPGQGLDWIGYIKPSTDNTHYNQKFKDKATLTADKSTSTAYMELSSLRSEDTAVYYCARSYGHYPLMHYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 57 - protein, 215 aa, synthetic construct; identical to SEQ ID NO: 49
  REGION 1..215: note = MYT4325 Light Chain

SEQ ID NO: 58 - protein, 215 aa, synthetic construct; identical to SEQ ID NO: 50
  REGION 1..215: note = MYT4325 Light Chain V205C

SEQ ID NO: 59 - protein, 442 aa, synthetic construct
  REGION 1..442: note = MYT4826 Heavy chain Triple hinge
QVQLVQSGAEVKKPGASVKVSCKASGYTFTHYYMHWVRQAPGQGLEWMGRVNPNRRHTTYNQKFEGRVTMTTDTSTSTAYMELRSLRSDDTAVYYCARANWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 442 aa, synthetic construct
  REGION 1..442: note = MYT4826 Heavy chain (triple hinge + LS mutation)
QVQLVQSGAEVKKPGASVKVSCKASGYTFTHYYMHWVRQAPGQGLEWMGRVNPNRRHTTYNQKFEGRVTMTTDTSTSTAYMELRSLRSDDTAVYYCARANWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 61 - protein, 442 aa, synthetic construct
  REGION 1..442: note = MYT4826 Heavy chain Triple hinge + YTE mutation
QVQLVQSGAEVKKPGASVKVSCKASGYTFTHYYMHWVRQAPGQGLEWMGRVNPNRRHTTYNQKFEGRVTMTTDTSTSTAYMELRSLRSDDTAVYYCARANWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 442 aa, synthetic construct
  REGION 1..442: note = MYT4826 Heavy chain Triple hinge only and A118
QVQLVQSGAEVKKPGASVKVSCKASGYTFTHYYMHWVRQAPGQGLEWMGRVNPNRRHTTYNQKFEGRVTMTTDTSTSTAYMELRSLRSDDTAVYYCARANWLDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 63 - protein, 442 aa, synthetic construct
  REGION 1..442: note = MYT4826 Heavy chain Triple hinge + LS mutation and A118C
QVQLVQSGAEVKKPGASVKVSCKASGYTFTHYYMHWVRQAPGQGLEWMGRVNPNRRHTTYNQKFEGRVTMTTDTSTSTAYMELRSLRSDDTAVYYCARANWLDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 64 - protein, 442 aa, synthetic construct
  REGION 1..442: note = MYT4826 Heavy chain Triple hinge + YTE mutation and A118C
QVQLVQSGAEVKKPGASVKVSCKASGYTFTHYYMHWVRQAPGQGLEWMGRVNPNRRHTTYNQKFEGRVTMTTDTSTSTAYMELRSLRSDDTAVYYCARANWLDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 65 - protein, 215 aa, synthetic construct
  REGION 1..215: note = MYT4826 Light Chain
DIQMTQSPSSLSASVGDRVTITCSVSSSVSSIYLHWYQQKPGKAPKLLIYSTSNLASGVPSRFSGSGSGTDFTLTISSLQPEDFATYYCQVYSGYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 215 aa, synthetic construct
  REGION 1..215: note = MYT4826 Light Chain V205C
DIQMTQSPSSLSASVGDRVTITCSVSSSVSSIYLHWYQQKPGKAPKLLIYSTSNLASGVPSRFSGSGSGTDFTLTISSLQPEDFATYYCQVYSGYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 67 - protein, 442 aa, synthetic construct
  REGION 1..442: note = MYT4837 Heavy chain Triple hinge
QVQLVQSGAEVKKPGASVKVSCKASGYTFTHYYMHWVRQAPGQGLEWMGRVNPHRRHTTYNQKFEGRVTMTTDTSTSTAYMELRSLRSDDTAVYYCARANWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 68 - protein, 442 aa, synthetic construct
  REGION 1..442: note = MYT4837 Heavy chain Triple hinge + LS mutation
QVQLVQSGAEVKKPGASVKVSCKASGYTFTHYYMHWVRQAPGQGLEWMGRVNPHRRHTTYNQKFEGRVTMTTDTSTSTAYMELRSLRSDDTAVYYCARANWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 69 - protein, 442 aa, synthetic construct
  REGION 1..442: note = MYT4837 Heavy chain (Triple hinge + YTE mutation
QVQLVQSGAEVKKPGASVKVSCKASGYTFTHYYMHWVRQAPGQGLEWMGRVNPHRRHTTYNQKFEGRVTMTTDTSTSTAYMELRSLRSDDTAVYYCARANWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 442 aa, synthetic construct
  REGION 1..442: note = MYT4837 Heavy chain Triple hinge and A118C
QVQLVQSGAEVKKPGASVKVSCKASGYTFTHYYMHWVRQAPGQGLEWMGRVNPHRRHTTYNQKFEGRVTMTTDTSTSTAYMELRSLRSDDTAVYYCARANWLDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 442 aa, synthetic construct
  REGION 1..442: note = MYT4837 Heavy chain Triple hinge + LS mutation and A118C
QVQLVQSGAEVKKPGASVKVSCKASGYTFTHYYMHWVRQAPGQGLEWMGRVNPHRRHTTYNQKFEGRVTMTTDTSTSTAYMELRSLRSDDTAVYYCARANWLDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 72 - protein, 442 aa, synthetic construct
  REGION 1..442: note = MYT4837 Heavy chain (triple hinge + YTE mutation and A118C)
QVQLVQSGAEVKKPGASVKVSCKASGYTFTHYYMHWVRQAPGQGLEWMGRVNPHRRHTTYNQKFEGRVTMTTDTSTSTAYMELRSLRSDDTAVYYCARANWLDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 215 aa, synthetic construct; identical to SEQ ID NO: 65
  REGION 1..215: note = MYT4837 Light Chain

SEQ ID NO: 74 - protein, 215 aa, synthetic construct; identical to SEQ ID NO: 66
  REGION 1..215: note = MYT4837 Light Chain V205C

SEQ ID NO: 75 - protein, 442 aa, synthetic construct
  REGION 1..442: note = MYT4849 Heavy chain Triple hinge
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 442 aa, synthetic construct
  REGION 1..442: note = MYT4849 Heavy chain Triple hinge + LS mutation
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 77 - protein, 442 aa, synthetic construct
  REGION 1..442: note = MYT4849 Heavy chain Triple hinge + YTE mutation
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 442 aa, synthetic construct
  REGION 1..442: note = MYT4849 Heavy chain Triple hinge and A118C
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 79 - protein, 442 aa, synthetic construct
  REGION 1..442: note = MYT4849 Heavy chain Triple hinge + LS mutation and A118C
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 80 - protein, 442 aa, synthetic construct
  REGION 1..442: note = MYT4849 Heavy chain Triple hinge + YTE mutation and A118C
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 81 - protein, 215 aa, synthetic construct
  REGION 1..215: note = MYT4849 Light Chain
DIQMTQSPSSLSASVGDRVTITCSVSSSVSSIHLHWYQQKPGKAPKLLIYHTSNLASGVPSRFSGSGSGTDFTLTISSLQPEDFATYYCQVYSGYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 215 aa, synthetic construct
  REGION 1..215: note = MYT4849 Light Chain V205C
DIQMTQSPSSLSASVGDRVTITCSVSSSVSSIHLHWYQQKPGKAPKLLIYHTSNLASGVPSRFSGSGSGTDFTLTISSLQPEDFATYYCQVYSGYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 83 - protein, 445 aa, synthetic construct
  REGION 1..445: note = MYT4942 Heavy chain Triple hinge
QVQLVQSGAEVKKPGASVKVSCKASGHIFTAYTMHWVRQAPGQGLEWMGWIKPNNGLANYAQKFQGRVTMTRDTSISTAYMELSRLRSDDTAVYYCARSEITHEFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 84 - protein, 445 aa, synthetic construct
  REGION 1..445: note = MYT4942 Heavy chain Triple hinge + LS mutation
QVQLVQSGAEVKKPGASVKVSCKASGHIFTAYTMHWVRQAPGQGLEWMGWIKPNNGLANYAQKFQGRVTMTRDTSISTAYMELSRLRSDDTAVYYCARSEITHEFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 85 - protein, 445 aa, synthetic construct
  REGION 1..445: note = MYT4942 Heavy chain Triple hinge + YTE mutation
QVQLVQSGAEVKKPGASVKVSCKASGHIFTAYTMHWVRQAPGQGLEWMGWIKPNNGLANYAQKFQGRVTMTRDTSISTAYMELSRLRSDDTAVYYCARSEITHEFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 86 - protein, 445 aa, synthetic construct
  REGION 1..445: note = MYT4942 Heavy chain Triple hinge and A118C
QVQLVQSGAEVKKPGASVKVSCKASGHIFTAYTMHWVRQAPGQGLEWMGWIKPNNGLANYAQKFQGRVTMTRDTSISTAYMELSRLRSDDTAVYYCARSEITHEFDHWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 87 - protein, 445 aa, synthetic construct
  REGION 1..445: note = MYT4942 Heavy chain Triple hinge + LS mutation and A118C
QVQLVQSGAEVKKPGASVKVSCKASGHIFTAYTMHWVRQAPGQGLEWMGWIKPNNGLANYAQKFQGRVTMTRDTSISTAYMELSRLRSDDTAVYYCARSEITHEFDHWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 88 - protein, 445 aa, synthetic construct
  REGION 1..445: note = MYT4942 Heavy chain Triple hinge + YTE mutation and A118C
QVQLVQSGAEVKKPGASVKVSCKASGHIFTAYTMHWVRQAPGQGLEWMGWIKPNNGLANYAQKFQGRVTMTRDTSISTAYMELSRLRSDDTAVYYCARSEITHEFDHWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 89 - protein, 218 aa, synthetic construct
  REGION 1..218: note = MYT4942 Light Chain
DIVMTQSPDSLAVSLGERATINCKSSESVDSYANSHLHWYQQKPGQPPKLLIYRASTRESGVPDRFSGSGSGTDFTLTISSLQAEDVAVYYCQQSKEDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 218 aa, synthetic construct
  REGION 1..218: note = MYT4942 Light Chain V205C
DIVMTQSPDSLAVSLGERATINCKSSESVDSYANSHLHWYQQKPGQPPKLLIYRASTRESGVPDRFSGSGSGTDFTLTISSLQAEDVAVYYCQQSKEDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 91 - protein, 442 aa, synthetic construct
  REGION 1..442: note = MYT5309 Heavy chain Triple hinge
QVQLVQSGAEVKKPGASVKVSCKASGYTFTHYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 442 aa, synthetic construct
  REGION 1..442: note = MYT5309 Heavy chain Triple hinge + LS mutation
QVQLVQSGAEVKKPGASVKVSCKASGYTFTHYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 93 - protein, 442 aa, synthetic construct
  REGION 1..442: note = MYT5309 Heavy chain Triple hinge + YTE mutation
QVQLVQSGAEVKKPGASVKVSCKASGYTFTHYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 94 - protein, 442 aa, synthetic construct
  REGION 1..442: note = MYT5309 Heavy chain Triple hinge and A118C
QVQLVQSGAEVKKPGASVKVSCKASGYTFTHYYMHWVRQAPGQGLEWMGRVNPNRRGTTYNQKFEGRVTMTTDTSTSTAYMELRSLRSDDTAVYYCARANWLDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 95 - protein, 442 aa, synthetic construct
  REGION 1..442: note = MYT5309 Heavy chain Triple hinge + LS mutation and A118C
QVQLVQSGAEVKKPGASVKVSCKASGYTFTHYYMHWVRQAPGQGLEWMGRVNPNRRGTTYNQKFEGRVTMTTDTSTSTAYMELRSLRSDDTAVYYCARANWLDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 96 - protein, 442 aa, synthetic construct
  REGION 1..442: note = MYT5309 Heavy chain Triple hinge + YTE mutation and A118C
QVQLVQSGAEVKKPGASVKVSCKASGYTFTHYYMHWVRQAPGQGLEWMGRVNPNRRGTTYNQKFEGRVTMTTDTSTSTAYMELRSLRSDDTAVYYCARANWLDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 97 - protein, 215 aa, synthetic construct
  REGION 1..215: note = MYT5309 Light Chain
DIQMTQSPSSLSASVGDRVTITCSVSSSVSSIYLHWYQQKPGKAPKLLIYSTSNLASGVPSRFSGSGSGTDFTLTISSLQPEDFATYYCQHYSGYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 215 aa, synthetic construct
  REGION 1..215: note = MYT5309 Light Chain V205C
DIQMTQSPSSLSASVGDRVTITCSVSSSVSSIYLHWYQQKPGKAPKLLIYSTSNLASGVPSRFSGSGSGTDFTLTISSLQPEDFATYYCQHYSGYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 99 - protein, 446 aa, synthetic construct
  REGION 1..446: note = MYT5344 Heavy chain Triple hinge
QVQLVQSGAEVKKPGASVKVSCKASGYTFTSYWMHWVKQAPGQGLDWIGYIKHSTDNTEYNQKFKDKATLTADKSTSTAYMELSSLRSEDTAVYYCARSYGNYPLMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 100 - protein, 446 aa, synthetic construct
  REGION 1..446: note = MYT5344 Heavy chain Triple hinge + LS mutation
QVQLVQSGAEVKKPGASVKVSCKASGYTFTSYWMHWVKQAPGQGLDWIGYIKHSTDNTEYNQKFKDKATLTADKSTSTAYMELSSLRSEDTAVYYCARSYGNYPLMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 101 - protein, 446 aa, synthetic construct
  REGION 1..446: note = MYT5344 Heavy chain Triple hinge + YTE mutation
QVQLVQSGAEVKKPGASVKVSCKASGYTFTSYWMHWVKQAPGQGLDWIGYIKHSTDNTEYNQKFKDKATLTADKSTSTAYMELSSLRSEDTAVYYCARSYGNYPLMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 102 - protein, 446 aa, synthetic construct
  REGION 1..446: note = MYT5344 Heavy chain Triple hinge and A118C
QVQLVQSGAEVKKPGASVKVSCKASGYTFTSYWMHWVKQAPGQGLDWIGYIKHSTDNTEYNQKFKDKATLTADKSTSTAYMELSSLRSEDTAVYYCARSYGNYPLMDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 103 - protein, 446 aa, synthetic construct
  REGION 1..446: note = MYT5344 Heavy chain Triple hinge + LS mutation and A118C
QVQLVQSGAEVKKPGASVKVSCKASGYTFTSYWMHWVKQAPGQGLDWIGYIKHSTDNTEYNQKFKDKATLTADKSTSTAYMELSSLRSEDTAVYYCARSYGNYPLMDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 104 - protein, 446 aa, synthetic construct
  REGION 1..446: note = MYT5344 Heavy chain Triple hinge + YTE mutation and A118C
QVQLVQSGAEVKKPGASVKVSCKASGYTFTSYWMHWVKQAPGQGLDWIGYIKHSTDNTEYNQKFKDKATLTADKSTSTAYMELSSLRSEDTAVYYCARSYGNYPLMDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 105 - protein, 215 aa, synthetic construct
  REGION 1..215: note = MYT5344 Light Chain
QIVLTQSPAILSLSPGERATLSCSASSSVTSHYLYWYQQKPGSSPKLLIYSTSNLASGVPARFSGSGSGTSYTLTISSLEAEDAASYFCHQWSSYPPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 215 aa, synthetic construct
  REGION 1..215: note = MYT5344 Light Chain V205C
QIVLTQSPAILSLSPGERATLSCSASSSVTSHYLYWYQQKPGSSPKLLIYSTSNLASGVPARFSGSGSGTSYTLTISSLEAEDAASYFCHQWSSYPPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 107 - protein, 446 aa, synthetic construct; identical to SEQ ID NO: 35
  REGION 1..446: note = MYT5367 Heavy chain Triple hinge

SEQ ID NO: 108 - protein, 446 aa, synthetic construct; identical to SEQ ID NO: 36
  REGION 1..446: note = MYT5367 Heavy chain Triple hinge + LS mutation

SEQ ID NO: 109 - protein, 446 aa, synthetic construct; identical to SEQ ID NO: 37
  REGION 1..446: note = MYT5367 Heavy chain Triple hinge + YTE mutation

SEQ ID NO: 110 - protein, 446 aa, synthetic construct; identical to SEQ ID NO: 38
  REGION 1..446: note = MYT5367 Heavy chain Triple hinge and A118C

SEQ ID NO: 111 - protein, 446 aa, synthetic construct; identical to SEQ ID NO: 39
  REGION 1..446: note = MYT5367 Heavy chain Triple hinge + LS mutation and A118C

SEQ ID NO: 112 - protein, 446 aa, synthetic construct; identical to SEQ ID NO: 40
  REGION 1..446: note = MYT5367 Heavy chain Triple hinge + YTE mutation and A118C

SEQ ID NO: 113 - protein, 215 aa, synthetic construct
  REGION 1..215: note = MYT5367 Light Chain
QIVLTQSPAILSLSPGERATLSCSASSSVTSNYLYWYQQKPGSSPKLLIYSTSNLASGVPARFSGSGSGTSYTLTISSLEAEDAASYFCHQWSHYHPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 215 aa, synthetic construct
  REGION 1..215: note = MYT5367 Light Chain V205C
QIVLTQSPAILSLSPGERATLSCSASSSVTSNYLYWYQQKPGSSPKLLIYSTSNLASGVPARFSGSGSGTSYTLTISSLEAEDAASYFCHQWSHYHPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 115 - protein, 442 aa, synthetic construct
  REGION 1..442: note = MYT4827 Heavy Chain Triple hinge
QVQLVQSGAEVKKPGASVKVSCKASGYTFTHYYMHWVRQAPGQGLEWMGRVNPNRRGTTYNQKFEGRVTMTTDTSTSTAYMELRSLRSDDTAVYYCARAHWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 116 - protein, 442 aa, synthetic construct
  REGION 1..442: note = MYT4827 Heavy Chain Triple hinge + LS mutation
QVQLVQSGAEVKKPGASVKVSCKASGYTFTHYYMHWVRQAPGQGLEWMGRVNPNRRGTTYNQKFEGRVTMTTDTSTSTAYMELRSLRSDDTAVYYCARAHWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 117 - protein, 442 aa, synthetic construct
  REGION 1..442: note = MYT4827 Heavy Chain Triple hinge + YTE mutation
QVQLVQSGAEVKKPGASVKVSCKASGYTFTHYYMHWVRQAPGQGLEWMGRVNPNRRGTTYNQKFEGRVTMTTDTSTSTAYMELRSLRSDDTAVYYCARAHWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 118 - protein, 442 aa, synthetic construct
  REGION 1..442: note = MYT4827 Heavy Chain Triple hinge and A118C
QVQLVQSGAEVKKPGASVKVSCKASGYTFTHYYMHWVRQAPGQGLEWMGRVNPNRRGTTYNQKFEGRVTMTTDTSTSTAYMELRSLRSDDTAVYYCARAHWLDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 119 - protein, 442 aa, synthetic construct
  REGION 1..442: note = MYT4827 Heavy Chain Triple hinge + LS mutation and A118C
QVQLVQSGAEVKKPGASVKVSCKASGYTFTHYYMHWVRQAPGQGLEWMGRVNPNRRGTTYNQKFEGRVTMTTDTSTSTAYMELRSLRSDDTAVYYCARAHWLDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 120 - protein, 442 aa, synthetic construct
  REGION 1..442: note = MYT4827 Heavy Chain Triple hinge + YTE mutation and A118C
QVQLVQSGAEVKKPGASVKVSCKASGYTFTHYYMHWVRQAPGQGLEWMGRVNPNRRGTTYNQKFEGRVTMTTDTSTSTAYMELRSLRSDDTAVYYCARAHWLDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 121 - protein, 215 aa, synthetic construct; identical to SEQ ID NO: 65
  REGION 1..215: note = MYT4827 Light Chain

SEQ ID NO: 122 - protein, 215 aa, synthetic construct; identical to SEQ ID NO: 66
  REGION 1..215: note = MYT4827 Light Chain V205C

SEQ ID NO: 123 - protein, 446 aa, synthetic construct
  REGION 1..446: note = MYT4312 Heavy Chain Triple hinge
QVQLVQSGAEVKKPGASVKVSCKASGYTFTSYWMHWVKQAPGQGLDWIGHIKPSTDNTEYNQKFKDKATLTADKSTSTAYMELSSLRSEDTAVYYCARSYGNYPLMHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 124 - protein, 446 aa, synthetic construct
  REGION 1..446: note = MYT4312 Heavy Chain Triple hinge + LS mutation
QVQLVQSGAEVKKPGASVKVSCKASGYTFTSYWMHWVKQAPGQGLDWIGHIKPSTDNTEYNQKFKDKATLTADKSTSTAYMELSSLRSEDTAVYYCARSYGNYPLMHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 125 - protein, 446 aa, synthetic construct
  REGION 1..446: note = MYT4312 Heavy Chain Triple hinge + YTE mutation
QVQLVQSGAEVKKPGASVKVSCKASGYTFTSYWMHWVKQAPGQGLDWIGHIKPSTDNTEYNQKFKDKATLTADKSTSTAYMELSSLRSEDTAVYYCARSYGNYPLMHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 126 - protein, 446 aa, synthetic construct
  REGION 1..446: note = MYT4312 Heavy Chain Triple hinge and A118C
QVQLVQSGAEVKKPGASVKVSCKASGYTFTSYWMHWVKQAPGQGLDWIGHIKPSTDNTEYNQKFKDKATLTADKSTSTAYMELSSLRSEDTAVYYCARSYGNYPLMHYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 127 - protein, 446 aa, synthetic construct
  REGION 1..446: note = MYT4312 Heavy Chain Triple hinge + LS mutation and A118C
QVQLVQSGAEVKKPGASVKVSCKASGYTFTSYWMHWVKQAPGQGLDWIGHIKPSTDNTEYNQKFKDKATLTADKSTSTAYMELSSLRSEDTAVYYCARSYGNYPLMHYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 128 - protein, 446 aa, synthetic construct
  REGION 1..446: note = MYT4312 Heavy Chain (triple hinge + YTE mutation and A118C)
QVQLVQSGAEVKKPGASVKVSCKASGYTFTSYWMHWVKQAPGQGLDWIGHIKPSTDNTEYNQKFKDKATLTADKSTSTAYMELSSLRSEDTAVYYCARSYGNYPLMHYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 129 - protein, 215 aa, synthetic construct; identical to SEQ ID NO: 49
  REGION 1..215: note = MYT4312 Light Chain

SEQ ID NO: 130 - protein, 215 aa, synthetic construct; identical to SEQ ID NO: 50
  REGION 1..215: note = MYT4312 Light Chain V205C

SEQ ID NO: 131 - protein, 445 aa, synthetic construct
  REGION 1..445: note = MYT4953 Heavy Chain Triple hinge
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCHRSEITHEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 132 - protein, 445 aa, synthetic construct
  REGION 1..445: note = MYT4953 Heavy Chain Triple hinge + LS mutation
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCHRSEITHEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 133 - protein, 445 aa, synthetic construct
  REGION 1..445: note = MYT4953 Heavy Chain Triple hinge + YTE mutation
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCHRSEITHEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 134 - protein, 445 aa, synthetic construct
  REGION 1..445: note = MYT4953 Heavy Chain Triple hinge and A118C
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCHRSEITHEFDYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 135 - protein, 445 aa, synthetic construct
  REGION 1..445: note = MYT4953 Heavy Chain Triple hinge + LS mutation and A118C
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCHRSEITHEFDYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 136 - protein, 445 aa, synthetic construct
  REGION 1..445: note = MYT4953 Heavy Chain Triple hinge + YTE mutation and A118C
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCHRSEITHEFDYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 137 - protein, 218 aa, synthetic construct
  REGION 1..218: note = MYT4953 Light Chain
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 218 aa, synthetic construct
  REGION 1..218: note = MYT4953 Light Chain V205C
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 139 - protein, 445 aa, synthetic construct
  REGION 1..445: note = MYT4940 Heavy Chain Triple hinge
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARHEITTEFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 140 - protein, 445 aa, synthetic construct
  REGION 1..445: note = MYT4940 Heavy Chain (Triple hinge + LS mutation)
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARHEITTEFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 141 - protein, 445 aa, synthetic construct
  REGION 1..445: note = MYT4940 Heavy Chain Triple hinge + YTE mutation
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARHEITTEFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 142 - protein, 445 aa, synthetic construct
  REGION 1..445: note = MYT4940 Heavy Chain Triple hinge and A118C
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARHEITTEFDHWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 143 - protein, 445 aa, synthetic construct
  REGION 1..445: note = MYT4940 Heavy Chain Triple hinge + LS mutation and A118C
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARHEITTEFDHWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 144 - protein, 445 aa, synthetic construct
  REGION 1..445: note = MYT4940 Heavy Chain Triple hinge + YTE mutation and A118C
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARHEITTEFDHWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 145 - protein, 218 aa, synthetic construct; identical to SEQ ID NO: 89
  REGION 1..218: note = MYT4940 Light Chain

SEQ ID NO: 146 - protein, 218 aa, synthetic construct; identical to SEQ ID NO: 90
  REGION 1..218: note = MYT4940 Light Chain V205C

SEQ ID NO: 147 - protein, 446 aa, synthetic construct
  REGION 1..446: note = MYT4888 Heavy Chain Triple hinge
QVQLVQSGAEVKKPGASVKVSCKASGYTFTSYWMHWVKQAPGQGLDWIGYIKPSTDNTEYNQKFKDKATLTADKSTSTAYMELSSLRSEDTAVYYCARSYGNYPLHDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 148 - protein, 446 aa, synthetic construct
  REGION 1..446: note = MYT4888 Heavy Chain Triple hinge + LS mutation
QVQLVQSGAEVKKPGASVKVSCKASGYTFTSYWMHWVKQAPGQGLDWIGYIKPSTDNTEYNQKFKDKATLTADKSTSTAYMELSSLRSEDTAVYYCARSYGNYPLHDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 149 - protein, 446 aa, synthetic construct
  REGION 1..446: note = MYT4888 Heavy Chain Triple hinge + YTE mutation
QVQLVQSGAEVKKPGASVKVSCKASGYTFTSYWMHWVKQAPGQGLDWIGYIKPSTDNTEYNQKFKDKATLTADKSTSTAYMELSSLRSEDTAVYYCARSYGNYPLHDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 150 - protein, 446 aa, synthetic construct
  REGION 1..446: note = MYT4888 Heavy Chain Triple hinge and A118C
QVQLVQSGAEVKKPGASVKVSCKASGYTFTSYWMHWVKQAPGQGLDWIGYIKPSTDNTEYNQKFKDKATLTADKSTSTAYMELSSLRSEDTAVYYCARSYGNYPLHDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 151 - protein, 446 aa, synthetic construct
  REGION 1..446: note = MYT4888 Heavy Chain Triple hinge + LS mutation and A118C
QVQLVQSGAEVKKPGASVKVSCKASGYTFTSYWMHWVKQAPGQGLDWIGYIKPSTDNTEYNQKFKDKATLTADKSTSTAYMELSSLRSEDTAVYYCARSYGNYPLHDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 152 - protein, 446 aa, synthetic construct
  REGION 1..446: note = MYT4888 Heavy Chain Triple hinge + YTE mutation and A118C
QVQLVQSGAEVKKPGASVKVSCKASGYTFTSYWMHWVKQAPGQGLDWIGYIKPSTDNTEYNQKFKDKATLTADKSTSTAYMELSSLRSEDTAVYYCARSYGNYPLHDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 153 - protein, 215 aa, synthetic construct; identical to SEQ ID NO: 49
  REGION 1..215: note = MYT4888 Light Chain

SEQ ID NO: 154 - protein, 215 aa, synthetic construct; identical to SEQ ID NO: 50
  REGION 1..215: note = MYT4888 Light Chain V205C

SEQ ID NO: 155 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 156 - protein, 327 aa, synthetic construct
  REGION 1..327: note = Heavy Chain Constant Domain A118C
CSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 157 - protein, 107 aa, synthetic construct [sequence omitted]

SEQ ID NO: 158 - protein, 107 aa, synthetic construct
  REGION 1..107: note = Light Chain Constant Domain V205C
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 159 - protein, 118 aa, synthetic construct
  REGION 1..118: note = Telisotuzumab Heavy chain variable region
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSS

SEQ ID NO: 160 - protein, 111 aa, synthetic construct; identical to SEQ ID NO: 30
  REGION 1..111: note = Telisotuzumab Light chain variable region

SEQ ID NO: 161 - protein, 115 aa, synthetic construct [sequence omitted]

SEQ ID NO: 162 - protein, 108 aa, synthetic construct [sequence omitted]

SEQ ID NO: 163 - protein, 119 aa, synthetic construct
  REGION 1..119: note = P3D12 anti-cMET heavy chain variable region
QVQLVQSGAEVKKPGASVKVSCKASGYTFTSYWMHWVKQAPGQGLDWIGYIKPSTDNTEYNQKFKDKATLTADKSTSTAYMELSSLRSEDTAVYYCARSYGNYPLMDYWGQGTTVTVSS

SEQ ID NO: 164 - protein, 108 aa, synthetic construct; identical to SEQ ID NO: 8
  REGION 1..108: note = 3D12 anti-cMET light chain variable region

SEQ ID NO: 165 - protein, 445 aa, synthetic construct
  REGION 1..445: note = Telisotuzumab Heavy chain Triple Hinge
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 166 - protein, 445 aa, synthetic construct
  REGION 1..445: note = Telisotuzumab Heavy chain Triple Hinge + LS
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 167 - protein, 445 aa, synthetic construct
  REGION 1..445: note = Telisotuzumab Heavy chain Triple Hinge + YTE
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 168 - protein, 445 aa, synthetic construct
  REGION 1..445: note = Telisotuzumab Heavy chain Triple Hinge and A118C
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 169 - protein, 445 aa, synthetic construct
  REGION 1..445: note = Telisotuzumab Heavy chain Triple Hinge + LS and A118C
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 170 - protein, 445 aa, synthetic construct
  REGION 1..445: note = Telisotuzumab Heavy chain Triple Hinge + YTE and A118C
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 171 - protein, 218 aa, synthetic construct; identical to SEQ ID NO: 137
  REGION 1..218: note = Telisotuzumab Light chain

SEQ ID NO: 172 - protein, 218 aa, synthetic construct; identical to SEQ ID NO: 138
  REGION 1..218: note = Telisotuzumab Light chain V205C

SEQ ID NO: 173 - protein, 442 aa, synthetic construct; identical to SEQ ID NO: 75
  REGION 1..442: note = Emibetuzumab heavy chain with Triple Hinge

SEQ ID NO: 174 - protein, 442 aa, synthetic construct; identical to SEQ ID NO: 76
  REGION 1..442: note = Emibetuzumab heavy chain with Triple Hinge and LS

SEQ ID NO: 175 - protein, 442 aa, synthetic construct; identical to SEQ ID NO: 77
  REGION 1..442: note = Emibetuzumab heavy chain with Triple Hinge and YTE

SEQ ID NO: 176 - protein, 442 aa, synthetic construct; identical to SEQ ID NO: 78
  REGION 1..442: note = Emibetuzumab heavy chain with Triple Hinge and A118C

SEQ ID NO: 177 - protein, 442 aa, synthetic construct; identical to SEQ ID NO: 79
  REGION 1..442: note = Emibetuzumab heavy chain with Triple Hinge and LS and A118C

SEQ ID NO: 178 - protein, 442 aa, synthetic construct; identical to SEQ ID NO: 80
  REGION 1..442: note = Emibetuzumab heavy chain with Triple Hinge and YTE and A118C

SEQ ID NO: 179 - protein, 215 aa, synthetic construct; identical to SEQ ID NO: 65
  REGION 1..215: note = Emibetuzumab light chain

SEQ ID NO: 180 - protein, 215 aa, synthetic construct; identical to SEQ ID NO: 66
  REGION 1..215: note = Emibetuzumab light chain V205C

SEQ ID NO: 181 - protein, 446 aa, synthetic construct
  REGION 1..446: note = P3D12 anti-cMET heavy chain with Triple Hinge
QVQLVQSGAEVKKPGASVKVSCKASGYTFTSYWMHWVKQAPGQGLDWIGYIKPSTDNTEYNQKFKDKATLTADKSTSTAYMELSSLRSEDTAVYYCARSYGNYPLMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 182 - protein, 446 aa, synthetic construct
  REGION 1..446: note = P3D12 anti-cMET heavy chain with Triple Hinge and LS
QVQLVQSGAEVKKPGASVKVSCKASGYTFTSYWMHWVKQAPGQGLDWIGYIKPSTDNTEYNQKFKDKATLTADKSTSTAYMELSSLRSEDTAVYYCARSYGNYPLMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 183 - protein, 446 aa, synthetic construct
  REGION 1..446: note = P3D12 anti-cMET heavy chain with Triple Hinge and YTE
QVQLVQSGAEVKKPGASVKVSCKASGYTFTSYWMHWVKQAPGQGLDWIGYIKPSTDNTEYNQKFKDKATLTADKSTSTAYMELSSLRSEDTAVYYCARSYGNYPLMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 184 - protein, 446 aa, synthetic construct
  REGION 1..446: note = P3D12 anti-cMET heavy chain with Triple Hinge and A118C
QVQLVQSGAEVKKPGASVKVSCKASGYTFTSYWMHWVKQAPGQGLDWIGYIKPSTDNTEYNQKFKDKATLTADKSTSTAYMELSSLRSEDTAVYYCARSYGNYPLMDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 185 - protein, 446 aa, synthetic construct
  REGION 1..446: note = P3D12 anti-cMET heavy chain with Triple Hinge and LS and A118C
QVQLVQSGAEVKKPGASVKVSCKASGYTFTSYWMHWVKQAPGQGLDWIGYIKPSTDNTEYNQKFKDKATLTADKSTSTAYMELSSLRSEDTAVYYCARSYGNYPLMDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 186 - protein, 446 aa, synthetic construct
  REGION 1..446: note = P3D12 anti-cMET heavy chain with Triple Hinge and YTE and A118C
QVQLVQSGAEVKKPGASVKVSCKASGYTFTSYWMHWVKQAPGQGLDWIGYIKPSTDNTEYNQKFKDKATLTADKSTSTAYMELSSLRSEDTAVYYCARSYGNYPLMDYWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 187 - protein, 215 aa, synthetic construct; identical to SEQ ID NO: 49
  REGION 1..215: note = P3D12 anti-cMET light chain

SEQ ID NO: 188 - protein, 215 aa, synthetic construct; identical to SEQ ID NO: 50
  REGION 1..215: note = P3D12 anti-cMET light chain V205C

SEQ ID NO: 189 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 190 - protein, 111 aa, synthetic construct
  REGION 1..111: note = Light Chain of Telisotuzumab IgG histidine scanning variant #1
DIVMTQSPDSLAVSLGERATINCKSSESVDSYANSFLHWYQQKPGQPPKLLIYRASTREHGVPDRFSGSGSGTDFTLTISSLQAEDVAVYYCQQSKEDPLTFGGGTKVEIK

SEQ ID NO: 191 - protein, 111 aa, synthetic construct
  REGION 1..111: note = Light Chain of Telisotuzumab IgG histidine scanning variant #2
DIVMTQSPDSLAVSLGERATINCKSSESVDSYAHSFLHWYQQKPGQPPKLLIYRASTRESGVPDRFSGSGSGTDFTLTISSLQAEDVAVYYCQQSKEDPLTFGGGTKVEIK

SEQ ID NO: 192 - protein, 111 aa, synthetic construct
  REGION 1..111: note = Light Chain of Telisotuzumab IgG histidine scanning variant #3
DIVMTQSPDSLAVSLGERATINCKSSESVDSYANSFLHWYQQKPGQPPKLLIYHASTRESGVPDRFSGSGSGTDFTLTISSLQAEDVAVYYCQQSKEDPLTFGGGTKVEIK

SEQ ID NO: 193 - protein, 118 aa, synthetic construct
  REGION 1..118: note = Heavy Chain of Telisotuzumab IgG histidine scanning variant #1
QVQLVQSGAEVKKPGASVKVSCKASGHIFTAYTMHWVRQAPGQGLEWMGWIKPNNGLANYAQKFQGRVTMTRDTSISTAYMELSRLRSDDTAVYYCARSEITTEFDYWGQGTLVTVSS

SEQ ID NO: 194 - protein, 118 aa, synthetic construct
  REGION 1..118: note = Heavy Chain of Telisotuzumab IgG histidine scanning variant #2
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARHEITTEFDYWGQGTLVTVSS

SEQ ID NO: 195 - protein, 118 aa, synthetic construct
  REGION 1..118: note = Heavy Chain of Telisotuzumab IgG histidine scanning variant #3
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITHEFDYWGQGTLVTVSS

SEQ ID NO: 196 - protein, 118 aa, synthetic construct
  REGION 1..118: note = Heavy Chain of Telisotuzumab IgG histidine scanning variant #4
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDHWGQGTLVTVSS

SEQ ID NO: 197 - protein, 118 aa, synthetic construct
  REGION 1..118: note = Heavy Chain Combinations of Telisotuzumab IgG histidine scanning variant #5
QVQLVQSGAEVKKPGASVKVSCKASGHIFTAYTMHWVRQAPGQGLEWMGWIKPNNGLANYAQKFQGRVTMTRDTSISTAYMELSRLRSDDTAVYYCARHEITTEFDYWGQGTLVTVSS

SEQ ID NO: 198 - protein, 118 aa, synthetic construct
  REGION 1..118: note = Heavy Chain Combinations of Telisotuzumab IgG histidine scanning variant #6
QVQLVQSGAEVKKPGASVKVSCKASGHIFTAYTMHWVRQAPGQGLEWMGWIKPNNGLANYAQKFQGRVTMTRDTSISTAYMELSRLRSDDTAVYYCARSEITHEFDYWGQGTLVTVSS

SEQ ID NO: 199 - protein, 118 aa, synthetic construct
  REGION 1..118: note = Heavy Chain Combinations of Telisotuzumab IgG histidine scanning variant #7
QVQLVQSGAEVKKPGASVKVSCKASGHIFTAYTMHWVRQAPGQGLEWMGWIKPNNGLANYAQKFQGRVTMTRDTSISTAYMELSRLRSDDTAVYYCARSEITTEFDHWGQGTLVTVSS

SEQ ID NO: 200 - protein, 118 aa, synthetic construct
  REGION 1..118: note = Heavy Chain Combinations of Telisotuzumab IgG histidine scanning variant #8
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARHEITHEFDYWGQGTLVTVSS

SEQ ID NO: 201 - protein, 118 aa, synthetic construct
  REGION 1..118: note = Heavy Chain Combinations of Telisotuzumab IgG histidine scanning variant #9
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITHEFDHWGQGTLVTVSS

SEQ ID NO: 202 - protein, 118 aa, synthetic construct
  REGION 1..118: note = Heavy Chain Combinations of Telisotuzumab IgG histidine scanning variant #10
QVQLVQSGAEVKKPGASVKVSCKASGHIFTAYTMHWVRQAPGQGLEWMGWIKPNNGLANYAQKFQGRVTMTRDTSISTAYMELSRLRSDDTAVYYCARHEITHEFDYWGQGTLVTVSS

SEQ ID NO: 203 - protein, 118 aa, synthetic construct
  REGION 1..118: note = Heavy Chain Combinations of Telisotuzumab IgG histidine scanning variant #11
QVQLVQSGAEVKKPGASVKVSCKASGHIFTAYTMHWVRQAPGQGLEWMGWIKPNNGLANYAQKFQGRVTMTRDTSISTAYMELSRLRSDDTAVYYCARHEITTEFDHWGQGTLVTVSS

SEQ ID NO: 204 - protein, 118 aa, synthetic construct
  REGION 1..118: note = Heavy Chain Combinations of Telisotuzumab IgG histidine scanning variant #12
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARHEITHEFDHWGQGTLVTVSS

SEQ ID NO: 205 - protein, 111 aa, synthetic construct
  REGION 1..111: note = Light Chain Combinations of Telisotuzumab IgG histidine scanning variant #1
DIVMTQSPDSLAVSLGERATINCKSSESVDSYAHSHLHWYQQKPGQPPKLLIYRASTRESGVPDRFSGSGSGTDFTLTISSLQAEDVAVYYCQQSKEDPLTFGGGTKVEIK

SEQ ID NO: 206 - protein, 111 aa, synthetic construct
  REGION 1..111: note = Light Chain Combinations of Telisotuzumab IgG histidine scanning variant #2
DIVMTQSPDSLAVSLGERATINCKSSESVDSYAHSFLHWYQQKPGQPPKLLIYHASTRESGVPDRFSGSGSGTDFTLTISSLQAEDVAVYYCQQSKEDPLTFGGGTKVEIK

SEQ ID NO: 207 - protein, 111 aa, synthetic construct
  REGION 1..111: note = Light Chain Combinations of Telisotuzumab IgG histidine scanning variant #3
DIVMTQSPDSLAVSLGERATINCKSSESVDSYAHSFLHWYQQKPGQPPKLLIYRASTREHGVPDRFSGSGSGTDFTLTISSLQAEDVAVYYCQQSKEDPLTFGGGTKVEIK

SEQ ID NO: 208 - protein, 111 aa, synthetic construct
  REGION 1..111: note = Light Chain Combinations of Telisotuzumab IgG histidine scanning variant #4
DIVMTQSPDSLAVSLGERATINCKSSESVDSYANSHLHWYQQKPGQPPKLLIYHASTRESGVPDRFSGSGSGTDFTLTISSLQAEDVAVYYCQQSKEDPLTFGGGTKVEIK

SEQ ID NO: 209 - protein, 111 aa, synthetic construct
  REGION 1..111: note = Light Chain Combinations of Telisotuzumab IgG histidine scanning variant #5
DIVMTQSPDSLAVSLGERATINCKSSESVDSYANSHLHWYQQKPGQPPKLLIYRASTREHGVPDRFSGSGSGTDFTLTISSLQAEDVAVYYCQQSKEDPLTFGGGTKVEIK

SEQ ID NO: 210 - protein, 111 aa, synthetic construct
  REGION 1..111: note = Light Chain Combinations of Telisotuzumab IgG histidine scanning variant #6
DIVMTQSPDSLAVSLGERATINCKSSESVDSYANSFLHWYQQKPGQPPKLLIYHASTREHGVPDRFSGSGSGTDFTLTISSLQAEDVAVYYCQQSKEDPLTFGGGTKVEIK

SEQ ID NO: 211 - protein, 111 aa, synthetic construct
  REGION 1..111: note = Light Chain Combinations of Telisotuzumab IgG histidine scanning variant #7
DIVMTQSPDSLAVSLGERATINCKSSESVDSYAHSHLHWYQQKPGQPPKLLIYHASTRESGVPDRFSGSGSGTDFTLTISSLQAEDVAVYYCQQSKEDPLTFGGGTKVEIK

SEQ ID NO: 212 - protein, 111 aa, synthetic construct
  REGION 1..111: note = Light Chain Combinations of Telisotuzumab IgG histidine scanning variant #8
DIVMTQSPDSLAVSLGERATINCKSSESVDSYAHSHLHWYQQKPGQPPKLLIYRASTREHGVPDRFSGSGSGTDFTLTISSLQAEDVAVYYCQQSKEDPLTFGGGTKVEIK

SEQ ID NO: 213 - protein, 111 aa, synthetic construct
  REGION 1..111: note = Light Chain Combinations of Telisotuzumab IgG histidine scanning variant #9
DIVMTQSPDSLAVSLGERATINCKSSESVDSYAHSFLHWYQQKPGQPPKLLIYHASTREHGVPDRFSGSGSGTDFTLTISSLQAEDVAVYYCQQSKEDPLTFGGGTKVEIK

SEQ ID NO: 214 - protein, 111 aa, synthetic construct
  REGION 1..111: note = Light Chain Combinations of Telisotuzumab IgG histidine scanning variant #10
DIVMTQSPDSLAVSLGERATINCKSSESVDSYANSHLHWYQQKPGQPPKLLIYHASTREHGVPDRFSGSGSGTDFTLTISSLQAEDVAVYYCQQSKEDPLTFGGGTKVEIK

SEQ ID NO: 215 - protein, 108 aa, synthetic construct
  REGION 1..108: note = Light Chain of Emibetuzumab IgG histidine scanning variant #1
DIQMTQSPSSLSASVGDRVTITCSVSSSVSSIHLHWYQQKPGKAPKLLIYSTSNLASGVPSRFSGSGSGTDFTLTISSLQPEDFATYYCQVYSGYPLTFGGGTKVEIK

SEQ ID NO: 216 - protein, 115 aa, synthetic construct
  REGION 1..115: note = Heavy Chain of Emibetuzumab IgG histidine scanning variant #1
QVQLVQSGAEVKKPGASVKVSCKASGYTFTDYYMHWVRQAPGQGLEWMGRVHPNRRGTTYNQKFEGRVTMTTDTSTSTAYMELRSLRSDDTAVYYCARANWLDYWGQGTTVTVSS

SEQ ID NO: 217 - protein, 115 aa, synthetic construct
  REGION 1..115: note = Heavy Chain Combinations of Emibetuzumab IgG histidine scanning variant #2
QVQLVQSGAEVKKPGASVKVSCKASGYTFTDYYMHWVRQAPGQGLEWMGRVHPHRRGTTYNQKFEGRVTMTTDTSTSTAYMELRSLRSDDTAVYYCARANWLDYWGQGTTVTVSS

SEQ ID NO: 218 - protein, 115 aa, synthetic construct
  REGION 1..115: note = Heavy Chain Combinations of Emibetuzumab IgG histidine scanning variant #3
QVQLVQSGAEVKKPGASVKVSCKASGYTFTDYYMHWVRQAPGQGLEWMGRVHPNRRHTTYNQKFEGRVTMTTDTSTSTAYMELRSLRSDDTAVYYCARANWLDYWGQGTTVTVSS

SEQ ID NO: 219 - protein, 115 aa, synthetic construct
  REGION 1..115: note = Heavy Chain Combinations of Emibetuzumab IgG histidine scanning variant #4
QVQLVQSGAEVKKPGASVKVSCKASGYTFTDYYMHWVRQAPGQGLEWMGRVNPHRRHTTYNQKFEGRVTMTTDTSTSTAYMELRSLRSDDTAVYYCARANWLDYWGQGTTVTVSS

SEQ ID NO: 220 - protein, 115 aa, synthetic construct
  REGION 1..115: note = Heavy Chain Combinations of Emibetuzumab IgG histidine scanning variant #5
QVQLVQSGAEVKKPGASVKVSCKASGYTFTDYYMHWVRQAPGQGLEWMGRVNPHRRGTTYNQKFEGRVTMTTDTSTSTAYMELRSLRSDDTAVYYCARAHWLDYWGQGTTVTVSS

SEQ ID NO: 221 - protein, 115 aa, synthetic construct
  REGION 1..115: note = Heavy Chain Combinations of Emibetuzumab IgG histidine scanning variant #6
QVQLVQSGAEVKKPGASVKVSCKASGYTFTDYYMHWVRQAPGQGLEWMGRVHPHRRHTTYNQKFEGRVTMTTDTSTSTAYMELRSLRSDDTAVYYCARANWLDYWGQGTTVTVSS

SEQ ID NO: 222 - protein, 115 aa, synthetic construct
  REGION 1..115: note = Heavy Chain Combinations of Emibetuzumab IgG histidine scanning variant #7
QVQLVQSGAEVKKPGASVKVSCKASGYTFTDYYMHWVRQAPGQGLEWMGRVHPHRRGTTYNQKFEGRVTMTTDTSTSTAYMELRSLRSDDTAVYYCARAHWLDYWGQGTTVTVSS

SEQ ID NO: 223 - protein, 108 aa, synthetic construct
  REGION 1..108: note = Light Chain of Emibetuzumab IgG histidine scanning variant #1
DIQMTQSPSSLSASVGDRVTITCSVSSSVSSIYLHWYQQKPGKAPKLLIYSTSNLASGVPSRFSGSGSGTDFTLTISSLQPEDFATYYCQVHSGYPLTFGGGTKVEIK

SEQ ID NO: 224 - protein, 108 aa, synthetic construct
  REGION 1..108: note = Light Chain of Emibetuzumab IgG histidine scanning variant #2
DIQMTQSPSSLSASVGDRVTITCSVSSSVSSIYLHWYQQKPGKAPKLLIYSTSNLASGVPSRFSGSGSGTDFTLTISSLQPEDFATYYCQVYSGHPLTFGGGTKVEIK

SEQ ID NO: 225 - protein, 118 aa, synthetic construct
  REGION 1..118: note = Heavy Chain of hucMET27Gv1.3 IgG
EVQLVESGGGLVQPGGSLRLSCAASGFTFSSYDMSWVRQAPGKGLEWVATINSNGVSIYYPDSVKGRFTISRDNAKNSLYLQMNSLRAEDTAVYYCAREEITTEMDYWGQGTLVTVSS

SEQ ID NO: 226 - protein, 111 aa, synthetic construct
  REGION 1..111: note = Light Chain Combinations of hucMET27Gv1.3 IgG histidine scanning variant #1
EIVLTQSPATLSLSPGERATLSCRASESVDSYGNSHIHWYQQKPGQAPRLLIYRASNLESGIPARFSGSGSGTDFTLTISSLEPEDFAVYYCQQSNEEHLTFGQGTKVELK

SEQ ID NO: 227 - protein, 119 aa, synthetic construct
  REGION 1..119: note = Heavy Chain of P3D12 IgG histidine scanning variant #1
QVQLVQSGAEVKKPGASVKVSCKASGYTFTSYWMAWVKQAPGQGLDWIGYIKPSTDNTEYNQKFKDKATLTADKSTSTAYMELSSLRSEDTAVYYCARSYGNYPLMDYWGQGTTVTVSS

SEQ ID NO: 228 - protein, 119 aa, synthetic construct
  REGION 1..119: note = Heavy Chain of P3D12 IgG histidine scanning variant #2
QVQLVQSGAEVKKPGASVKVSCKASGYTFTSYWMHWVKQAPGQGLDWIGYIKPSTDNTEYNQKFKDKATLTADKSTSTAYMELSSLRSEDTAVYYCARSHGNYPLMDYWGQGTTVTVSS

SEQ ID NO: 229 - protein, 119 aa, synthetic construct
  REGION 1..119: note = Heavy Chain Combinations of P3D12 IgG histidine scanning variant #1
QVQLVQSGAEVKKPGASVKVSCKASGYTHTSYWMHWVKQAPGQGLDWIGYIKPSTDNTHYNQKFKDKATLTADKSTSTAYMELSSLRSEDTAVYYCARSYGHYPLMDYWGQGTTVTVSS

SEQ ID NO: 230 - protein, 119 aa, synthetic construct
  REGION 1..119: note = Heavy Chain Combinations of P3D12 IgG histidine scanning variant #2
QVQLVQSGAEVKKPGASVKVSCKASGYTHTSYWMHWVKQAPGQGLDWIGYIKPSTDNTHYNQKFKDKATLTADKSTSTAYMELSSLRSEDTAVYYCARSYGNYPLMHYWGQGTTVTVSS

SEQ ID NO: 231 - protein, 119 aa, synthetic construct
  REGION 1..119: note = Heavy Chain Combinations of P3D12 IgG histidine scanning variant #3
QVQLVQSGAEVKKPGASVKVSCKASGYTFTSYWMHWVKQAPGQGLDWIGHIKPSTDNTEYNQKFKDKATLTADKSTSTAYMELSSLRSEDTAVYYCARSYGHYPLMHYWGQGTTVTVSS

SEQ ID NO: 232 - protein, 108 aa, synthetic construct
  REGION 1..108: note = Light Chain of P3D12 IgG histidine scanning variant #1
QIVLTQSPAILSLSPGERATLSCSASSSVTSNYLYWYQQKPGSSPKLLIYSTSNLASGVPARFSGSGSGTSYTLTISSLEAEDAASYFCHQWSSYPHTFGSGTKLEIK

SEQ ID NO: 233 - protein, 108 aa, synthetic construct
  REGION 1..108: note = Light Chain Combinations of P3D12 IgG histidine scanning variant #1
QIVLTQSPAILSLSPGERATLSCSASSSVTSNYLYWYQQKPGSSPKLLIYSTSNLASGVPARFSGSGSGTSYTLTISSLEAEDAASYFCHQWSHHHPTFGSGTKLEIK

SEQ ID NO: 234 - protein, 118 aa, synthetic construct
  REGION 1..118: note = Heavy Chain Combinations of Telisotuzumab IgG histidine scanning variant #13
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCAHSEITHEFDHWGQGTLVTVSS

SEQ ID NO: 235 - protein, 111 aa, synthetic construct
  REGION 1..111: note = Light Chain of hucMET27Gv1.3 IgG
EIVLTQSPATLSLSPGERATLSCRASESVDSYGNSFIHWYQQKPGQAPRLLIYRASNLESGIPARFSGSGSGTDFTLTISSLEPEDFAVYYCQQSNEEPLTFGQGTKVELK

SEQ ID NO: 236 - protein, 10 aa, synthetic construct
  REGION 1..10: note = Q397 VH CDR1
GYTFTDYYMH

SEQ ID NO: 237 - protein, 17 aa, synthetic construct
  REGION 1..17: note = Q397 VH CDR2
RVNPNRRGTTYNQKFEG

SEQ ID NO: 238 - protein, 8 aa, synthetic construct
  REGION 1..8: note = Q397 VH CDR3
ARANWLDY

SEQ ID NO: 239 - protein, 12 aa, synthetic construct
  REGION 1..12: note = Q397 VL CDR1
SVSSSVSSIHLH

SEQ ID NO: 240 - protein, 7 aa, synthetic construct
  REGION 1..7: note = Q397 VL CDR2
HTSNLAS

SEQ ID NO: 241 - protein, 9 aa, synthetic construct
  REGION 1..9: note = Q397 VL CDR3
QVYSGYPLT

SEQ ID NO: 242 - protein, 119 aa, synthetic construct
QVQLVQSGAEVKKPGASVKVSCKASGYTHTSYWMHWVKQAPGQGLDWIGYIKPSTDNTEYNQKFKDKATLTADKSTSTAYMELSSLRSEDTAVYYCARSYGNYPLMHYWGQGTTVTVSS

SEQ ID NO: 243 - protein, 119 aa, synthetic construct
QVQLVQSGAEVKKPGASVKVSCKASGYTFTSYWMHWVKQAPGQGLDWIGHIKPSTDNTHYNQKFKDKATLTADKSTSTAYMELSSLRSEDTAVYYCARSYGNYPLMDYWGQGTTVTVSS

SEQ ID NO: 244 - protein, 119 aa, synthetic construct
QVQLVQSGAEVKKPGASVKVSCKASGYTFTSYWMHWVKQAPGQGLDWIGHIKPSTDNTEYNQKFKDKATLTADKSTSTAYMELSSLRSEDTAVYYCARSYGHYPLMDYWGQGTTVTVSS

SEQ ID NO: 245 - protein, 119 aa, synthetic construct
QVQLVQSGAEVKKPGASVKVSCKASGYTHTSYWMHWVKQAPGQGLDWIGHIKPSTDNTEYNQKFKDKATLTADKSTSTAYMELSSLRSEDTAVYYCARSYGNYPLMHYWGQGTTVTVSS

SEQ ID NO: 246 - protein, 119 aa, synthetic construct
QVQLVQSGAEVKKPGASVKVSCKASGYTFTSYWMHWVKQAPGQGLDWIGHIKPSTDNTHYNQKFKDKATLTADKSTSTAYMELSSLRSEDTAVYYCARSYGHYPLMDYWGQGTTVTVSS

SEQ ID NO: 247 - protein, 119 aa, synthetic construct
QVQLVQSGAEVKKPGASVKVSCKASGYTFTSYWMHWVKQAPGQGLDWIGHIKPSTDNTHYNQKFKDKATLTADKSTSTAYMELSSLRSEDTAVYYCARSYGNYPLMHYWGQGTTVTVSS

SEQ ID NO: 248 - protein, 108 aa, synthetic construct
DIQMTQSPSSLSASVGDRVTITCSVSSSVSSIHLHWYQQKPGKAPKLLIYSTSNLASGVPSRFSGSGSGTDFTLTISSLQPEDFATYYCQHYSGYPLTFGGGTKVEIK

SEQ ID NO: 249 - protein, 108 aa, synthetic construct
DIQMTQSPSSLSASVGDRVTITCSVSSSVSSIYLHWYQQKPGKAPKLLIYHTSNLASGVPSRFSGSGSGTDFTLTISSLQPEDFATYYCQVYSGHPLTFGGGTKVEIK

SEQ ID NO: 250 - protein, 108 aa, synthetic construct
DIQMTQSPSSLSASVGDRVTITCSVSSSVSSIYLHWYQQKPGKAPKLLIYSTSNLASGVPSRFSGSGSGTDFTLTISSLQPEDFATYYCQHYSGHPLTFGGGTKVEIK

SEQ ID NO: 251 - protein, 108 aa, synthetic construct
DIQMTQSPSSLSASVGDRVTITCSVSSSVSSIHLHWYQQKPGKAPKLLIYHTSNLASGVPSRFSGSGSGTDFTLTISSLQPEDFATYYCQHYSGYPLTFGGGTKVEIK